SEQUENCE LISTING (22 sequences)

SEQ ID NO: 1 - protein, 393 aa, Homo sapiens [sequence omitted]

SEQ ID NO: 2 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 3 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 4 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 5 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 6 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 7 - DNA, 88 bp, Artificial Sequence
  Synthetic oligonucleotide
acctgaggtttttccgaagagatgttgggtcattggccagaagggtgttaaccaggccgaagagctgcatcacacgctcatcctggcg

SEQ ID NO: 8 - DNA, 93 bp, Artificial Sequence
  Synthetic oligonucleotide
cgacatcgcgatggcccagctcctcagccaggtccacgggcagacggccccaggcatcgcgcacgtccagccgcgccccggcccggtgcagca

SEQ ID NO: 9 - DNA, 101 bp, Artificial Sequence
  Synthetic oligonucleotide
ggcagcgtcgtgcacgggtcgggtgagagtggcggggtcggcgcagttgggctccgcgccgtggagcagcagcagctccgccactcgggcgctgcccatca

SEQ ID NO: 10 - DNA, 93 bp, Artificial Sequence
  Synthetic oligonucleotide
attccaattcccctgcaaacttcgtcctccagagtcgcccgccatcccctgctcccgctgcagaccctctacccacctggatcggcctccgac

SEQ ID NO: 11 - DNA, 103 bp, Artificial Sequence
  Synthetic oligonucleotide
gcactgcaggctgcagaccttgttgcccgcgtcctcctggcactcgggcagctcgcacgcctcctcgatcagcggcggggggatgtcgcgcccggccccaccc

SEQ ID NO: 12 - DNA, 101 bp, Artificial Sequence
  Synthetic oligonucleotide
gtggcgccctcgaagccctgcccgagagggaagacaggacggtgtcggggtgggccaccccccgcccccccgccacctcacacccagccctcggccagcag

SEQ ID NO: 13 - DNA, 86 bp, Artificial Sequence
  Synthetic oligonucleotide
tccccaatctggtccaggcaggtggcgtcgttctggcacgggttcgagacgcactcgttgacgtcgatctcgcatcgggggcccgt

SEQ ID NO: 14 - DNA, 94 bp, Artificial Sequence
  Synthetic oligonucleotide
cccagcgtgttgatgcacttgcccgcatgctcgcaggggttggcacctggcgagggcacacgggtgagaggctgctccaggcaccctggcccct

SEQ ID NO: 15 - DNA, 103 bp, Artificial Sequence
  Synthetic oligonucleotide
ggccgggcccgtgtaccccgaggggcaggtgcagatggccttgccattgacagggttggtgtcgcagttggagccctcgttacaggggttgctgatgcatgcg

SEQ ID NO: 16 - DNA, 104 bp, Artificial Sequence
  Synthetic oligonucleotide
tgtgcggccatggggacactcgcagtagaaggaggccacacggtcatggcaggtggcgccgtggaagcaggcggcgctggcacagtcatcaatgttctcgctgc

SEQ ID NO: 17 - DNA, 90 bp, Artificial Sequence
  Synthetic oligonucleotide
gcctcgaagggcaggcactggccaccgttggcgcaggggttggaggcgcacgggtcagcctgctggcacgatttccctggagacaagggg

SEQ ID NO: 18 - DNA, 93 bp, Artificial Sequence
  Synthetic oligonucleotide
acttggtgttgttgatggcaaacacacacaggaagccctccccggtgcgcatgtactggtcccgcatggcgctgtactcctcctggccggcgg

SEQ ID NO: 19 - DNA, 86 bp, Artificial Sequence
  Synthetic oligonucleotide
gggtcgtattcgtccacaaaatggttctggatcagctggatggtcagcgcactcttgcccacaccgccggcgcccaccaccaccag

SEQ ID NO: 20 - DNA, 89 bp, Artificial Sequence
  Synthetic oligonucleotide
ctcttcctctgtgcgccggtctctcccaggacaggcacaaacacgcacctcaaagctgttccgtcccagtagattaccactactcagga

SEQ ID NO: 21 - DNA, 89 bp, Artificial Sequence
  Synthetic oligonucleotide
aggatgggcctccggttcatgccgcccatgcaggaactgttacacatgtagttgtagtggatggtggtacagtcagagccaacctagga

SEQ ID NO: 22 - DNA, 80 bp, Artificial Sequence
  Synthetic oligonucleotide
tgtttctgtcatccaaatactccacacgcaaatttccttccactcggataagatgctgaggaggggccagacctaagagc

(19) United States
(12) Patent Application Publication
(10) Pub. No.: US 2022/0162682 A1
(43) Pub. Date: May 26, 2022

(54) ASSESSMENT OF MUTATION BURDEN IN SKIN

(71) Applicant: DermTech, Inc., La Jolla, CA (US)

(72) Inventors: John Daniel DOBAK, III, La Jolla, CA (US); Burkhard JANSEN, La Jolla, CA (US); Zuxu YAO, San Diego, CA (US); Michael HOWELL, La Jolla, CA (US)

(21) Appl. No.: 17/534,177

(22) Filed: Nov. 23, 2021

Related U.S. Application Data
(60) Provisional application No. 63/117,946, filed on Nov. 24, 2020.

(51) Int. Cl. C12Q 1/6827; C12Q 1/6806; C12Q 1/6869; G01N 1/04

(52) U.S. Cl. CPC C12Q 1/6827; C12Q 1/6806; G01N 2001/028; G01N 1/04; C12Q 2600/158; C12Q 1/6869

(57) ABSTRACT

Disclosed herein is a method of quantifying mutation burden of skin based on genomic mutations. In some instances, also described herein are methods of reducing skin cancer risk, such as that caused by UV damage or other environmental factor.

### CROSS-REFERENCE

This application claims the benefit of U.S. provisional application No. 63/117,946, filed Nov. 24, 2020, which is incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### INCORPORATION BY REFERENCE OF SEQUENCE LISTING

The present application is being filed along with a Sequence Listing in electronic format. The Sequence Listing is provided as a file entitled “44503_731_201_sequence_listing.txt,” created Nov. 23, 2021, which is 77,732 bytes in size. The information in the electronic format of the Sequence Listing is incorporated by reference in its entirety.

### BACKGROUND

Skin diseases are some of the most common human illnesses and represent an important global burden in healthcare. Existing methods for assessing risk of such common skin diseases (such as cancer) suffer from invasiveness, low sensitivity, high cost, extended analysis times, or late-stage detection. Therefore, there exists a need in the art for non-invasive methods of assessing skin disease risk and providing early treatment interventions to prevent such diseases from manifesting.

### SUMMARY

Provided herein are methods for quantifying mutation burden. Provided herein are methods for quantifying a mutation burden in a subject, comprising: obtaining a sample from the subject by non-invasive sampling, wherein the sample comprises a one or more of skin cells; detecting at least one nucleic acid mutation in the sample; and quantifying the mutation burden based on presence, quantity, or absence of the at least one nucleic acid mutation. Further provided herein are methods wherein the non-invasive sampling comprises use of an adhesive tape. Further provided herein are methods wherein the sample comprises fewer than 1 gram of cellular material collected. Further provided herein are methods wherein the sample comprises 1 picogram-1 gram of cellular material collected. Further provided herein are methods wherein the sample comprises no more than 20 milligrams of cellular material collected. Further provided herein are methods wherein the sample comprises 1 picogram to 20 milligrams of cellular material collected. Further provided herein are methods wherein the sample comprises 1 picogram-500 micrograms of cellular material collected. Further provided herein are methods wherein the sample comprises skin cells from no more than the superficial about 0.1 mm of skin. Further provided herein are methods wherein the sample comprises skin cells from the superficial 10-20 μm of skin. Further provided herein are methods wherein the sample comprises skin cells from fewer than about 100 cell layers. Further provided herein are methods wherein the sample comprises skin cells from 1 to 50 cell layers. Further provided herein are methods wherein the sample comprises cellular material collected using one or more adhesive tapes. Further provided herein are methods wherein the sample comprises skin cells from 1 to 5 cell layers. Further provided herein are methods wherein the sample comprises skin cells obtained no deeper than the stratum germinativum. Further provided herein are methods wherein the sample comprises skin cells obtained from a skin surface area of 10-300 mm². Further provided herein are methods wherein the sample comprises a majority of skin sampled from a layer of skin exposed to an environmental factor. Further provided herein are methods wherein the environmental factor is ultraviolet (UV) light. Further provided herein are methods wherein the environmental factor is a chemical mutagen. Further provided herein are methods wherein the method further comprises detecting colonization of the one or more skin cells. Further provided herein are methods wherein the mutation burden comprises a ratio of the skin cells comprising the at least one nucleic acid mutation compared to a total number of cells in the sample. Further provided herein are methods wherein quantifying the mutation burden comprises detecting a copy number of at least 2 for the at least one nucleic acid mutation. Further provided herein are methods wherein the sample obtained by the non-invasive sampling comprises an increased percentage of cells contacted with the environmental factor compared to a percentage of cells contacted with the environmental factor in a sample obtained by standard biopsy. Further provided herein are methods wherein the method detects the at least one nucleic acid mutation in the sample obtained by the non-invasive sampling at an increased sensitivity compared to a sensitivity of detecting the at least one nucleic acid mutation in a sample obtained by standard biopsy. Further provided herein are methods wherein the number of nucleic acid mutations per mm² of skin collected comprises at least 25 mutations. Further provided herein are methods wherein the method detects the at least one nucleic acid mutation in the sample obtained by the non-invasive sampling with a sensitivity of at least 3.0% Further provided herein are methods wherein the method detects the at least one nucleic acid mutation in the sample obtained by the non-invasive sampling with a sensitivity of at least 1.0% Further provided herein are methods wherein the quantifying the mutation burden comprises detecting a variant allele frequency comprising the at least one nucleic acid mutation. Further provided herein are methods wherein the method comprises detecting 5-5,000 nucleic acid mutations in the sample. Further provided herein are methods wherein the method comprises detecting 2-25 nucleic acid mutations in the sample. Further provided herein are methods wherein the method comprises detecting at least 5 nucleic acid mutations in the sample. Further provided herein are methods wherein the method comprises detecting at least 10 nucleic acid mutations in the sample. Further provided herein are methods wherein the at least one mutation is present in at least 1% of the cells in the sample. Further provided herein are methods wherein the at least one mutation is present in at least 5% of the cells in the sample. Further provided herein are methods wherein the at least one mutation is present in at least 10% of the cells in the sample. Further provided herein are methods wherein the at least one nucleic acid mutation is present in TP53, NOTCH1, NOTCH2, NOTCH3, RBM10, PPP2R1A, GNAS, CTNNB1, PIK3CA, PPP6C, HRAS, KRAS, MTOR, SMAD3, LMNA, FGFR3, ZNF750, EPAS1, RPL22, ALDH2, CBFA2T3, CCND1, FAT1, FH, KLF4, CIC, RAC1, PTCH1, or TPM4. Further provided herein are methods wherein the at least one nucleic acid mutation is present in TP53. Further provided herein are methods wherein the at least one nucleic acid mutation is a mutation induced by UV light. Further provided herein are methods wherein the mutation induced by UV light is a C>T mutation. Further provided herein are methods wherein the mutation induced by UV light is a G>A mutation. Further provided herein are methods wherein the sample comprises cells of p53 immunopositive patches (PIPs). Further provided herein are methods wherein the method comprises detecting the at least one nucleic acid mutation in the cells of PIPs. Further provided herein are methods wherein the at least one nucleic acid mutation is present in at least one nucleic acid mutation in a MAPK pathway gene. Further provided herein are methods wherein the gene of MAPK pathway comprises BRAF, CBL, MAP2K1, NF1, or RAS. Further provided herein are methods wherein quantifying the mutation burden comprises detecting the at least one nucleic acid mutation in a cell cycle regulator. Further provided herein are methods wherein the cell cycle regulator is CDKN2A. Further provided herein are methods wherein the cell cycle regulator is PPP6C. Further provided herein are methods wherein the at least one nucleic acid mutation is present in an RNA processing gene. Further provided herein are methods wherein the RNA processing gene is DDX3X. Further provided herein are methods wherein the at least one nucleic acid mutation in present in a PI3K pathway gene. Further provided herein are methods wherein the PI3K pathway gene comprises XIAP, AKT1, TWIST1, BAD, CDKN1A, ABL1, CDH1, TP53, CASP3, PAK1, GAPDH, PIK3CA, FAS, AKT2, FRAP1, FOXO1A, PTK2, CASP9, PTEN, CCND1, NFKB1, GSK3B, MDM2, or CDKN1B. Further provided herein are methods wherein the at least one nucleic acid mutation is present in a chromatin remodeling gene. Further provided herein are methods wherein the chromatin remodeling gene is ARID2. Further provided herein are methods wherein the at least one nucleic acid mutation is a driver mutation. Further provided herein are methods wherein the at least one nucleic acid mutation is a passenger mutation. Further provided herein are methods wherein the at least one nucleic acid mutation is present in a transcription regulation region of a gene. Further provided herein are methods wherein the transcription regulation region of the gene comprises an enhancer, a silencer, an insulator, an operator, aa promoter, a 5′ untranslated region (5′ UTR), or a 3′ untranslated region (3′UTR). Further provided herein are methods wherein the transcription regulation region comprises the promoter. Further provided herein are methods wherein the non-invasive sampling is performed on skin from the subject's head. Further provided herein are methods wherein the non-invasive sampling is performed on skin from the subject's face. Further provided herein are methods wherein the one or more skin cells comprises melanocytes. Further provided herein are methods wherein the one or more skin cells comprise keratinocytes. Further provided herein are methods wherein the subject does not exhibit symptoms of cancer. Further provided herein are methods wherein the cancer is skin cancer. Further provided herein are methods wherein the method further comprises comparing the mutation burden with a reference comprising nucleic acid sequence data obtained from a non-cancerous skin sample. Further provided herein are methods wherein the method further comprises comparing the mutation burden with a reference comprising nucleic acid sequence data obtained from a skin sample not exposed to UV light. Further provided herein are methods wherein the method further comprises calculating a quantitative burden based on the mutation burden. Further provided herein are methods wherein the method further comprises providing to the subject a report or a recommendation based on the quantitative burden of the subject.

Provided herein are methods of reducing skin cancer risk comprising: calculating a quantitative burden based on the mutation burden described herein; and providing a treatment recommendation based on the quantitative burden. Further provided herein are methods wherein the quantitative burden is categorized as low, medium, or high. Further provided herein are methods wherein calculating the quantitative burden comprises use of machine learning. Further provided herein are methods wherein calculating the quantitative burden comprises weighting each mutation of the mutation burden. Further provided herein are methods wherein calculating the quantitative burden comprises correlating each mutation of the mutation burden with skin cancer risk. Further provided herein are methods wherein the treatment recommendation comprises use of sun protection sunscreens, supplements, or photolyase treatment. Further provided herein are methods wherein the treatment recommendation comprises use retinoids, light peel, or photodynamic therapy (PDT). Further provided herein are methods wherein the treatment recommendation comprises moderate or deep peel.

Provided herein are systems configured to perform a method described herein, said system comprising: an apparatus for performing non-invasive skin sample collection; a nucleic acid sequencing platform; and an assay for detecting the at least one nucleic acid mutation. Further provided herein are systems wherein the system detects 5-25 nucleic acid mutations. Further provided herein are systems wherein the system detects the at least one nucleic acid mutation with a sensitivity of at least 5%. Further provided herein are systems wherein the system detects the at least one nucleic acid mutation with a sensitivity of at least 1.0%. Further provided herein are systems wherein the system is configured to detect the a least one nucleic acid mutation by qPCR. Further provided herein are systems wherein the system is configured to detect the a least one nucleic acid mutation by allele-specific qPCR. Further provided herein are systems wherein the allele-specific qPCR comprises amplification of an allele comprising the at least one nucleic acid mutation. Further provided herein are systems wherein the system is configured to detect the at least one nucleic acid mutation by MALDI-TOF mass spectrometry, sequencing by synthesis, nanopore sequencing, ddPCR, sanger sequencing, or real-time PCR. Further provided herein are systems wherein the system is configured to detect the at least one nucleic acid mutation by MALDI-TOF mass spectrometry. Further provided herein are systems wherein the system is configured to detect two or more nucleic acid mutations. Further provided herein are systems wherein the system is configured to detect at least 5 nucleic acid mutations. Further provided herein are systems wherein the system is configured to detect at least 10 nucleic acid mutations. Further provided herein are systems wherein the system is configured to detect at least 40 nucleic acid mutations. Further provided herein are systems wherein the system is configured to detect 5-5000 nucleic acid mutations. Further provided herein are systems wherein the system is configured to detect nucleic acid mutations in at least one of TP53, NOTCH1, NOTCH2, CDKN2A, HRAS, or MTOR.

Provided herein are methods for quantifying a epigenetic burden in a subject, comprising: obtaining a sample from the subject by non-invasive sampling, wherein the sample comprises a one or more skin cells; detecting at least epigenetic modification in the sample; and quantifying the epigenetic burden based on presence, quantity, or absence of the at least one epigenetic modification. Further provided herein are methods wherein the at least one epigenetic modification comprises methylation in a CpG island of a gene or a transcription regulation region of the gene. Further provided herein are methods wherein the at least one epigenetic modification comprises 5-methylcytosine. Further provided herein are methods wherein the gene is KRT1, KRT5, KRT6, KRT14, KRT15, KRT16, KRT17, or KRT80. Further provided herein are methods wherein the at least one epigenetic modification comprises N6-methyladenine.

Provided herein are methods for quantifying a mutation burden in a subject, comprising: quantifying the mutation burden based on the presence, quantity, or absence of at least one nucleic acid mutation in a sample, wherein the sample comprises one or more of skin cells obtained from the subject by non-invasive sampling. Further provided herein are methods further comprising treating the subject. Further provided herein are methods wherein treating the subject comprises application or recommendation of sun protection sunscreens, supplements, retinoids, photolyase treatment, photodynamic therapy (PDT), or a skin peal. Further provided herein are methods wherein treating the subject comprises generation of report.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A depicts a plot of mutations in sun-exposed skins as a function of age for samples A-D. Separate bars for the each sample indicate Age (top), VAF (sum) (middle), and Mut. No (bottom). The x-axis is labeled Mutation No. and VAF (%) from 0 to 100 at 20 unit intervals.

FIG. 1B depicts a plot of mutations in sun-exposed skins as a function of age for samples E-H. Separate bars for the each sample indicate Age (top), VAF (sum) (middle), and Mut. No (bottom). The x-axis is labeled Mutation No. and VAF (%) from 0 to 100 at 10 unit intervals.

FIG. 2A depicts a plot of mutation detection in normal skin from healthy volunteers. Separate bars for the each sample indicate Age (top), VAF (sum) (middle), and Mut. No (bottom). The x-axis is labeled Mutation No. and VAF (%) from 0 to 100 at 20 unit intervals.

FIG. 2B depicts a plot of mutation detection in contralateral normal skin samples. Separate bars for the each sample indicate Age (top), VAF (sum) (middle), and Mut. No (bottom). The x-axis is labeled Mutation No. and VAF (%) from 0 to 100 at 20 unit intervals.

FIG. 3A depicts a plot of mutation count per test skin area (2.8 cm²) vs. age. The x-axis is labeled Age from 0 to 100 at 10 unit intervals. The y-axis is labeled Mut count (per 2.8 cm²) from −5 to 15 at 5 unit intervals. Exposed (grey diamonds); exposed outliers (black diamonds); and less-exposed (shaded square) are labeled.

FIG. 3B depicts a plot of total mutation burden (sum of variant allele frequency, VAF) vs. age. The x-axis is labeled Age from 0 to 100 at 10 unit intervals. The y-axis is labeled VAF (%, sum) from −5 to 40 at 5 unit intervals. Exposed (grey diamonds); exposed outliers (black diamonds); and less-exposed (shaded square) are labeled.

FIG. 3C depicts a plot of UV score vs. age. The x-axis is labeled Age from 0 to 100 at 10 unit intervals. The y-axis is labeled UV Score (VAF*Mut count) from −100 to 500 at 100 intervals. Exposed (grey diamonds); and less-exposed (shaded square) are labeled.

FIG. 3D depicts a plot of mutation burden (averaged VAF) score vs. age. The x-axis is labeled Age from 0 to 100 at 10 unit intervals. The y-axis is labeled VAF (%, average) from −2 to 12 at 2 intervals. Exposed (grey diamonds); exposed outliers (black diamonds); and less-exposed (shaded square) are labeled.

FIG. 3E depicts a plot of mutation scores (VAF) vs. age. Two outliers are labeled to contrast with accumulated mutations ‘normal’ for age groups.

FIG. 3F depicts a plot of UV damage scores and average mutation number vs. UV exposure. The x-axis is labeled UV exposure (left to right: none (0), low (0.75), moderate (1.6), high (3.3)). The y-axis is labeled 0 to 80 at 10 unit intervals. White bars correspond to UV damage score, black bars indicate average mutation #.

FIG. 4A depicts a plot of mutation count vs. nine skin samples and two sample pools obtained from analysis of a panel of 16 mutation targets. The x-axis is labeled LC: left cheek; RC: right cheek; LT: left temple; RT: right temple; LPA: left post auricular; RPA: right post auricular; FO: central forehead; NO: nose; Pool1: pooled skin samples from LC, RC, LT and RT; Pool2: pooled skin samples from LPA, RPA, FO and NO. The y-axis is labeled Mut count from 0 to 10 at 1 unit intervals.

FIG. 4B depicts a plot of mutation count vs. nine skin samples (labeled with patient initials) obtained from analysis of a panel of 16 mutation targets. The y-axis is labeled Mut count from 0 to 10 at 1 unit intervals. The x-axis represents different patient samples A-I.

FIG. 5A is a plot showing a total genomic DNA (gDNA) comparison across a variety of non-invasively sampled skin sites. The x-axis is labeled Site: CF: Centre Forehead; RF: Right Forehead; LF: Left forehead; NO: Nose; RC: Right Cheek; LC: Left Cheek, RT: Right Temple, LT: Left Temple. The y-axis is labeled gDNA Yield (pg) from 0.1 to 100,000 on a base 10 logarithmic scale.

FIG. 5B is a table including a comparison of total genomic DNA yield from each of a variety of skin sites using non-invasive sampling for 84 total subjects. Headings include site, n. of subjects extracted, no. of subjects with input <1 ng, QNS (%), total yield mean (pg), total yield median (pg) and total yield SEM (pg).

FIG. 6A graphically depicts mean numbers of mutations detected per subject by different facial sites with the standard error of the mean. The x-axis is labeled Site: CF: Centre Forehead; RF: Right Forehead; LF: Left forehead; NO: Nose; RC: Right Cheek; LC: Left Cheek, RT: Right Temple, LT: Left Temple. The y-axis is labeled Mutations detected per subject from 0 to 4 at 1 unit intervals.

FIG. 6B graphically depicts sums of the variant allele frequency of UV damage and cancer related mutations per subject at different facial sites. The y-axis is labeled Log 10 (VAF Sum+1) from 0.0 to 1.0 at 0.5 unit intervals.

FIG. 7A includes an example image of kit packaging. The packaging provides contact information for user questions.

FIG. 7B includes an example image of kit packaging, instructions, skin collection devices, and areas for placement of the skin collection devices before and after skin collection. The instructions are illustrated as: 1. Activate your kit online by entering your activation code at LuminateDNA.com/activate; 2. Clean forehead, nose, and cheek-bone collection areas with provided alcohol prep pad; 3. Use provided gauze pad to dry all four collection areas; 4. Remove first Smart Sticker from the Luminate SkinPrint Collector; 5. Press Smart Sticker firmly on the collection area. Then gently lift the Smart Sticker from the skin; 6. Place a used smart sticker on the lower panel. Repeat steps 3-6 for each remaining sticker. Two on the forehead, two on the nose, and two on each cheekbone; 7. Place the completed Luminate SkinPrint Collector into foil bag. Place foil bag in box; 8. Use included label to reseal box and ship our sample to the Gene Lab.

FIG. 7C includes further details that may be included in a kit described herein.

FIG. 8 illustrates a computer system that is programmed or otherwise configured to operate some systems or methods described herein.

### DETAILED DESCRIPTION

Described herein are methods and systems for quantifying mutation burden. Described herein are methods and systems for quantifying epigenetic changes. The mutation burden and/or epigenetic changes quantification in some instances is predictive of cancer risk. Further described herein are methods for quantifying mutation burden and/or epigenetic changes in skin samples using non-invasive sampling. Further described herein are systems and devices for high-throughput analysis of mutations and/or epigenetic changes in skin samples. Further described herein are systems and methods for high-throughput analysis of the skin microbiome.

Exposure of skin to environmental factors may cause an increase in mutation or epigenetic changes which over time, may lead to more serious conditions. Such mutations include both permissive, passenger mutations and driver mutations which promote cell proliferation, in some instances leading to cancer. A single cell comprising a driver in some instances will expand by clonal expansion to form a mutated cell population. Such populations in some instances appear normal and function normally, but contain abnormal genetic mutations. As additional mutations are acquired, such cells in some instances become visible lesions such as actinic keratosis and squamous cell cancer. In some embodiments, disclosed herein is a method of determining a mutation burden in cells. In some instances, the cells are skin cells. In some instances, also described herein is a method of monitoring a mutation burden related to future development of a skin cancer. In some embodiments, disclosed herein is a method of utilizing the presence of one or more mutations to quantify a mutation burden. In some instances, amount and type of mutations are quantified over time to monitor skin health and/or treatments.

### Markers of Disease Risk

Disclosed herein are methods of identifying and measuring markers associated with increased risk of disease. In some embodiments, such markers are nucleic acid mutations present in genetic material of a subject. In some instances, methods described herein quantify the mutation burden of a sample obtained from a subject by analysis of mutations. In some instances, a mutation burden is quantified using a sample obtained using a non-invasive sampling method described herein. Such markers in some instances are influenced by exposure to environmental factors (e.g., UV light, chemicals, or other factor). In some instances a marker of disease risk is indicative of a proliferative disease. In some instances a marker of disease risk is indicative of skin cancer (e.g., basal cell carcinoma (BCC), squamous cell carcinoma (SCC), or melanoma).

### Environmental Factors

An environmental factor may comprise electromagnetic radiation or chemical substance which modulates diseases risk. In some instances, the environmental factor is ultraviolent (UV) light. UV light generally disproportionately impacts specific areas of skin which are commonly exposed to UV light, such as the face, neck, or head. In some instances, the environmental factor is a chemical mutagen which causes mutations in skin. In some instances, the environmental factor is short-wavelength radiation (e.g., x-ray, gamma-ray, etc.) which causes genetic mutations. Such environmental factors also in some instances produce epigenetic changes to genomic material of exposed skin cells. In some instances, mutation burden is modulated by exposure to environmental factors described herein. In some embodiments, environmental factors manifest a disease or condition on the skin. In some instances, environmental factors comprise chemical exposure, air pollutants, water contamination, ingestion of a mutagen, or UV.

In some embodiments, the environmental factor comprises UV. Ultraviolet (UV) rays present one of the greatest risk factors for developing a skin cancer. The UV rays comprise 3 main types, UVA, UVB, and UVC. About 95% of the UV radiation is UVA rays, and which penetrates deep into the skin layer, leading to DNA damage by creating free radicals via reactive oxygen species and decreasing the activity of antigen present cells of the epidermis. UVB rays, also known as sunburn rays, are generally associated with skin cancer due the ability to induce formation of cyclobutane pyrimidine dimers and pyrimidine (6-4) photoproducts. In some instances, UV rays induce C to T and G to A mutations in genomic DNA. In some instances, UV rays come from the sun. In some embodiments, UV rays exposure occurs by a source other than the sun. In some embodiments, a method described herein comprises quantifying the mutation burden in a skin region that is exposed to UV. In some cases, also described herein include a method monitoring the mutation burden of the skin region that has been exposed to by UV, for about 1 week, 2 weeks, 3 weeks, 1 month, 2 months, 6 months, years or more. In some cases, also described herein include a method monitoring the mutation burden of the skin region that has been exposed to by UV for 1-5 years, 1-2 years, 1 week-6 weeks, 1 week-4 weeks, 1 week-2 weeks, 1 week-6 months, 1 week to 3 months, or 1 week-1 year. In some cases, also described herein include a method monitoring the cumulative mutation burden of the skin region that has been exposed to by UV over time.

An environmental factor may include chemical substances. In some embodiments, the chemical substance comprises a reactive oxygen species, deaminating agent, polyaromatic hydrocarbon, alkylating agent, bromide/bromine containing agent, sodium azide, psoralen (typically combined with UV), or benzene-containing agents. In some embodiments, the chemical substance is present in a formulation used to treat a skin disorder. In some embodiments, the chemical substance is present in a formulation used to treat a skin disorder such as acne, HSV, hives, rosacea, eczema, psoriasis, keratosis pilaris, melanoma, or lupus. In some instances, the chemical substance comprises a retinoid, such as isotretinoin. In some instances, a chemical substance comprises one or more of oxybenzone, benzophenone-1, benzophenone-8, OD-PABA, 4-methylbenzylidene camphor, 3-benzylidene camphor, nano-titanium dioxide, nano-zinc oxide, octinoxate, and octocrylene. In some instances, a chemical substance comprises one or more of coal tar, parabens, triclosan, formaldehyde, phthalates, and asbestos. In some instances, a chemical substance comprises ethylene oxide, 1,4-dioxane, retinol, quaternium-15, DMDM hydantoin, imidazolidinyl urea, diazolidinyl urea, sodium hydroxymethylglycinate, 2-bromo-2-nitropropane-1,3 diol, sodium lauryl sulfate, sodium laureth sulfate, triclosan, triclocarban, BHA, BHT, EDTA, ethanolamines (e.g., mea/dea/tea), methylisothiazolinone, methylchloroisothiazolinone, toluene, lead, octinoxate, oxybenzone, avobenzone, and benzalkonium chloride.

### Genetic Mutations

Described herein are methods of quantifying mutation burden from skin samples. In some instances, the mutation burden comprises one or more mutations. In some instances, mutations are present in genomic DNA. In some instances, mutations comprise substitutions, deletions, or additions. In some embodiments, a mutation includes a substitution. In some embodiments, a mutations comprises a deletion. In some embodiments, a mutation comprises an insertion. In some embodiments, a mutation includes an insertion. In some embodiments, a mutation comprises a chemical change to a nucleobase. For example, the mutation may include a dimerization such as a thymine dimer. In some embodiments, a mutation comprises a frameshift mutation. In some embodiments, a mutation comprises a translocation. In some instances, mutations are present in coding regions. In some instances, mutations are present in non-coding regions. In some instances, mutations are present in genes. In some instances, mutations are present in transcription factors binding sites, promoters, terminators or other regulatory element. In some instances mutations are present in the same gene. In some instances, mutations are present in multiple genes. In some instances, genetic mutations are obtained using non-invasive sampling techniques.

Some embodiments include multiple mutations. For example, multiple mutations may be measured, detected, or used in the methods described herein. Some embodiments include quantifying mutation burden based on multiple mutations. Some embodiments include quantifying mutation burden based on a first mutation and based on a second mutation. In some instances, a mutation comprises a driver mutation. In some instances, a mutation comprises a mutation in a proto-oncogene. In some instances, a mutation comprises a mutation in a tumor suppressor gene.

Mutations may be present at any abundance in a given cell population. In some instances, the cell population is comprised of different cell types. In some instances, mutations are analyzed as a function of specific cell types. In some instances, the cell population is comprised of keratinocytes, melanocytes, fibroblasts, antigen presenting cells (Langerhans cells, dendritic cells), and/or inflammatory cells (T cells, B cells). In some instances, the cell population is comprised of at least one of keratinocytes, melanocytes, fibroblasts, antigen presenting cells (Langerhans cells, dendritic cells), or inflammatory cells (T cells, B cells). In some instances, the cell population comprises a comparator sample. In some instances, a comparator sample is a bulk sample from a population of individuals, a sample which has been exposed to none or low amounts of an environmental factor in the same or different individual, or a sample obtained from a different area of skin on the same or different individual. The abundance of a mutation in a sample in some instances is expressed as a percentage of cells comprising the mutation or a ratio of cells comprising the mutation to cells without the mutation from the same cell type, skin location, individual, or sample. In some instances, a mutation is present at a rate in the cells of the sample. In some instances, a mutation is present at a rate of about 10%, 8%, 6%, 5%, 4% 3%, 2%, 1%, 0.5%, 0.2%, 0.1%, 0.08%, 0.05%, or about 0.01%. In some instances, a mutation is present at a rate of at least 10%, 8%, 6%, 5%, 4% 3%, 2%, 1%, 0.5%, 0.2%, 0.1%, 0.08%, 0.05%, or at least 0.01%. In some instances, a mutation is present at a rate of no more than 10%, 8%, 6%, 5%, 4% 3%, 2%, 1%, 0.5%, 0.2%, 0.1%, 0.08%, 0.05%, or no more than 0.01%. In some instances, a mutation is present at a rate of 1%-5%, 1%-4%, 1%-3%, 0.5%-5%, 0.5%-1%, 0.5%-2%, 2%-10%, 5%-10%, or 4%-10%. In some instances, a mutation is present in a sample at a ratio of the number of cells comprising a mutation relative to the number of total cells in the sample (e.g., mutations/cell). In some instances, a mutation is present in a sample at a ratio of at least 1:5, 1:10, 1:15, 1:20, 1:50, 1:70, 1:100, or 1:200. In some instances, a mutation is present in a sample at a ratio of no more than 1:5, 1:5, 1:15, 1:20, 1:50, 1:70, 1:100 or 1:200. In some instances, a mutation is present in a sample at a ratio of 1:3-1:100, 1:5-1:100, 1:10-1:100, 1:20-1:500, 1:20-1:200, 1:20-1:100, 1:20-1:200, or 1:30-1:200. In some instances, the abundance of a mutation determines the sensitivity needed to detect the mutation. In some instances, the methods described herein detect mutations with a sensitivity of about 0.1%, 0.2%, 0.5%, 1%, 1.5%, 2%, 3%, 4%, 5%, 7%, 10%, or about 15%. In some instances, the methods described herein detect mutations with a sensitivity of at least 0.1%, 0.2%, 0.5%, 1%, 1.5%, 2%, 3%, 4%, 5%, 7%, 10%, at least 15%. In some instances, the methods described herein detect mutations with a sensitivity of no more than 0.1%, 0.2%, 0.5%, 1%, 1.5%, 2%, 3%, 4%, 5%, 7%, 10%, or no more than 15%. In some instances, the methods described herein detect mutations with a sensitivity of about 0.1%-10%, 0.1-1%, 0.5-5%, 0.5-3%, 1%-10%, 1%-5%, 0.5-20%, or 1%-15%.

Mutations may be present in a gene at any copy number in a cell. In some instances, a mutation is present in a gene at one, two, three, four, five, six, seven, ten, or even more than 10 copies in a cell. In some instances, a mutation is present in a gene in at least two copies in a cell. Mutations may be present in a gene at any allele frequency in a cell. In some instances, a mutation is present at an allele frequency of at one, two, three, four, five, six, seven, ten, or even more than 10 copies in a cell. In some instances, a mutation is present at an allele frequency of at least two copies in a cell.

Some embodiments include more than one mutation. For example, the method may include measuring, detecting, receiving, or using mutations. In some embodiments, detecting comprises determining the presence or absence of one or more mutations. Some embodiments include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more mutations. Some embodiments include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more mutations, or a range of mutations defined by any two of the aforementioned integers. For example, some embodiments include measuring the frequency of about 10 mutations. Some embodiments include measuring the frequency of about 20 mutations. Some embodiments include measuring the frequency of about 30 mutations. Some embodiments include measuring the frequency of about 40 mutations. Some embodiments include measuring the frequency of 50 mutations. Some embodiments include measuring the frequency of 1-4 mutations. Some embodiments include measuring the frequency of 1-7 mutations. Some embodiments include measuring the frequency of 1-10 mutations. Some embodiments include measuring the frequency of 1-100 mutations. Some embodiments include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 mutations. Some embodiments include no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 mutations.

Mutations described herein may be measured using any method known in the art. In some instances, mutations are identified using PCR. In some instances, mutations are identified using Sanger sequencing. In some instances, mutations are identified using Next Generation Sequencing or sequencing by synthesis. In some instances, mutations are identified using nanopore sequencing. In some instances, mutations are identified using real time PCR (qPCR). In some instances, mutations are identified using digital PCR (ddPCR). In some instances, mutations are identified using single molecule (SMRT) sequencing. In some instances, mutations are identified using mass analysis. In some instances, 10, 100, 1000, 10,000, or more than 10,000 samples are assayed in parallel.

Mutations may be assessed using a genomic measurement. Genomic data may be generated by any of a variety of methods. Generating genomic data may include using a detection reagent that binds to a genetic material such as DNA or histones and yields a detectable signal. After use of a detection reagent that binds to genetic material and yields a detectable signal, a readout may be obtained that is indicative of the presence, absence or amount of the genetic material. Generating genomic data may include concentrating, filtering, or centrifuging a sample. In some instances, specific sequences of genomic DNA are enriched or amplified with target-specific primers, such as those which target specific genes, promoters, or other DNA sequences.

Some examples of methods for generating DNA sequence data include use of sequencing, microarray analysis (e.g. a SNP microarray), hybridization, polymerase chain reaction, or electrophoresis, or a combination thereof. DNA sequence data may be generated by sequencing a subject's DNA. Sequencing may include massive parallel sequencing. Examples of massive parallel sequencing techniques include pyrosequencing, sequencing by reversible terminator chemistry, sequencing-by-ligation mediated by ligase enzymes, or phospholinked fluorescent nucleotides or real-time sequencing. Generating genomic data may include preparing a sample or template for sequencing. Some template preparation methods include use of amplified templates originating from single DNA molecules, or single DNA molecule templates. Examples of amplification methods include emulsion PCR, rolling circle, or solid-phase amplification.

Some embodiments relate to a mutation burden assessment comprising a method as described herein. For example, the mutation burden assessment may include the measurement of one or more mutations and determining risk of developing skin cancer. The mutation burden assessment may be initiated by consumers, cosmetologists or clinicians depending on the nature of the environmental exposure (e.g. UV damage related accelerated aging, testing, or recommendations of anti-aging products including sunscreens with or without repair enzymes). The mutation burden assessment may be initiated based on the presence of physical evidence of mutation burden such as sun damaged skin, wrinkles, pigment changes, loss of elastosis, or emerging lesions related to UV damage (e.g. actinic keratoses). In some instances, a mutation burden assessment comprises an evaluation of disease risk. In some instances, the disease risk is skin cancer.

In some embodiments, the mutation burden assessment is performed or initiated by a medical professional on a subject. In some cases, a clinician would be assessing a patient and determining if the mutation burden assessment is indicated. In some embodiments, the mutation burden assessment includes a determination of sun exposure based on the subject's medical history. In some cases, the clinician gets a report of high risk patients. In some cases, a patient file is flagged for a mutation burden assessment based on medical history (e.g., actinic keratoses a skin cancer such as basal cell carcinoma (BCC), squamous cell carcinoma (SCC), melanoma, and/or solar lentigo). In some embodiments, the clinician orders the test yearly, or more often depending on subjects.

In some embodiments, the mutation burden assessment is performed or initiated by the subject. For example, the mutation burden assessment may be an annual screening test sent to the patient, or that the patient initiates and sends to a diagnostic lab or to a clinician. For example, the subject may receive skin sampling patches that the subject uses to collect his or her own skin samples, and sends to the laboratory or clinician. In some embodiments, the patient is sent a kit, on an annual basis for example, after having been identified by a medical record, algorithm, healthcare professional, or clinician. In some embodiments, the patient is simply concerned and orders the test.

In some embodiments, the need for a mutation burden assessment is determined by a computer or algorithm. In some embodiments, photography or images are used to demonstrate sun damage, and a need for the subject to have a mutation burden assessment. Some embodiments include a combination of criteria from a patient health file that be algorithmically identified and to whom a kit may be automatically sent, or may be flagged to be sent a communication, or placed on a high-risk list for insurers. In some embodiments, the need for a mutation burden assessment is determined using a mobile communication device such as a cell phone. For example, the subject may take a picture on a cell phone, the image may be analyzed, and a recommendation to have a mutation burden assessment may be returned to the subject. In some instances, an automated system provides a reminder to the subject to provide a sample using the kit.

Some embodiments include monitoring a subject using a method as described herein. For example, the mutation burden may be determined multiple times based on at least one mutation at separate time points. Some embodiments include comparing mutation burden in sequentially obtained samples. In some embodiments, a kit is provided that includes a space kit for “before” and “after” samples differentially labeled, useful for those undergoing specific treatments. In some embodiments, the multiple mutation burden skin assessments are performed about a month or more apart. Some embodiments include performing the assessment again after 1 month, 2 months, 3 months, 4 months, 5 months, 6 months, 7 month, 8 months, 9 months, 10 months, 11 months, 12 months, or more, or a range of months including any two of the aforementioned numbers of months. Some embodiments include performing the assessment again after at least 30 days. In some instances, assessments are at intervals which correspond to approximate skin cell turnover. performed Some embodiments include testing sequentially, or may include looking for incremental changes in mutation burden. Some embodiments include performing a method as provided herein to determine the presence or extent of skin damage before and/or after (e.g. 30 or more days after) a laser treatment, chemical peel or other treatment. In some cases, the mutation burden skin assessment is used to determine a pass/fail, or to show a positive or negative impact of a particular skin treatment. For example, a pass or improvement may include an increase or decrease in one or more target genes, such as a 2×, 5×, or 10× improvement in the up/downregulation of the target gene(s). In some instances, a pass or improvement may include an increase or decrease in one or more target genes of 1.1×, 1.2×, 1.3×, 1.5×, 1.7×, 2×, 3×, 5×, 10×, 15×, 20×, or 25× improvement in the up/downregulation of the target gene(s). In some instances, a pass or improvement may include an increase or decrease in one or more target genes of 1.1-10×, 1.1-5×, 1.1-2×, 1.5-4×, 1.5-10×, 1.8-10×, 1.8-5×, 2-10×, 2-20×, 2-5×, 5-10×, or 5-10×.

Disclosed herein, in certain embodiments, is a method of monitoring mutation burden. In some instances a method comprises one or more steps of: obtaining a sample from the subject by non-invasive sampling, detecting at least one nucleic acid mutation in the sample; and quantifying the mutation burden based on presence, quantity, or absence of the at least one nucleic acid mutation. In some embodiments, the sample comprises a one or more of skin cells. Some embodiments include isolating nucleic acids from a first skin sample obtained from a subject at a first time. A skin sample obtained in some instances comprises skin cells obtained from multiple collection devices (e.g., tapes or other non-invasive device). In some instances, a skin sample comprises skin cells obtained from 1, 2, 3, 4, 5, 6, or more than 6 collection devices. In some instances, a skin sample comprises skin cells obtained from 1-20, 1-15, 1-10, 1-8, 1-6, 1-4, 2-10, 2-20, 3-12, 3-6, 5-10, 5-7, 8-10, or 10-15 collection devices. In some instances, skin cells are obtained from multiple collection devices are pooled. In some instances, skin cells from multiple collection devices are obtained from essentially the same area of skin. In some embodiments, the nucleic acids are isolated from the first skin sample by applying an adhesive patch to a skin region of the subject in a manner sufficient to adhere skin sample cells to the adhesive patch, and removing the adhesive patch from the first skin sample in a manner sufficient to retain the adhered skin sample cells to the adhesive patch. Some embodiments include detecting one or more mutations in the first skin sample. Some embodiments include determining a first mutation burden in the first skin sample based on the one or more mutations. Some embodiments include isolating nucleic acids from a skin sample obtained from the subject at a second time. Some embodiments include detecting one or more mutations in the second skin sample. In some embodiments, the nucleic acids are isolated from the second skin sample by applying an adhesive patch to a skin region of the subject in a manner sufficient to adhere skin sample cells to the adhesive patch, and removing the adhesive patch from the second skin sample in a manner sufficient to retain the adhered skin sample cells to the adhesive patch. Some embodiments include determining a second mutation burden in the second skin sample based on one or more mutations. Some embodiments include comparing the second mutation burden to the first mutation burden. Some embodiments include providing a skin treatment to the subject after the first skin sample is obtained, and before the second skin sample is obtained. In some embodiments, the skin treatment comprises a sunscreen. The treatment in some instances is a sunscreen or a lip balm, but is not limited to such embodiments. Some embodiments include providing a second skin treatment to the subject. Some embodiments include providing a second skin treatment to the subject after second skin sample is obtained. Some embodiments include providing a second skin treatment to the subject after second skin sample is obtained, based on the second mutation burden of the second skin sample compared to the first mutation burden in the first skin sample. Some embodiments include providing a second skin treatment to the subject after the second skin sample is obtained, when there is a mutation burden above a threshold, or greater than a control amount. Some embodiments include not providing a second skin treatment to the subject after the second skin sample is obtained, when the mutation burden is below a threshold, or lower than a control amount. Some embodiments include not providing a second skin treatment to the subject after the second skin sample is obtained, when the mutation burden is above a threshold, or greater than a control amount. Some embodiments include providing a second skin treatment to the subject after the second skin sample is obtained, when the mutation burden is below a threshold, or lower than a control amount.

Mutations described herein may be present in a gene. In some instances, the gene is a gene which drives increased cell proliferation. In some instances, the gene is TP53, NOTCH1, NOTCH2, NOTCH3, RBM10, PPP2R1A, GNAS, CTNNB1, PIK3CA, PPP6C, HRAS, KRAS, MTOR, SMAD3, LMNA, FGFR3, ZNF750, EPAS1, RPL22, ALDH2, CBFA2T3, CCND1, FAT1, FH, KLF4, CIC, RAC1, PTCH1, or TPM4. In some instances, the mutation is a C to T or G to A substitution. In some instances, the gene is a gene included in Tables 1-5.

In some embodiments, the one or more mutations are present in a MAPK pathway gene. In some embodiments, the MAPK pathway gene includes but is not limited to BRAF, CBL, MAP2K1, NF1, or RAS.

In some embodiments, the one or more mutations are present in a cell cycle regulator. In some embodiments, the cell cycle regulator is a cyclin-dependent kinase (CDK) family gene. In some embodiments, the cell cycle regulator includes but is not limited to TP53, CDKN2A, or PPP6C.

In some embodiments, the one or more mutations comprise a mutation included in Tables 1-5. In some embodiments, the one or more mutations comprise at least 5, 10, 15, 20, 25, 30, 35, 40, 45, 50, 60, 70, 80, 90, or at least 100 mutations included in Tables 1-5.

In some embodiments, the one or more mutations comprise a mutation from a gene included in Table 5. For example, the one or more mutations may include a mutation in CDKN2A, NOTCH1, or TP53. The one or more mutations may include a mutation in CDKN2A. The one or more mutations may include a mutation in NOTCH1. The one or more mutations may include a mutation in one of TP53. The one or more mutations may include a mutation in one of CDKN2A, NOTCH1, or TP53. The one or more mutations may include a mutation in two of CDKN2A, NOTCH1, or TP53. The one or more mutations may include a mutation in all three of CDKN2A, NOTCH1, or TP53.

In some embodiments, the one or more mutations comprise a mutation included in Table 5. For example, the one or more mutations may include CDKN2A 148C>T, CDKN2A 242C>T, NOTCH1 1057C>T, NOTCH1 1093C>T, NOTCH1 1154C>T, NOTCH1 1171C>T_ASO, NOTCH1 1172C>T, NOTCH1 1348G>A, NOTCH1 1363G>A, NOTCH1 1393G>A, NOTCH1 1400G>A, NOTCH1 4357G>T, NOTCH2 337C>T, TP53 586C>T, TP53 733G>A, TP53 741 742DELINSTT_ASO, TP53 742C>T_ASO, TP53 743G>A, TP53 749C>T, TP53 796G>A, TP53 832C>T, TP53 833C>T, TP53 839G>A, TP53 844C>T, or TP53 856G>A. The one or more mutations may include 1, 2, 3, 4, 5, 6, 7, 8, 9, 10, 11, 12, 13, 14, 15, 16, 17, 18, 19, 20, 21, 22, 23, 24, or 25 of the mutations included in Table 5, or a range defined by any two of the aforementioned integers of the mutations included in Table 5. The one or more mutations may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, of the mutations included in Table 5. The one or more mutations may include no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 21, no more than 22, no more than 23, no more than 24, or no more than 25, of the mutations included in Table 5.

A mutation may be present in a cell cycle regulator. In some embodiments, the cell cycle regulator is cellular tumor antigen p53 (TP53). In some embodiments, at least one mutation in TP53 comprises G245S, R280K, R248L, G266R, P250L, C238F, R248Q, R248 W, R282 W, R196*, R286K, P278S, P278L, or R248 W. In some embodiments, at least one mutation in TP53 comprises G245S, R280K, R248L, G266R, P250L, or C238F. In some embodiments, at least one mutation in TP53 comprises R248Q, R248 W, R282 W, R196*, R286K, or P278S. In some embodiments, at least one mutation in TP53 comprises P278L, or R248 W. In some embodiments, at least one mutation in TP53 comprises c.733G>A, c.839G>A, c.743G>T, c.796G>A, c.749C>T, c.713G>T, c.743G>A, c.742C>T, c.844C>T, c.586G>A, c.856C>T, c.832C>T, c.833C>T, or c.741 742delinsTT. In some embodiments, at least one mutation in TP53 comprises c.733G>A, c.839G>A, c.743G>T, c.796G>A, c.749C>T, or c.713G>T. In some embodiments, at least one mutation in TP53 comprises c.743G>A, c.742C>T, c.844C>T, c.586G>A, c.856C>T, or c.832C>T. In some embodiments, at least one mutation in TP53 comprises c.833C>T, or c.741_742delinsTT. In some embodiments, the mutation is reflected in a TP53 amino acid sequence. The mutation in TP53 may be relative to the amino acid sequence in SEQ ID NO: 1.

In some embodiments, the at least one mutation includes a mutation at TP53 586C, TP53 733G, TP53 741, TP53 742C, TP53 743G, TP53 749C, TP53 796G, TP53 832C, TP53 833C, TP53 839G, TP53 844C, or TP53 856G. In some embodiments, the at least one mutation includes a mutation at TP53 586C, TP53 733G, TP53 741, TP53 742C, TP53 743G, TP53 749C, TP53 796G, TP53 832C, TP53 833C, TP53 839G, TP53 844C, and TP53 856G. In some embodiments, the at least one mutation includes a mutation at TP53 586C. In some embodiments, the at least one mutation includes a mutation at TP53 733G. In some embodiments, the at least one mutation includes a mutation at TP53 741. In some embodiments, the at least one mutation includes a mutation at TP53 742C. In some embodiments, the at least one mutation includes a mutation at TP53 743G. In some embodiments, the at least one mutation includes a mutation at TP53 749C. In some embodiments, the at least one mutation includes a mutation at TP53 796G. In some embodiments, the at least one mutation includes a mutation at TP53 832C. In some embodiments, the at least one mutation includes a mutation at TP53 833C. In some embodiments, the at least one mutation includes a mutation at TP53 839G. In some embodiments, the at least one mutation includes a mutation at TP53 844C. In some embodiments, the at least one mutation includes a mutation at TP53 856G.

In some embodiments, the at least one mutation comprises TP53 586C>T, TP53 733G>A, TP53 741 742DELINSTT_ASO, TP53 742C>T_ASO, TP53 743G>A, TP53 749C>T, TP53 796G>A, TP53 832C>T, TP53 833C>T, TP53 839G>A, TP53 844C>T, or TP53 856G>A. In some embodiments, the at least one mutation comprises TP53 586C>T, TP53 733G>A, TP53 741 742DELINSTT_ASO, TP53 742C>T_ASO, TP53 743G>A, TP53 749C>T, TP53 796G>A, TP53 832C>T, TP53 833C>T, TP53 839G>A, TP53 844C>T, and TP53 856G>A. In some embodiments, the at least one mutation comprises TP53 586C>T. In some embodiments, the at least one mutation comprises TP53 733G>A. In some embodiments, the at least one mutation comprises TP53 741 742DELINSTT_ASO. In some embodiments, the at least one mutation comprises TP53 742C>T_ASO. In some embodiments, the at least one mutation comprises TP53 743G>A. In some embodiments, the at least one mutation comprises TP53 749C>T. In some embodiments, the at least one mutation comprises TP53 796G>A. In some embodiments, the at least one mutation comprises TP53 832C>T. In some embodiments, the at least one mutation comprises TP53 833C>T. In some embodiments, the at least one mutation comprises TP53 839G>A. In some embodiments, the at least one mutation comprises TP53 844C>T. In some embodiments, the at least one mutation comprises TP53 856G>A.

In some embodiments, the cell cycle regulator is cyclin-dependent kinase inhibitor 2A (CDKN2A). In some embodiments, at least one mutation in CDKN2A comprises R58*, P144L, R80*, W110*, P81L, or Q50*. In some embodiments, at least one mutation in CDKN2A comprises c.172C>T, c.341C>T, c.283C>T, c.330G>A, c.242C>T, c.148C>T, or c.171_172delinsTT. In some embodiments, the mutation is reflected in a CDKN2A amino acid sequence. The mutation in CDKN2A may be relative to the amino acid sequence in SEQ ID NO: 2.

In some embodiments, the at least one mutation includes a mutation at CDKN2A 148C or CDKN2A 242C. In some embodiments, the at least one mutation includes mutations at CDKN2A 148C and CDKN2A 242C. In some embodiments, at least one mutation is at CDKN2A 148C. In some embodiments, at least one mutation is at CDKN2A 242C.

In some embodiments, the at least one mutation comprises CDKN2A 148C>T or CDKN2A 242C>T. In some embodiments, the at least one mutation includes CDKN2A 148C>T and CDKN2A 242C>T. In some embodiments, the at least one mutation includes CDKN2A 148C>T. In some embodiments, the at least one mutation includes CDKN2A 242C>T.

The at least one mutation may be present in a NOTCH family gene. In some embodiments, the NOTCH family gene includes but is not limited to NOTCH1 (which encodes neurogenic locus notch homolog protein 1) or NOTCH2 (which encodes neurogenic locus notch homolog protein 2). In some embodiments, the at least one mutation is present in NOTCH1. In some embodiments, the at least one mutation comprises NOTCH1 is E455K, P391S, C467F, P460S, C467Y, G427D, D352N, S137L, P391L, 5385, P460L, or E1453*. In some embodiments, the at least one mutation in NOTCH1 is R365C, E450K, E424K, R353C, or A465T. In some embodiments, the mutation is reflected in a NOTCH1 amino acid sequence. The mutation in NOTCH1 may be relative to the amino acid sequence in SEQ ID NO: 3.

In some embodiments, at least one mutation is at NOTCH1 1057C, NOTCH1 1093C, NOTCH1 1154C, NOTCH1 1171C, NOTCH1 1172C, NOTCH1 1348G, NOTCH1 1363G, NOTCH1 1393G, NOTCH1 1400G, NOTCH1 4357G, or NOTCH2 337C. In some embodiments, the at least one mutation includes mutations at NOTCH1 1057C, NOTCH1 1093C, NOTCH1 1154C, NOTCH1 1171C, NOTCH1 1172C, NOTCH1 1348G, NOTCH1 1363G, NOTCH1 1393G, NOTCH1 1400G, NOTCH1 4357G, and NOTCH2 337C. In some embodiments, at least one mutation is at NOTCH1 1057C. In some embodiments, at least one mutation is at NOTCH1 1093C. In some embodiments, at least one mutation is at NOTCH1 1154C. In some embodiments, at least one mutation is at NOTCH1 1171C. In some embodiments, at least one mutation is at NOTCH1 1172C. In some embodiments, at least one mutation is at NOTCH1 1348G. In some embodiments, at least one mutation is at NOTCH1 1363G. In some embodiments, at least one mutation is at NOTCH1 1393G. In some embodiments, at least one mutation is at NOTCH1 1400G. In some embodiments, at least one mutation is at NOTCH1 4357G. In some embodiments, at least one mutation is at NOTCH2 337C.

In some embodiments, the at least one mutation comprises NOTCH1 1057C>T, NOTCH1 1093C>T, NOTCH1 1154C>T, NOTCH1 1171C>T_ASO, NOTCH1 1172C>T, NOTCH1 1348G>A, NOTCH1 1363G>A, NOTCH1 1393G>A, NOTCH1 1400G>A, NOTCH1 4357G>T, or NOTCH2 337C>T. In some embodiments, the at least one mutation comprises NOTCH1 1057C>T, NOTCH1 1093C>T, NOTCH1 1154C>T, NOTCH1 1171C>T_ASO, NOTCH1 1172C>T, NOTCH1 1348G>A, NOTCH1 1363G>A, NOTCH1 1393G>A, NOTCH1 1400G>A, NOTCH1 4357G>T, and NOTCH2 337C>T. In some embodiments, the at least one mutation comprises NOTCH1 1057C>T. In some embodiments, the at least one mutation comprises NOTCH1 1093C>T. In some embodiments, the at least one mutation comprises NOTCH1 1154C>T. In some embodiments, the at least one mutation comprises NOTCH1 1171C>T_ASO. In some embodiments, the at least one mutation comprises NOTCH1 1172C>T. In some embodiments, the at least one mutation comprises NOTCH1 1348G>A. In some embodiments, the at least one mutation comprises NOTCH1 1363G>A. In some embodiments, the at least one mutation comprises NOTCH1 1393G>A. In some embodiments, the at least one mutation comprises NOTCH1 1400G>A. In some embodiments, the at least one mutation comprises NOTCH1 4357G>T. In some embodiments, the at least one mutation comprises NOTCH2 337C>T.

In some embodiments, the at least one mutation is present in NOTCH2. In some embodiments, the at least one mutation in NOTCH2 comprises R113*. In some embodiments, the at least one mutation in NOTCH1 comprises c.1363G>A, c/1171C>T, c.1400G>T, c.1378C>T, c.1400G>T, c.1280G>A, c.1054G>A, c.410C>T, c.1172C>T, c.1154C>T, c.1379C>T, or c.4357G>T. In some embodiments, the at least one mutation in NOTCH1 comprises c.1093C>T, c.1348G>A, c.1270G>A, or c.1057C>T. In some embodiments, the at least one mutation in NOTCH1 comprises c.1393G>A or c.4015-1G>A. In some embodiments, the at least one mutation in NOTCH2 comprises c.337C>T. In some embodiments, the mutation is reflected in a NOTCH2 amino acid sequence. The mutation in NOTCH2 may be relative to the amino acid sequence in SEQ ID NO: 4.

The at least one mutation may be present in an MTOR pathway gene. In some embodiments, the MTOR pathway gene includes but is not limited to MTOR, AKT, AKT1 (v-akt murine thymoma viral oncogene homolog 1), AKT1S1 (AKT1 substrate 1 (proline-rich)), ATG13 (autophagy related 13), BNIP3 (BCL2/adenovirus E1B 19 kDa interacting protein 3), BRAF (B-Raf proto-oncogene, serine/threonine kinase), CCNE1 (cyclin E1), CDK2 (cyclin-dependent kinase 2), CLIP1 (CAP-GLY domain containing linker protein 1), CYCS (cytochrome c, somatic), DDIT4 (DNA-damage-inducible transcript 4), DEPTOR (DEP domain containing MTOR-interacting protein), EEF2 (eukaryotic translation elongation factor 2), EIF4A1 (eukaryotic translation initiation factor 4A1), EIF4B (eukaryotic translation initiation factor 4B), EIF4E (eukaryotic translation initiation factor 4E), EIF4EBP1 (eukaryotic translation initiation factor 4E binding protein 1), FBXW11 (F-box and WD repeat domain containing 11), HRAS (Harvey rat sarcoma viral oncogene homolog), IKBKB (inhibitor of kappa light polypeptide gene enhancer in B-cells, kinase beta), IRS1 (insulin receptor substrate 1), MAP2K1 (mitogen-activated protein kinase 1), MAP2K2 (mitogen-activated protein kinase 2), MAPK1 (mitogen-activated protein kinase 1), MAPK3 (mitogen-activated protein kinase 3), MAPKAP1 (mitogen-activated protein kinase associated protein 1), MLST8 (MTOR associated protein, LST8 homolog), MTOR (mechanistic target of rapamycin (serine/threonine kinase)), NRAS (neuroblastoma RAS viral (v-ras) oncogene homolog), PDCD4 (programmed cell death 4 (neoplastic transformation inhibitor)), PDPK1 (3-phosphoinositide dependent protein kinase 1), PLD1 (phospholipase D1, phosphatidylcholine-specific), PLD2 (phospholipase D2), PML (promyelocytic leukemia), POLDIP3 (polymerase (DNA-directed), delta interacting protein 3), PPARGC1A (peroxisome proliferator-activated receptor gamma, coactivator 1 alpha), PRKCA (protein kinase C, alpha), PRR5 (proline rich 5 (renal)), PXN (paxillin), RAC1 (ras-related C3 botulinum toxin substrate 1 (rho family, small GTP binding protein Rac1)), RAF1 (Raf-1 proto-oncogene, serine/threonine kinase), RB1CC1 (RB1-inducible coiled-coil 1), RHEB (Ras homolog enriched in brain), RHOA (ras homolog family member A), RICTOR (RPTOR independent companion of MTOR, complex 2), RPS6KA1 (ribosomal protein S6 kinase, 90 kDa, polypeptide 1), RPS6KB1 (ribosomal protein S6 kinase, 70 kDa, polypeptide 1), RPTOR (regulatory associated protein of MTOR, complex 1), RRAGA (Ras-related GTP binding A), RRAGB (Ras-related GTP binding B), RRAGC (Ras-related GTP binding C), RRAGD (Ras-related GTP binding D), RRN3 (RRN3 RNA polymerase I transcription factor homolog), SFN (stratifin), SGK1 (serum/glucocorticoid regulated kinase 1), SREBF1 (sterol regulatory element binding transcription factor 1), SSPO (SCO-spondin), TSC1 (tuberous sclerosis 1), TSC2 (tuberous sclerosis 2), ULK1 (unc-51 like autophagy activating kinase 1), ULK2 (unc-51 like autophagy activating kinase 2), YWHAB (tyrosine 3-monooxygenase/tryptophan 5-monooxygenase activation protein, beta), YWHAE (tyrosine 3-monooxygenase/tryptophan 5-monooxygenase activation protein, epsilon), YWHAG (tyrosine 3-monooxygenase/tryptophan 5-monooxygenase activation protein, gamma), YWHAH (tyrosine 3-monooxygenase/tryptophan 5-monooxygenase activation protein, eta), YWHAQ (tyrosine 3-monooxygenase/tryptophan 5-monooxygenase activation protein, theta), YWHAZ (tyrosine 3-monooxygenase/tryptophan 5-monooxygenase activation protein, zeta), or YY1 (YY1 transcription factor).

In some embodiments, the at least one mutation is present in MTOR (which encodes serine/threonine-protein kinase mTOR). In some embodiments, the at least one mutation in MTOR comprises S2215F. In some embodiments, the at least one mutation in MTOR comprises c.6644C>T. In some embodiments, the mutation is reflected in a MTOR amino acid sequence. The mutation in MTOR may be relative to the amino acid sequence in SEQ ID NO: 5.

The at least one mutation may be present in an HRAS pathway gene. In some embodiments, the HRAS pathway gene includes but is not limited to HRAS (which encodes GTPase HRas). In some embodiments, the at least one mutation is present in HRAS. In some embodiments, the at least one mutation in HRAS comprises G12D, Q61L, or G13D. In some embodiments, the at least one mutation in HRAS comprises c.35G>A, c.182A>T, or c.38G>A. In some embodiments, the mutation is reflected in a HRAS amino acid sequence. The mutation in HRAS may be relative to the amino acid sequence in SEQ ID NO: 6.

In some embodiments, the one or more mutations are present in an RNA processing gene. In some embodiments, the RNA processing gene includes but is not limited to DDX3X.

In some embodiments, the one or more mutations are present in a PI3K pathway gene. In some embodiments, the one or more mutations are present in a PI3KCA family gene. In some instances, the PI3KCA family gene includes but is not limited to XIAP (BIRC4) (X-linked inhibitor of apoptosis), AKT1 (v-akt murine thymoma viral oncogene homolog 1), TWIST1 (Twist homolog 1 (Drosophila)), BAD (BCL2-associated agonist of cell death), CDKN1A (p21) (Cyclin-dependent kinase inhibitor 1A (p21, Cip1))), ABL1 (v-abl Abelson murine leukemia viral oncogene homolog 1), CDH1 (Cadherin 1, type 1, E-cadherin), TP53 (Tumor protein p53), CASP3 (Caspase 3, apoptosis-related cysteine peptidase), PAK1 (p21/Cdc42/Rac1-activated kinase 1), GAPDH (Glyceraldehyde-3-phosphate dehydrogenase), PIK3CA (Phosphoinositide-3-kinase, catalytic, α-polypeptide), FAS (TNF receptor superfamily, member 6), AKT2 (v-akt murine thymoma viral oncogene homolog 2), FRAP1 (mTOR) (FK506 binding protein 12-rapamycin associated protein 1), FOXO1A (Forkhead box 01), PTK2 (FAK) (PTK2 protein tyrosine kinase 2), CASP9 (Caspase 9, apoptosis-related cysteine peptidase), PTEN (Phosphatase and tensin homolog), CCND1 (Cyclin D1), NFKB1 (Nuclear factor κ-light polypeptide gene enhancer B-cells 1), GSK3B (Glycogen synthase kinase 3-β), MDM2 (Mdm2 p53 binding protein homolog (mouse)), or CDKN1B (p2′7) (Cyclin-dependent kinase inhibitor 1B (p27, Kip1)).

In some embodiments, the one or more mutations are present in a chromatin remodeling gene. In some embodiments, the chromatin remodeling gene includes but is not limited to ARID2.

In some embodiments, the one or more mutations are present in a transcription regulation region of a gene. In some embodiments, the region comprises a promoter. In some embodiments, the region comprises a terminator. In some embodiments, the region comprises a Kozak consensus sequence, stem loop structures or internal ribosome entry site. In some instances, the region comprises an enhancer, a silencer, an insulator, an operator, aa promoter, a 5′ untranslated region (5′ UTR), or a 3′ untranslated region (3′UTR).

Mutations described herein may be identified phenotypically. In some instances, mutations are identified using staining techniques. In some instances, the staining technique is an immunogenic staining technique. In some instances, samples comprise cells having p53 immunopositive patches (PIPs). In some instances, the one or more mutations are present in PIPs.

In some embodiments, the one or more mutations are included in Table 1, which includes mutations that may be associated with cancer. The mutations in Table 1 are catalogued at cancer.sanger.ac.uk under the COSMIC IDs provided in the table (as of Nov. 22, 2021, e.g. COSMIC release v94—28^{th} May 2021), the details of which are incorporated by reference herein in their entirety. The mutations in the table are further based on ENSEMBL (release 93) gene annotation for GRCh38. The mutations in in Table 1 may be resultant from UV light or sun damage, and therefore may be useful as indicators of UV damage using the methods described herein. Any one or more of the aspects in Table 1 such as genes, mutations, or mutation locations may be used in a kit or method described herein. For example, any one or more genes, locations, DNA changes, or amino acid (AA) changes in Table 1 may be useful in quantifying a mutation burden.

TABLE 1

| COSMIC ID | GENE | Location (GRCh38) | DNAchange | AA change |
|---|---|---|---|---|
| COSV58688166 | CDKN2A | 9:21974749-21974749 | c.79G>T | p.E27* |
| COSV58692592 | CDKN2A | 9:21974746-21974746 | c.82G>A | p.V28M |
| COSV58709780 | CDKN2A | 9:21974745-21974745 | c.83T>G | p.V28G |
| COSV58726603 | CDKN2A | 9:21974744-21974748 | c.80_84delinsCAC | p.E27Afs*16 |
| COSV58687989 | CDKN2A | 9:21974744-21974744 | c.84G>A | p.V28= |
| COSV58704956 | CDKN2A | 9:21974742-21974742 | c.86G>A | p.R29Q |
| COSV58724172 | CDKN2A | 9:21974740-21974740 | c.88G>C | p.A30P |
| COSV58705525 | CDKN2A | 9:21974739-21974739 | c.89C>T | p.A30V |
| COSV58696430 | CDKN2A | 9:21974739-21974739 | c.89C>A | p.A30E |
| COSV58719942 | CDKN2A | 9:21974738-21974738 | c.90G>A | p.A30= |
| COSV58726989 | CDKN2A | 9:21974736-21974736 | c.92T>C | p.L31P |
| COSV105166625 | CDKN2A | 9:21974733-21974733 | c.95T>G | p.L32R |
| COSV58685103 | CDKN2A | 9:21974733-21974733 | c.95T>C | p.L32P |
| COSV58684533 | CDKN2A | 9:21974733-21974733 | c.95T>A | p.L32Q |
| COSV58699713 | CDKN2A | 9:21974731-21974766 | c.62_97delinsA | p.A21Efs*11 |
| COSV58682729 | CDKN2A | 9:21974731-21974731 | c.97G>T | p.E33* |
| COSV58728935 | CDKN2A | 9:21974729-21974729 | c.99G>T | p.E33D |
| COSV58686137 | CDKN2A | 9:21974727-21974727 | c.101C>T | p.A34V |
| COSV58725090 | CDKN2A | 9:21974726-21974726 | c.102G>C | p.A34= |
| COSV100445269 | CDKN2A | 9:21974726-21974726 | c.102G>A | p.A34= |
| COSV58712281 | CDKN2A | 9:21974725-21974725 | c.103G>T | p.G35W |
| COSV58691471 | CDKN2A | 9:21974725-21974725 | c.103G>A | p.G35R |
| COSV58690447 | CDKN2A | 9:21974724-21974724 | c.104G>T | p.G35V |
| COSV58690595 | CDKN2A | 9:21974724-21974724 | c.104G>C | p.G35A |
| COSV58726978 | CDKN2A | 9:21974724-21974724 | c.104G>A | p.G35E |
| COSV58724219 | CDKN2A | 9:21974722-21974722 | c.106G>A | p.A36T |
| COSV58688149 | CDKN2A | 9:21974721-21974721 | c.107C>G | p.A36G |
| COSV58697841 | CDKN2A | 9:21974719-21974719 | c.109C>T | p.L37= |
| COSV58730077 | CDKN2A | 9:21974715-21974715 | c.113C>T | p.P38L |
| COSV58695857 | CDKN2A | 9:21974715-21974715 | c.113C>A | p.P38H |
| COSV58712824 | CDKN2A | 9:21974712-21974712 | c.116A>T | p.N391 |
| COSV58728606 | CDKN2A | 9:21974711-21974711 | c.117C>G | p.N39K |
| COSV58701836 | CDKN2A | 9:21974710-21974710 | c.118G>T | p.A40S |
| COSV58702577 | CDKN2A | 9:21974709-21974709 | c.119C>T | p.A40V |
| COSV58690934 | CDKN2A | 9:21974704-21974704 | c.124A>T | p.N42Y |
| COSV58725223 | CDKN2A | 9:21974704-21974704 | c.124A>G | p.N42D |
| COSV58703253 | CDKN2A | 9:21974704-21974704 | c.124A>C | p.N42H |
| COSV58690205 | CDKN2A | 9:21974703-21974703 | c.125A>T | p.N421 |
| COSV58691614 | CDKN2A | 9:21974703-21974703 | c.125A>C | p.N42T |
| COSV58723457 | CDKN2A | 9:21974700-21974700 | c.128G>T | p.S431 |
| COSV58729318 | CDKN2A | 9:21974699-21974699 | c.129T>A | p.S43R |
| COSV58716776 | CDKN2A | 9:21974697-21974719 | c.109_131delinsGTGCG | p.L37_Y44delinsVR |
| COSV58713866 | CDKN2A | 9:21974697-21974697 | c.131A>C | p.Y44S |
| COSV100446627 | CDKN2A | 9:21974696-21974696 | c.132C>T | p.Y44= |
| COSV58696346 | CDKN2A | 9:21974696-21974696 | c.132C>G | p.Y44* |
| COSV58689440 | CDKN2A | 9:21974696-21974696 | c.132C>A | p.Y44* |
| COSV104413730 | CDKN2A | 9:21974695-21974695 | c.133G>C | p.G45R |
| COSV58729064 | CDKN2A | 9:21974695-21974695 | c.133G>A | p.G45S |
| COSV58685320 | CDKN2A | 9:21974694-21974694 | c.134G>A | p.G45D |
| COSV58723246 | CDKN2A | 9:21974688-21974688 | c.140G>T | p.R47M |
| COSV58718173 | CDKN2A | 9:21974686-21974686 | c.142C>T | p.P48S |
| COSV58685231 | CDKN2A | 9:21974685-21974688 | c.140_143delinsTT | p.R47_P48delinsM |
| COSV58686028 | CDKN2A | 9:21974685-21974686 | c.142_143delinsTT | p.P48L |
| COSV58683273 | CDKN2A | 9:21974685-21974685 | c.143C>T | p.P48L |
| COSV58688196 | CDKN2A | 9:21974685-21974685 | c.143C>G | p.P48R |
| COSV58723344 | CDKN2A | 9:21974684-21974684 | c.144G>A | p.P48= |
| COSV58696067 | CDKN2A | 9:21974682-21974682 | c.146T>G | p.I49S |
| COSV58687879 | CDKN2A | 9:21974682-21974682 | c.146T>C | p.I49T |
| COSV58710655 | CDKN2A | 9:21974682-21974682 | c.146T>A | p.I49N |
| COSV58686131 | CDKN2A | 9:21974681-21974681 | c.147C>T | p.I49= |
| COSV58716997 | CDKN2A | 9:21974681-21974681 | c.147C>G | p.I49M |
| COSV58696145 | CDKN2A | 9:21974680-21974681 | c.147_148delinsTT | p.Q50* |
| COSV58717062 | CDKN2A | 9:21974680-21974681 | c.147_148delinsAT | p.Q50* |
| COSV58683786 | CDKN2A | 9:21974680-21974680 | c.148C>T | p.Q50* |
| COSV58715821 | CDKN2A | 9:21974679-21974679 | c.149A>T | p.Q50L |
| COSV58683689 | CDKN2A | 9:21974679-21974679 | c.149A>G | p.Q50R |
| COSV58692109 | CDKN2A | 9:21974678-21974678 | c.150G>T | p.Q50H |
| COSV58703332 | CDKN2A | 9:21974678-21974678 | c.150G>C | p.Q50H |
| COSV58697920 | CDKN2A | 9:21974678-21974678 | c.150G>A | p.Q50= |
| COSV58695751 | CDKN2A | 9:21971208-21971208 | c.151G>A | p.V51I |
| COSV58721040 | CDKN2A | 9:21971207-21971207 | c.152T>C | p.V51A |
| COSV58694886 | CDKN2A | 9:21971207-21971207 | c.152T>A | p.V51D |
| COSV58683436 | CDKN2A | 9:21971205-21971205 | c.154A>T | p.M52L |
| COSV58696062 | CDKN2A | 9:21971205-21971205 | c.154A>G | p.M52V |
| COSV58688565 | CDKN2A | 9:21971204-21971204 | c.155T>G | p.M52R |
| COSV58725322 | CDKN2A | 9:21971204-21971204 | c.155T>A | p.M52K |
| COSV58722669 | CDKN2A | 9:21971203-21971203 | c.156delinsTA | p.M52Ifs*68 |
| COSV58687020 | CDKN2A | 9:21971203-21971203 | c.156G>C | p.M52I |
| COSV58728864 | CDKN2A | 9:21971201-21971201 | c.158T>C | p.M53T |
| COSV58683319 | CDKN2A | 9:21971200-21971200 | c.159G>A | p.M53I |
| COSV58696903 | CDKN2A | 9:21971198-21971198 | c.161T>A | p.M54K |
| COSV58728906 | CDKN2A | 9:21971196-21971197 | c.162163delinsA | p.M54Ifs*92 |
| COSV58714242 | CDKN2A | 9:21971196-21971196 | c.163G>T | p.G55C |
| COSV58691553 | CDKN2A | 9:21971196-21971196 | c.163G>C | p.G55R |
| COSV58683339 | CDKN2A | 9:21971195-21971195 | c.164G>T | p.G55V |
| COSV58685474 | CDKN2A | 9:21971195-21971195 | c.164G>A | p.G55D |
| COSV100446209 | CDKN2A | 9:21971194-21971194 | c.165C>T | p.G55= |
| COSV58724759 | CDKN2A | 9:21971192-21971192 | c.167G>A | p.S56N |
| COSV58684312 | CDKN2A | 9:21971191-21971191 | c.168C>T | p.S56= |
| COSV58685036 | CDKN2A | 9:21971190-21971190 | c.169G>T | p.A57S |
| COSV58685696 | CDKN2A | 9:21971190-21971190 | c.169G>C | p.A57P |
| COSV58703844 | CDKN2A | 9:21971190-21971190 | c.169G>A | p.A57T |
| COSV58698193 | CDKN2A | 9:21971189-21971190 | c.169_170delinsTT | p.A57F |
| COSV58687535 | CDKN2A | 9:21971189-21971189 | c.170C>T | p.A57V |
| COSV58702469 | CDKN2A | 9:21971188-21971190 | c.169_171delinsTT | p.A57Ffs*89 |
| COSV58705214 | CDKN2A | 9:21971188-21971188 | c.171C>T | p.A57= |
| COSV58687724 | CDKN2A | 9:21971188-21971188 | c.171C>A | p.A57= |
| COSV58723792 | CDKN2A | 9:21971187-21971189 | c.170_172delinsTTT | p.A57_R58delinsV* |
| COSV58683779 | CDKN2A | 9:21971187-21971188 | c.171_172delinsTT | p.R58* |
| COSV58687698 | CDKN2A | 9:21971187-21971188 | c.171_172delinsAT | p.R58* |
| COSV58682666 | CDKN2A | 9:21971187-21971187 | c.172C>T | p.R58* |
| COSV99053472 | CDKN2A | 9:21971187-21971187 | c.172C>G | p.R58G |
| COSV58721549 | CDKN2A | 9:21971187-21971187 | c.172C>A | p.R58= |
| COSV58694506 | CDKN2A | 9:21971186-21971186 | c.173G>A | p.R58Q |
| COSV99053470 | CDKN2A | 9:21971185-21971185 | c.174A>G | p.R58= |
| COSV58726570 | CDKN2A | 9:21971184-21971184 | c.175G>A | p.V59M |
| COSV58728660 | CDKN2A | 9:21971183-21971183 | c.176T>G | p.V59G |
| COSV58704370 | CDKN2A | 9:21971181-21971181 | c.178G>T | p.A60S |
| COSV58685686 | CDKN2A | 9:21971180-21971180 | c.179C>T | p.A60V |
| COSV58692021 | CDKN2A | 9:21971180-21971180 | c.179C>A | p.A60E |
| COSV58724207 | CDKN2A | 9:21971179-21971179 | c.180G>A | p.A60= |
| COSV58683250 | CDKN2A | 9:21971178-21971178 | c.181G>T | p.E61* |
| COSV58721420 | CDKN2A | 9:21971177-21971177 | c.182A>G | p.E61G |
| COSV58687325 | CDKN2A | 9:21971174-21971174 | c.185T>C | p.L62P |
| COSV58701071 | CDKN2A | 9:21971172-21971172 | c.187C>G | p.L63V |
| COSV58686181 | CDKN2A | 9:21971171-21971171 | c.188T>G | p.L63R |
| COSV58716983 | CDKN2A | 9:21971171-21971171 | c.188T>C | p.L63P |
| COSV58694651 | CDKN2A | 9:21971171-21971171 | c.188T>A | p.L63Q |
| COSV105166891 | CDKN2A | 9:21971170-21971170 | c.189G>T | p.L63= |
| COSV58729783 | CDKN2A | 9:21971168-21971168 | c.191T>C | p.L64P |
| COSV58725538 | CDKN2A | 9:21971167-21971167 | c.192G>A | p.L64= |
| COSV58707086 | CDKN2A | 9:21971165-21971165 | c.194T>C | p.L65P |
| COSV58687581 | CDKN2A | 9:21971164-21971164 | c.195C>G | p.L65= |
| COSV58690423 | CDKN2A | 9:21971163-21971173 | c.186_196delinsCT | p.L63_H66delinsY |
| COSV58727758 | CDKN2A | 9:21971163-21971163 | c.196C>T | p.H66Y |
| COSV58702034 | CDKN2A | 9:21971162-21971162 | c.197A>T | p.H66L |
| COSV58691340 | CDKN2A | 9:21971162-21971162 | c.197A>G | p.H66R |
| COSV58717748 | CDKN2A | 9:21971162-21971162 | c.197A>C | p.H66P |
| COSV58729876 | CDKN2A | 9:21971161-21971163 | c.196_198delinsTAG | p.H66* |
| COSV58712216 | CDKN2A | 9:21971160-21971160 | c.199G>T | p.G67C |
| COSV58685808 | CDKN2A | 9:21971160-21971160 | c.199G>A | p.G67S |
| COSV58699814 | CDKN2A | 9:21971159-21971159 | c.200G>T | p.G67V |
| COSV58697876 | CDKN2A | 9:21971158-21971158 | c.201C>T | p.G67= |
| COSV58694929 | CDKN2A | 9:21971157-21971178 | c.181_202delinsAC | p.E61Tfs*52 |
| COSV58684018 | CDKN2A | 9:21971157-21971157 | c.202G>A | p.A68T |
| COSV58689853 | CDKN2A | 9:21971156-21971156 | c.203C>T | p.A68V |
| COSV58700174 | CDKN2A | 9:21971156-21971156 | c.203C>G | p.A68G |
| COSV58684557 | CDKN2A | 9:21971156-21971156 | c.203C>A | p.A68E |
| COSV58720624 | CDKN2A | 9:21971155-21971155 | c.204G>A | p.A68= |
| COSV58690247 | CDKN2A | 9:21971154-21971155 | c.204_205delinsTT | p.E69* |
| COSV58683264 | CDKN2A | 9:21971154-21971154 | c.205G>T | p.E69* |
| COSV58724303 | CDKN2A | 9:21971154-21971154 | c.205G>C | p.E69Q |
| COSV58727969 | CDKN2A | 9:21971154-21971154 | c.205G>A | p.E69K |
| COSV58725369 | CDKN2A | 9:21971153-21971153 | c.206A>T | p.E69V |
| COSV58725528 | CDKN2A | 9:21971152-21971153 | c.206_207delinsTA | p.E69V |
| COSV58717098 | CDKN2A | 9:21971152-21971152 | c.207G>C | p.E69D |
| COSV105166727 | CDKN2A | 9:21971152-21971152 | c.207G>A | p.E69= |
| COSV58722633 | CDKN2A | 9:21971151-21971151 | c.208C>T | p.P70S |
| COSV58727797 | CDKN2A | 9:21971151-21971151 | c.208C>G | p.P70A |
| COSV58701883 | CDKN2A | 9:21971150-21971150 | c.209C>T | p.P70L |
| COSV58725664 | CDKN2A | 9:21971149-21971149 | c.210C>T | p.P70= |
| COSV58710079 | CDKN2A | 9:21971148-21971148 | c.211A>G | p.N71D |
| COSV58705792 | CDKN2A | 9:21971147-21971147 | c.212A>T | p.N71I |
| COSV58721900 | CDKN2A | 9:21971146-21971146 | c.213delinsGGTCG | p.N71Kfs*50 |
| COSV58713592 | CDKN2A | 9:21971146-21971146 | c.213C>T | p.N71= |
| COSV58699200 | CDKN2A | 9:21971146-21971146 | c.213C>G | p.N71K |
| COSV58695272 | CDKN2A | 9:21971146-21971146 | c.213C>A | p.N71K |
| COSV58721845 | CDKN2A | 9:21971145-21971145 | c.214T>G | p.C72G |
| COSV58722593 | CDKN2A | 9:21971145-21971145 | c.214T>A | p.C72S |
| COSV58726670 | CDKN2A | 9:21971144-21971144 | c.215G>A | p.C72Y |
| COSV58691985 | CDKN2A | 9:21971143-21971143 | c.216C>T | p.C72= |
| COSV58683179 | CDKN2A | 9:21971143-21971143 | c.216C>A | p.C72* |
| COSV58691514 | CDKN2A | 9:21971142-21971142 | c.217G>T | p.A73S |
| COSV58683423 | CDKN2A | 9:21971142-21971142 | c.217G>C | p.A73P |
| COSV58721993 | CDKN2A | 9:21971142-21971142 | c.217G>A | p.A73T |
| COSV58698822 | CDKN2A | 9:21971141-21971141 | c.218C>T | p.A73V |
| COSV58727809 | CDKN2A | 9:21971141-21971141 | c.218C>G | p.A73G |
| COSV58729115 | CDKN2A | 9:21971140-21971140 | c.219C>T | p.A73= |
| COSV58683415 | CDKN2A | 9:21971139-21971139 | c.220G>T | p.D74Y |
| COSV58688841 | CDKN2A | 9:21971139-21971139 | c.220G>A | p.D74N |
| COSV58689077 | CDKN2A | 9:21971138-21971138 | c.221A>T | p.D74V |
| COSV104609655 | CDKN2A | 9:21971138-21971138 | c.221A>G | p.D74G |
| COSV58684939 | CDKN2A | 9:21971138-21971138 | c.221A>C | p.D74A |
| COSV58723764 | CDKN2A | 9:21971137-21971137 | c.222C>T | p.D74= |
| COSV58728532 | CDKN2A | 9:21971137-21971137 | c.222C>A | p.D74E |
| COSV58721085 | CDKN2A | 9:21971136-21971136 | c.223C>T | p.P75S |
| COSV100446518 | CDKN2A | 9:21971136-21971136 | c.223C>A | p.P75T |
| COSV58724826 | CDKN2A | 9:21971135-21971136 | c.223_224delinsT | p.P75Sfs*71 |
| COSV58688496 | CDKN2A | 9:21971135-21971135 | c.224C>T | p.P75L |
| COSV58729801 | CDKN2A | 9:21971135-21971135 | c.224C>A | p.P75H |
| COSV58683530 | CDKN2A | 9:21971134-21971134 | c.225C>T | p.P75= |
| COSV58692257 | CDKN2A | 9:21971134-21971134 | c.225C>A | p.P75= |
| COSV104609546 | CDKN2A | 9:21971133-21971133 | c.226G>T | p.A76S |
| COSV58691212 | CDKN2A | 9:21971133-21971133 | c.226G>C | p.A76P |
| COSV58684967 | CDKN2A | 9:21971133-21971133 | c.226G>A | p.A76T |
| COSV58704592 | CDKN2A | 9:21971132-21971132 | c.227C>T | p.A76V |
| COSV58725112 | CDKN2A | 9:21971132-21971132 | c.227C>G | p.A76G |
| COSV58707302 | CDKN2A | 9:21971131-21971131 | c.228C>T | p.A76= |
| COSV58727079 | CDKN2A | 9:21971129-21971129 | c.230C>G | p.T77S |
| COSV58691541 | CDKN2A | 9:21971126-21971126 | c.233T>A | p.L78H |
| COSV58723617 | CDKN2A | 9:21971124-21971124 | c.235A>G | p.T79A |
| COSV58714755 | CDKN2A | 9:21971124-21971124 | c.235A>C | p.T79P |
| COSV58683990 | CDKN2A | 9:21971123-21971123 | c.236C>T | p.T79I |
| COSV58686008 | CDKN2A | 9:21971122-21971122 | c.237C>T | p.T79= |
| COSV58727031 | CDKN2A | 9:21971122-21971122 | c.237C>G | p.T79= |
| COSV58686001 | CDKN2A | 9:21971121-21971122 | c.237_238delinsTT | p.R80* |
| COSV58682746 | CDKN2A | 9:21971121-21971121 | c.238C>T | p.R80* |
| COSV58721651 | CDKN2A | 9:21971121-21971121 | c.238C>A | p.R80= |
| COSV58724838 | CDKN2A | 9:21971120-21971120 | c.239G>T | p.R80L |
| COSV58698367 | CDKN2A | 9:21971120-21971120 | c.239G>A | p.R80Q |
| COSV58687028 | CDKN2A | 9:21971118-21971118 | c.241C>T | p.P81S |
| COSV104609644 | CDKN2A | 9:21971118-21971118 | c.241C>G | p.P81A |
| COSV58720289 | CDKN2A | 9:21971118-21971118 | c.241C>A | p.P81T |
| COSV58683884 | CDKN2A | 9:21971117-21971117 | c.242C>T | p.P81L |
| COSV58717232 | CDKN2A | 9:21971117-21971117 | c.242C>G | p.P81R |
| COSV58697231 | CDKN2A | 9:21971117-21971117 | c.242C>A | p.P81H |
| COSV58718874 | CDKN2A | 9:21971116-21971117 | c.242_243delinsTT | p.P81L |
| COSV58688708 | CDKN2A | 9:21971116-21971116 | c.243C>G | p.P81= |
| COSV58715254 | CDKN2A | 9:21971115-21971115 | c.244G>T | p.V82L |
| COSV58723757 | CDKN2A | 9:21971115-21971115 | c.244G>C | p.V82L |
| COSV58682703 | CDKN2A | 9:21971115-21971115 | c.244G>A | p.V82M |
| COSV58688645 | CDKN2A | 9:21971114-21971114 | c.245T>G | p.V82G |
| COSV58710340 | CDKN2A | 9:21971114-21971114 | c.245T>C | p.V82A |
| COSV58696370 | CDKN2A | 9:21971114-21971114 | c.245T>A | p.V82E |
| COSV58722238 | CDKN2A | 9:21971113-21971113 | c.246G>A | p.V82= |
| COSV58685601 | CDKN2A | 9:21971112-21971113 | c.246_247delinsCA | p.H83N |
| COSV58682852 | CDKN2A | 9:21971112-21971112 | c.247C>T | p.H83Y |
| COSV58690009 | CDKN2A | 9:21971112-21971112 | c.247C>G | p.H83D |
| COSV58685578 | CDKN2A | 9:21971112-21971112 | c.247C>A | p.H83N |
| COSV58690350 | CDKN2A | 9:21971111-21971111 | c.248A>T | p.H83L |
| COSV58689512 | CDKN2A | 9:21971111-21971111 | c.248A>G | p.H83R |
| COSV58703585 | CDKN2A | 9:21971111-21971111 | c.248A>C | p.H83P |
| COSV58706968 | CDKN2A | 9:21971110-21971111 | c.248_249delinsCT | p.H83P |
| COSV58719805 | CDKN2A | 9:21971110-21971110 | c.249C>G | p.H83Q |
| COSV58684711 | CDKN2A | 9:21971110-21971110 | c.249C>A | p.H83Q |
| COSV58683210 | CDKN2A | 9:21971109-21971109 | c.250G>T | p.D84Y |
| COSV58694349 | CDKN2A | 9:21971109-21971109 | c.250G>C | p.D84H |
| COSV58683289 | CDKN2A | 9:21971109-21971109 | c.250G>A | p.D84N |
| COSV58705912 | CDKN2A | 9:21971108-21971108 | c.251A>T | p.D84V |
| COSV58688961 | CDKN2A | 9:21971108-21971108 | c.251A>G | p.D84G |
| COSV58702489 | CDKN2A | 9:21971108-21971108 | c.251A>C | p.D84A |
| COSV58691161 | CDKN2A | 9:21971107-21971107 | c.252C>T | p.D84= |
| COSV58683653 | CDKN2A | 9:21971106-21971106 | c.253G>C | p.A85P |
| COSV58684450 | CDKN2A | 9:21971106-21971106 | c.253G>A | p.A85T |
| COSV58703816 | CDKN2A | 9:21971105-21971105 | c.254C>T | p.A85V |
| COSV105166977 | CDKN2A | 9:21971103-21971103 | c.256G>A | p.A86T |
| COSV58729271 | CDKN2A | 9:21971102-21971102 | c.257C>T | p.A86V |
| COSV58685895 | CDKN2A | 9:21971102-21971102 | c.257C>A | p.A86D |
| COSV58718452 | CDKN2A | 9:21971100-21971100 | c.259C>T | p.R87W |
| COSV58725854 | CDKN2A | 9:21971099-21971099 | c.260G>T | p.R87L |
| COSV58683633 | CDKN2A | 9:21971099-21971099 | c.260G>C | p.R87P |
| COSV58728617 | CDKN2A | 9:21971099-21971099 | c.260G>A | p.R87Q |
| COSV58692705 | CDKN2A | 9:21971097-21971098 | c.261_262delinsAA | p.E88K |
| COSV58683071 | CDKN2A | 9:21971097-21971097 | c.262G>T | p.E88* |
| COSV58683670 | CDKN2A | 9:21971097-21971097 | c.262G>A | p.E88K |
| COSV58729766 | CDKN2A | 9:21971096-21971096 | c.263A>T | p.E88V |
| COSV58728274 | CDKN2A | 9:21971096-21971096 | c.263A>G | p.E88G |
| COSV58728388 | CDKN2A | 9:21971096-21971096 | c.263A>C | p.E88A |
| COSV58702221 | CDKN2A | 9:21971095-21971103 | c.256_264delinsT | p.A86Wfs*31 |
| COSV58724863 | CDKN2A | 9:21971095-21971095 | c.264G>T | p.E88D |
| COSV58712874 | CDKN2A | 9:21971095-21971095 | c.264G>A | p.E88= |
| COSV58691394 | CDKN2A | 9:21971094-21971095 | c.264_265delinsAA | p.G89S |
| COSV58684636 | CDKN2A | 9:21971094-21971094 | c.265G>T | p.G89C |
| COSV58713110 | CDKN2A | 9:21971094-21971094 | c.265G>A | p.G89S |
| COSV58690485 | CDKN2A | 9:21971093-21971093 | c.266G>T | p.G89V |
| COSV100448493 | CDKN2A | 9:21971093-21971093 | c.266G>A | p.G89D |
| COSV58725503 | CDKN2A | 9:21971092-21971092 | c.267C>T | p.G89= |
| COSV58718295 | CDKN2A | 9:21971091-21971091 | c.268T>C | p.F90L |
| COSV58691125 | CDKN2A | 9:21971090-21971090 | c.269T>C | p.F90S |
| COSV58687900 | CDKN2A | 9:21971089-21971089 | c.270C>T | p.F90= |
| COSV58696860 | CDKN2A | 9:21971089-21971089 | c.270C>G | p.F90L |
| COSV58691501 | CDKN2A | 9:21971087-21971087 | c.272T>A | p.L91Q |
| COSV58729901 | CDKN2A | 9:21971086-21971086 | c.273G>A | p.L91= |
| COSV58690716 | CDKN2A | 9:21971085-21971085 | c.274G>T | p.D92Y |
| COSV58729199 | CDKN2A | 9:21971082-21971082 | c.277A>G | p.T93A |
| COSV58684508 | CDKN2A | 9:21971081-21971081 | c.278C>T | p.T93M |
| COSV58721866 | CDKN2A | 9:21971081-21971081 | c.278C>G | p.T93R |
| COSV58684689 | CDKN2A | 9:21971081-21971081 | c.278C>A | p.T93K |
| COSV58723227 | CDKN2A | 9:21971080-21971080 | c.279G>A | p.T93= |
| COSV58728376 | CDKN2A | 9:21971076-21971076 | c.283G>T | p.V95L |
| COSV58685874 | CDKN2A | 9:21971076-21971076 | c.283G>A | p.V95M |
| COSV58728592 | CDKN2A | 9:21971075-21971075 | c.284T>C | p.V95A |
| COSV58724121 | CDKN2A | 9:21971074-21971076 | c.283_285delinsCTC | p.V95L |
| COSV58684740 | CDKN2A | 9:21971069-21971069 | c.290T>G | p.L97R |
| COSV58691600 | CDKN2A | 9:21971069-21971069 | c.290T>C | p.L97P |
| COSV58697396 | CDKN2A | 9:21971067-21971067 | c.292C>T | p.H98Y |
| COSV58696031 | CDKN2A | 9:21971066-21971066 | c.293A>G | p.H98R |
| COSV58683927 | CDKN2A | 9:21971066-21971066 | c.293A>C | p.H98P |
| COSV58686124 | CDKN2A | 9:21971065-21971065 | c.294C>T | p.H98= |
| COSV58726012 | CDKN2A | 9:21971064-21971064 | c.295C>T | p.R99W |
| COSV58689257 | CDKN2A | 9:21971063-21971063 | c.296G>C | p.R99P |
| COSV58708694 | CDKN2A | 9:21971063-21971063 | c.296G>A | p.R99Q |
| COSV58722201 | CDKN2A | 9:21971062-21971062 | c.297G>A | p.R99= |
| COSV58727235 | CDKN2A | 9:21971061-21971062 | c.297_298delinsAC | p.A100P |
| COSV58722643 | CDKN2A | 9:21971061-21971061 | c.298G>T | p.A100S |
| COSV58704405 | CDKN2A | 9:21971061-21971061 | c.298G>C | p.A100P |
| COSV58694488 | CDKN2A | 9:21971060-21971060 | c.299C>T | p.A100V |
| COSV58691635 | CDKN2A | 9:21971059-21971059 | c.300C>T | p.A100= |
| COSV58692144 | CDKN2A | 9:21971058-21971058 | c.301G>T | p.G101W |
| COSV58692305 | CDKN2A | 9:21971057-21971057 | c.302G>T | p.G101V |
| COSV58685713 | CDKN2A | 9:21971056-21971056 | c.303G>A | p.G101= |
| COSV58704200 | CDKN2A | 9:21971055-21971055 | c.304G>C | p.A102P |
| COSV58708391 | CDKN2A | 9:21971055-21971055 | c.304G>A | p.A102T |
| COSV58689032 | CDKN2A | 9:21971054-21971054 | c.305C>T | p.A102V |
| COSV58690567 | CDKN2A | 9:21971054-21971054 | c.305C>A | p.A102E |
| COSV58724994 | CDKN2A | 9:21971053-21971053 | c.306G>C | p.A102= |
| COSV58728253 | CDKN2A | 9:21971053-21971053 | c.306G>A | p.A102= |
| COSV58705361 | CDKN2A | 9:21971052-21971052 | c.307C>T | p.R103W |
| COSV58721964 | CDKN2A | 9:21971051-21971052 | c.307_308delinsA | p.R103Sfs*43 |
| COSV58727273 | CDKN2A | 9:21971051-21971051 | c.308G>A | p.R103Q |
| COSV58728702 | CDKN2A | 9:21971050-21971050 | c.309G>A | p.R103= |
| COSV58688932 | CDKN2A | 9:21971048-21971048 | c.311T>G | p.L104R |
| COSV58726869 | CDKN2A | 9:21971046-21971046 | c.313G>A | p.D105N |
| COSV58684238 | CDKN2A | 9:21971044-21971044 | c.315C>T | p.D105= |
| COSV58691382 | CDKN2A | 9:21971044-21971044 | c.315C>A | p.D105E |
| COSV58721981 | CDKN2A | 9:21971043-21971043 | c.316G>A | p.V106M |
| COSV58712542 | CDKN2A | 9:21971041-21971041 | c.318G>A | p.V106= |
| COSV58716033 | CDKN2A | 9:21971040-21971040 | c.319C>T | p.R107C |
| COSV58693705 | CDKN2A | 9:21971039-21971039 | c.320G>A | p.R107H |
| COSV58684483 | CDKN2A | 9:21971038-21971038 | c.321C>T | p.R107= |
| COSV58726216 | CDKN2A | 9:21971037-21971037 | c.322delinsAA | p.D108Kfs*12 |
| COSV58682998 | CDKN2A | 9:21971037-21971037 | c.322G>T | p.D108Y |
| COSV58690035 | CDKN2A | 9:21971037-21971037 | c.322G>C | p.D108H |
| COSV58690777 | CDKN2A | 9:21971037-21971037 | c.322G>A | p.D108N |
| COSV58682804 | CDKN2A | 9:21971036-21971036 | c.323A>G | p.D108G |
| COSV58719034 | CDKN2A | 9:21971036-21971036 | c.323A>C | p.D108A |
| COSV58702696 | CDKN2A | 9:21971034-21971034 | c.325G>A | p.A109T |
| COSV58723784 | CDKN2A | 9:21971032-21971032 | c.327C>T | p.A109= |
| COSV58684388 | CDKN2A | 9:21971031-21971031 | c.328T>C | p.W110R |
| COSV58682976 | CDKN2A | 9:21971030-21971030 | c.329G>A | p.W110* |
| COSV58726998 | CDKN2A | 9:21971029-21971030 | c.329_330delinsAA | p.W110* |
| COSV58708986 | CDKN2A | 9:21971029-21971029 | c.330G>C | p.W110C |
| COSV58682827 | CDKN2A | 9:21971029-21971029 | c.330G>A | p.W110* |
| COSV58698973 | CDKN2A | 9:21971028-21971029 | c.330_331delinsAA | p.W110_G111delins* |
| COSV58718063 | CDKN2A | 9:21971028-21971028 | c.331G>A | p.G111S |
| COSV58728739 | CDKN2A | 9:21971027-21971027 | c.332G>A | p.G111D |
| COSV58699867 | CDKN2A | 9:21971026-21971026 | c.333C>T | p.G111= |
| COSV58684425 | CDKN2A | 9:21971025-21971025 | c.334C>T | p.R112C |
| COSV58723857 | CDKN2A | 9:21971025-21971025 | c.334C>G | p.R112G |
| COSV58709681 | CDKN2A | 9:21971025-21971025 | c.334C>A | p.R112S |
| COSV58697439 | CDKN2A | 9:21971024-21971024 | c.335G>C | p.R112P |
| COSV58704986 | CDKN2A | 9:21971024-21971024 | c.335G>A | p.R112H |
| COSV105166999 | CDKN2A | 9:21971023-21971024 | c.335_336delinsCC | p.R112P |
| COSV100446087 | CDKN2A | 9:21971022-21971022 | c.337C>T | p.L113= |
| COSV58728357 | CDKN2A | 9:21971022-21971022 | c.337C>A | p.L113M |
| COSV58727128 | CDKN2A | 9:21971021-21971021 | c.338T>C | p.L113P |
| COSV58728163 | CDKN2A | 9:21971020-21971020 | c.339G>T | p.L113= |
| COSV58704478 | CDKN2A | 9:21971019-21971019 | c.340C>T | p.P114S |
| COSV58697613 | CDKN2A | 9:21971018-21971019 | c.340_341delinsTT | p.P114F |
| COSV58683051 | CDKN2A | 9:21971018-21971018 | c.341C>T | p.P114L |
| COSV58690307 | CDKN2A | 9:21971018-21971018 | c.341C>A | p.P114H |
| COSV58685522 | CDKN2A | 9:21971017-21971018 | c.341_342delinsTT | p.P114L |
| COSV58690277 | CDKN2A | 9:21971017-21971017 | c.342C>T | p.P114= |
| COSV58683376 | CDKN2A | 9:21971016-21971016 | c.343G>T | p.V115L |
| COSV58728667 | CDKN2A | 9:21971015-21971015 | c.344T>A | p.V115E |
| COSV58684854 | CDKN2A | 9:21971013-21971013 | c.346G>T | p.D116Y |
| COSV58728438 | CDKN2A | 9:21971013-21971013 | c.346G>A | p.D116N |
| COSV58726748 | CDKN2A | 9:21971012-21971012 | c.347A>T | p.D116V |
| COSV58691575 | CDKN2A | 9:21971006-21971006 | c.353C>T | p.A118V |
| COSV100446147 | CDKN2A | 9:21971006-21971006 | c.353C>A | p.A118D |
| COSV58695431 | CDKN2A | 9:21971004-21971005 | c.354_355delinsCT | p.E119* |
| COSV58688112 | CDKN2A | 9:21971004-21971004 | c.355G>T | p.E119* |
| COSV58725006 | CDKN2A | 9:21971004-21971004 | c.355G>C | p.E119Q |
| COSV58685972 | CDKN2A | 9:21971002-21971006 | c.353_357delinsA | p.A118Efs*27 |
| COSV100445869 | CDKN2A | 9:21971002-21971002 | c.357G>T | p.E119D |
| COSV58683444 | CDKN2A | 9:21971001-21971001 | c.358G>T | p.E120* |
| COSV58717600 | CDKN2A | 9:21971001-21971001 | c.358G>A | p.E120K |
| COSV58725072 | CDKN2A | 9:21971000-21971000 | c.359A>C | p.E120A |
| COSV58724199 | CDKN2A | 9:21970996-21970996 | c.363G>A | p.L121= |
| COSV58703978 | CDKN2A | 9:21970995-21970995 | c.364G>T | p.G122C |
| COSV58686424 | CDKN2A | 9:21970995-21970995 | c.364G>A | p.G122S |
| COSV58689831 | CDKN2A | 9:21970994-21970994 | c.365G>T | p.G122V |
| COSV58700502 | CDKN2A | 9:21970994-21970994 | c.365G>A | p.G122D |
| COSV58727286 | CDKN2A | 9:21970993-21970993 | c.366C>T | p.G122= |
| COSV58728851 | CDKN2A | 9:21970993-21970993 | c.366C>A | p.G122= |
| COSV58709909 | CDKN2A | 9:21970992-21970992 | c.367C>A | p.H123N |
| COSV58721567 | CDKN2A | 9:21970990-21970990 | c.369T>A | p.H123Q |
| COSV58703374 | CDKN2A | 9:21970989-21970989 | c.370C>T | p.R124C |
| COSV58687202 | CDKN2A | 9:21970988-21970988 | c.371G>A | p.R124H |
| COSV58703805 | CDKN2A | 9:21970986-21970986 | c.373G>A | p.D125N |
| COSV58725817 | CDKN2A | 9:21970984-21970984 | c.375T>C | p.D125= |
| COSV58683405 | CDKN2A | 9:21970984-21970984 | c.375T>A | p.D125E |
| COSV58698939 | CDKN2A | 9:21970983-21970983 | c.376G>T | p.V126F |
| COSV58727425 | CDKN2A | 9:21970983-21970983 | c.376G>A | p.V126I |
| COSV58705336 | CDKN2A | 9:21970982-21970982 | c.377T>C | p.V126A |
| COSV58684293 | CDKN2A | 9:21970982-21970982 | c.377T>A | p.V126D |
| COSV58704249 | CDKN2A | 9:21970981-21970981 | c.378C>T | p.V126= |
| COSV58713816 | CDKN2A | 9:21970980-21970980 | c.379G>T | p.A127S |
| COSV58725910 | CDKN2A | 9:21970979-21970979 | c.380C>T | p.A127V |
| COSV58692167 | CDKN2A | 9:21970977-21970977 | c.382C>T | p.R128W |
| COSV58715027 | CDKN2A | 9:21970976-21970976 | c.383G>A | p.R128Q |
| COSV58702773 | CDKN2A | 9:21970975-21970975 | c.384G>A | p.R128= |
| COSV58728095 | CDKN2A | 9:21970974-21970974 | c.385T>C | p.Y129H |
| COSV58723959 | CDKN2A | 9:21970973-21970973 | c.386A>G | p.Y129C |
| COSV58699973 | CDKN2A | 9:21970972-21970973 | c.386_387delinsTT | p.Y129F |
| COSV58689871 | CDKN2A | 9:21970972-21970972 | c.387C>G | p.Y129* |
| COSV58684005 | CDKN2A | 9:21970972-21970972 | c.387C>A | p.Y129* |
| COSV58696112 | CDKN2A | 9:21970970-21970970 | c.389T>G | p.L130R |
| COSV58702833 | CDKN2A | 9:21970970-21970970 | c.389T>C | p.L130P |
| COSV58685390 | CDKN2A | 9:21970970-21970970 | c.389T>A | p.L130Q |
| COSV58693476 | CDKN2A | 9:21970969-21970969 | c.390G>A | p.L130= |
| COSV58687466 | CDKN2A | 9:21970968-21970968 | c.391C>T | p.R131C |
| COSV58727189 | CDKN2A | 9:21970967-21970967 | c.392G>T | p.R131L |
| COSV58714221 | CDKN2A | 9:21970967-21970967 | c.392G>C | p.R131P |
| COSV58688371 | CDKN2A | 9:21970967-21970967 | c.392G>A | p.R131H |
| COSV58728519 | CDKN2A | 9:21970966-21970966 | c.393C>G | p.R131= |
| COSV58722913 | CDKN2A | 9:21970965-21970965 | c.394G>C | p.A132P |
| COSV58696727 | CDKN2A | 9:21970965-21970965 | c.394G>A | p.A132T |
| COSV58686118 | CDKN2A | 9:21970964-21970964 | c.395C>T | p.A132V |
| COSV58718015 | CDKN2A | 9:21970963-21970963 | c.396G>A | p.A132= |
| COSV58697887 | CDKN2A | 9:21970958-21970958 | c.401C>T | p.A134V |
| COSV58723773 | CDKN2A | 9:21970955-21970955 | c.404G>C | p.G135A |
| COSV58712253 | CDKN2A | 9:21970955-21970955 | c.404G>A | p.G135E |
| COSV58697897 | CDKN2A | 9:21970954-21970954 | c.405G>T | p.G135= |
| COSV58696417 | CDKN2A | 9:21970953-21970953 | c.406G>A | p.G136S |
| COSV58684212 | CDKN2A | 9:21970952-21970952 | c.407G>A | p.G136D |
| COSV58728083 | CDKN2A | 9:21970951-21970951 | c.408C>T | p.G136= |
| COSV58727113 | CDKN2A | 9:21970950-21970950 | c.409A>G | p.T137A |
| COSV58727225 | CDKN2A | 9:21970950-21970950 | c.409A>C | p.T137P |
| COSV58726399 | CDKN2A | 9:21970949-21970949 | c.410C>T | p.T137I |
| COSV58725253 | CDKN2A | 9:21970948-21970948 | c.411C>G | p.T137= |
| COSV58724312 | CDKN2A | 9:21970946-21970946 | c.413G>T | p.R138I |
| COSV58725135 | CDKN2A | 9:21970946-21970946 | c.413G>C | p.R138T |
| COSV58716642 | CDKN2A | 9:21970946-21970946 | c.413G>A | p.R138K |
| COSV58723384 | CDKN2A | 9:21970943-21970943 | c.416G>A | p.G139D |
| COSV58727089 | CDKN2A | 9:21970941-21970941 | c.418A>T | p.S140C |
| COSV58694691 | CDKN2A | 9:21970940-21970940 | c.419G>A | p.S140N |
| COSV58728221 | CDKN2A | 9:21970935-21970935 | c.424C>T | p.H142Y |
| COSV58729050 | CDKN2A | 9:21970933-21970933 | c.426T>A | p.H142Q |
| COSV58727412 | CDKN2A | 9:21970932-21970932 | c.427G>A | p.A143T |
| COSV58702610 | CDKN2A | 9:21970930-21970930 | c.429C>T | p.A143= |
| COSV58697519 | CDKN2A | 9:21970928-21970928 | c.431G>A | p.R144H |
| COSV53036489 | NOTCH1 | 9:136523182-136523182 | c.410C>T | p.S137L |
| COSV53035530 | NOTCH1 | 9:136523179-136523179 | c.413G>A | p.C138Y |
| COSV53040495 | NOTCH1 | 9:136523177-136523177 | c.415C>T | p.Q139* |
| COSV53035508 | NOTCH1 | 9:136523177-136523177 | c.415C>A | p.Q139K |
| COSV53025017 | NOTCH1 | 9:136523169-136523169 | c.423T>C | p.A141= |
| COSV53049484 | NOTCH1 | 9:136523168-136523168 | c.424G>A | p.D142N |
| COSV53078439 | NOTCH1 | 9:136523167-136523167 | c.425A>G | p.D142G |
| COSV105113177 | NOTCH1 | 9:136523163-136523163 | c.429G>A | p.P143= |
| COSV104586581 | NOTCH1 | 9:136523161-136523161 | c.431G>A | p.C144Y |
| COSV104374516 | NOTCH1 | 9:136523159-136523159 | c.433G>A | p.A145T |
| COSV105112064 | NOTCH1 | 9:136523151-136523151 | c.441C>T | p.N147= |
| COSV53042659 | NOTCH1 | 9:136523150-136523150 | c.442C>T | p.P148S |
| COSV53085776 | NOTCH1 | 9:136523144-136523144 | c.448G>A | p.A150T |
| COSV53074230 | NOTCH1 | 9:136523128-136523128 | c.464G>T | p.C155F |
| COSV53036368 | NOTCH1 | 9:136523128-136523128 | c.464G>C | p.C155S |
| COSV99488292 | NOTCH1 | 9:136523128-136523128 | c.464G>A | p.C155Y |
| COSV53036444 | NOTCH1 | 9:136523123-136523123 | c.469C>T | p.P157S |
| COSV53031988 | NOTCH1 | 9:136523122-136523122 | c.470C>T | p.P157L |
| COSV105112838 | NOTCH1 | 9:136523121-136523122 | c.470_471delinsTT | p.P157L |
| COSV53094874 | NOTCH1 | 9:136523094-136523094 | c.498C>G | p.C166W |
| COSV99493685 | NOTCH1 | 9:136523086-136523086 | c.506G>A | p.S169N |
| COSV99072341 | NOTCH1 | 9:136518741-136518741 | c.949G>A | p.G317S |
| COSV105113225 | NOTCH1 | 9:136518729-136518729 | c.961T>C | p.C321R |
| COSV53055317 | NOTCH1 | 9:136518727-136518727 | c.963C>T | p.C321= |
| COSV53025404 | NOTCH1 | 9:136518726-136518726 | c.964G>A | p.V322M |
| COSV53109961 | NOTCH1 | 9:136518722-136518722 | c.968G>T | p.C323F |
| COSV53066614 | NOTCH1 | 9:136518717-136518717 | c.973A>G | p.N325D |
| COSV53054737 | NOTCH1 | 9:136518715-136518715 | c.975C>G | p.N325K |
| COSV53059152 | NOTCH1 | 9:136518715-136518715 | c.975C>A | p.N325K |
| COSV53077942 | NOTCH1 | 9:136518714-136518714 | c.976G>A | p.G326S |
| COSV53026016 | NOTCH1 | 9:136518713-136518713 | c.977G>T | p.G326V |
| COSV53034613 | NOTCH1 | 9:136518713-136518713 | c.977G>A | p.G326D |
| COSV99072340 | NOTCH1 | 9:136518711-136518711 | c.979T>C | p.W327R |
| COSV53080451 | NOTCH1 | 9:136518709-136518710 | c.980_981delinsAA | p.W327* |
| COSV53036206 | NOTCH1 | 9:136518709-136518709 | c.981G>C | p.W327C |
| COSV53103733 | NOTCH1 | 9:136518708-136518708 | c.982A>G | p.T328A |
| COSV53040400 | NOTCH1 | 9:136518695-136518695 | c.995G>A | p.C332Y |
| COSV53084514 | NOTCH1 | 9:136518692-136518692 | c.998G>C | p.S333T |
| COSV53108859 | NOTCH1 | 9:136518691-136518691 | c.999C>G | p.S333R |
| COSV53105375 | NOTCH1 | 9:136518685-136518685 | c.1005C>G | p.N335K |
| COSV105113431 | NOTCH1 | 9:136518681-136518681 | c.1009G>C | p.D337H |
| COSV53064749 | NOTCH1 | 9:136518680-136518680 | c.1010A>T | p.D337V |
| COSV105113489 | NOTCH1 | 9:136518678-136518678 | c.1012G>T | p.D338Y |
| COSV53052736 | NOTCH1 | 9:136518677-136518677 | c.1013A>G | p.D338G |
| COSV53047973 | NOTCH1 | 9:136518676-136518676 | c.1014C>G | p.D338E |
| COSV53094847 | NOTCH1 | 9:136518674-136518674 | c.1016G>T | p.C339F |
| COSV53051317 | NOTCH1 | 9:136518671-136518671 | c.1019C>T | p.A340V |
| COSV53049464 | NOTCH1 | 9:136518671-136518671 | c.1019C>A | p.A340D |
| COSV105112655 | NOTCH1 | 9:136518669-136518669 | c.1021A>G | p.S341G |
| COSV99072335 | NOTCH1 | 9:136518668-136518668 | c.1022G>A | p.S341N |
| COSV53088057 | NOTCH1 | 9:136518666-136518666 | c.1024G>A | p.A342T |
| COSV53051861 | NOTCH1 | 9:136518664-136518664 | c.1026C>T | p.A342= |
| COSV53079946 | NOTCH1 | 9:136518659-136518659 | c.1031G>T | p.C344F |
| COSV53065356 | NOTCH1 | 9:136518652-136518652 | c.1038C>T | p.H346= |
| COSV53031159 | NOTCH1 | 9:136518651-136518651 | c.1039G>T | p.G347C |
| COSV53032363 | NOTCH1 | 9:136518651-136518651 | c.1039G>A | p.G347S |
| COSV53040084 | NOTCH1 | 9:136518648-136518648 | c.1042G>A | p.A348T |
| COSV53038739 | NOTCH1 | 9:136518647-136518647 | c.1043C>T | p.A348V |
| COSV53083758 | NOTCH1 | 9:136518647-136518647 | c.1043C>A | p.A348D |
| COSV53026075 | NOTCH1 | 9:136518645-136518645 | c.1045A>C | p.T349P |
| COSV53042860 | NOTCH1 | 9:136518644-136518644 | c.1046C>T | p.T349I |
| COSV53072604 | NOTCH1 | 9:136518636-136518636 | c.1054G>T | p.D352Y |
| COSV53045203 | NOTCH1 | 9:136518636-136518636 | c.1054G>A | p.D352N |
| COSV53050840 | NOTCH1 | 9:136518635-136518635 | c.1055A>T | p.D352V |
| COSV53032942 | NOTCH1 | 9:136518633-136518633 | c.1057C>T | p.R353C |
| COSV53074787 | NOTCH1 | 9:136518632-136518632 | c.1058G>T | p.R353L |
| COSV53058129 | NOTCH1 | 9:136518632-136518632 | c.1058G>A | p.R353H |
| COSV53099030 | NOTCH1 | 9:136518620-136518624 | c.1066_1070delinsGCCTC | p.S356_F357delinsAS |
| COSV53037359 | NOTCH1 | 9:136518620-136518620 | c.1070T>C | p.F357S |
| COSV53034256 | NOTCH1 | 9:136518619-136518619 | c.1071C>G | p.F357L |
| COSV53084495 | NOTCH1 | 9:136518615-136518615 | c.1075T>C | p.C359R |
| COSV104540467 | NOTCH1 | 9:136518614-136518614 | c.1076G>A | p.C359Y |
| COSV53091985 | NOTCH1 | 9:136518613-136518614 | c.1076_1077delinsTT | p.C359F |
| COSV53032142 | NOTCH1 | 9:136518612-136518612 | c.1078G>T | p.E360* |
| COSV99485191 | NOTCH1 | 9:136518603-136518603 | c.1087C>T | p.H363Y |
| COSV53076760 | NOTCH1 | 9:136518597-136518598 | c.1092_1093delinsTT | p.R365C |
| COSV53025487 | NOTCH1 | 9:136518597-136518597 | c.1093C>T | p.R365C |
| COSV53045576 | NOTCH1 | 9:136518597-136518597 | c.1093C>A | p.R365S |
| COSV53040012 | NOTCH1 | 9:136518292-136518292 | c.1100G>T | p.G367V |
| COSV53049760 | NOTCH1 | 9:136518292-136518292 | c.1100G>C | p.G367A |
| COSV53078720 | NOTCH1 | 9:136518292-136518292 | c.1100G>A | p.G367D |
| COSV53079374 | NOTCH1 | 9:136518289-136518289 | c.1103T>A | p.L368Q |
| COSV53039654 | NOTCH1 | 9:136518288-136518288 | c.1104G>T | p.L368= |
| COSV53039439 | NOTCH1 | 9:136518277-136518277 | c.1115T>G | p.L372R |
| COSV105112510 | NOTCH1 | 9:136518277-136518277 | c.1115T>C | p.L372P |
| COSV53038982 | NOTCH1 | 9:136518272-136518272 | c.1120G>T | p.D374Y |
| COSV53025666 | NOTCH1 | 9:136518272-136518272 | c.1120G>A | p.D374N |
| COSV53067377 | NOTCH1 | 9:136518265-136518265 | c.1127G>T | p.C376F |
| COSV53042058 | NOTCH1 | 9:136518265-136518265 | c.1127G>A | p.C376Y |
| COSV53034718 | NOTCH1 | 9:136518264-136518264 | c.1128C>A | p.C376* |
| COSV53103305 | NOTCH1 | 9:136518257-136518257 | c.1135A>G | p.N379D |
| COSV53074387 | NOTCH1 | 9:136518245-136518245 | c.1147G>A | p.E383K |
| COSV53045049 | NOTCH1 | 9:136518243-136518243 | c.1149G>A | p.E383= |
| COSV53053354 | NOTCH1 | 9:136518238-136518238 | c.1154C>T | p.S385F |
| COSV53078940 | NOTCH1 | 9:136518238-136518238 | c.1154C>A | p.S385Y |
| COSV53037298 | NOTCH1 | 9:136518237-136518237 | c.1155C>A | p.S385= |
| COSV53051930 | NOTCH1 | 9:136518235-136518235 | c.1157A>C | p.N386T |
| COSV53087072 | NOTCH1 | 9:136518234-136518234 | c.1158C>G | p.N386K |
| COSV53031364 | NOTCH1 | 9:136518229-136518229 | c.1163A>G | p.D388G |
| COSV53087199 | NOTCH1 | 9:136518227-136518227 | c.1165A>C | p.T389P |
| COSV53060926 | NOTCH1 | 9:136518224-136518224 | c.1168A>G | p.N390D |
| COSV53060312 | NOTCH1 | 9:136518223-136518223 | c.1169A>G | p.N390S |
| COSV53090186 | NOTCH1 | 9:136518222-136518222 | c.1170C>G | p.N390K |
| COSV53043280 | NOTCH1 | 9:136518221-136518221 | c.1171C>T | p.P391S |
| COSV53047338 | NOTCH1 | 9:136518221-136518221 | c.1171C>G | p.P391A |
| COSV53069823 | NOTCH1 | 9:136518220-136518220 | c.1172C>T | p.P391L |
| COSV53025109 | NOTCH1 | 9:136518217-136518217 | c.1175T>C | p.V392A |
| COSV99492449 | NOTCH1 | 9:136518212-136518212 | c.1180G>T | p.G394C |
| COSV105112794 | NOTCH1 | 9:136518211-136518212 | c.1180_1181delinsAA | p.G394N |
| COSV99494013 | NOTCH1 | 9:136518211-136518211 | c.1181G>A | p.G394D |
| COSV53040908 | NOTCH1 | 9:136518208-136518208 | c.1184A>T | p.K395M |
| COSV53047809 | NOTCH1 | 9:136518199-136518199 | c.1193G>T | p.C398F |
| COSV104586375 | NOTCH1 | 9:136518199-136518199 | c.1193G>A | p.C398Y |
| COSV53079357 | NOTCH1 | 9:136518197-136518197 | c.1195A>G | p.T399A |
| COSV53059579 | NOTCH1 | 9:136518197-136518197 | c.1195A>C | p.T399P |
| COSV53069003 | NOTCH1 | 9:136518196-136518196 | c.1196C>A | p.T399N |
| COSV53076619 | NOTCH1 | 9:136518194-136518194 | c.1198T>G | p.C400G |
| COSV53068984 | NOTCH1 | 9:136518194-136518194 | c.1198T>C | p.C400R |
| COSV53078862 | NOTCH1 | 9:136518193-136518193 | c.1199G>T | p.C400F |
| COSV53087880 | NOTCH1 | 9:136518190-136518190 | c.1202C>T | p.P401L |
| COSV53082370 | NOTCH1 | 9:136518190-136518190 | c.1202C>A | p.P401H |
| COSV53047891 | NOTCH1 | 9:136518189-136518189 | c.1203C>T | p.P401= |
| COSV53081435 | NOTCH1 | 9:136518187-136518187 | c.1205C>T | p.S402L |
| COSV53036803 | NOTCH1 | 9:136518187-136518187 | c.1205C>A | p.S402* |
| COSV53059923 | NOTCH1 | 9:136518181-136518181 | c.1211A>G | p.Y404C |
| COSV53036098 | NOTCH1 | 9:136518176-136518176 | c.1216G>T | p.G406C |
| COSV53064471 | NOTCH1 | 9:136518176-136518176 | c.1216G>A | p.G406S |
| COSV53025929 | NOTCH1 | 9:136518170-136518170 | c.1222G>A | p.A408T |
| COSV53085676 | NOTCH1 | 9:136518166-136518166 | c.1226G>T | p.C409F |
| COSV104586474 | NOTCH1 | 9:136518161-136518161 | c.1231C>T | p.Q411* |
| COSV53069197 | NOTCH1 | 9:136518155-136518155 | c.1237G>A | p.V413M |
| COSV53040637 | NOTCH1 | 9:136518153-136518153 | c.1239G>A | p.V413= |
| COSV53085649 | NOTCH1 | 9:136518147-136518147 | c.1245G>T | p.E415D |
| COSV104541529 | NOTCH1 | 9:136518144-136518144 | c.1248C>G | p.C416W |
| COSV53077478 | NOTCH1 | 9:136518137-136518137 | c.1255G>T | p.G419C |
| COSV53052320 | NOTCH1 | 9:136517929-136517929 | c.1264C>T | p.P422S |
| COSV53085870 | NOTCH1 | 9:136517928-136517929 | c.1264_1265delinsTT | p.P422F |
| COSV53052301 | NOTCH1 | 9:136517928-136517928 | c.1265C>T | p.P422L |
| COSV53072183 | NOTCH1 | 9:136517926-136517926 | c.1267T>G | p.C423G |
| COSV53051148 | NOTCH1 | 9:136517925-136517925 | c.1268G>A | p.C423Y |
| COSV53036462 | NOTCH1 | 9:136517924-136517924 | c.1269C>G | p.C423W |
| COSV53054024 | NOTCH1 | 9:136517923-136517923 | c.1270G>C | p.E424Q |
| COSV53031725 | NOTCH1 | 9:136517923-136517923 | c.1270G>A | p.E424K |
| COSV53091911 | NOTCH1 | 9:136517916-136517916 | c.1277C>A | p.A426E |
| COSV53077459 | NOTCH1 | 9:136517914-136517914 | c.1279G>T | p.G427C |
| COSV53042252 | NOTCH1 | 9:136517913-136517913 | c.1280G>A | p.G427D |
| COSV53074477 | NOTCH1 | 9:136517907-136517907 | c.1286G>T | p.C429F |
| COSV53051339 | NOTCH1 | 9:136517907-136517907 | c.1286G>A | p.C429Y |
| COSV53085414 | NOTCH1 | 9:136517904-136517904 | c.1289T>C | p.I430T |
| COSV53091749 | NOTCH1 | 9:136517904-136517904 | c.1289T>A | p.I430N |
| COSV53073544 | NOTCH1 | 9:136517901-136517901 | c.1292A>G | p.N431S |
| COSV53049560 | NOTCH1 | 9:136517900-136517900 | c.1293C>G | p.N431K |
| COSV53032980 | NOTCH1 | 9:136517898-136517898 | c.1295C>T | p.T432M |
| COSV53037156 | NOTCH1 | 9:136517892-136517892 | c.1301G>A | p.G434D |
| COSV53053410 | NOTCH1 | 9:136517886-136517886 | c.1307T>G | p.F436C |
| COSV53082954 | NOTCH1 | 9:136517880-136517880 | c.1313G>A | p.C438Y |
| COSV53039987 | NOTCH1 | 9:136517879-136517879 | c.1314C>G | p.C438W |
| COSV53053276 | NOTCH1 | 9:136517878-136517878 | c.1315C>T | p.Q439* |
| COSV104586315 | NOTCH1 | 9:136517875-136517875 | c.1318T>G | p.C440G |
| COSV53037970 | NOTCH1 | 9:136517874-136517874 | c.1319G>T | p.C440F |
| COSV53045435 | NOTCH1 | 9:136517874-136517874 | c.1319G>C | p.C440S |
| COSV53066899 | NOTCH1 | 9:136517873-136517873 | c.1320T>G | p.C440W |
| COSV53049863 | NOTCH1 | 9:136517869-136517869 | c.1324C>T | p.Q442* |
| COSV53028703 | NOTCH1 | 9:136517866-136517866 | c.1327G>A | p.G443S |
| COSV53028689 | NOTCH1 | 9:136517865-136517865 | c.1328G>A | p.G443D |
| COSV105112485 | NOTCH1 | 9:136517862-136517862 | c.1331A>G | p.Y444C |
| COSV53073768 | NOTCH1 | 9:136517858-136517858 | c.1335G>T | p.T445= |
| COSV53057433 | NOTCH1 | 9:136517854-136517854 | c.1339C>T | p.P447S |
| COSV53053163 | NOTCH1 | 9:136517853-136517853 | c.1340C>T | p.P447L |
| COSV105112995 | NOTCH1 | 9:136517852-136517852 | c.1341C>A | p.P447= |
| COSV105112793 | NOTCH1 | 9:136517851-136517852 | c.1341_1342delinsTT | p.R448* |
| COSV53081503 | NOTCH1 | 9:136517851-136517851 | c.1342C>T | p.R448* |
| COSV53065420 | NOTCH1 | 9:136517850-136517850 | c.1343G>A | p.R448Q |
| COSV99489494 | NOTCH1 | 9:136517847-136517847 | c.1346G>A | p.C449Y |
| COSV105112061 | NOTCH1 | 9:136517846-136517846 | c.1347C>G | p.C449W |
| COSV53029104 | NOTCH1 | 9:136517845-136517845 | c.1348G>A | p.E450K |
| COSV53087027 | NOTCH1 | 9:136517839-136517839 | c.1354G>A | p.D452N |
| COSV53071116 | NOTCH1 | 9:136517837-136517837 | c.1356C>A | p.D452E |
| COSV53098013 | NOTCH1 | 9:136517835-136517835 | c.1358T>G | p.V453G |
| COSV53027071 | NOTCH1 | 9:136517830-136517830 | c.1363G>A | p.E455K |
| COSV53090874 | NOTCH1 | 9:136517827-136517827 | c.1366T>C | p.C456R |
| COSV53053723 | NOTCH1 | 9:136517827-136517827 | c.1366T>A | p.C456S |
| COSV53053704 | NOTCH1 | 9:136517826-136517826 | c.1367G>T | p.C456F |
| COSV53046494 | NOTCH1 | 9:136517826-136517826 | c.1367G>A | p.C456Y |
| COSV53031646 | NOTCH1 | 9:136517824-136517824 | c.1369G>A | p.V457I |
| COSV53049738 | NOTCH1 | 9:136517820-136517820 | c.1373C>T | p.S458L |
| COSV53045958 | NOTCH1 | 9:136517819-136517819 | c.1374G>A | p.S458= |
| COSV53039240 | NOTCH1 | 9:136517815-136517815 | c.1378C>T | p.P460S |
| COSV53040052 | NOTCH1 | 9:136517814-136517815 | c.1378_1379delinsTT | p.P460L |
| COSV53031860 | NOTCH1 | 9:136517814-136517814 | c.1379C>T | p.P460L |
| COSV105112480 | NOTCH1 | 9:136517811-136517811 | c.1382G>T | p.C461F |
| COSV53063613 | NOTCH1 | 9:136517811-136517811 | c.1382G>A | p.C461Y |
| COSV53098937 | NOTCH1 | 9:136517810-136517810 | c.1383C>G | p.C461W |
| COSV53031844 | NOTCH1 | 9:136517805-136517805 | c.1388A>G | p.N463S |
| COSV53038596 | NOTCH1 | 9:136517804-136517804 | c.1389C>T | p.N463= |
| COSV53030190 | NOTCH1 | 9:136517800-136517800 | c.1393G>A | p.A465T |
| COSV53051404 | NOTCH1 | 9:136517797-136517797 | c.1396A>G | p.T466A |
| COSV53063671 | NOTCH1 | 9:136517796-136517796 | c.1397C>T | p.T466I |
| COSV53030917 | NOTCH1 | 9:136517793-136517793 | c.1400G>T | p.C467F |
| COSV53042420 | NOTCH1 | 9:136517793-136517793 | c.1400G>A | p.C467Y |
| COSV105112820 | NOTCH1 | 9:136517789-136517789 | c.1404G>T | p.L468= |
| COSV53033401 | NOTCH1 | 9:136517788-136517788 | c.1405G>T | p.D469Y |
| COSV53042039 | NOTCH1 | 9:136517788-136517788 | c.1405G>A | p.D469N |
| COSV53076003 | NOTCH1 | 9:136517787-136517787 | c.1406A>G | p.D469G |
| COSV53106129 | NOTCH1 | 9:136517786-136517786 | c.1407C>G | p.D469E |
| COSV99493323 | NOTCH1 | 9:136517785-136517785 | c.1408C>T | p.Q470* |
| COSV53026237 | NOTCH1 | 9:136517785-136517785 | c.1408C>G | p.Q470E |
| COSV53028008 | NOTCH1 | 9:136517782-136517782 | c.1411A>T | p.I471F |
| COSV53046945 | NOTCH1 | 9:136517781-136517781 | c.1412T>C | p.I471T |
| COSV53031876 | NOTCH1 | 9:136517781-136517781 | c.1412T>A | p.I471N |
| COSV53045090 | NOTCH1 | 9:136517778-136517779 | c.1414_1415delinsAA | p.G472K |
| COSV53033055 | NOTCH1 | 9:136517778-136517778 | c.1415G>T | p.G472V |
| COSV53039134 | NOTCH1 | 9:136517778-136517778 | c.1415G>A | p.G472E |
| COSV53066272 | NOTCH1 | 9:136517777-136517777 | c.1416G>A | p.G472= |
| COSV99486411 | NOTCH1 | 9:136517773-136517773 | c.1420T>G | p.F474V |
| COSV53042637 | NOTCH1 | 9:136517771-136517771 | c.1422C>A | p.F474L |
| COSV99493281 | NOTCH1 | 9:136517767-136517767 | c.1426T>A | p.C476S |
| COSV53054868 | NOTCH1 | 9:136517766-136517766 | c.1427G>T | p.C476F |
| COSV53040234 | NOTCH1 | 9:136517760-136517760 | c.1433G>T | p.C478F |
| COSV53069801 | NOTCH1 | 9:136517760-136517760 | c.1433G>A | p.C478Y |
| COSV53042618 | NOTCH1 | 9:136517753-136517754 | c.1439_1440delinsTT | p.P480L |
| COSV53033864 | NOTCH1 | 9:136517752-136517752 | c.1441G>A | p.G481S |
| COSV53062730 | NOTCH1 | 9:136505880-136505880 | c.4016G>C | p.G1339A |
| COSV104374540 | NOTCH1 | 9:136505878-136505878 | c.4018T>G | p.F1340V |
| COSV53038404 | NOTCH1 | 9:136505876-136505876 | c.4020C>T | p.F1340= |
| COSV53095071 | NOTCH1 | 9:136505874-136505874 | c.4022A>G | p.E1341G |
| COSV53082167 | NOTCH1 | 9:136505868-136505868 | c.4028C>T | p.A1343V |
| COSV53044777 | NOTCH1 | 9:136505865-136505865 | c.4031C>T | p.T1344M |
| COSV53106537 | NOTCH1 | 9:136505860-136505860 | c.4036G>A | p.E1346K |
| COSV53095051 | NOTCH1 | 9:136505859-136505859 | c.4037A>G | p.E1346G |
| COSV53090789 | NOTCH1 | 9:136505858-136505858 | c.4038G>T | p.E1346D |
| COSV53080051 | NOTCH1 | 9:136505857-136505857 | c.4039A>G | p.N1347D |
| COSV53057468 | NOTCH1 | 9:136505851-136505851 | c.4045G>A | p.A1349T |
| COSV104586618 | NOTCH1 | 9:136505840-136505840 | c.4056C>T | p.C1352= |
| COSV53059192 | NOTCH1 | 9:136505830-136505830 | c.4066C>T | p.R1356C |
| COSV53058547 | NOTCH1 | 9:136505826-136505826 | c.4070G>A | p.C1357Y |
| COSV105112056 | NOTCH1 | 9:136505819-136505819 | c.4077C>T | p.N1359= |
| COSV53037865 | NOTCH1 | 9:136505818-136505818 | c.4078G>A | p.G1360S |
| COSV104586413 | NOTCH1 | 9:136505651-136505651 | c.4245G>A | p.L1415= |
| COSV53046015 | NOTCH1 | 9:136505650-136505650 | c.4246T>C | p.C1416R |
| COSV104586412 | NOTCH1 | 9:136505645-136505645 | c.4251C>T | p.P1417= |
| COSV53090665 | NOTCH1 | 9:136505644-136505644 | c.4252G>T | p.A1418S |
| COSV53075287 | NOTCH1 | 9:136505644-136505644 | c.4252G>A | p.A1418T |
| COSV53030799 | NOTCH1 | 9:136505641-136505641 | c.4255A>G | p.K1419E |
| COSV104586411 | NOTCH1 | 9:136505626-136505627 | c.4269_4270delinsAC | p.LL1423= |
| COSV53051121 | NOTCH1 | 9:136505625-136505625 | c.4271T>C | p.L1424S |
| COSV53103397 | NOTCH1 | 9:136505621-136505621 | c.4275C>G | p.C1425W |
| COSV105112181 | NOTCH1 | 9:136505615-136505615 | c.4281C>T | p.I1427= |
| COSV53089446 | NOTCH1 | 9:136505614-136505614 | c.4282C>T | p.L1428= |
| COSV53064436 | NOTCH1 | 9:136505604-136505611 | c.4285_4292delinsA | p.D1429Tfs*14 |
| COSV99483920 | NOTCH1 | 9:136505600-136505600 | c.4296C>T | p.F1432= |
| COSV53032088 | NOTCH1 | 9:136505599-136505599 | c.4297G>C | p.G1433R |
| COSV53063632 | NOTCH1 | 9:136505598-136505598 | c.4298G>A | p.G1433E |
| COSV104586410 | NOTCH1 | 9:136505597-136505599 | c.4297_4299delinsACA | p.G1433T |
| COSV53038876 | NOTCH1 | 9:136505597-136505597 | c.4299G>T | p.G1433= |
| COSV53049340 | NOTCH1 | 9:136505596-136505596 | c.4300G>T | p.G1434C |
| COSV53029905 | NOTCH1 | 9:136505595-136505595 | c.4301G>A | p.G1434D |
| COSV53049135 | NOTCH1 | 9:136505592-136505592 | c.4304G>T | p.G1435V |
| COSV104586409 | NOTCH1 | 9:136505591-136505591 | c.4305G>C | p.G1435= |
| COSV105112815 | NOTCH1 | 9:136505591-136505591 | c.4305G>A | p.G1435= |
| COSV104586408 | NOTCH1 | 9:136505588-136505588 | c.4308C>T | p.A1436= |
| COSV53042337 | NOTCH1 | 9:136505587-136505587 | c.4309G>A | p.G1437R |
| COSV53057451 | NOTCH1 | 9:136505583-136505583 | c.4313G>A | p.R1438H |
| COSV104586406 | NOTCH1 | 9:136505576-136505576 | c.4320C>T | p.I1440= |
| COSV53101711 | NOTCH1 | 9:136505575-136505575 | c.4321C>T | p.P1441S |
| COSV104586619 | NOTCH1 | 9:136505573-136505573 | c.4323C>T | p.P1441= |
| COSV53108939 | NOTCH1 | 9:136505572-136505572 | c.4324C>T | p.P1442S |
| COSV53045529 | NOTCH1 | 9:136505571-136505571 | c.4325C>T | p.P1442L |
| COSV53078391 | NOTCH1 | 9:136505570-136505570 | c.4326G>A | p.P1442= |
| COSV104586510 | NOTCH1 | 9:136505568-136505568 | c.4328C>T | p.P1443L |
| COSV53074835 | NOTCH1 | 9:136505565-136505565 | c.4331T>A | p.L1444Q |
| COSV104586405 | NOTCH1 | 9:136505561-136505561 | c.4335C>T | p.I1445= |
| COSV53103193 | NOTCH1 | 9:136505560-136505560 | c.4336G>T | p.E1446* |
| COSV53026218 | NOTCH1 | 9:136505560-136505560 | c.4336G>A | p.E1446K |
| COSV53061678 | NOTCH1 | 9:136505557-136505557 | c.4339G>A | p.E1447K |
| COSV99072426 | NOTCH1 | 9:136505555-136505555 | c.4341G>C | p.E1447D |
| COSV53033003 | NOTCH1 | 9:136505554-136505554 | c.4342G>A | p.A1448T |
| COSV53063706 | NOTCH1 | 9:136505553-136505553 | c.4343C>T | p.A1448V |
| COSV104586403 | NOTCH1 | 9:136505552-136505552 | c.4344G>C | p.A1448= |
| COSV53058956 | NOTCH1 | 9:136505550-136505550 | c.4346G>A | p.C1449Y |
| COSV99493158 | NOTCH1 | 9:136505549-136505549 | c.4347C>T | p.C1449= |
| COSV53087643 | NOTCH1 | 9:136505548-136505548 | c.4348G>A | p.E1450K |
| COSV53039788 | NOTCH1 | 9:136505543-136505543 | c.4353G>A | p.L1451= |
| COSV53027218 | NOTCH1 | 9:136505542-136505542 | c.4354C>T | p.P1452S |
| COSV53109824 | NOTCH1 | 9:136505541-136505541 | c.4355C>T | p.P1452L |
| COSV104586402 | NOTCH1 | 9:136505540-136505540 | c.4356C>T | p.P1452= |
| COSV53102019 | NOTCH1 | 9:136505539-136505539 | c.4357G>T | p.E1453* |
| COSV53071679 | NOTCH1 | 9:136505535-136505535 | c.4361G>T | p.C1454F |
| COSV53075468 | NOTCH1 | 9:136505533-136505533 | c.4363C>T | p.Q1455* |
| COSV53054141 | NOTCH1 | 9:136505532-136505532 | c.4364A>C | p.Q1455P |
| COSV104586401 | NOTCH1 | 9:136505529-136505529 | c.4367A>T | p.E1456V |
| COSV53103267 | NOTCH1 | 9:136505526-136505526 | c.4370A>G | p.D1457G |
| COSV53090512 | NOTCH1 | 9:136505525-136505525 | c.4371C>T | p.D1457= |
| COSV53107172 | NOTCH1 | 9:136505523-136505523 | c.4373C>T | p.A1458V |
| COSV53051082 | NOTCH1 | 9:136505523-136505523 | c.4373C>A | p.A1458E |
| COSV104586550 | NOTCH1 | 9:136505522-136505522 | c.4374G>T | p.A1458= |
| COSV104586400 | NOTCH1 | 9:136505522-136505522 | c.4374G>A | p.A1458= |
| COSV104586319 | NOTCH1 | 9:136505520-136505520 | c.4376G>A | p.G1459D |
| COSV104586399 | NOTCH1 | 9:136505516-136505516 | c.4380C>T | p.N1460= |
| COSV53095015 | NOTCH1 | 9:136505514-136505514 | c.4382A>G | p.K1461R |
| COSV53091156 | NOTCH1 | 9:136505505-136505505 | c.4391G>T | p.S1464I |
| COSV53096547 | NOTCH1 | 9:136505505-136505505 | c.4391G>A | p.S1464N |
| COSV104586398 | NOTCH1 | 9:136505495-136505495 | c.4401C>T | p.C1467= |
| COSV104586397 | NOTCH1 | 9:136505492-136505492 | c.4404C>T | p.N1468= |
| COSV104586396 | NOTCH1 | 9:136505489-136505489 | c.4407C>T | p.N1469= |
| COSV53081256 | NOTCH1 | 9:136505485-136505485 | c.4411G>A | p.A1471T |
| COSV105112729 | NOTCH1 | 9:136505484-136505484 | c.4412C>T | p.A1471V |
| COSV53066064 | NOTCH1 | 9:136505483-136505483 | c.4413G>A | p.A1471= |
| COSV104586395 | NOTCH1 | 9:136505480-136505480 | c.4416C>T | p.C1472= |
| COSV53096455 | NOTCH1 | 9:136505479-136505479 | c.4417G>T | p.G1473C |
| COSV53042314 | NOTCH1 | 9:136505479-136505479 | c.4417G>A | p.G1473S |
| COSV53026999 | NOTCH1 | 9:136505475-136505475 | c.4421G>A | p.W1474* |
| COSV99493707 | NOTCH1 | 9:136505474-136505474 | c.4422G>C | p.W1474C |
| COSV53076536 | NOTCH1 | 9:136505474-136505474 | c.4422G>A | p.W1474* |
| COSV53032798 | NOTCH1 | 9:136505472-136505472 | c.4424A>C | p.D1475A |
| COSV53054812 | NOTCH1 | 9:136505470-136505470 | c.4426G>A | p.G1476S |
| COSV105113250 | NOTCH1 | 9:136505468-136505468 | c.4428C>T | p.G1476= |
| COSV105823013 | NOTCH1 | 9:136505466-136505466 | c.4430G>T | p.G1477V |
| COSV53103241 | NOTCH1 | 9:136505461-136505461 | c.4435T>G | p.C1479G |
| COSV53103230 | NOTCH1 | 9:136505461-136505461 | c.4435T>C | p.C1479R |
| COSV99484676 | NOTCH1 | 9:136505450-136505450 | c.4446C>A | p.N1482K |
| COSV53042152 | NOTCH1 | 9:136505449-136505449 | c.4447T>C | p.F1483L |
| COSV53054701 | NOTCH1 | 9:136505446-136505446 | c.4450A>G | p.N1484D |
| COSV53081900 | NOTCH1 | 9:136505445-136505445 | c.4451A>G | p.N1484S |
| COSV53063688 | NOTCH1 | 9:136505443-136505443 | c.4453G>A | p.D1485N |
| COSV63879653 | MTOR | 1:11124626-11124626 | c.6534C>T | p.N2178= |
| COSV63868214 | MTOR | 1:11124608-11124608 | c.6552C>T | p.F2184= |
| COSV104421631 | MTOR | 1:11124607-11124607 | c.6553C>T | p.L2185F |
| COSV63878707 | MTOR | 1:11124592-11124592 | c.6568G>A | p.E2190K |
| COSV63877620 | MTOR | 1:11124589-11124589 | c.6571G>T | p.D2191Y |
| COSV63870516 | MTOR | 1:11124574-11124574 | c.6586G>T | p.E2196* |
| COSV63873702 | MTOR | 1:11124568-11124568 | c.6592G>T | p.V2198L |
| COSV63870550 | MTOR | 1:11124566-11124566 | c.6594G>C | p.V2198= |
| COSV100815724 | MTOR | 1:11124565-11124565 | c.6595A>G | p.M2199V |
| COSV63871134 | MTOR | 1:11124557-11124557 | c.6603C>G | p.L2201= |
| COSV63870285 | MTOR | 1:11124556-11124556 | c.6604T>C | p.F2202L |
| COSV63873138 | MTOR | 1:11124553-11124553 | c.6607G>A | p.G2203S |
| COSV63874670 | MTOR | 1:11124540-11124540 | c.6620C>T | p.T2207I |
| COSV105275752 | MTOR | 1:11124539-11124539 | c.6621C>T | p.T2207= |
| COSV63879465 | MTOR | 1:11124536-11124536 | c.6624T>C | p.L2208= |
| COSV63879045 | MTOR | 1:11124535-11124535 | c.6625C>T | p.L2209= |
| COSV63868849 | MTOR | 1:11124535-11124535 | c.6625C>G | p.L2209V |
| COSV63870065 | MTOR | 1:11124532-11124532 | c.6628G>C | p.A2210P |
| COSV105275634 | MTOR | 1:11124528-11124528 | c.6632A>T | p.N2211I |
| COSV99058146 | MTOR | 1:11124528-11124528 | c.6632A>G | p.N2211S |
| COSV63870254 | MTOR | 1:11124526-11124526 | c.6634G>T | p.D2212Y |
| COSV63871622 | MTOR | 1:11124517-11124517 | c.6643T>C | p.S2215P |
| COSV63868278 | MTOR | 1:11124516-11124516 | c.6644C>T | p.S2215F |
| COSV63868313 | MTOR | 1:11124516-11124516 | c.6644C>A | p.S2215Y |
| COSV63878553 | MTOR | 1:11124514-11124514 | c.6646C>A | p.L2216I |
| COSV63869468 | MTOR | 1:11124502-11124502 | c.6658C>T | p.L2220F |
| COSV52689110 | Tp53 | 17:7674971-7674971 | c.560G>T | p.G187V |
| COSV52661518 | Tp53 | 17:7674971-7674971 | c.560G>A | p.G187D |
| COSV52908827 | Tp53 | 17:7674970-7674970 | c.561T>C | p.G187= |
| COSV52787107 | Tp53 | 17:7674969-7674969 | c.562C>G | p.L188V |
| COSV52830689 | Tp53 | 17:7674969-7674969 | c.562C>A | p.L188M |
| COSV53331740 | Tp53 | 17:7674968-7674969 | c.562_563delinsTA | p.L188* |
| COSV53839074 | Tp53 | 17:7674968-7674968 | c.563T>A | p.L188Q |
| COSV53164619 | Tp53 | 17:7674966-7674966 | c.565G>T | p.A189S |
| COSV52875466 | Tp53 | 17:7674966-7674966 | c.565G>C | p.A189P |
| COSV52908808 | Tp53 | 17:7674966-7674966 | c.565G>A | p.A189T |
| COSV53050047 | Tp53 | 17:7674965-7674965 | c.566C>T | p.A189V |
| COSV52815388 | Tp53 | 17:7674965-7674965 | c.566C>G | p.A189G |
| COSV53757315 | Tp53 | 17:7674965-7674965 | c.566C>A | p.A189D |
| COSV52730721 | Tp53 | 17:7674964-7674964 | c.567C>T | p.A189= |
| COSV52843475 | Tp53 | 17:7674963-7674963 | c.568C>T | p.P190S |
| COSV52949919 | Tp53 | 17:7674963-7674963 | c.568C>G | p.P190A |
| COSV52672087 | Tp53 | 17:7674963-7674963 | c.568C>A | p.P190T |
| COSV52689701 | Tp53 | 17:7674962-7674963 | c.568_569delinsTT | p.P190F |
| COSV52851212 | Tp53 | 17:7674962-7674963 | c.568_569delinsAA | p.P190N |
| COSV52664064 | Tp53 | 17:7674962-7674962 | c.569C>T | p.P190L |
| COSV52987047 | Tp53 | 17:7674962-7674962 | c.569C>G | p.P190R |
| COSV53313892 | Tp53 | 17:7674962-7674962 | c.569C>A | p.P190H |
| COSV52772342 | Tp53 | 17:7674961-7674961 | c.570T>G | p.P190= |
| COSV53135048 | Tp53 | 17:7674960-7674960 | c.571C>T | p.P191S |
| COSV52814271 | Tp53 | 17:7674960-7674960 | c.571C>G | p.P191A |
| COSV52926316 | Tp53 | 17:7674960-7674960 | c.571C>A | p.P191T |
| C05V52729903 | Tp53 | 17:7674959-7674959 | c.572C>T | p.P191L |
| C05V53423237 | Tp53 | 17:7674959-7674959 | c.572C>G | p.P191R |
| C05V52875587 | Tp53 | 17:7674959-7674959 | c.572C>A | p.P191H |
| C05V53772983 | Tp53 | 17:7674958-7674958 | c.573T>A | p.P191= |
| COSV52660737 | Tp53 | 17:7674957-7674957 | c.574C>T | p.Q192* |
| COSV52978302 | Tp53 | 17:7674957-7674957 | c.574C>A | p.Q192K |
| COSV53416426 | Tp53 | 17:7674956-7674956 | c.575A>T | p.Q192L |
| COSV52839714 | Tp53 | 17:7674956-7674956 | c.575A>G | p.Q192R |
| COSV53020321 | Tp53 | 17:7674955-7674955 | c.576G>T | p.Q192H |
| COSV53577379 | Tp53 | 17:7674955-7674955 | c.576G>C | p.Q192H |
| COSV53164604 | Tp53 | 17:7674955-7674955 | c.576G>A | p.Q192= |
| COSV53132577 | Tp53 | 17:7674954-7674955 | c.576_577delinsTT | p.Q192_H193delinsHY |
| COSV104587961 | Tp53 | 17:7674954-7674955 | c.576_577delinsCT | p.Q192_H193delinsHY |
| COSV52688299 | Tp53 | 17:7674954-7674954 | c.577C>T | p.H193Y |
| COSV52675896 | Tp53 | 17:7674954-7674954 | c.577C>G | p.H193D |
| COSV52757395 | Tp53 | 17:7674954-7674954 | c.577C>A | p.H193N |
| COSV52663304 | Tp53 | 17:7674953-7674953 | c.578A>T | p.H193L |
| COSV52662414 | Tp53 | 17:7674953-7674953 | c.578A>G | p.H193R |
| COSV52681707 | Tp53 | 17:7674953-7674953 | c.578A>C | p.H193P |
| COSV53086641 | Tp53 | 17:7674952-7674952 | c.579T>C | p.H193= |
| COSV52924326 | Tp53 | 17:7674952-7674952 | c.579T>A | p.H193Q |
| COSV52677226 | Tp53 | 17:7674951-7674951 | c.580C>T | p.L194F |
| COSV52796536 | Tp53 | 17:7674951-7674951 | c.580C>G | p.L194V |
| COSV53623808 | Tp53 | 17:7674951-7674951 | c.580C>A | p.L194I |
| COSV52679257 | Tp53 | 17:7674950-7674950 | c.581T>G | p.L194R |
| COSV52663681 | Tp53 | 17:7674950-7674950 | c.581T>C | p.L194P |
| COSV52677924 | Tp53 | 17:7674950-7674950 | c.581T>A | p.L194H |
| COSV52772013 | Tp53 | 17:7674949-7674949 | c.582T>G | p.L194= |
| COSV53143214 | Tp53 | 17:7674949-7674949 | c.582T>C | p.L194= |
| COSV52864400 | Tp53 | 17:7674949-7674949 | c.582T>A | p.L194= |
| COSV52677568 | Tp53 | 17:7674948-7674948 | c.583A>T | p.I195F |
| COSV52809310 | Tp53 | 17:7674948-7674948 | c.583A>C | p.I195L |
| COSV52661172 | Tp53 | 17:7674947-7674947 | c.584T>G | p.I195S |
| COSV52664264 | Tp53 | 17:7674947-7674947 | c.584T>C | p.I195T |
| COSV52720899 | Tp53 | 17:7674947-7674947 | c.584T>A | p.I195N |
| COSV53510837 | Tp53 | 17:7674946-7674954 | c.577_585delinsGCCCCT | p.H193_I195delinsAP |
| COSV52905626 | Tp53 | 17:7674946-7674947 | c.584_585delinsAA | p.I195K |
| COSV53427713 | Tp53 | 17:7674946-7674946 | c.585C>G | p.I195M |
| COSV52701243 | Tp53 | 17:7674945-7674946 | c.585_586delinsTT | p.R196* |
| COSV52663748 | Tp53 | 17:7674945-7674945 | c.586C>T | p.R196* |
| COSV53128268 | Tp53 | 17:7674945-7674945 | c.586C>G | p.R196G |
| COSV53125285 | Tp53 | 17:7674945-7674945 | c.586C>A | p.R196= |
| COSV52667931 | Tp53 | 17:7674944-7674944 | c.587G>T | p.R196L |
| COSV52678297 | Tp53 | 17:7674944-7674944 | c.587G>C | p.R196P |
| COSV52674826 | Tp53 | 17:7674944-7674944 | c.587G>A | p.R196Q |
| COSV53312881 | Tp53 | 17:7674943-7674943 | c.588A>G | p.R196= |
| COSV53025311 | Tp53 | 17:7674943-7674943 | c.588A>C | p.R196= |
| COSV52927251 | Tp53 | 17:7674942-7674942 | c.589G>T | p.V197L |
| COSV53163944 | Tp53 | 17:7674942-7674942 | c.589G>C | p.V197L |
| COSV52711079 | Tp53 | 17:7674942-7674942 | c.589G>A | p.V197M |
| COSV53345657 | Tp53 | 17:7674941-7674942 | c.589_590delinsAG | p.V197R |
| COSV52661145 | Tp53 | 17:7674941-7674941 | c.590T>G | p.V197G |
| COSV53120183 | Tp53 | 17:7674941-7674941 | c.590T>C | p.V197A |
| COSV52810143 | Tp53 | 17:7674941-7674941 | c.590T>A | p.V197E |
| COSV52994800 | Tp53 | 17:7674940-7674940 | c.591G>A | p.V197= |
| COSV53572572 | Tp53 | 17:7674939-7674940 | c.591_592delinsTT | p.E198* |
| COSV52678088 | Tp53 | 17:7674939-7674939 | c.592G>T | p.E198* |
| COSV53175941 | Tp53 | 17:7674939-7674939 | c.592G>C | p.E198Q |
| COSV52771688 | Tp53 | 17:7674939-7674939 | c.592G>A | p.E198K |
| COSV104370336 | Tp53 | 17:7674938-7674938 | c.593A>T | p.E198V |
| COSV53349193 | Tp53 | 17:7674938-7674938 | c.593A>G | p.E198G |
| COSV52710769 | Tp53 | 17:7674936-7674936 | c.595G>T | p.G199* |
| COSV52760685 | Tp53 | 17:7674936-7674936 | c.595G>A | p.G199R |
| COSV53000529 | Tp53 | 17:7674935-7674936 | c.595_596delinsAC | p.G199T |
| COSV52679535 | Tp53 | 17:7674935-7674935 | c.596G>T | p.G199V |
| COSV53326529 | Tp53 | 17:7674935-7674935 | c.596G>C | p.G199A |
| COSV52661319 | Tp53 | 17:7674935-7674935 | c.596G>A | p.G199E |
| COSV53235388 | Tp53 | 17:7674934-7674934 | c.597A>G | p.G199= |
| COSV104370335 | Tp53 | 17:7674933-7674933 | c.598A>T | p.N200Y |
| COSV52688316 | Tp53 | 17:7674933-7674933 | c.598A>G | p.N200D |
| COSV52840642 | Tp53 | 17:7674932-7674932 | c.599A>T | p.N200I |
| COSV52677283 | Tp53 | 17:7674932-7674932 | c.599A>G | p.N200S |
| COSV53742336 | Tp53 | 17:7674932-7674932 | c.599A>C | p.N200T |
| COSV53168739 | Tp53 | 17:7674930-7674930 | c.601T>G | p.L201V |
| COSV53577228 | Tp53 | 17:7674930-7674930 | c.601T>C | p.L201= |
| COSV53313610 | Tp53 | 17:7674929-7674930 | c.601_602delinsCC | p.L201P |
| COSV53037685 | Tp53 | 17:7674929-7674929 | c.602T>C | p.L201S |
| COSV52837759 | Tp53 | 17:7674929-7674929 | c.602T>A | p.L201* |
| COSV52783767 | Tp53 | 17:7674928-7674928 | c.603G>T | p.L201F |
| COSV52676241 | Tp53 | 17:7674928-7674928 | c.603G>C | p.L201F |
| COSV52773550 | Tp53 | 17:7674927-7674928 | c.603_604delinsTT | p.L201_R202delinsFC |
| COSV52891929 | Tp53 | 17:7674927-7674927 | c.604C>T | p.R202C |
| COSV52950017 | Tp53 | 17:7674927-7674927 | c.604C>G | p.R202G |
| COSV52688630 | Tp53 | 17:7674927-7674927 | c.604C>A | p.R202S |
| COSV52700248 | Tp53 | 17:7674926-7674926 | c.605G>T | p.R202L |
| COSV53682901 | Tp53 | 17:7674926-7674926 | c.605G>C | p.R202P |
| COSV52677615 | Tp53 | 17:7674926-7674926 | c.605G>A | p.R202H |
| COSV52713441 | Tp53 | 17:7674925-7674926 | c.605_606delinsCG | p.R202P |
| COSV53578083 | Tp53 | 17:7674925-7674925 | c.606T>C | p.R202= |
| COSV52761693 | Tp53 | 17:7674924-7674924 | c.607G>T | p.V203L |
| COSV52965683 | Tp53 | 17:7674924-7674924 | c.607G>A | p.V203M |
| COSV52838983 | Tp53 | 17:7674923-7674923 | c.608T>C | p.V203A |
| COSV52661085 | Tp53 | 17:7674923-7674923 | c.608T>A | p.V203E |
| COSV52831648 | Tp53 | 17:7674922-7674922 | c.609G>T | p.V203= |
| COSV52909000 | Tp53 | 17:7674922-7674922 | c.609G>A | p.V203= |
| COSV53117470 | Tp53 | 17:7674921-7674922 | c.609_610delinsCT | p.E204* |
| COSV52679869 | Tp53 | 17:7674921-7674921 | c.610G>T | p.E204* |
| COSV53424574 | Tp53 | 17:7674921-7674921 | c.610G>C | p.E204Q |
| COSV53267121 | Tp53 | 17:7674921-7674921 | c.610G>A | p.E204K |
| COSV53008436 | Tp53 | 17:7674920-7674920 | c.611A>T | p.E204V |
| COSV53283399 | Tp53 | 17:7674920-7674920 | c.611A>G | p.E204G |
| COSV52878039 | Tp53 | 17:7674919-7674919 | c.612G>C | p.E204D |
| COSV52750941 | Tp53 | 17:7674919-7674919 | c.612G>A | p.E204= |
| COSV52760580 | Tp53 | 17:7674918-7674918 | c.613T>G | p.Y205D |
| COSV52806341 | Tp53 | 17:7674918-7674918 | c.613T>C | p.Y205H |
| COSV52877975 | Tp53 | 17:7674918-7674918 | c.613T>A | p.Y205N |
| COSV52688506 | Tp53 | 17:7674917-7674917 | c.614A>T | p.Y205F |
| COSV52665440 | Tp53 | 17:7674917-7674917 | c.614A>G | p.Y205C |
| COSV52677268 | Tp53 | 17:7674917-7674917 | c.614A>C | p.Y205S |
| COSV52808763 | Tp53 | 17:7674916-7674916 | c.615T>G | p.Y205* |
| COSV52840738 | Tp53 | 17:7674916-7674916 | c.615T>A | p.Y205* |
| COSV52853973 | Tp53 | 17:7674914-7674914 | c.617T>A | p.L206* |
| COSV53438297 | Tp53 | 17:7674913-7674913 | c.618G>C | p.L206F |
| COSV53153155 | Tp53 | 17:7674913-7674913 | c.618G>A | p.L206= |
| COSV52839530 | Tp53 | 17:7674912-7674912 | c.619G>T | p.D207Y |
| COSV52713825 | Tp53 | 17:7674911-7674911 | c.620A>G | p.D207G |
| COSV52677254 | Tp53 | 17:7674911-7674911 | c.620A>C | p.D207A |
| COSV52808981 | Tp53 | 17:7674910-7674910 | c.621T>C | p.D207= |
| COSV52925956 | Tp53 | 17:7674910-7674910 | c.621T>A | p.D207E |
| COSV52919029 | Tp53 | 17:7674909-7674909 | c.622G>T | p.D208Y |
| COSV53587568 | Tp53 | 17:7674909-7674909 | c.622G>C | p.D208H |
| COSV53016463 | Tp53 | 17:7674909-7674909 | c.622G>A | p.D208N |
| COSV53097846 | Tp53 | 17:7674908-7674909 | c.622_623delinsAT | p.D208I |
| COSV52685550 | Tp53 | 17:7674908-7674908 | c.623A>T | p.D208V |
| COSV52744439 | Tp53 | 17:7674908-7674908 | c.623A>G | p.D208G |
| COSV53327097 | Tp53 | 17:7674907-7674907 | c.624C>G | p.D208E |
| COSV52761304 | Tp53 | 17:7674907-7674907 | c.624C>A | p.D208E |
| COSV52741360 | Tp53 | 17:7674906-7674906 | c.625A>T | p.R209* |
| COSV53599366 | Tp53 | 17:7674906-7674906 | c.625A>G | p.R209G |
| COSV52840233 | Tp53 | 17:7674906-7674906 | c.625A>C | p.R209= |
| COSV52680014 | Tp53 | 17:7674905-7674905 | c.626G>T | p.R209I |
| COSV52741118 | Tp53 | 17:7674905-7674905 | c.626G>C | p.R209T |
| COSV52987587 | Tp53 | 17:7674905-7674905 | c.626G>A | p.R209K |
| COSV53153688 | Tp53 | 17:7674904-7674904 | c.627A>T | p.R209S |
| COSV52660880 | Tp53 | 17:7674903-7674903 | c.628A>G | p.N210D |
| COSV53683227 | Tp53 | 17:7674903-7674903 | c.628A>C | p.N210H |
| COSV53339346 | Tp53 | 17:7674902-7674902 | c.629A>G | p.N210S |
| COSV52979868 | Tp53 | 17:7674902-7674902 | c.629A>C | p.N210T |
| COSV52937432 | Tp53 | 17:7674901-7674901 | c.630C>T | p.N210= |
| COSV53588238 | Tp53 | 17:7674901-7674901 | c.630C>G | p.N210K |
| COSV52795736 | Tp53 | 17:7674900-7674900 | c.631A>G | p.T211A |
| COSV52688982 | Tp53 | 17:7674900-7674900 | c.631A>C | p.T211P |
| COSV52665474 | Tp53 | 17:7674899-7674899 | c.632C>T | p.T211I |
| COSV52751752 | Tp53 | 17:7674899-7674899 | c.632C>G | p.T2115 |
| C05V52987532 | Tp53 | 17:7674899-7674899 | c.632C>A | p.T211N |
| C05V52730558 | Tp53 | 17:7674898-7674898 | c.633T>C | p.T211= |
| C05V52978317 | Tp53 | 17:7674898-7674898 | c.633T>A | p.T211= |
| C05V52746659 | Tp53 | 17:7674897-7674897 | c.634T>G | p.F212V |
| COSV53210373 | Tp53 | 17:7674897-7674897 | c.634T>C | p.F212L |
| C05V52729961 | Tp53 | 17:7674897-7674897 | c.634T>A | p.F2121 |
| C05V53362733 | Tp53 | 17:7674896-7674896 | c.635T>C | p.F212S |
| C05V53438998 | Tp53 | 17:7674896-7674896 | c.635T>A | p.F212Y |
| C05V52729299 | Tp53 | 17:7674895-7674895 | c.636T>A | p.F212L |
| C05V52665560 | Tp53 | 17:7674894-7674894 | c.637C>T | p.R213* |
| C05V52782151 | Tp53 | 17:7674894-7674894 | c.637C>G | p.R213G |
| C05V52740638 | Tp53 | 17:7674894-7674894 | c.637C>A | p.R213= |
| C05V52739752 | Tp53 | 17:7674893-7674894 | c.637_638delinsT | p.R213Yfs*34 |
| C05V52668963 | Tp53 | 17:7674893-7674893 | c.638G>T | p.R213L |
| C05V52693072 | Tp53 | 17:7674893-7674893 | c.638G>C | p.R213P |
| C05V52665093 | Tp53 | 17:7674893-7674893 | c.638G>A | p.R213Q |
| C05V53387229 | Tp53 | 17:7674892-7674894 | c.637_639delinsTGG | p.R213W |
| C05V99386904 | Tp53 | 17:7674892-7674893 | c.638_639delinsAG | p.R213Q |
| C05V52679610 | Tp53 | 17:7674892-7674892 | c.639A>G | p.R213= |
| C05V52978286 | Tp53 | 17:7674891-7674891 | c.640C>T | p.H214Y |
| C05V52851768 | Tp53 | 17:7674891-7674891 | c.640C>G | p.H214D |
| C05V52834506 | Tp53 | 17:7674890-7674890 | c.641A>T | p.H214L |
| C05V52670202 | Tp53 | 17:7674890-7674890 | c.641A>G | p.H214R |
| C05V52732998 | Tp53 | 17:7674890-7674890 | c.641A>C | p.H214P |
| COSV52806263 | Tp53 | 17:7674889-7674889 | c.642T>G | p.H214Q |
| COSV52926654 | Tp53 | 17:7674889-7674889 | c.642T>C | p.H214= |
| COSV53527694 | Tp53 | 17:7674889-7674889 | c.642T>A | p.H214Q |
| COSV52689203 | Tp53 | 17:7674888-7674888 | c.643A>T | p.S215C |
| COSV52675946 | Tp53 | 17:7674888-7674888 | c.643A>G | p.S215G |
| COSV52876251 | Tp53 | 17:7674888-7674888 | c.643A>C | p.S215R |
| COSV53044231 | Tp53 | 17:7674887-7674889 | c.642_644delinsAAA | p.H214_S215delinsQN |
| COSV52679681 | Tp53 | 17:7674887-7674887 | c.644G>T | p.S215I |
| COSV52700292 | Tp53 | 17:7674887-7674887 | c.644G>C | p.S215T |
| COSV52686793 | Tp53 | 17:7674887-7674887 | c.644G>A | p.S215N |
| COSV52783847 | Tp53 | 17:7674886-7674886 | c.645T>G | p.S215R |
| COSV52752255 | Tp53 | 17:7674886-7674886 | c.645T>C | p.S215= |
| COSV52798196 | Tp53 | 17:7674886-7674886 | c.645T>A | p.S215R |
| COSV52751864 | Tp53 | 17:7674885-7674885 | c.646G>T | p.V216L |
| COSV53384298 | Tp53 | 17:7674885-7674885 | c.646G>C | p.V216L |
| COSV52671096 | Tp53 | 17:7674885-7674885 | c.646G>A | p.V216M |
| COSV52866969 | Tp53 | 17:7674884-7674884 | c.647T>G | p.V216G |
| COSV52856938 | Tp53 | 17:7674884-7674884 | c.647T>C | p.V216A |
| COSV52676908 | Tp53 | 17:7674884-7674884 | c.647T>A | p.V216E |
| COSV53163407 | Tp53 | 17:7674882-7674882 | c.649G>T | p.V217L |
| COSV53713958 | Tp53 | 17:7674882-7674882 | c.649G>A | p.V217M |
| COSV52675866 | Tp53 | 17:7674881-7674881 | c.650T>G | p.V217G |
| COSV53142750 | Tp53 | 17:7674881-7674881 | c.650T>C | p.V217A |
| COSV53037841 | Tp53 | 17:7674881-7674881 | c.650T>A | p.V217E |
| COSV53387338 | Tp53 | 17:7674880-7674880 | c.651G>A | p.V217= |
| COSV52993305 | Tp53 | 17:7674879-7674880 | c.651_652delinsAA | p.V218M |
| COSV53209108 | Tp53 | 17:7674879-7674879 | c.652G>T | p.V218L |
| COSV52795822 | Tp53 | 17:7674879-7674879 | c.652G>A | p.V218M |
| COSV52690974 | Tp53 | 17:7674878-7674878 | c.653T>G | p.V218G |
| COSV52761003 | Tp53 | 17:7674878-7674878 | c.653T>C | p.V218A |
| COSV52712839 | Tp53 | 17:7674878-7674878 | c.653T>A | p.V218E |
| COSV53387326 | Tp53 | 17:7674877-7674877 | c.654G>A | p.V218= |
| COSV52839958 | Tp53 | 17:7674876-7674876 | c.655C>T | p.P219S |
| COSV53438329 | Tp53 | 17:7674876-7674876 | c.655C>A | p.P219T |
| COSV52965264 | Tp53 | 17:7674875-7674876 | c.655_656delinsTG | p.P219C |
| COSV52787654 | Tp53 | 17:7674875-7674875 | c.656C>T | p.P219L |
| COSV53243996 | Tp53 | 17:7674875-7674875 | c.656C>G | p.P219R |
| COSV53017313 | Tp53 | 17:7674875-7674875 | c.656C>A | p.P219H |
| COSV52926388 | Tp53 | 17:7674874-7674875 | c.656_657delinsTT | p.P219L |
| COSV52757494 | Tp53 | 17:7674874-7674875 | c.656_657delinsT | p.P219Lfs*28 |
| COSV52965002 | Tp53 | 17:7674874-7674874 | c.657C>T | p.P219= |
| COSV53107694 | Tp53 | 17:7674874-7674874 | c.657C>G | p.P219= |
| COSV53515706 | Tp53 | 17:7674874-7674874 | c.657C>A | p.P219= |
| COSV52691656 | Tp53 | 17:7674873-7674873 | c.658T>G | p.Y220D |
| COSV52760651 | Tp53 | 17:7674873-7674873 | c.658T>C | p.Y220H |
| COSV52700454 | Tp53 | 17:7674873-7674873 | c.658T>A | p.Y220N |
| COSV52661282 | Tp53 | 17:7674872-7674872 | c.659A>G | p.Y220C |
| COSV52677517 | Tp53 | 17:7674872-7674872 | c.659A>C | p.Y220S |
| COSV52775381 | Tp53 | 17:7674871-7674871 | c.660T>G | p.Y220* |
| COSV53289049 | Tp53 | 17:7674871-7674871 | c.660T>A | p.Y220* |
| COSV52735606 | Tp53 | 17:7674870-7674870 | c.661G>T | p.E221* |
| COSV52783951 | Tp53 | 17:7674870-7674870 | c.661G>A | p.E221K |
| COSV53268129 | Tp53 | 17:7674869-7674869 | c.662A>G | p.E221G |
| COSV53625223 | Tp53 | 17:7674868-7674868 | c.663G>T | p.E221D |
| COSV52740414 | Tp53 | 17:7674868-7674868 | c.663G>C | p.E221D |
| COSV53106992 | Tp53 | 17:7674868-7674868 | c.663G>A | p.E221= |
| COSV52839398 | Tp53 | 17:7674867-7674867 | c.664C>T | p.P222S |
| COSV52774086 | Tp53 | 17:7674867-7674867 | c.664C>G | p.P222A |
| COSV53220372 | Tp53 | 17:7674867-7674867 | c.664C>A | p.P222T |
| COSV53526100 | Tp53 | 17:7674866-7674867 | c.664_665delinsTT | p.P222L |
| COSV52909202 | Tp53 | 17:7674866-7674866 | c.665C>T | p.P222L |
| COSV52774327 | Tp53 | 17:7674866-7674866 | c.665C>A | p.P222Q |
| COSV53267642 | Tp53 | 17:7674865-7674865 | c.666G>T | p.P222= |
| COSV52826471 | Tp53 | 17:7674865-7674865 | c.666G>C | p.P222= |
| COSV53563768 | Tp53 | 17:7674865-7674865 | c.666G>A | p.P222= |
| COSV52733140 | Tp53 | 17:7674864-7674864 | c.667C>T | p.P223S |
| COSV53486511 | Tp53 | 17:7674864-7674864 | c.667C>G | p.P223A |
| COSV105033206 | Tp53 | 17:7674863-7674878 | c.653_668delinsG | p.V218_P223delinsG |
| COSV52728555 | Tp53 | 17:7674863-7674863 | c.668C>T | p.P223L |
| COSV52699245 | Tp53 | 17:7674863-7674863 | c.668C>G | p.P223R |
| COSV53251283 | Tp53 | 17:7674863-7674863 | c.668C>A | p.P223H |
| COSV52978406 | Tp53 | 17:7674862-7674862 | c.669T>G | p.P223= |
| COSV52978457 | Tp53 | 17:7674862-7674862 | c.669T>C | p.P223= |
| COSV53685625 | Tp53 | 17:7674862-7674862 | c.669T>A | p.P223= |
| COSV52765100 | Tp53 | 17:7674861-7674861 | c.670G>T | p.E224* |
| COSV53452066 | Tp53 | 17:7674861-7674861 | c.670G>C | p.E224Q |
| COSV52760587 | Tp53 | 17:7674861-7674861 | c.670G>A | p.E224K |
| COSV53220560 | Tp53 | 17:7674860-7674860 | c.671delinsTCT | p.E224Vfs*24 |
| COSV52667177 | Tp53 | 17:7674860-7674860 | c.671A>T | p.E224V |
| COSV53233165 | Tp53 | 17:7674860-7674860 | c.671A>G | p.E224G |
| COSV52678333 | Tp53 | 17:7674859-7674859 | c.672G>T | p.E224D |
| COSV52721244 | Tp53 | 17:7674859-7674859 | c.672G>C | p.E224D |
| COSV52681634 | Tp53 | 17:7674859-7674859 | c.672G>A | p.E224= |
| COSV52863714 | Tp53 | 17:7674290-7674290 | c.673G>T | p.V225F |
| COSV52950260 | Tp53 | 17:7674290-7674290 | c.673G>A | p.V225I |
| COSV53402053 | Tp53 | 17:7674289-7674289 | c.674T>G | p.V225G |
| COSV52937679 | Tp53 | 17:7674289-7674289 | c.674T>C | p.V225A |
| COSV52808287 | Tp53 | 17:7674289-7674289 | c.674T>A | p.V225D |
| COSV52891821 | Tp53 | 17:7674288-7674288 | c.675T>C | p.V225= |
| COSV52674807 | Tp53 | 17:7674287-7674287 | c.676G>A | p.G226S |
| COSV52965428 | Tp53 | 17:7674286-7674286 | c.677G>T | p.G226V |
| COSV53510385 | Tp53 | 17:7674286-7674286 | c.677G>C | p.G226A |
| COSV52797216 | Tp53 | 17:7674286-7674286 | c.677G>A | p.G226D |
| COSV52688852 | Tp53 | 17:7674285-7674285 | c.678C>T | p.G226= |
| COSV52994759 | Tp53 | 17:7674285-7674285 | c.678C>A | p.G226= |
| COSV105023817 | Tp53 | 17:7674284-7674284 | c.679T>C | p.S227P |
| COSV52661835 | Tp53 | 17:7674283-7674283 | c.680C>T | p.S227F |
| COSV53410104 | Tp53 | 17:7674283-7674283 | c.680C>A | p.S227Y |
| COSV53096994 | Tp53 | 17:7674282-7674282 | c.681T>G | p.S227= |
| COSV53684875 | Tp53 | 17:7674282-7674282 | c.681T>C | p.S227= |
| COSV52809847 | Tp53 | 17:7674282-7674282 | c.681T>A | p.S227= |
| COSV52663871 | Tp53 | 17:7674281-7674281 | c.682G>T | p.D228Y |
| COSV53160330 | Tp53 | 17:7674281-7674281 | c.682G>C | p.D228H |
| COSV53107584 | Tp53 | 17:7674281-7674281 | c.682G>A | p.D228N |
| COSV52751913 | Tp53 | 17:7674280-7674280 | c.683A>T | p.D228V |
| COSV52701795 | Tp53 | 17:7674280-7674280 | c.683A>G | p.D228G |
| COSV53311916 | Tp53 | 17:7674280-7674280 | c.683A>C | p.D228A |
| COSV52864128 | Tp53 | 17:7674279-7674279 | c.684C>T | p.D228= |
| COSV52662428 | Tp53 | 17:7674279-7674279 | c.684C>G | p.D228E |
| COSV53050371 | Tp53 | 17:7674278-7674278 | c.685T>G | p.C229G |
| COSV52741302 | Tp53 | 17:7674278-7674278 | c.685T>C | p.C229R |
| COSV52662163 | Tp53 | 17:7674278-7674278 | c.685T>A | p.C229S |
| COSV52816928 | Tp53 | 17:7674277-7674277 | c.686G>T | p.C229F |
| COSV53136088 | Tp53 | 17:7674277-7674277 | c.686G>C | p.C229S |
| COSV52670028 | Tp53 | 17:7674277-7674277 | c.686G>A | p.C229Y |
| COSV53757768 | Tp53 | 17:7674276-7674276 | c.687T>C | p.C229= |
| COSV52903842 | Tp53 | 17:7674276-7674276 | c.687T>A | p.C229* |
| COSV52783174 | Tp53 | 17:7674275-7674275 | c.688A>T | p.T230S |
| COSV52787639 | Tp53 | 17:7674275-7674275 | c.688A>G | p.T230A |
| COSV52678462 | Tp53 | 17:7674275-7674275 | c.688A>C | p.T230P |
| COSV52756528 | Tp53 | 17:7674274-7674274 | c.689C>T | p.T230I |
| COSV52740649 | Tp53 | 17:7674274-7674274 | c.689C>A | p.T230N |
| COSV53715467 | Tp53 | 17:7674273-7674273 | c.690C>A | p.T230= |
| COSV99391357 | Tp53 | 17:7674272-7674274 | c.689_691delinsACG | p.T230_T231delinsNA |
| COSV52760462 | Tp53 | 17:7674272-7674272 | c.691A>T | p.T231S |
| COSV52826742 | Tp53 | 17:7674272-7674272 | c.691A>G | p.T231A |
| COSV53214710 | Tp53 | 17:7674272-7674272 | c.691A>C | p.T231P |
| COSV53283457 | Tp53 | 17:7674271-7674271 | c.692C>T | p.T231I |
| COSV52729626 | Tp53 | 17:7674271-7674271 | c.692C>A | p.T231N |
| COSV52775800 | Tp53 | 17:7674270-7674270 | c.693C>T | p.T231= |
| COSV52880748 | Tp53 | 17:7674270-7674270 | c.693C>A | p.T231= |
| COSV52704131 | Tp53 | 17:7674269-7674269 | c.694A>T | p.I232F |
| COSV52677046 | Tp53 | 17:7674269-7674269 | c.694A>G | p.I232V |
| COSV53187960 | Tp53 | 17:7674269-7674269 | c.694A>C | p.I232L |
| COSV52829354 | Tp53 | 17:7674268-7674268 | c.695T>G | p.I232S |
| COSV52661257 | Tp53 | 17:7674268-7674268 | c.695T>C | p.I232T |
| COSV52760307 | Tp53 | 17:7674268-7674268 | c.695T>A | p.I232N |
| COSV105030456 | Tp53 | 17:7674267-7674267 | c.696C>T | p.I232= |
| COSV53649861 | Tp53 | 17:7674267-7674267 | c.696C>A | p.I232= |
| COSV52751346 | Tp53 | 17:7674266-7674266 | c.697C>T | p.H233Y |
| COSV53109205 | Tp53 | 17:7674266-7674266 | c.697C>G | p.H233D |
| COSV53087526 | Tp53 | 17:7674265-7674265 | c.698A>T | p.H233L |
| COSV53458339 | Tp53 | 17:7674265-7674265 | c.698A>G | p.H233R |
| COSV53273483 | Tp53 | 17:7674265-7674265 | c.698A>C | p.H233P |
| COSV52850817 | Tp53 | 17:7674264-7674264 | c.699C>G | p.H233Q |
| COSV52730255 | Tp53 | 17:7674263-7674263 | c.700T>G | p.Y234D |
| COSV52694920 | Tp53 | 17:7674263-7674263 | c.700T>C | p.Y234H |
| COSV52730114 | Tp53 | 17:7674263-7674263 | c.700T>A | p.Y234N |
| COSV53538355 | Tp53 | 17:7674262-7674263 | c.700_70ldelinsCG | p.Y234R |
| COSV53142556 | Tp53 | 17:7674262-7674262 | c.701A>T | p.Y234F |
| COSV52661201 | Tp53 | 17:7674262-7674262 | c.701A>G | p.Y234C |
| COSV52686167 | Tp53 | 17:7674262-7674262 | c.701A>C | p.Y234S |
| COSV53124617 | Tp53 | 17:7674261-7674261 | c.702C>T | p.Y234= |
| COSV52908090 | Tp53 | 17:7674261-7674261 | c.702C>G | p.Y234* |
| COSV52783916 | Tp53 | 17:7674261-7674261 | c.702C>A | p.Y234* |
| COSV52980321 | Tp53 | 17:7674260-7674260 | c.703A>T | p.N235Y |
| COSV52678348 | Tp53 | 17:7674260-7674260 | c.703A>G | p.N235D |
| COSV53037756 | Tp53 | 17:7674260-7674260 | c.703A>C | p.N235H |
| COSV52987012 | Tp53 | 17:7674259-7674259 | c.704A>T | p.N235I |
| COSV52700907 | Tp53 | 17:7674259-7674259 | c.704A>G | p.N235S |
| COSV52979785 | Tp53 | 17:7674259-7674259 | c.704A>C | p.N235T |
| COSV53685564 | Tp53 | 17:7674258-7674259 | c.704_705delinsTG | p.N235M |
| COSV52672888 | Tp53 | 17:7674257-7674257 | c.706T>G | p.Y236D |
| COSV52783032 | Tp53 | 17:7674257-7674257 | c.706T>C | p.Y236H |
| COSV52675514 | Tp53 | 17:7674257-7674257 | c.706T>A | p.Y236N |
| COSV52662150 | Tp53 | 17:7674256-7674256 | c.707A>G | p.Y236C |
| COSV52735723 | Tp53 | 17:7674256-7674256 | c.707A>C | p.Y236S |
| COSV105032296 | Tp53 | 17:7674255-7674256 | c.707_708delinsTA | p.Y236L |
| COSV105024167 | Tp53 | 17:7674255-7674256 | c.707_708delinsCT | p.Y236S |
| COSV53025106 | Tp53 | 17:7674255-7674255 | c.708C>T | p.Y236= |
| COSV52713084 | Tp53 | 17:7674255-7674255 | c.708C>G | p.Y236* |
| COSV52728511 | Tp53 | 17:7674255-7674255 | c.708C>A | p.Y236* |
| COSV53325802 | Tp53 | 17:7674254-7674255 | c.708_709delinsAT | p.Y236_M237delins* |
| COSV52875518 | Tp53 | 17:7674254-7674254 | c.709A>T | p.M237L |
| COSV52701833 | Tp53 | 17:7674254-7674254 | c.709A>G | p.M237V |
| COSV53037741 | Tp53 | 17:7674253-7674253 | c.710T>G | p.M237R |
| COSV53037559 | Tp53 | 17:7674253-7674253 | c.710T>C | p.M237T |
| COSV52682586 | Tp53 | 17:7674253-7674253 | c.710T>A | p.M237K |
| COSV52681050 | Tp53 | 17:7674252-7674252 | c.711G>T | p.M237I |
| COSV52751406 | Tp53 | 17:7674252-7674252 | c.711G>C | p.M237I |
| COSV52661887 | Tp53 | 17:7674252-7674252 | c.711G>A | p.M237I |
| COSV52711932 | Tp53 | 17:7674251-7674251 | c.712T>G | p.C238G |
| COSV52707471 | Tp53 | 17:7674251-7674251 | c.712T>C | p.C238R |
| COSV52699956 | Tp53 | 17:7674251-7674251 | c.712T>A | p.C238S |
| COSV52706816 | Tp53 | 17:7674250-7674250 | c.713G>T | p.C238F |
| COSV52804264 | Tp53 | 17:7674250-7674250 | c.713G>C | p.C238S |
| COSV52661646 | Tp53 | 17:7674250-7674250 | c.713G>A | p.C238Y |
| COSV52680696 | Tp53 | 17:7674249-7674249 | c.714T>G | p.C238W |
| COSV53234319 | Tp53 | 17:7674249-7674249 | c.714T>C | p.C238= |
| COSV52840491 | Tp53 | 17:7674249-7674249 | c.714T>A | p.C238* |
| COSV99392595 | Tp53 | 17:7674248-7674249 | c.714_715delinsGG | p.C238_N239delinsWD |
| COSV52979974 | Tp53 | 17:7674248-7674248 | c.715A>T | p.N239Y |
| COSV52664075 | Tp53 | 17:7674248-7674248 | c.715A>G | p.N239D |
| COSV53345856 | Tp53 | 17:7674248-7674248 | c.715A>C | p.N239H |
| COSV52966384 | Tp53 | 17:7674247-7674247 | c.716A>T | p.N239I |
| COSV52661127 | Tp53 | 17:7674247-7674247 | c.716A>G | p.N239S |
| COSV52664176 | Tp53 | 17:7674247-7674247 | c.716A>C | p.N239T |
| COSV52994467 | Tp53 | 17:7674246-7674248 | c.715_717delinsTAA | p.N239* |
| COSV53728610 | Tp53 | 17:7674246-7674248 | c.715_717delinsT | p.N239* |
| COSV52752124 | Tp53 | 17:7674246-7674246 | c.717C>T | p.N239= |
| COSV52877093 | Tp53 | 17:7674246-7674246 | c.717C>G | p.N239K |
| COSV52983691 | Tp53 | 17:7674246-7674246 | c.717C>A | p.N239K |
| COSV53535661 | Tp53 | 17:7674245-7674248 | c.715_718delinsT | p.N239_S240delinsC |
| COSV53328299 | Tp53 | 17:7674245-7674245 | c.718delinsTGTTCCT | p.S240delinsCSC |
| COSV53263148 | Tp53 | 17:7674245-7674245 | c.718A>T | p.S240C |
| COSV52677032 | Tp53 | 17:7674245-7674245 | c.718A>G | p.S240G |
| COSV53415278 | Tp53 | 17:7674245-7674245 | c.718A>C | p.S240R |
| COSV52673339 | Tp53 | 17:7674244-7674245 | c.718_719delinsTT | p.S240F |
| COSV52783089 | Tp53 | 17:7674244-7674244 | c.719G>T | p.S240I |
| COSV52979072 | Tp53 | 17:7674244-7674244 | c.719G>C | p.S240T |
| COSV53233898 | Tp53 | 17:7674243-7674245 | c.718_720delinsCCC | p.S240P |
| COSV52795438 | Tp53 | 17:7674243-7674243 | c.720T>G | p.S240R |
| COSV53135249 | Tp53 | 17:7674243-7674243 | c.720T>C | p.S240= |
| COSV52783209 | Tp53 | 17:7674243-7674243 | c.720T>A | p.S240R |
| COSV52675302 | Tp53 | 17:7674242-7674242 | c.721T>G | p.S241A |
| COSV52670786 | Tp53 | 17:7674242-7674242 | c.721T>C | p.S241P |
| COSV53004514 | Tp53 | 17:7674242-7674242 | c.721T>A | p.S241T |
| COSV52860261 | Tp53 | 17:7674241-7674253 | c.710_722delinsCCA | p.M237Tfs*7 |
| COSV52661688 | Tp53 | 17:7674241-7674241 | c.722C>T | p.S241F |
| COSV52662386 | Tp53 | 17:7674241-7674241 | c.722C>G | p.S241C |
| COSV52713934 | Tp53 | 17:7674241-7674241 | c.722C>A | p.S241Y |
| COSV52761074 | Tp53 | 17:7674240-7674241 | c.722_723delinsTT | p.S241F |
| COSV53211006 | Tp53 | 17:7674240-7674240 | c.723C>T | p.S241= |
| COSV53153033 | Tp53 | 17:7674240-7674240 | c.723C>G | p.S241= |
| COSV53025058 | Tp53 | 17:7674240-7674240 | c.723C>A | p.S241= |
| COSV52760397 | Tp53 | 17:7674239-7674239 | c.724T>G | p.C242G |
| COSV52660956 | Tp53 | 17:7674239-7674239 | c.724T>C | p.C242R |
| COSV52677021 | Tp53 | 17:7674239-7674239 | c.724T>A | p.C242S |
| COSV52677418 | Tp53 | 17:7674238-7674238 | c.725G>T | p.C242F |
| COSV52689710 | Tp53 | 17:7674238-7674238 | c.725G>C | p.C242S |
| COSV52661189 | Tp53 | 17:7674238-7674238 | c.725G>A | p.C242Y |
| COSV53067450 | Tp53 | 17:7674237-7674238 | c.725_726delinsTT | p.C242F |
| COSV53692793 | Tp53 | 17:7674237-7674238 | c.725_726delinsCG | p.C242S |
| COSV52677195 | Tp53 | 17:7674237-7674237 | c.726C>T | p.C242= |
| COSV52704436 | Tp53 | 17:7674237-7674237 | c.726C>G | p.C242W |
| COSV53034631 | Tp53 | 17:7674237-7674237 | c.726C>A | p.C242* |
| COSV53383240 | Tp53 | 17:7674236-7674237 | c.726_727delinsGT | p.C242_M243delinsWL |
| COSV52713381 | Tp53 | 17:7674236-7674236 | c.727A>T | p.M243L |
| COSV52851538 | Tp53 | 17:7674236-7674236 | c.727A>G | p.M243V |
| COSV52699676 | Tp53 | 17:7674236-7674236 | c.727A>C | p.M243L |
| COSV52815103 | Tp53 | 17:7674235-7674235 | c.728T>G | p.M243R |
| COSV52676629 | Tp53 | 17:7674235-7674235 | c.728T>C | p.M243T |
| COSV53135089 | Tp53 | 17:7674235-7674235 | c.728T>A | p.M243K |
| COSV105017908 | Tp53 | 17:7674234-7674234 | c.729G>T | p.M243I |
| COSV52840414 | Tp53 | 17:7674234-7674234 | c.729G>C | p.M243I |
| COSV52668406 | Tp53 | 17:7674234-7674234 | c.729G>A | p.M243I |
| COSV52954801 | Tp53 | 17:7674233-7674234 | c.729_730delinsTT | p.M243_G244delinsIC |
| COSV53535424 | Tp53 | 17:7674233-7674234 | c.729_730delinsAA | p.M243_G244delinsIS |
| COSV52668392 | Tp53 | 17:7674233-7674233 | c.730G>T | p.G244C |
| COSV52808251 | Tp53 | 17:7674233-7674233 | c.730G>C | p.G244R |
| COSV52676997 | Tp53 | 17:7674233-7674233 | c.730G>A | p.G244S |
| COSV53679324 | Tp53 | 17:7674232-7674233 | c.730_731delinsTT | p.G244F |
| COSV52724858 | Tp53 | 17:7674232-7674232 | c.731G>T | p.G244V |
| COSV52839584 | Tp53 | 17:7674232-7674232 | c.731G>C | p.G244A |
| COSV52661058 | Tp53 | 17:7674232-7674232 | c.731G>A | p.G244D |
| COSV53423873 | Tp53 | 17:7674231-7674232 | c.731_732delinsAA | p.G244E |
| COSV52773862 | Tp53 | 17:7674231-7674231 | c.732C>T | p.G244= |
| COSV52755988 | Tp53 | 17:7674231-7674231 | c.732C>G | p.G244= |
| COSV52677306 | Tp53 | 17:7674231-7674231 | c.732C>A | p.G244= |
| COSV52661744 | Tp53 | 17:7674230-7674230 | c.733G>T | p.G245C |
| COSV52713951 | Tp53 | 17:7674230-7674230 | c.733G>C | p.G245R |
| COSV52661877 | Tp53 | 17:7674230-7674230 | c.733G>A | p.G245S |
| COSV53636745 | Tp53 | 17:7674229-7674230 | c.733_734delinsTT | p.G245F |
| COSV53563472 | Tp53 | 17:7674229-7674230 | c.733_734delinsCT | p.G245L |
| COSV53396351 | Tp53 | 17:7674229-7674230 | c.733_734delinsCA | p.G245H |
| COSV52837315 | Tp53 | 17:7674229-7674230 | c.733_734delinsAA | p.G245N |
| COSV52666323 | Tp53 | 17:7674229-7674229 | c.734G>T | p.G245V |
| COSV52745465 | Tp53 | 17:7674229-7674229 | c.734G>C | p.G245A |
| COSV52667838 | Tp53 | 17:7674229-7674229 | c.734G>A | p.G245D |
| COSV53649292 | Tp53 | 17:7674228-7674229 | c.734_735delinsTT | p.G245V |
| COSV52700518 | Tp53 | 17:7674228-7674229 | c.734_735delinsAA | p.G245E |
| COSV52730464 | Tp53 | 17:7674228-7674228 | c.735C>T | p.G245= |
| COSV52884891 | Tp53 | 17:7674228-7674228 | c.735C>A | p.G245= |
| COSV53345811 | Tp53 | 17:7674227-7674232 | c.731_736delinsCCGCC | p.G244Afs*3 |
| COSV52707693 | Tp53 | 17:7674227-7674227 | c.736A>T | p.M246L |
| COSV52664850 | Tp53 | 17:7674227-7674227 | c.736A>G | p.M246V |
| COSV52949209 | Tp53 | 17:7674227-7674227 | c.736A>C | p.M246L |
| COSV52778210 | Tp53 | 17:7674226-7674226 | c.737T>G | p.M246R |
| COSV52693504 | Tp53 | 17:7674226-7674226 | c.737T>C | p.M246T |
| COSV52668375 | Tp53 | 17:7674226-7674226 | c.737T>A | p.M246K |
| COSV53038458 | Tp53 | 17:7674225-7674225 | c.738G>T | p.M246I |
| COSV52772732 | Tp53 | 17:7674225-7674225 | c.738G>C | p.M246I |
| COSV52735934 | Tp53 | 17:7674225-7674225 | c.738G>A | p.M246I |
| COSV53210485 | Tp53 | 17:7674224-7674224 | c.739A>T | p.N247Y |
| COSV53124306 | Tp53 | 17:7674224-7674224 | c.739A>G | p.N247D |
| COSV52732730 | Tp53 | 17:7674223-7674223 | c.740A>T | p.N247I |
| COSV53026017 | Tp53 | 17:7674223-7674223 | c.740A>G | p.N247S |
| COSV52675458 | Tp53 | 17:7674223-7674223 | c.740A>C | p.N247T |
| COSV52730035 | Tp53 | 17:7674222-7674223 | c.740_74ldelinsTA | p.N247I |
| COSV52808615 | Tp53 | 17:7674222-7674222 | c.741C>T | p.N247= |
| COSV52772996 | Tp53 | 17:7674222-7674222 | c.741C>A | p.N247K |
| COSV52752087 | Tp53 | 17:7674221-7674222 | c.741_742delinsTT | p.R248W |
| COSV52979949 | Tp53 | 17:7674221-7674222 | c.741_742delinsAT | p.N247_R248delinsKW |
| COSV52662035 | Tp53 | 17:7674221-7674221 | c.742C>T | p.R248W |
| COSV52797251 | Tp53 | 17:7674221-7674221 | c.742C>G | p.R248G |
| COSV53097881 | Tp53 | 17:7674221-7674221 | c.742C>A | p.R248= |
| COSV52802526 | Tp53 | 17:7674220-7674221 | c.742_743delinsTA | p.R248* |
| COSV52675468 | Tp53 | 17:7674220-7674220 | c.743G>T | p.R248L |
| COSV52661091 | Tp53 | 17:7674220-7674220 | c.743G>C | p.R248P |
| COSV52661580 | Tp53 | 17:7674220-7674220 | c.743G>A | p.R248Q |
| COSV52782547 | Tp53 | 17:7674219-7674223 | c.740_744delinsTTCCC | p.N247_R248delinsIP |
| COSV53339151 | Tp53 | 17:7674219-7674221 | c.742_744delinsTGC | p.R248C |
| COSV52783716 | Tp53 | 17:7674219-7674220 | c.743_744delinsTT | p.R248L |
| COSV52772144 | Tp53 | 17:7674219-7674220 | c.743_744delinsAA | p.R248Q |
| COSV53372256 | Tp53 | 17:7674219-7674219 | c.744G>T | p.R248= |
| COSV53050697 | Tp53 | 17:7674219-7674219 | c.744G>C | p.R248= |
| COSV52782654 | Tp53 | 17:7674219-7674219 | c.744G>A | p.R248= |
| COSV52662247 | Tp53 | 17:7674218-7674218 | c.745A>T | p.R249W |
| COSV52676356 | Tp53 | 17:7674218-7674218 | c.745A>G | p.R249G |
| COSV52892337 | Tp53 | 17:7674218-7674218 | c.745A>C | p.R249= |
| COSV53242760 | Tp53 | 17:7674217-7674218 | c.745_746delinsGT | p.R249V |
| COSV52668050 | Tp53 | 17:7674217-7674217 | c.746G>T | p.R249M |
| COSV52697169 | Tp53 | 17:7674217-7674217 | c.746G>C | p.R249T |
| COSV52666526 | Tp53 | 17:7674217-7674217 | c.746G>A | p.R249K |
| COSV53496711 | Tp53 | 17:7674216-7674218 | c.745_747delinsGGT | p.R249G |
| COSV53279053 | Tp53 | 17:7674216-7674217 | c.746_747delinsTT | p.R249I |
| COSV52661594 | Tp53 | 17:7674216-7674216 | c.747G>T | p.R249S |
| COSV52668882 | Tp53 | 17:7674216-7674216 | c.747G>C | p.R249S |
| COSV52827572 | Tp53 | 17:7674216-7674216 | c.747G>A | p.R249= |
| COSV53425938 | Tp53 | 17:7674215-7674216 | c.747_748delinsTT | p.R249_P250delinsSS |
| COSV52661859 | Tp53 | 17:7674215-7674215 | c.748C>T | p.P250S |
| COSV52771856 | Tp53 | 17:7674215-7674215 | c.748C>G | p.P250A |
| COSV52796404 | Tp53 | 17:7674215-7674215 | c.748C>A | p.P250T |
| COSV52891623 | Tp53 | 17:7674214-7674215 | c.748_749delinsTT | p.P250F |
| COSV52987233 | Tp53 | 17:7674214-7674215 | c.748_749delinsAA | p.P250N |
| COSV52687307 | Tp53 | 17:7674214-7674214 | c.749C>T | p.P250L |
| COSV52661102 | Tp53 | 17:7674214-7674214 | c.749C>A | p.P250H |
| COSV52980567 | Tp53 | 17:7674213-7674214 | c.749_750delinsTT | p.P250L |
| COSV53623927 | Tp53 | 17:7674213-7674214 | c.749_750delinsTG | p.P250L |
| COSV52978598 | Tp53 | 17:7674213-7674214 | c.749_750delinsAG | p.P250Q |
| COSV52876092 | Tp53 | 17:7674213-7674213 | c.750C>T | p.P250= |
| COSV104587932 | Tp53 | 17:7674212-7674215 | c.748_751delinsTCCCT | p.P250Sfs*14 |
| COSV52677403 | Tp53 | 17:7674212-7674212 | c.751A>T | p.I251F |
| COSV52772679 | Tp53 | 17:7674212-7674212 | c.751A>G | p.I251V |
| COSV52702261 | Tp53 | 17:7674212-7674212 | c.751A>C | p.I251L |
| COSV52728902 | Tp53 | 17:7674211-7674211 | c.752T>G | p.I251S |
| COSV52816233 | Tp53 | 17:7674211-7674211 | c.752T>C | p.I251T |
| COSV52741592 | Tp53 | 17:7674211-7674211 | c.752T>A | p.I251N |
| COSV53310789 | Tp53 | 17:7674210-7674211 | c.752_753delinsAT | p.I251N |
| COSV53202810 | Tp53 | 17:7674210-7674210 | c.753C>G | p.I251M |
| COSV52978618 | Tp53 | 17:7674210-7674210 | c.753C>A | p.I251= |
| COSV52807474 | Tp53 | 17:7674209-7674210 | c.753_754delinsGT | p.I251_L252delinsMF |
| COSV52730003 | Tp53 | 17:7674209-7674209 | c.754C>T | p.L252F |
| COSV52764738 | Tp53 | 17:7674208-7674208 | c.755T>C | p.L252P |
| COSV52950153 | Tp53 | 17:7674208-7674208 | c.755T>A | p.L252H |
| COSV53099988 | Tp53 | 17:7674207-7674207 | c.756C>T | p.L252= |
| COSV53368158 | Tp53 | 17:7674207-7674207 | c.756C>G | p.L252= |
| COSV53120416 | Tp53 | 17:7674207-7674207 | c.756C>A | p.L252= |
| COSV52906834 | Tp53 | 17:7674206-7674206 | c.757A>T | p.T253S |
| COSV52783299 | Tp53 | 17:7674206-7674206 | c.757A>G | p.T253A |
| COSV52873550 | Tp53 | 17:7674206-7674206 | c.757A>C | p.T253P |
| COSV52863408 | Tp53 | 17:7674205-7674205 | c.758C>T | p.T2531 |
| COSV52756133 | Tp53 | 17:7674205-7674205 | c.758C>G | p.T253S |
| COSV52784543 | Tp53 | 17:7674205-7674205 | c.758C>A | p.T253N |
| COSV52677640 | Tp53 | 17:7674204-7674204 | c.759C>T | p.T253= |
| COSV52825280 | Tp53 | 17:7674203-7674203 | c.760A>T | p.I254F |
| COSV52784505 | Tp53 | 17:7674203-7674203 | c.760A>G | p.I254V |
| COSV52872554 | Tp53 | 17:7674203-7674203 | c.760A>C | p.I254L |
| COSV52827538 | Tp53 | 17:7674202-7674203 | c.760_761delinsGA | p.I254D |
| COSV52714188 | Tp53 | 17:7674202-7674202 | c.761T>G | p.I254S |
| COSV52767322 | Tp53 | 17:7674202-7674202 | c.761T>C | p.I254T |
| COSV52674699 | Tp53 | 17:7674202-7674202 | c.761T>A | p.I254N |
| COSV53833684 | Tp53 | 17:7674201-7674201 | c.762C>G | p.I254M |
| COSV53510655 | Tp53 | 17:7674201-7674201 | c.762C>A | p.I254= |
| COSV52663197 | Tp53 | 17:7674200-7674200 | c.763A>T | p.I255F |
| COSV52713501 | Tp53 | 17:7674200-7674200 | c.763A>G | p.I255V |
| COSV52685038 | Tp53 | 17:7674199-7674199 | c.764T>G | p.I255S |
| COSV52712740 | Tp53 | 17:7674199-7674199 | c.764T>C | p.I255T |
| COSV52714133 | Tp53 | 17:7674199-7674199 | c.764T>A | p.I255N |
| COSV53599946 | Tp53 | 17:7674198-7674199 | c.764_765delinsCT | p.I255T |
| COSV53124648 | Tp53 | 17:7674198-7674198 | c.765C>T | p.I255= |
| COSV53233921 | Tp53 | 17:7674198-7674198 | c.765C>G | p.I255M |
| COSV53651156 | Tp53 | 17:7674198-7674198 | c.765C>A | p.I255= |
| COSV52979358 | Tp53 | 17:7674197-7674197 | c.766A>T | p.T256S |
| COSV53050299 | Tp53 | 17:7674197-7674197 | c.766A>G | p.T256A |
| COSV52801994 | Tp53 | 17:7674197-7674197 | c.766A>C | p.T256P |
| COSV52987484 | Tp53 | 17:7674196-7674196 | c.767C>T | p.T2561 |
| COSV53518652 | Tp53 | 17:7674196-7674196 | c.767C>G | p.T256R |
| COSV52837901 | Tp53 | 17:7674196-7674196 | c.767C>A | p.T256K |
| COSV52838755 | Tp53 | 17:7674195-7674195 | c.768A>G | p.T256= |
| COSV52717368 | Tp53 | 17:7674194-7674194 | c.769C>G | p.L257V |
| COSV52682484 | Tp53 | 17:7674193-7674193 | c.770T>G | p.L257R |
| COSV52757928 | Tp53 | 17:7674193-7674193 | c.770T>C | p.L257P |
| COSV52703253 | Tp53 | 17:7674193-7674193 | c.770T>A | p.L257Q |
| COSV53267831 | Tp53 | 17:7674192-7674192 | c.771G>A | p.L257= |
| COSV52848710 | Tp53 | 17:7674191-7674192 | c.771_772delinsAT | p.E258* |
| COSV53551282 | Tp53 | 17:7674191-7674192 | c.771_772delinsAA | p.E258K |
| COSV52740428 | Tp53 | 17:7674191-7674191 | c.772G>T | p.E258* |
| COSV52828352 | Tp53 | 17:7674191-7674191 | c.772G>C | p.E258Q |
| COSV52684909 | Tp53 | 17:7674191-7674191 | c.772G>A | p.E258K |
| COSV53439210 | Tp53 | 17:7674190-7674191 | c.772_773delinsTT | p.E258L |
| COSV52665450 | Tp53 | 17:7674190-7674190 | c.773A>T | p.E258V |
| COSV52661460 | Tp53 | 17:7674190-7674190 | c.773A>G | p.E258G |
| COSV52688395 | Tp53 | 17:7674190-7674190 | c.773A>C | p.E258A |
| COSV52752502 | Tp53 | 17:7674189-7674189 | c.774A>T | p.E258D |
| COSV52980614 | Tp53 | 17:7674189-7674189 | c.774A>C | p.E258D |
| COSV52985832 | Tp53 | 17:7674188-7674189 | c.774_775delinsTC | p.E258_D259delinsDH |
| COSV52675732 | Tp53 | 17:7674188-7674188 | c.775G>T | p.D259Y |
| COSV52962390 | Tp53 | 17:7674188-7674188 | c.775G>C | p.D259H |
| COSV52662086 | Tp53 | 17:7674188-7674188 | c.775G>A | p.D259N |
| COSV53124867 | Tp53 | 17:7674187-7674188 | c.775_776delinsAG | p.D259S |
| COSV52679505 | Tp53 | 17:7674187-7674187 | c.776A>T | p.D259V |
| COSV52677009 | Tp53 | 17:7674187-7674187 | c.776A>G | p.D259G |
| COSV53161508 | Tp53 | 17:7674186-7674189 | c.774_777delinsG | p.D259del |
| COSV53744712 | Tp53 | 17:7674186-7674187 | c.776_777delinsTT | p.D259V |
| COSV53387181 | Tp53 | 17:7674186-7674186 | c.777C>T | p.D259= |
| COSV53209408 | Tp53 | 17:7674186-7674186 | c.777C>A | p.D259E |
| COSV52950491 | Tp53 | 17:7674185-7674185 | c.778T>G | p.S260A |
| COSV53107447 | Tp53 | 17:7674185-7674185 | c.778T>C | p.S260P |
| COSV52661443 | Tp53 | 17:7674184-7674184 | c.779C>T | p.S260F |
| COSV52760769 | Tp53 | 17:7674184-7674184 | c.779C>G | p.S260C |
| COSV52978505 | Tp53 | 17:7674184-7674184 | c.779C>A | p.S260Y |
| COSV53068418 | Tp53 | 17:7674183-7674183 | c.780C>T | p.S260= |
| COSV53684557 | Tp53 | 17:7674182-7674182 | c.781A>T | p.S261C |
| COSV52700972 | Tp53 | 17:7674182-7674182 | c.781A>G | p.S261G |
| COSV53759588 | Tp53 | 17:7674182-7674182 | c.781A>C | p.S261R |
| COSV99450624 | Tp53 | 17:7674181-7674181 | c.782G>T | p.S261I |
| COSV52929094 | Tp53 | 17:7674181-7674181 | c.782G>C | p.S261T |
| COSV53339950 | Tp53 | 17:7674181-7674181 | c.782G>A | p.S261N |
| COSV53107603 | Tp53 | 17:7673837-7673837 | c.783T>G | p.S261R |
| COSV52890892 | Tp53 | 17:7673837-7673837 | c.783T>C | p.S261= |
| COSV52761919 | Tp53 | 17:7673837-7673837 | c.783T>A | p.S261R |
| COSV53512448 | Tp53 | 17:7673836-7673836 | c.784G>C | p.G262R |
| COSV52978722 | Tp53 | 17:7673836-7673836 | c.784G>A | p.G262S |
| COSV52950001 | Tp53 | 17:7673835-7673836 | c.784_785delinsCA | p.G262H |
| COSV52677105 | Tp53 | 17:7673835-7673835 | c.785G>T | p.G262V |
| COSV52952688 | Tp53 | 17:7673835-7673835 | c.785G>A | p.G262D |
| COSV52782687 | Tp53 | 17:7673834-7673834 | c.786T>C | p.G262= |
| COSV52849374 | Tp53 | 17:7673833-7673834 | c.786_787delinsAC | p.N263H |
| COSV52761623 | Tp53 | 17:7673833-7673833 | c.787A>G | p.N263D |
| COSV53098356 | Tp53 | 17:7673833-7673833 | c.787A>C | p.N263H |
| COSV53208998 | Tp53 | 17:7673832-7673832 | c.788A>T | p.N263I |
| COSV53210322 | Tp53 | 17:7673831-7673831 | c.789T>G | p.N263K |
| COSV52700343 | Tp53 | 17:7673830-7673830 | c.790C>T | p.L264= |
| COSV53098291 | Tp53 | 17:7673830-7673830 | c.790C>G | p.L264V |
| COSV53038101 | Tp53 | 17:7673830-7673830 | c.790C>A | p.L264I |
| COSV53882767 | Tp53 | 17:7673829-7673831 | c.789_791delinsA | p.N263Kfs*8 |
| COSV52890118 | Tp53 | 17:7673829-7673829 | c.791T>G | p.L264R |
| COSV53470181 | Tp53 | 17:7673829-7673829 | c.791T>C | p.L264P |
| COSV104369527 | Tp53 | 17:7673829-7673829 | c.791T>A | p.L264Q |
| COSV52838930 | Tp53 | 17:7673827-7673827 | c.793C>T | p.L265= |
| COSV52741199 | Tp53 | 17:7673827-7673827 | c.793C>A | p.L265M |
| COSV52706540 | Tp53 | 17:7673826-7673826 | c.794T>G | p.L265R |
| COSV52732531 | Tp53 | 17:7673826-7673826 | c.794T>C | p.L265P |
| COSV53696163 | Tp53 | 17:7673826-7673826 | c.794T>A | p.L265Q |
| COSV53424969 | Tp53 | 17:7673825-7673826 | c.794_795delinsCT | p.L265P |
| COSV52661636 | Tp53 | 17:7673825-7673825 | c.795G>C | p.L265= |
| COSV52895662 | Tp53 | 17:7673825-7673825 | c.795G>A | p.L265= |
| COSV105023136 | Tp53 | 17:7673824-7673825 | c.795_796delinsTT | p.G266* |
| COSV52778488 | Tp53 | 17:7673824-7673825 | c.795_796delinsAA | p.G266R |
| COSV52670619 | Tp53 | 17:7673824-7673824 | c.796G>T | p.G266* |
| COSV52751844 | Tp53 | 17:7673824-7673824 | c.796G>C | p.G266R |
| COSV52672762 | Tp53 | 17:7673824-7673824 | c.796G>A | p.G266R |
| COSV52975168 | Tp53 | 17:7673823-7673824 | c.796_797delinsAC | p.G266T |
| COSV52781281 | Tp53 | 17:7673823-7673824 | c.796_797delinsAA | p.G266K |
| COSV52666760 | Tp53 | 17:7673823-7673823 | c.797G>T | p.G266V |
| COSV53585299 | Tp53 | 17:7673823-7673823 | c.797G>C | p.G266A |
| COSV52664019 | Tp53 | 17:7673823-7673823 | c.797G>A | p.G266E |
| COSV53424760 | Tp53 | 17:7673822-7673822 | c.798A>T | p.G266= |
| COSV52700790 | Tp53 | 17:7673822-7673822 | c.798A>G | p.G266= |
| COSV52678166 | Tp53 | 17:7673821-7673821 | c.799C>T | p.R267W |
| COSV52736883 | Tp53 | 17:7673821-7673821 | c.799C>G | p.R267G |
| COSV52827473 | Tp53 | 17:7673821-7673821 | c.799C>A | p.R267= |
| COSV52675189 | Tp53 | 17:7673820-7673820 | c.800G>T | p.R267L |
| COSV52691512 | Tp53 | 17:7673820-7673820 | c.800G>C | p.R267P |
| COSV52734317 | Tp53 | 17:7673820-7673820 | c.800G>A | p.R267Q |
| COSV53289699 | Tp53 | 17:7673819-7673820 | c.800_801delinsTT | p.R267L |
| COSV53164528 | Tp53 | 17:7673819-7673819 | c.801G>T | p.R267= |
| COSV52712702 | Tp53 | 17:7673819-7673819 | c.801G>C | p.R267= |
| COSV53387047 | Tp53 | 17:7673819-7673819 | c.801G>A | p.R267= |
| COSV52760372 | Tp53 | 17:7673818-7673818 | c.802A>C | p.N268H |
| COSV52670902 | Tp53 | 17:7673817-7673817 | c.803A>T | p.N268I |
| COSV52978206 | Tp53 | 17:7673817-7673817 | c.803A>G | p.N268S |
| COSV53086682 | Tp53 | 17:7673816-7673816 | c.804C>T | p.N268= |
| COSV53241270 | Tp53 | 17:7673816-7673816 | c.804C>G | p.N268K |
| COSV53135942 | Tp53 | 17:7673815-7673815 | c.805A>T | p.S269C |
| COSV52713319 | Tp53 | 17:7673815-7673815 | c.805A>G | p.S269G |
| COSV52734814 | Tp53 | 17:7673814-7673814 | c.806G>T | p.S269I |
| COSV53252245 | Tp53 | 17:7673814-7673814 | c.806G>C | p.S269T |
| COSV52675910 | Tp53 | 17:7673814-7673814 | c.806G>A | p.S269N |
| COSV53165079 | Tp53 | 17:7673813-7673813 | c.807C>T | p.S269= |
| COSV104587891 | Tp53 | 17:7673813-7673813 | c.807C>G | p.S269R |
| COSV53562993 | Tp53 | 17:7673813-7673813 | c.807C>A | p.S269R |
| COSV52697968 | Tp53 | 17:7673812-7673812 | c.808T>G | p.F270V |
| COSV52691720 | Tp53 | 17:7673812-7673812 | c.808T>C | p.F270L |
| COSV52744290 | Tp53 | 17:7673812-7673812 | c.808T>A | p.F270I |
| COSV52661272 | Tp53 | 17:7673811-7673811 | c.809T>G | p.F270C |
| COSV52676663 | Tp53 | 17:7673811-7673811 | c.809T>C | p.F270S |
| COSV52688116 | Tp53 | 17:7673811-7673811 | c.809T>A | p.F270Y |
| COSV52809003 | Tp53 | 17:7673810-7673810 | c.810T>G | p.F270L |
| COSV53009334 | Tp53 | 17:7673810-7673810 | c.810T>A | p.F270L |
| COSV52746468 | Tp53 | 17:7673809-7673809 | c.811G>T | p.E271* |
| COSV52666332 | Tp53 | 17:7673809-7673809 | c.811G>C | p.E271Q |
| COSV52700326 | Tp53 | 17:7673809-7673809 | c.811G>A | p.E271K |
| COSV53424338 | Tp53 | 17:7673808-7673809 | c.811_812delinsCC | p.E271P |
| COSV52661418 | Tp53 | 17:7673808-7673808 | c.812A>T | p.E271V |
| COSV52925998 | Tp53 | 17:7673808-7673808 | c.812A>G | p.E271G |
| COSV53313144 | Tp53 | 17:7673807-7673809 | c.811_813delinsTAA | p.E271* |
| COSV52849794 | Tp53 | 17:7673807-7673807 | c.813G>T | p.E271D |
| COSV52892313 | Tp53 | 17:7673807-7673807 | c.813G>C | p.E271D |
| COSV52661621 | Tp53 | 17:7673807-7673807 | c.813G>A | p.E271= |
| COSV52701127 | Tp53 | 17:7673806-7673806 | c.814G>T | p.V272L |
| COSV52677483 | Tp53 | 17:7673806-7673806 | c.814G>C | p.V272L |
| COSV52661812 | Tp53 | 17:7673806-7673806 | c.814G>A | p.V272M |
| COSV53278592 | Tp53 | 17:7673805-7673806 | c.814_815delinsAA | p.V272K |
| COSV52787833 | Tp53 | 17:7673805-7673805 | c.815T>G | p.V272G |
| COSV52701102 | Tp53 | 17:7673805-7673805 | c.815T>C | p.V272A |
| COSV52773741 | Tp53 | 17:7673805-7673805 | c.815T>A | p.V272E |
| COSV53313340 | Tp53 | 17:7673804-7673804 | c.816G>T | p.V272= |
| COSV53438396 | Tp53 | 17:7673804-7673804 | c.816G>C | p.V272= |
| COSV52918960 | Tp53 | 17:7673804-7673804 | c.816G>A | p.V272= |
| COSV52662066 | Tp53 | 17:7673803-7673803 | c.817C>T | p.R273C |
| COSV52670856 | Tp53 | 17:7673803-7673803 | c.817C>G | p.R273G |
| COSV52707054 | Tp53 | 17:7673803-7673803 | c.817C>A | p.R273S |
| COSV53242513 | Tp53 | 17:7673802-7673803 | c.817_818delinsTA | p.R273Y |
| COSV52664805 | Tp53 | 17:7673802-7673802 | c.818G>T | p.R273L |
| COSV52676050 | Tp53 | 17:7673802-7673802 | c.818G>C | p.R273P |
| COSV52660980 | Tp53 | 17:7673802-7673802 | c.818G>A | p.R273H |
| COSV52808677 | Tp53 | 17:7673801-7673803 | c.817_819delinsTCG | p.R273S |
| COSV99384939 | Tp53 | 17:7673801-7673802 | c.818_819delinsAG | p.R273Q |
| COSV53716789 | Tp53 | 17:7673801-7673801 | c.819T>C | p.R273= |
| COSV52707923 | Tp53 | 17:7673800-7673800 | c.820G>T | p.V274F |
| COSV52708386 | Tp53 | 17:7673800-7673800 | c.820G>C | p.V274L |
| COSV52728497 | Tp53 | 17:7673800-7673800 | c.820G>A | p.V274I |
| COSV99036826 | Tp53 | 17:7673799-7673803 | c.817_821delinsGACCC | p.R273_V274delinsDP |
| COSV52761348 | Tp53 | 17:7673799-7673799 | c.821T>G | p.V274G |
| COSV52709979 | Tp53 | 17:7673799-7673799 | c.821T>C | p.V274A |
| COSV52662012 | Tp53 | 17:7673799-7673799 | c.821T>A | p.V274D |
| COSV52751885 | Tp53 | 17:7673798-7673798 | c.822T>G | p.V274= |
| COSV53327229 | Tp53 | 17:7673798-7673798 | c.822T>C | p.V274= |
| COSV52771673 | Tp53 | 17:7673797-7673797 | c.823T>G | p.C275G |
| COSV52700773 | Tp53 | 17:7673797-7673797 | c.823T>C | p.C275R |
| COSV104585223 | Tp53 | 17:7673797-7673797 | c.823T>A | p.C275S |
| COSV52663595 | Tp53 | 17:7673796-7673796 | c.824G>T | p.C275F |
| COSV52772708 | Tp53 | 17:7673796-7673796 | c.824G>C | p.C275S |
| COSV52661919 | Tp53 | 17:7673796-7673796 | c.824G>A | p.C275Y |
| COSV99066541 | Tp53 | 17:7673795-7673796 | c.824_825delinsTC | p.C275F |
| COSV52759232 | Tp53 | 17:7673795-7673795 | c.825T>G | p.C275W |
| COSV53086963 | Tp53 | 17:7673795-7673795 | c.825T>C | p.C275= |
| COSV52912941 | Tp53 | 17:7673795-7673795 | c.825T>A | p.C275* |
| COSV52823875 | Tp53 | 17:7673794-7673794 | c.826G>T | p.A276S |
| COSV52731052 | Tp53 | 17:7673794-7673794 | c.826G>C | p.A276P |
| COSV52978993 | Tp53 | 17:7673794-7673794 | c.826G>A | p.A276T |
| COSV53306726 | Tp53 | 17:7673793-7673794 | c.826_827delinsTT | p.A276F |
| COSV52704969 | Tp53 | 17:7673793-7673793 | c.827C>T | p.A276V |
| COSV52749634 | Tp53 | 17:7673793-7673793 | c.827C>G | p.A276G |
| COSV52761908 | Tp53 | 17:7673793-7673793 | c.827C>A | p.A276D |
| COSV105031956 | Tp53 | 17:7673792-7673793 | c.827_828delinsAT | p.A276D |
| COSV52814873 | Tp53 | 17:7673792-7673792 | c.828C>T | p.A276= |
| COSV52796700 | Tp53 | 17:7673792-7673792 | c.828C>A | p.A276= |
| COSV53255025 | Tp53 | 17:7673791-7673793 | c.827_829delinsTC | p.A276Vfs*69 |
| COSV52661564 | Tp53 | 17:7673791-7673791 | c.829T>G | p.C277G |
| COSV53188691 | Tp53 | 17:7673791-7673791 | c.829T>C | p.C277R |
| COSV52693726 | Tp53 | 17:7673790-7673790 | c.830G>T | p.C277F |
| COSV53107923 | Tp53 | 17:7673790-7673790 | c.830G>C | p.C277S |
| COSV52826087 | Tp53 | 17:7673790-7673790 | c.830G>A | p.C277Y |
| COSV52783531 | Tp53 | 17:7673789-7673789 | c.831T>G | p.C277W |
| COSV53164903 | Tp53 | 17:7673789-7673789 | c.831T>C | p.C277= |
| COSV52676368 | Tp53 | 17:7673789-7673789 | c.831T>A | p.C277* |
| COSV52684662 | Tp53 | 17:7673788-7673788 | c.832C>T | p.P278S |
| COSV52668132 | Tp53 | 17:7673788-7673788 | c.832C>G | p.P278A |
| COSV52679740 | Tp53 | 17:7673788-7673788 | c.832C>A | p.P278T |
| COSV52710607 | Tp53 | 17:7673787-7673788 | c.832_833delinsTT | p.P278F |
| COSV52678063 | Tp53 | 17:7673787-7673787 | c.833C>T | p.P278L |
| COSV52661225 | Tp53 | 17:7673787-7673787 | c.833C>G | p.P278R |
| COSV52665769 | Tp53 | 17:7673787-7673787 | c.833C>A | p.P278H |
| COSV52729764 | Tp53 | 17:7673786-7673786 | c.834T>C | p.P278= |
| COSV53486085 | Tp53 | 17:7673785-7673785 | c.835G>T | p.G279W |
| COSV53165376 | Tp53 | 17:7673785-7673785 | c.835G>C | p.G279R |
| COSV52705216 | Tp53 | 17:7673785-7673785 | c.835G>A | p.G279R |
| COSV52821965 | Tp53 | 17:7673784-7673785 | c.835_836delinsTA | p.G279* |
| COSV52851240 | Tp53 | 17:7673784-7673784 | c.836G>T | p.G279V |
| COSV52677880 | Tp53 | 17:7673784-7673784 | c.836G>A | p.G279E |
| COSV53008567 | Tp53 | 17:7673783-7673784 | c.836_837delinsTA | p.G279V |
| COSV52920251 | Tp53 | 17:7673783-7673784 | c.836_837delinsAA | p.G279E |
| COSV53696473 | Tp53 | 17:7673783-7673783 | c.837G>T | p.G279= |
| COSV53098982 | Tp53 | 17:7673783-7673783 | c.837G>A | p.G279= |
| COSV52708728 | Tp53 | 17:7673782-7673782 | c.838A>T | p.R280* |
| COSV52662754 | Tp53 | 17:7673782-7673782 | c.838A>G | p.R280G |
| COSV52687987 | Tp53 | 17:7673781-7673781 | c.839G>T | p.R280I |
| COSV52677783 | Tp53 | 17:7673781-7673781 | c.839G>C | p.R280T |
| COSV52666248 | Tp53 | 17:7673781-7673781 | c.839G>A | p.R280K |
| COSV52801834 | Tp53 | 17:7673780-7673780 | c.840A>T | p.R280S |
| COSV52678283 | Tp53 | 17:7673780-7673780 | c.840A>G | p.R280= |
| COSV52782181 | Tp53 | 17:7673780-7673780 | c.840A>C | p.R280S |
| COSV52678614 | Tp53 | 17:7673779-7673779 | c.841G>T | p.D281Y |
| COSV52674651 | Tp53 | 17:7673779-7673779 | c.841G>C | p.D281H |
| COSV52671054 | Tp53 | 17:7673779-7673779 | c.841G>A | p.D281N |
| COSV52815868 | Tp53 | 17:7673778-7673778 | c.842A>T | p.D281V |
| COSV52694391 | Tp53 | 17:7673778-7673778 | c.842A>G | p.D281G |
| COSV52729448 | Tp53 | 17:7673778-7673778 | c.842A>C | p.D281A |
| COSV53578831 | Tp53 | 17:7673777-7673779 | c.841_843delinsCGG | p.D281R |
| COSV52719382 | Tp53 | 17:7673777-7673777 | c.843C>T | p.D281= |
| COSV52664212 | Tp53 | 17:7673777-7673777 | c.843C>G | p.D281E |
| COSV52688790 | Tp53 | 17:7673777-7673777 | c.843C>A | p.D281E |
| COSV52740774 | Tp53 | 17:7673776-7673777 | c.843_844delinsTT | p.R282W |
| COSV52677446 | Tp53 | 17:7673776-7673777 | c.843_844delinsGT | p.D281_R282delinsEW |
| COSV52711481 | Tp53 | 17:7673776-7673777 | c.843_844delinsAT | p.D281_R282delinsEW |
| COSV52662048 | Tp53 | 17:7673776-7673776 | c.844C>T | p.R282W |
| COSV52701296 | Tp53 | 17:7673776-7673776 | c.844C>G | p.R282G |
| COSV52662222 | Tp53 | 17:7673776-7673776 | c.844C>A | p.R282= |
| COSV53068318 | Tp53 | 17:7673775-7673775 | c.845G>T | p.R282L |
| COSV52773873 | Tp53 | 17:7673775-7673775 | c.845G>C | p.R282P |
| COSV52689673 | Tp53 | 17:7673775-7673775 | c.845G>A | p.R282Q |
| COSV53470390 | Tp53 | 17:7673774-7673775 | c.845_846delinsAT | p.R282H |
| COSV53156241 | Tp53 | 17:7673774-7673774 | c.846G>C | p.R282= |
| COSV53037261 | Tp53 | 17:7673774-7673774 | c.846G>A | p.R282= |
| COSV52676681 | Tp53 | 17:7673773-7673773 | c.847C>T | p.R283C |
| COSV53235263 | Tp53 | 17:7673773-7673773 | c.847C>G | p.R283G |
| COSV52751436 | Tp53 | 17:7673773-7673773 | c.847C>A | p.R283S |
| COSV52964971 | Tp53 | 17:7673772-7673772 | c.848G>T | p.R283L |
| COSV52693421 | Tp53 | 17:7673772-7673772 | c.848G>C | p.R283P |
| COSV52700134 | Tp53 | 17:7673772-7673772 | c.848G>A | p.R283H |
| COSV53232948 | Tp53 | 17:7673771-7673771 | c.849C>T | p.R283= |
| COSV52772770 | Tp53 | 17:7673771-7673771 | c.849C>G | p.R283= |
| COSV52907973 | Tp53 | 17:7673771-7673771 | c.849C>A | p.R283= |
| COSV53272353 | Tp53 | 17:7673770-7673770 | c.850A>T | p.T284S |
| COSV52809281 | Tp53 | 17:7673770-7673770 | c.850A>G | p.T284A |
| COSV52896079 | Tp53 | 17:7673770-7673770 | c.850A>C | p.T284P |
| COSV52908002 | Tp53 | 17:7673769-7673769 | c.851C>T | p.T284I |
| COSV53098088 | Tp53 | 17:7673768-7673768 | c.852A>T | p.T284= |
| COSV52670664 | Tp53 | 17:7673767-7673767 | c.853G>T | p.E285* |
| COSV53650254 | Tp53 | 17:7673767-7673767 | c.853G>C | p.E285Q |
| COSV52661732 | Tp53 | 17:7673767-7673767 | c.853G>A | p.E285K |
| COSV52676438 | Tp53 | 17:7673766-7673766 | c.854A>T | p.E285V |
| COSV53038615 | Tp53 | 17:7673766-7673766 | c.854A>G | p.E285G |
| COSV53625652 | Tp53 | 17:7673766-7673766 | c.854A>C | p.E285A |
| COSV53494044 | Tp53 | 17:7673765-7673767 | c.853_855delinsAAA | p.E285K |
| COSV52933457 | Tp53 | 17:7673765-7673765 | c.855G>A | p.E285= |
| COSV53018324 | Tp53 | 17:7673764-7673765 | c.855_856delinsTA | p.E285_E286delinsDK |
| COSV53017074 | Tp53 | 17:7673764-7673765 | c.855_856delinsAA | p.E286K |
| COSV52687201 | Tp53 | 17:7673764-7673764 | c.856G>T | p.E286* |
| COSV52688214 | Tp53 | 17:7673764-7673764 | c.856G>C | p.E286Q |
| COSV52664318 | Tp53 | 17:7673764-7673764 | c.856G>A | p.E286K |
| COSV53382736 | Tp53 | 17:7673763-7673766 | c.854_857delinsGG | p.E285Gfs*20 |
| COSV52748431 | Tp53 | 17:7673763-7673763 | c.857A>T | p.E286V |
| COSV52661405 | Tp53 | 17:7673763-7673763 | c.857A>G | p.E286G |
| COSV53044704 | Tp53 | 17:7673763-7673763 | c.857A>C | p.E286A |
| COSV53438884 | Tp53 | 17:7673762-7673762 | c.858A>T | p.E286D |
| COSV53622887 | Tp53 | 17:7673762-7673762 | c.858A>G | p.E286= |
| COSV53463163 | Tp53 | 17:7673762-7673762 | c.858A>C | p.E286D |
| COSV52679410 | Tp53 | 17:7673761-7673761 | c.859G>T | p.E287* |
| COSV52765316 | Tp53 | 17:7673761-7673761 | c.859G>C | p.E287Q |
| COSV52730707 | Tp53 | 17:7673761-7673761 | c.859G>A | p.E287K |
| COSV52849820 | Tp53 | 17:7673760-7673760 | c.860A>T | p.E287V |
| COSV52839202 | Tp53 | 17:7673760-7673760 | c.860A>G | p.E287G |
| COSV53296827 | Tp53 | 17:7673760-7673760 | c.860A>C | p.E287A |
| COSV52883064 | Tp53 | 17:7673759-7673759 | c.861G>T | p.E287D |
| COSV52684280 | Tp53 | 17:7673759-7673759 | c.861G>C | p.E287D |
| COSV52840253 | Tp53 | 17:7673759-7673759 | c.861G>A | p.E287= |
| COSV52761170 | Tp53 | 17:7673758-7673758 | c.862A>T | p.N288Y |
| COSV53175642 | Tp53 | 17:7673758-7673758 | c.862A>G | p.N288D |
| COSV104581610 | Tp53 | 17:7673758-7673758 | c.862A>C | p.N288H |
| COSV53675191 | Tp53 | 17:7673757-7673770 | c.850_863delinsT | p.T284Ffs*57 |
| COSV52712952 | Tp53 | 17:7673757-7673757 | c.863A>G | p.N288S |
| COSV53705435 | Tp53 | 17:7673756-7673756 | c.864T>A | p.N288K |
| COSV52729747 | Tp53 | 17:7673755-7673755 | c.865C>T | p.L289F |
| COSV52714152 | Tp53 | 17:7673755-7673755 | c.865C>G | p.L289V |
| COSV53770961 | Tp53 | 17:7673754-7673754 | c.866T>G | p.L289R |
| COSV52783014 | Tp53 | 17:7673754-7673754 | c.866T>C | p.L289P |
| COSV53026071 | Tp53 | 17:7673753-7673753 | c.867C>T | p.L289= |
| COSV52823665 | Tp53 | 17:7673752-7673755 | c.865_868delinsTTT | p.L289Ffs*56 |
| COSV52761356 | Tp53 | 17:7673752-7673752 | c.868C>T | p.R290C |
| COSV53509681 | Tp53 | 17:7673752-7673752 | c.868C>A | p.R290S |
| COSV53296796 | Tp53 | 17:7673751-7673751 | c.869G>T | p.R290L |
| COSV52683585 | Tp53 | 17:7673751-7673751 | c.869G>A | p.R290H |
| COSV52918863 | Tp53 | 17:7673750-7673750 | c.870C>T | p.R290= |
| COSV53637296 | Tp53 | 17:7673750-7673750 | c.870C>G | p.R290= |
| COSV53741507 | Tp53 | 17:7673750-7673750 | c.870C>A | p.R290= |
| COSV52809147 | Tp53 | 17:7673749-7673749 | c.871A>T | p.K291* |
| COSV52984891 | Tp53 | 17:7673749-7673749 | c.871A>G | p.K291E |
| COSV52761043 | Tp53 | 17:7673749-7673749 | c.871A>C | p.K291Q |
| COSV53772723 | Tp53 | 17:7673748-7673748 | c.872A>T | p.K291M |
| COSV52890873 | Tp53 | 17:7673748-7673748 | c.872A>G | p.K291R |
| COSV53142652 | Tp53 | 17:7673748-7673748 | c.872A>C | p.K291T |
| COSV52760527 | Tp53 | 17:7673747-7673747 | c.873G>C | p.K291N |
| COSV52774013 | Tp53 | 17:7673747-7673747 | c.873G>A | p.K291= |
| COSV53059791 | Tp53 | 17:7673746-7673746 | c.874A>T | p.K292* |
| COSV52676695 | Tp53 | 17:7673746-7673746 | c.874A>G | p.K292E |
| COSV99066499 | Tp53 | 17:7673745-7673745 | c.875A>T | p.K292I |
| COSV53008143 | Tp53 | 17:7673745-7673745 | c.875A>G | p.K292R |
| COSV53067424 | Tp53 | 17:7673745-7673745 | c.875A>C | p.K292T |
| COSV53160519 | Tp53 | 17:7673744-7673752 | c.868_876delinsG | p.R290Gfs*13 |
| COSV53188887 | Tp53 | 17:7673744-7673744 | c.876A>T | p.K292N |
| COSV52795860 | Tp53 | 17:7673744-7673744 | c.876A>G | p.K292= |
| COSV53037721 | Tp53 | 17:7673744-7673744 | c.876A>C | p.K292N |
| COSV52730603 | Tp53 | 17:7673743-7673743 | c.877G>T | p.G293W |
| COSV52689066 | Tp53 | 17:7673743-7673743 | c.877G>C | p.G293R |
| COSV52752246 | Tp53 | 17:7673743-7673743 | c.877G>A | p.G293R |
| COSV53471097 | Tp53 | 17:7673742-7673742 | c.878G>T | p.G293V |
| COSV53206735 | Tp53 | 17:7673742-7673742 | c.878G>C | p.G293A |
| COSV53260189 | Tp53 | 17:7673742-7673742 | c.878G>A | p.G293E |
| COSV52761595 | Tp53 | 17:7673741-7673741 | c.879G>C | p.G293= |
| COSV52751109 | Tp53 | 17:7673741-7673741 | c.879G>A | p.G293= |
| COSV53259568 | Tp53 | 17:7673740-7673741 | c.879_880delinsTT | p.E294* |
| COSV53851434 | Tp53 | 17:7673740-7673740 | c.880delinsTT | p.E294Lfs*12 |
| COSV52689734 | Tp53 | 17:7673740-7673740 | c.880G>T | p.E294* |
| COSV52701760 | Tp53 | 17:7673740-7673740 | c.880G>C | p.E294Q |
| COSV52773998 | Tp53 | 17:7673740-7673740 | c.880G>A | p.E294K |
| COSV53326585 | Tp53 | 17:7673739-7673739 | c.881A>T | p.E294V |
| COSV52661395 | Tp53 | 17:7673739-7673739 | c.881A>G | p.E294G |
| COSV52661825 | Tp53 | 17:7673738-7673738 | c.882G>T | p.E294D |
| COSV52937308 | Tp53 | 17:7673738-7673738 | c.882G>A | p.E294= |
| COSV52863694 | Tp53 | 17:7673737-7673737 | c.883C>T | p.P295S |
| COSV53238647 | Tp53 | 17:7673737-7673737 | c.883C>G | p.P295A |
| COSV52782749 | Tp53 | 17:7673736-7673736 | c.884C>T | p.P295L |
| COSV53637179 | Tp53 | 17:7673736-7673736 | c.884C>G | p.P295R |
| COSV52926525 | Tp53 | 17:7673736-7673736 | c.884C>A | p.P295H |
| COSV52851077 | Tp53 | 17:7673735-7673735 | c.885T>C | p.P295= |
| COSV52949796 | Tp53 | 17:7673734-7673734 | c.886C>T | p.H296Y |
| COSV53210146 | Tp53 | 17:7673734-7673734 | c.886C>A | p.H296N |
| COSV52661430 | Tp53 | 17:7673733-7673733 | c.887A>T | p.H296L |
| COSV53423770 | Tp53 | 17:7673733-7673733 | c.887A>G | p.H296R |
| COSV53715451 | Tp53 | 17:7673733-7673733 | c.887A>C | p.H296P |
| COSV53551410 | Tp53 | 17:7673732-7673732 | c.888C>G | p.H296Q |
| COSV52850583 | Tp53 | 17:7673731-7673732 | c.888_889delinsTT | p.H297Y |
| COSV53110176 | Tp53 | 17:7673731-7673731 | c.889C>T | p.H297Y |
| COSV53729570 | Tp53 | 17:7673731-7673731 | c.889C>A | p.H297N |
| COSV52864048 | Tp53 | 17:7673730-7673730 | c.890A>C | p.H297P |
| COSV52661551 | Tp53 | 17:7673728-7673728 | c.892G>T | p.E298* |
| COSV52761493 | Tp53 | 17:7673728-7673728 | c.892G>C | p.E298Q |
| COSV53353479 | Tp53 | 17:7673728-7673728 | c.892G>A | p.E298K |
| COSV53009459 | Tp53 | 17:7673727-7673727 | c.893A>T | p.E298V |
| COSV53296033 | Tp53 | 17:7673727-7673727 | c.893A>C | p.E298A |
| COSV53542087 | Tp53 | 17:7673726-7673726 | c.894G>C | p.E298D |
| COSV53684282 | Tp53 | 17:7673726-7673726 | c.894G>A | p.E298= |
| COSV53695760 | Tp53 | 17:7673725-7673725 | c.895C>G | p.L299V |
| COSV53211244 | Tp53 | 17:7673724-7673724 | c.896T>C | p.L299P |
| COSV52839794 | Tp53 | 17:7673723-7673723 | c.897G>T | p.L299= |
| COSV53158815 | Tp53 | 17:7673722-7673722 | c.898C>T | p.P300S |
| COSV52713263 | Tp53 | 17:7673722-7673722 | c.898C>G | p.P300A |
| COSV52712660 | Tp53 | 17:7673721-7673721 | c.899C>T | p.P300L |
| COSV53743131 | Tp53 | 17:7673721-7673721 | c.899C>G | p.P300R |
| COSV53486330 | Tp53 | 17:7673720-7673720 | c.900C>A | p.P300= |
| COSV52978880 | Tp53 | 17:7673719-7673719 | c.901C>T | p.P301S |
| COSV53268568 | Tp53 | 17:7673719-7673719 | c.901C>G | p.P301A |
| COSV53407712 | Tp53 | 17:7673719-7673719 | c.901C>A | p.P301T |
| COSV53486315 | Tp53 | 17:7673718-7673718 | c.902C>T | p.P301L |
| COSV53267099 | Tp53 | 17:7673718-7673718 | c.902C>A | p.P301Q |
| COSV53163330 | Tp53 | 17:7673717-7673717 | c.903A>G | p.P301= |
| COSV52958630 | Tp53 | 17:7673716-7673716 | c.904G>C | p.G302R |
| COSV53097157 | Tp53 | 17:7673715-7673715 | c.905G>A | p.G302E |
| COSV104585646 | Tp53 | 17:7673714-7673716 | c.904_906delinsA | p.G302Kfs*3 |
| COSV52876415 | Tp53 | 17:7673714-7673714 | c.906G>T | p.G302= |
| COSV52761585 | Tp53 | 17:7673714-7673714 | c.906G>C | p.G302= |
| COSV99037454 | Tp53 | 17:7673714-7673714 | c.906G>A | p.G302= |
| COSV52729387 | Tp53 | 17:7673713-7673713 | c.907A>T | p.S303C |
| COSV53016490 | Tp53 | 17:7673712-7673712 | c.908G>C | p.S303T |
| COSV52752234 | Tp53 | 17:7673712-7673712 | c.908G>A | p.S303N |
| COSV52677891 | Tp53 | 17:7673710-7673710 | c.910A>G | p.T304A |
| COSV52772403 | Tp53 | 17:7673709-7673709 | c.911C>T | p.T3041 |
| COSV53758554 | Tp53 | 17:7673709-7673709 | c.911C>A | p.T304N |
| COSV52701088 | Tp53 | 17:7673708-7673708 | c.912T>G | p.T304= |
| COSV52766207 | Tp53 | 17:7673707-7673707 | c.913A>T | p.K305* |
| COSV52688866 | Tp53 | 17:7673707-7673707 | c.913A>G | p.K305E |
| COSV52783665 | Tp53 | 17:7673706-7673706 | c.914A>G | p.K305R |
| COSV53044660 | Tp53 | 17:7673706-7673706 | c.914A>C | p.K305T |
| COSV52688475 | Tp53 | 17:7673705-7673707 | c.913_915delinsTAA | p.K305* |
| COSV53068075 | Tp53 | 17:7673705-7673705 | c.915G>T | p.K305N |
| COSV53188252 | Tp53 | 17:7673705-7673705 | c.915G>C | p.K305N |
| COSV52702028 | Tp53 | 17:7673705-7673705 | c.915G>A | p.K305= |
| COSV52662281 | Tp53 | 17:7673704-7673704 | c.916C>T | p.R306* |
| COSV53852813 | Tp53 | 17:7673704-7673704 | c.916C>A | p.R306= |
| COSV105040157 | HRAS | 11:534318-534318 | c.5C>T | p.T2M |
| COSV54246761 | HRAS | 11:534318-534318 | c.5C>A | p.T2K |
| COSV54241514 | HRAS | 11:534316-534316 | c.7G>A | p.E3K |
| COSV54239461 | HRAS | 11:534313-534313 | c.10T>C | p.Y4H |
| COSV105040231 | HRAS | 11:534305-534305 | c.18G>A | p.L6= |
| COSV54242373 | HRAS | 11:534304-534304 | c.19G>C | p.V7L |
| COSV54241197 | HRAS | 11:534302-534302 | c.21G>A | p.V7= |
| COSV54241836 | HRAS | 11:534298-534298 | c.25G>A | p.V9M |
| COSV54243647 | HRAS | 11:534294-534294 | c.29G>C | p.G10A |
| COSV99062839 | HRAS | 11:534294-534294 | c.29G>A | p.G10D |
| COSV54247196 | HRAS | 11:534292-534292 | c.31G>T | p.A11S |
| COSV54247829 | HRAS | 11:534290-534290 | c.33C>T | p.A11= |
| COSV54236828 | HRAS | 11:534289-534289 | c.34G>T | p.G12C |
| COSV54236795 | HRAS | 11:534289-534289 | c.34G>C | p.G12R |
| COSV54237299 | HRAS | 11:534289-534289 | c.34G>A | p.G12S |
| COSV54245035 | HRAS | 11:534288-534289 | c.34_35delinsAA | p.G12N |
| COSV54236734 | HRAS | 11:534288-534288 | c.35G>T | p.G12V |
| COSV54238174 | HRAS | 11:534288-534288 | c.35G>C | p.G12A |
| COSV54236774 | HRAS | 11:534288-534288 | c.35G>A | p.G12D |
| COSV54241641 | HRAS | 11:534286-534286 | c.37G>T | p.G13C |
| COSV54236651 | HRAS | 11:534286-534286 | c.37G>C | p.G13R |
| COSV54236730 | HRAS | 11:534286-534286 | c.37G>A | p.G13S |
| COSV54241327 | HRAS | 11:534285-534286 | c.37_38delinsAA | p.G13N |
| COSV54237051 | HRAS | 11:534285-534285 | c.38G>T | p.G13V |
| COSV54237021 | HRAS | 11:534285-534285 | c.38G>A | p.G13D |
| COSV104539835 | HRAS | 11:534284-534286 | c.37_39delinsAAG | p.G13K |
| COSV54250075 | HRAS | 11:534284-534284 | c.39T>A | p.G13= |
| COSV54244292 | HRAS | 11:534282-534282 | c.41T>G | p.V14G |
| COSV54248101 | HRAS | 11:534281-534281 | c.42G>A | p.V14= |
| COSV54244967 | HRAS | 11:534280-534280 | c.43G>A | p.G15S |
| COSV54246154 | HRAS | 11:534279-534279 | c.44G>A | p.G15D |
| COSV54249283 | HRAS | 11:534276-534276 | c.47A>G | p.K16R |
| COSV54240452 | HRAS | 11:534276-534276 | c.47A>C | p.K16T |
| COSV54240358 | HRAS | 11:534274-534274 | c.49A>G | p.S17G |
| COSV99530182 | HRAS | 11:534273-534273 | c.50G>A | p.S17N |
| COSV54237030 | HRAS | 11:534271-534271 | c.52G>A | p.A18T |
| COSV54236884 | HRAS | 11:534270-534270 | c.53C>T | p.A18V |
| COSV54248213 | HRAS | 11:534264-534264 | c.59C>T | p.T20I |
| COSV105040222 | HRAS | 11:534260-534260 | c.63C>T | p.I21= |
| COSV54249870 | HRAS | 11:534259-534259 | c.64C>T | p.Q22* |
| COSV54249433 | HRAS | 11:534259-534259 | c.64C>A | p.Q22K |
| COSV54248682 | HRAS | 11:534248-534248 | c.75G>T | p.Q25H |
| COSV54243138 | HRAS | 11:534247-534247 | c.76A>G | p.N26D |
| COSV54237196 | HRAS | 11:534242-534242 | c.81T>C | p.H27= |
| COSV99529301 | HRAS | 11:534232-534232 | c.91G>T | p.E31* |
| COSV54245209 | HRAS | 11:534232-534232 | c.91G>A | p.E31K |
| COSV54240904 | HRAS | 11:534228-534228 | c.95A>G | p.Y32C |
| COSV54236910 | HRAS | 11:534227-534227 | c.96C>G | p.Y32* |
| COSV54238391 | HRAS | 11:534226-534226 | c.97G>T | p.D33Y |
| COSV54244417 | HRAS | 11:534226-534226 | c.97G>A | p.D33N |
| COSV54246151 | HRAS | 11:534223-534223 | c.100C>T | p.P34S |
| COSV54242895 | HRAS | 11:534217-534217 | c.106A>G | p.I36V |
| COSV54243103 | HRAS | 11:533934-533934 | c.122G>A | p.R41Q |
| COSV104372910 | HRAS | 11:533926-533926 | c.130G>A | p.V44M |
| COSV54248820 | HRAS | 11:533914-533914 | c.142G>A | p.G48R |
| COSV54243301 | HRAS | 11:533911-533911 | c.145G>A | p.E49K |
| COSV54237920 | HRAS | 11:533908-533908 | c.148A>G | p.T50A |
| COSV54242906 | HRAS | 11:533907-533907 | c.149C>T | p.T50M |
| COSV104372909 | HRAS | 11:533907-533907 | c.149C>A | p.T50K |
| COSV104372905 | HRAS | 11:533896-533896 | c.160G>A | p.D54N |
| COSV54248361 | HRAS | 11:533887-533887 | c.169G>A | p.D57N |
| COSV54243125 | HRAS | 11:533884-533884 | c.172A>G | p.T58A |
| COSV105040166 | HRAS | 11:533883-533884 | c.172_173delinsTT | p.T58F |
| COSV54240842 | HRAS | 11:533883-533883 | c.173C>T | p.T58I |
| COSV54244375 | HRAS | 11:533881-533881 | c.175G>A | p.A59T |
| COSV54241467 | HRAS | 11:533880-533880 | c.176C>A | p.A59D |
| COSV54242177 | HRAS | 11:533879-533879 | c.177C>T | p.A59= |
| COSV54245262 | HRAS | 11:533878-533878 | c.178G>A | p.G60S |
| COSV54239859 | HRAS | 11:533877-533877 | c.179G>A | p.G60D |
| COSV54240324 | HRAS | 11:533875-533875 | c.181C>T | p.Q61* |
| COSV54242140 | HRAS | 11:533875-533875 | c.181C>G | p.Q61E |
| COSV54236740 | HRAS | 11:533875-533875 | c.181C>A | p.Q61K |
| COSV54242436 | HRAS | 11:533874-533875 | c.181_182delinsAG | p.Q61R |
| COSV54236656 | HRAS | 11:533874-533874 | c.182A>T | p.Q61L |
| COSV54236691 | HRAS | 11:533874-533874 | c.182A>G | p.Q61R |
| COSV54238654 | HRAS | 11:533874-533874 | c.182A>C | p.Q61P |
| COSV54244391 | HRAS | 11:533873-533874 | c.182_183delinsTA | p.Q61L |
| COSV54243173 | HRAS | 11:533873-533874 | c.182_183delinsGT | p.Q61R |
| COSV54239592 | HRAS | 11:533873-533874 | c.182_183delinsGA | p.Q61R |
| COSV54236743 | HRAS | 11:533873-533873 | c.183G>T | p.Q61H |
| COSV54239883 | HRAS | 11:533873-533873 | c.183G>C | p.Q61H |
| COSV54242016 | HRAS | 11:533873-533873 | c.183G>A | p.Q61= |
| COSV54239585 | HRAS | 11:533871-533871 | c.185A>G | p.E62G |
| COSV54246293 | HRAS | 11:533866-533866 | c.190T>C | p.Y64H |
| COSV54248518 | HRAS | 11:533860-533860 | c.196G>A | p.A66T |
| COSV104539844 | HRAS | 11:533859-533859 | c.197C>A | p.A66D |
| COSV105040120 | HRAS | 11:533858-533858 | c.198C>T | p.A66= |
| COSV54240340 | HRAS | 11:533854-533854 | c.202C>T | p.R68W |
| COSV54250040 | HRAS | 11:533853-533853 | c.203G>A | p.R68Q |
| COSV54250145 | HRAS | 11:533851-533851 | c.205G>C | p.D69H |
| COSV54237939 | HRAS | 11:533851-533851 | c.205G>A | p.D69N |
| COSV54244845 | HRAS | 11:533840-533840 | c.216G>A | p.M72I |
| COSV54241667 | HRAS | 11:533839-533839 | c.217C>T | p.R73C |
| COSV54241530 | HRAS | 11:533838-533838 | c.218G>A | p.R73H |
| COSV54239294 | HRAS | 11:533836-533836 | c.220A>G | p.T74A |
| COSV54244012 | HRAS | 11:533836-533836 | c.220A>C | p.T74P |
| COSV54238067 | HRAS | 11:533834-533834 | c.222C>T | p.T74= |
| COSV54241987 | HRAS | 11:533832-533832 | c.224G>A | p.G75E |
| COSV54241978 | HRAS | 11:533831-533831 | c.225G>A | p.G75= |
| COSV54238971 | HRAS | 11:533822-533822 | c.234C>T | p.F78= |
| COSV54245079 | HRAS | 11:533817-533817 | c.239G>A | p.C80Y |
| COSV54246605 | HRAS | 11:533815-533815 | c.241G>A | p.V81M |
| COSV54250339 | HRAS | 11:533808-533808 | c.248C>A | p.A83D |
| COSV54239838 | HRAS | 11:533799-533799 | c.257A>G | p.N86S |
| COSV54249053 | HRAS | 11:533794-533794 | c.262A>G | p.K88E |
| COSV54245216 | HRAS | 11:533791-533791 | c.265T>A | p.S89T |
| COSV54245466 | HRAS | 11:533785-533785 | c.271G>C | p.E91Q |
| COSV54242442 | HRAS | 11:533782-533782 | c.274G>A | p.D92N |
| COSV104539846 | HRAS | 11:533779-533779 | c.277A>G | p.I93V |

Some embodiments include one or more mutations comprising a DNA change, an amino acid (AA) change, or a mutation at a location in TP53, CDKN2A, NOTCH1, MTOR, or HRAS, as disclosed in Table 1.

Some embodiments include one or more mutations comprising 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, 300, 400, 500, 600, 700, 800, 900, 1000, 1100, 1200, 1300, 1400, 1500, 1600, 1700, 1800, or 1839, DNA changes in Table 1, or a range defined by any two of the aforementioned numbers of DNA changes from Table 1. Some embodiments include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 250, at least 300, at least 400, at least 500, at least 600, at least 700, at least 800, at least 900, at least 1000, at least 1100, at least 1200, at least 1300, at least 1400, at least 1500, at least 1600, at least 1700, or at least 1800, of the DNA changes in Table 1. Some embodiments include less than 2, less than 3, less than 4, less than 5, less than 6, less than 7, less than 8, less than 9, less than 10, less than 11, less than 12, less than 13, less than 14, less than 15, less than 16, less than 17, less than 18, less than 19, less than 20, less than 21, less than 22, less than 23, less than 24, less than 25, less than 30, less than 35, less than 40, less than 45, less than 50, less than 55, less than 60, less than 65, less than 70, less than 75, less than 80, less than 85, less than 90, less than 95, less than 100, less than 150, less than 200, less than 250, less than 300, less than 400, less than 500, less than 600, less than 700, less than 800, less than 900, less than 1000, less than 1100, less than 1200, less than 1300, less than 1400, less than 1500, less than 1600, less than 1700, less than 1800, or less than 1839, of the DNA changes in Table 1.

Some embodiments include one or more mutations comprising 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, 300, 400, 500, 600, 700, 800, 900, 1000, 1100, 1200, 1300, 1400, 1500, 1600, 1700, 1800, or 1839, AA changes in Table 1, or a range defined by any two of the aforementioned numbers of AA changes from Table 1. Some embodiments include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 250, at least 300, at least 400, at least 500, at least 600, at least 700, at least 800, at least 900, at least 1000, at least 1100, at least 1200, at least 1300, at least 1400, at least 1500, at least 1600, at least 1700, or at least 1800, of the AA changes in Table 1. Some embodiments include less than 2, less than 3, less than 4, less than 5, less than 6, less than 7, less than 8, less than 9, less than 10, less than 11, less than 12, less than 13, less than 14, less than 15, less than 16, less than 17, less than 18, less than 19, less than 20, less than 21, less than 22, less than 23, less than 24, less than 25, less than 30, less than 35, less than 40, less than 45, less than 50, less than 55, less than 60, less than 65, less than 70, less than 75, less than 80, less than 85, less than 90, less than 95, less than 100, less than 150, less than 200, less than 250, less than 300, less than 400, less than 500, less than 600, less than 700, less than 800, less than 900, less than 1000, less than 1100, less than 1200, less than 1300, less than 1400, less than 1500, less than 1600, less than 1700, less than 1800, or less than 1839, of the AA changes in Table 1.

Some embodiments include one or more mutations at 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, 300, 400, 500, 600, 700, 800, 900, 1000, 1100, 1200, 1300, 1400, 1500, 1600, 1700, 1800, or 1839, locations in Table 1, or a range of locations from Table 1 defined by any two of the aforementioned integers. Some embodiments a mutation at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 250, at least 300, at least 400, at least 500, at least 600, at least 700, at least 800, at least 900, at least 1000, at least 1100, at least 1200, at least 1300, at least 1400, at least 1500, at least 1600, at least 1700, or at least 1800, of the locations in Table 1. Some embodiments include a mutation at less than 2, less than 3, less than 4, less than 5, less than 6, less than 7, less than 8, less than 9, less than 10, less than 11, less than 12, less than 13, less than 14, less than 15, less than 16, less than 17, less than 18, less than 19, less than 20, less than 21, less than 22, less than 23, less than 24, less than 25, less than 30, less than 35, less than 40, less than 45, less than 50, less than 55, less than 60, less than 65, less than 70, less than 75, less than 80, less than 85, less than 90, less than 95, less than 100, less than 150, less than 200, less than 250, less than 300, less than 400, less than 500, less than 600, less than 700, less than 800, less than 900, less than 1000, less than 1100, less than 1200, less than 1300, less than 1400, less than 1500, less than 1600, less than 1700, less than 1800, or less than 1839, of the locations in Table 1. A location may include a location in GRCh38, or a location in a TP53, CDKN2A, NOTCH1, MTOR, or HRAS gene or protein. For example, a mutation may be relative to GRCh38 at a location of GRCh38, or a mutation may be at a position as indicated in the DNA change column or the AA change column of Table 1.

Mutations may be caused by a variety of factors. In some embodiments, the factors include environmental factors. In some cases, mutations are caused by chemicals, air pollutants, water contamination, radiation, sun damage, or UV light. In some embodiments, a mutation is caused by a carcinogen. The mutation may result from an ingested substance. In some embodiments, a mutation is caused by exposure to radioactivity. In some embodiments, a mutation is caused by exposure to X-rays.

The one or more mutations may be detected through an amplification procedure such as polymerase chain reaction (PCR). The mutations may be detected as an amplicon. Some amplicon examples are shown in Table 2. Any of the amplicons or details in Table 2 may be used or included in the methods disclosed herein. The amplicon may be in relation to GRCh38.

TABLE 2

| TargetA | Genes | Chromosome | Start | Position | End | SEQ ID NO: |
|---|---|---|---|---|---|---|
| AMPL1198914 | MTOR | chr1 | 11124471 | 11124608 | + | 7 |
| AMPL1742798 | CDKN2A | chr9 | 21970955 | 21971097 | + | 8 |
| AMPL1742795 | CDKN2A | chr9 | 21971076 | 21971226 | + | 9 |
| AMPL1742799 | CDKN2A | chr9 | 21974577 | 21974719 | + | 10 |
| AMPL1742800 | NOTRCH1 | chr9 | 136505470 | 136505622 | + | 11 |
| AMPL1742807 | NOTRCH1 | chr9 | 136505840 | 136505990 | + | 12 |
| AMPL1742806 | NOTRCH1 | chr9 | 136517750 | 136517885 | + | 13 |
| AMPL1742809 | NOTRCH1 | chr9 | 136517867 | 136518010 | + | 14 |
| AMPL1136747 | NOTRCH1 | chr9 | 136518143 | 136518295 | + | 15 |
| AMPL1742803 | NOTRCH1 | chr9 | 136518567 | 136518720 | + | 16 |
| AMPL1742808 | NOTRCH1 | chr9 | 136523088 | 136523227 | + | 17 |
| AMPL1742805 | HRAS | chr11 | 533766 | 533908 | + | 18 |
| AMPL1121793 | HRAS | chr11 | 534197 | 534332 | + | 19 |
| AMPL1742804 | TP53 | chr17 | 7673734 | 7673872 | + | 20 |
| AMPL1423423 | TP53 | chr17 | 7674183 | 7674321 | + | 21 |
| AMPL1003288 | TP53 | chr17 | 7674875 | 7675004 | + | 22 |

### Biomarker Expression

Biomarkers may be assessed to determine skin damage, such as UV skin damage. The biomarkers may include RNA or protein biomarkers.

Skin samples obtained from the non-invasive methods and systems described herein may analyze proteins. In some instances, one or more proteins are indicative of an aging skin condition or exposure to environmental mutagens. In some instances, one or more proteins are upregulated or downregulated. In some instances, proteins are measured using mass spectrometry (e.g., LC-MS, MALDI-TOF), or binding assays (e.g., ELISA-based assay). In some instances, one or more of ORM1, LGALS3BP, A2M, B2M, DCD, Immunoglobulin mu heavy chain, HBA1, HBB, HP, SERPINC1, FGG, FGB, FGA, APOA2, APOA1, ELOVL7, ALOX15B, PLA2G4B, SERPINA3, CSTA, CST3, SERPINB1, SERPINB6, SPINT1, DAG1, S100A4, METLF, CP, SEMA7A, CDCl₄2, MUCL1, CPE, GPD2, CKM, LDHB, PYGL, CA2, CA6, NIT2, VCP, CLU, CCT8, TSN, GPC1, LMNA, PIP, SDCBP2, ANXA2, GV, TMPRSS13, RAB21, SMU1, SCGB1D2, NWD2, ATP6AP2, and C12orf10 are up-regulated in aging or mutagen-exposed skin. In some instances, one or more of ACP7, FAH, GPLD1, PSMA5, PSMB7, PLD3, EMAL4, MYH9, VASP, HARS, HARS2, AGO1, ECML1, VSIG8, CUTC, KCTD1, and SLC12A6 are downregulated in aging or mutagen-exposed skin.

The protein measurements may include a proteomic measurement. Proteomic data may be generated using mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. Some examples of methods for generating proteomic data include using mass spectrometry, a protein chip, or a reverse-phased protein microarray. Proteomic data may also be generated using a immunoassays such as enzyme-linked immunosorbent assays, western blots, dot blots, or immunohistochemistry. Generating proteomic data may involve use of an immunoassay panel. Proteins analyzed in some instances include one or more of proteins expressed by genes in Tables 1-5.

One way of obtaining proteomic data includes use of mass spectrometry. An example of a mass spectrometry method includes use of high resolution, two-dimensional electrophoresis to separate proteins from different samples in parallel, followed by selection or staining of differentially expressed proteins to be identified by mass spectrometry. Another method uses stable isotope tags to differentially label proteins from two different complex mixtures. The proteins within a complex mixture may be labeled isotopically and then digested to yield labeled peptides. Then the labeled mixtures may be combined, and the peptides may be separated by multidimensional liquid chromatography and analyzed by tandem mass spectrometry. A mass spectrometry method may include use of liquid chromatography-mass spectrometry (LC-MS), a technique that may combine physical separation capabilities of liquid chromatography (e.g., HPLC) with mass spectrometry.

Some embodiments include assessing RNA data such as transcriptomic data. Transcriptomic data may involve data about nucleotide transcripts such as RNA. Examples of RNA include messenger RNA (mRNA), ribosomal RNA (rRNA), signal recognition particle (SRP) RNA, transfer RNA (tRNA), small nuclear RNA (snRNA), small nucleolar RNA (snoRNA), long noncoding RNA (lncRNA), microRNA (miRNA), noncoding RNA (ncRNA), or piwi-interacting RNA (piRNA), or a combination thereof. The RNA may include mRNA. The RNA may include miRNA. Transcriptomic data may be distinguished by subtype, where each subtype includes a different type of RNA or transcript. For example, mRNA data may be included in one subtype, and data for one or more types of small non-coding RNAs such as miRNAs or piRNAs may be included in another subtype. A miRNA may include a 5p miRNA or a 3p miRNA.

Transcriptomic data may be generated by any of a variety of methods. Generating transcriptomic data may include using a detection reagent that binds to an RNA and yields a detectable signal. After use of a detection reagent that binds to an RNA and yields a detectable signal, a readout may be obtained that is indicative of the presence, absence or amount of the RNA. Generating transcriptomic data may include concentrating, filtering, or centrifuging a sample.

Transcriptomic data may include RNA sequence data. Some examples of methods for generating RNA sequence data include use of sequencing, microarray analysis, hybridization, polymerase chain reaction (PCR), or electrophoresis, or a combination thereof. A microarray may be used for generating transcriptomic data. PCR may be used for generating transcriptomic data. PCR may include quantitative PCR (qPCR). Such methods may include use of a detectable probe (e.g. a fluorescent probe) that intercalates with double-stranded nucleotides, or that binds to a target nucleotide sequence. PCR may include reverse transcriptase quantitative PCR (RT-qPCR). Generating transcriptomic data may involve use of a PCR panel.

RNA sequence data may be generated by sequencing a subject's RNA or by converting the subject's RNA into DNA (e.g. complementary DNA (cDNA)) first and sequencing the DNA. Sequencing may include massive parallel sequencing. Examples of massive parallel sequencing techniques include pyrosequencing, sequencing by reversible terminator chemistry, sequencing-by-ligation mediated by ligase enzymes, or phospholinked fluorescent nucleotides or real-time sequencing. Generating transcriptomic data may include preparing a sample or template for sequencing. A reverse transcriptase may be used to convert RNA into cDNA. Some template preparation methods include use of amplified templates originating from single RNA or cDNA molecules, or single RNA or cDNA molecule templates. Examples of amplification methods include emulsion PCR, rolling circle, or solid-phase amplification.

### Epigenetics

Epigenetic markers may be evaluated alone, or in combination with mutations. In some instances, a quantified burden is generated from at least one epigenetic marker. In some instances, the epigenetic markers an genomic modification. In some instances, the at least one genomic modification comprises methylation in a CpG island of a gene or a transcription regulation region of the gene. In some instances, the at least one epigenetic marker comprises 5-methylcytosine (“methylation”). In some instances, the at least one genomic modification comprises N6-methyladenine. In some instances, an epigenetic marker comprises chromatin remodeling. In some instances, chromatic remodeling comprises modification of histones. In some instances, modification of histones comprises methylation, acetylation, phosphorylation, ubiquitination, sumoylation, citrullination, or ADP-ribosylation. In some instances, the at least one genomic modification is correlated with increased exposure to environmental factors. In some instances, the at least one genomic modification is correlated with at least one additional genetic mutation. In some instances, mutation burden does not include epigenetic markers.

Epigenetic markers may be found within specific genes, near genes (e.g., promoter, terminator), or outside of genes. In some instance, at least one epigenetic markers is present in a keratin family gene. In some instances, the epigenetic marker is a proliferative marker in inflammatory diseases. In some instance, at least one epigenetic marker is present in KRT1, KRT5, KRT6, KRT14, KRT15, KRT16, KRT17, or KRT80.

Numerous methods are known in the art for resolving epigenetic markers. In some embodiments, the epigenetic markers is methylation of cytosine. In some instances, methylation sensitive endonucleases are used to identify such modifications. In some instances chemical or enzymatic differentiation of methylated vs. unmethylated bases is used (e.g., methyl C conversion to U using bisulfite). After conversion and comparison to untreated samples, methylation patterns are in some instances obtained using various sequencing and analysis techniques described herein.

Some examples of epigenetic data include DNA methylation data, DNA hydroxymethylation data, or histone modification data. Epigenetic data may include DNA methylation or hydroxymethylation. DNA methylation or hydroxymethylation may be measured in whole or at regions within the DNA. Methylated DNA may include methylated cytosine (e.g. 5-methylcytosine). Cytosine is often methylated at CpG sites and may be indicative of gene activation.

Epigenetic data may include histone modification data. Histone modification data may include the presence, absence, or amount of a histone modification. Examples of histone modifications include serotonylation, methylation, citrullination, acetylation, or phosphorylation. Some specific examples of histone modifications may include lysine methylation, glutamine serotonylation, arginine methylation, arginine citrullination, lysine acetylation, serine phosphorylation, threonine phosphorylation, or tyrosine phosphorylation. Histone modifications may be indicative of gene activation.

Epigenetic data may be obtained by a method such as sequencing, microarray analysis (e.g. a SNP microarray), hybridization, polymerase chain reaction, or electrophoresis, or a combination thereof. Epigenetic data may be generated by sequencing a subject's DNA. Sequencing may include massive parallel sequencing. Examples of massive parallel sequencing techniques include pyrosequencing, sequencing by reversible terminator chemistry, sequencing-by-ligation mediated by ligase enzymes, or phospholinked fluorescent nucleotides or real-time sequencing. Generating genomic data may include preparing a sample or template for sequencing. Some template preparation methods include use of amplified templates originating from single DNA molecules, or single DNA molecule templates. Examples of amplification methods include emulsion PCR, rolling circle, or solid-phase amplification.

An epigenetic measurement may include a DNA methylation assessment. DNA methylation can be detected by use of mass spectrometry, methylation-specific PCR, bisulfite sequencing, a HpaII tiny fragment enrichment by ligation-mediated PCR assay, a Glal hydrolysis and ligation adapter dependent PCR assay, a chromatin immunoprecipitation (ChIP) assay combined with a DNA microarray (a ChIP-on-chip assay), restriction landmark genomic scanning, methylated DNA immunoprecipitation, pyrosequencing of bisulfite treated DNA, discrimination using TET2/APO enzymatic workflows, a molecular break light assay for DNA adenine methyltransferase activity, methyl sensitive Southern blotting, methylCpG binding proteins, high resolution melt analysis, a methylation sensitive single nucleotide primer extension assay, another methylation assay, or a combination thereof.

### Skin Microbiomes

Skin samples obtained from the non-invasive methods and systems described herein may comprise non-human cellular material and/or nucleic acids. In some instances, samples comprise microorganisms. In some instances, samples comprise microbial cells or cellular material, proteins or protein subunits, nucleic acids, or nucleic acid fragments from fungi, protozoa, bacteria (Gram positive or Gram negative), yeast, virus, parasite, or other non-human microorganisms. In some instances, methods and systems described herein are used to characterize a skin microbiome. In some instances, the skin microbiome is analyzed to determine the presence of infection. In some instances, the skin microbiome is analyzed to determine general skin health. In one embodiment, a skin microbiome indicative of increased likelihood to develop a metabolic syndrome or a condition associated therewith comprises reduced bacterial community diversity, e.g., reduced number of different bacterial species, strains, or both. In one embodiment, determining that a skin microbiome comprises determining abundance of a species belonging to any family selected from: Streptococcaceae, Corynebacteriaceae, Staphylococcaceae, Micrococcaceae, Neisseriaceae, Pasteurellaceae, Prevotellaceae, Brevibacterium, Dermabacter, Malasezzia, Acidophilus, Epidermidis, Cutibacterium and Moraxellaceae, ratio of two or more species belonging to any one of the aforementioned families, or both. In some embodiments, a skin microbiome combined with mutation burden described herein are used to analyze skin. In some embodiments, a skin microbiome is indicative of increased likelihood to develop a disease or a condition. In some instances, the disease or condition is a metabolic disease or condition. In some instances, the microorganism comprises one or more of Streptococcaceae, Staphylococcaceae, Micrococcaceae, Neisseriaceae, Pasteurellaceae, Brevibacterium, Dermabacter, Malasezzia, Acidophilus, Epidermidis, Cutibacterium and Moraxellaceae. In some instances, the microorganism comprises one or more of Corynebacterium (e.g., C. kroppenstedtii) colonization, Staphylococcus, (e.g., S. aureus, S. epidermidis colonization, S. hominis colonization), or any combination thereof. In another embodiment, a skin microbiome indicative of increased likelihood to develop the metabolic syndrome or a condition associated therewith comprises colonization of one or more bacteria belonging to any family selected from: Streptococcaceae, Corynebacteriaceae, Staphylococcaceae, Micrococcaceae, Neisseriaceae, Pasteurellaceae, Prevotellaceae, Brevibacterium, Dermabacter, Malasezzia, Acidophilus, Epidermidis, Cutibacterium and Moraxellaceae. In another embodiment, a skin microbiome indicative of increased likelihood to develop the metabolic syndrome or a condition associated therewith comprises Corynebacterium colonization. In another embodiment, a skin microbiome indicative of increased likelihood to develop the metabolic syndrome or a condition associated therewith comprises Staphylococcus aureus colonization. In another embodiment, a skin microbiome indicative of increased likelihood to develop the metabolic syndrome or a condition associated therewith comprises high Corynebacterium kroppenstedtii colonization. In another embodiment, a skin microbiome indicative of increased likelihood to develop the metabolic syndrome or a condition associated therewith comprises high Staphylococcus aureus colonization. In another embodiment, a skin microbiome indicative of increased likelihood to develop the metabolic syndrome or a condition associated therewith comprises increased Corynebacterium, (e.g., C. kroppenstedtii, colonization), increased Staphylococcus, (e.g., S. aureus colonization, reduced S. epidermidis colonization, reduced S. hominis colonization), or any combination thereof. In another embodiment, a skin microbiome indicative of increased likelihood to develop the metabolic syndrome or a condition associated therewith comprises colonization of one or more bacteria belonging to any family selected from: Streptococcaceae, Corynebacteriaceae, Staphylococcaceae, Micrococcaceae, Neisseriaceae, Pasteurellaceae, Prevotellaceae, Brevibacterium, Dermabacter, Malasezzia, Acidophilus, Epidermidis, Cutibacterium and Moraxellaceae. In another embodiment, a skin microbiome indicative of increased likelihood to develop the metabolic syndrome or a condition associated therewith comprises Corynebacterium colonization. In another embodiment, a skin microbiome indicative of increased likelihood to develop the metabolic syndrome or a condition associated therewith comprises Staphylococcus aureus colonization. In another embodiment, a skin microbiome indicative of increased likelihood to develop the metabolic syndrome or a condition associated therewith comprises high Corynebacterium kroppenstedtii colonization. In another embodiment, a skin microbiome indicative of increased likelihood to develop the metabolic syndrome or a condition associated therewith comprises high Staphylococcus aureus colonization. In another embodiment, a skin microbiome indicative of increased likelihood to develop the metabolic syndrome or a condition associated therewith comprises increased Corynebacterium, e.g., (C. kroppenstedtii) colonization, increased Staphylococcus, (e.g., S. aureus colonization, reduced S. epidermidis colonization, reduced S. hominis colonization), or any combination thereof. In some instances, a microorganism detected using the non-invasive sampling systems and methods described herein comprises one or more of Staphylococcus epidermidis, Staphylococcus aureus, Staphylococcus warneri, Streptococcus pyogenes, Streptococcus mitis, Cutibacterium acnes, Corynebacterium spp., Acinetobacter johnsonii, and Pseudomonas aeruginosa. In some instances, a microorganism detected using the non-invasive sampling systems and methods described herein comprises one or more of Candida albicans, Rhodotorula rubra, Torulopsis and Trichosporon cutaneum, dermatophytes (skin living fungi) such as Microsporum gypseum, and Trichophyton rubrum and nondermatophyte fungi (opportunistic fungi that can live in skin) such as Rhizopus stolonifer, Trichosporon cutaneum, Fusarium, Scopulariopsis brevicaulis, Curvularia, Alternaria alternata, Paecilomyces, Aspergillus flavus and Penicillium. Microbiome analysis may comprise analysis of any one of bacteria, viruses, fungi, or other microorganism. In some instances, microbiome analysis provides information regarding skin hydration, sun protection, sensitivity response, antioxidant capacity, and firmness. In some instances, the amount of microorganisms from a non-invasive sample is analyzed, such as 1, 2, 3, 4, 5, 6, 7, 10, 12, 15 or more microorganisms is analyzed. In some instances, the amount of microorganisms from a non-invasive sample is analyzed, such as 1-10, 1-7, 2-7, 3-6, or 5-15 microorganisms is analyzed. In some instances, amounts, and types of microorganisms are measured using quantitative real-time PCR (qPCR). In some instance, ratios of different types of microorganisms are compared. In some instances one or more microorganisms Acidophilus, Epidermidis, S. Aureus, and C. Acnes are measured and analyzed.

### Quantitative Burden

Disclosed herein, in some embodiments, is a quantitative burden. In some embodiments, the quantitative burden is used in a method described herein. For example, the quantitative burden is calculated from a mutation burden. In some embodiments, the quantitative burden incorporates the presence of one or more mutations described herein. In some embodiments, the quantitative burden incorporates the number of identified mutations described herein for a specific patient, skin sample area, or sample location. Based on a patient's quantitative burden, they may be treated with, or recommended treatment with a skin treatment described herein. In some embodiments, the quantitative burden is generated with a computer or processor. In some embodiments, the quantitative burden is provided to a medical practitioner. In some embodiments, the quantitative burden is provided to a patient or subject.

In some embodiments, the quantitative burden comprises an integer indicative of disease risk. In some embodiments, the quantitative burden is indicative of a risk of future diseases such as skin cancer. In some embodiments, the quantitative burden is indicative of potential skin cancer. In some cases, a higher quantitative burden indicates a higher mutation burden or higher disease risk than a lower burden. In some cases, a lower quantitative burden indicates a lower mutation burden or less disease risk than a higher burden. Examples of quantitative burden values include integers from 1 to 10. In some embodiments, the quantitative burden is 1, 2, 3, 4, 5, 6, 7, 8, 9, or 10. In some embodiments, the quantitative burden is in a range defined by any two of: 1, 2, 3, 4, 5, 6, 7, 8, 9, or 10.

The quantitative burden may be quantitative (e.g., numeric or alphanumeric), with higher or lower resolution (e.g., 1-10 or high/medium/low), or qualitative (e.g., significant increase/decrease relative to a cohort), or the like. In some embodiments, the quantitative burden is quantitative. In some embodiments, the quantitative burden is numeric. In some embodiments, the quantitative burden is alphanumeric. In some embodiments, the quantitative burden is alphabetic. In some embodiments, the quantitative burden is a value or a range of values such as 1-10 or A-Z. In some embodiments, the quantitative burden is relative or general, for example: “low,” “medium,” or “high.” In some embodiments, the quantitative burden is relative to a control quantitative burden, or relative to a baseline (e.g. pre-exposure) quantitative burden.

In some embodiments, the quantitative burden is qualitative. In some embodiments, the quantitative burden is numeric. In some embodiments, the quantitative burden is “yes” or “no.” In some embodiments, the quantitative burden is “significant” or “insignificant.” In some embodiments, the quantitative burden is a significant increase or decrease relative to a control such as a cohort. In some embodiments, the quantitative burden is relative to a control quantitative burden, or relative to a baseline (e.g. pre-exposure) quantitative burden.

In some embodiments, the quantitative burden incorporates the presence or absence of one or more mutations. In some embodiments, an algorithm evaluates the various mutation types and frequency and make assumptions or recommendations when calculating the quantitative burden. In some embodiments, the algorithm uses mutation burden data, and/or patient parameters such as age, sex, skin type, history of sun damage, tanning bed use, smoking, sunburns.

In some embodiments, the quantitative burden incorporates an assessment of a subject's age, sex, skin type, history of sun damage, tanning bed use, smoking, or visible sunburn status. In some embodiments, the quantitative burden incorporates an assessment of a subject's age, smoking history, place of residence, occupation, or medical history. In some embodiments, the quantitative burden incorporates an assessment of a subject's age, gender, and/or skin condition. In some embodiments, the quantitative burden incorporates an assessment of a subject's smoking history. In some embodiments, the quantitative burden incorporates an assessment of a subject's place of residence. In some embodiments, the quantitative burden incorporates an assessment of a subject's occupation. In some embodiments, the quantitative burden incorporates an assessment of a subject's medical history. In some embodiments, the quantitative burden incorporates an assessment of a subject's skin condition. In some embodiments, the quantitative burden incorporates an assessment of a subject's history of sun damage. In some embodiments, the quantitative burden incorporates an assessment of a subject's tanning bed use. In some embodiments, the quantitative burden incorporates a visual assessment of a subject's skin damage. In some embodiments, the assessment of a subject's skin damage includes an image of the subject's skin. In some embodiments, the quantitative burden incorporates an assessment of a subject's erythema. In some embodiments, the assessment of a subject's erythema includes an erythema grade.

In some embodiments, the quantitative burden incorporates a subject's age. In some embodiments, the quantitative burden is normalized based on the subject's age. In some embodiments, the quantitative burden is increased based on the subject's age. In some embodiments, the quantitative burden is decreased based on the subject's age.

In some embodiments, the quantitative burden incorporates a subject's gender. In some embodiments, the quantitative burden is normalized based on the subject's gender. In some embodiments, the quantitative burden is increased based on the subject's gender. In some embodiments, the quantitative burden is decreased based on the subject's gender.

A quantitative burden may incorporate variables such as skin condition. In some embodiments, the quantitative burden incorporates an assessment of a subject's skin condition. In some embodiments, the skin condition is visually assessed and/or scored. In some embodiments, the quantitative burden is increased based on the subject's skin condition, such as a poor skin condition and/or erythema. In some embodiments, the quantitative burden is decreased based on the subject's skin condition, such as a good skin condition and/or lack of erythema. In some embodiments, the quantitative burden incorporates an assessment of a subject's skin type. For example, skin type may be used to categorize the level or pigmentation in skin. This level in some embodiments is used by an algorithm to generate the quantitative burden. Some embodiments of the methods described herein include analyzing a plurality of mutations (e.g. 2 or more mutations) using skin patch collection methodology for genomic analysis. Some embodiments include analyzing or algorithmically analyzing the mutational data by statistically analyzing the mutational data. Some embodiments include determining a correlation of at least two of the mutations. In some embodiments, the correlation is linear. In some embodiments, the correlation is logistic. In some embodiments, the correlation is exponential. In some embodiments, the correlation is a Pearson correlation. Some embodiments include classifying data using regression. In some embodiments, the regression is logistic. In some embodiments, the regression is linear. In some embodiments, the regression is exponential. Some embodiments include analyzing or algorithmically analyzing the mutation burden by statistically analyzing the mutation frequency data and/or other variables such as clinical parameters. In some embodiments, some of the mutations or other variables are correlated with each other, and their statistical dependence is considered when analyzing the data. In some embodiments, the analysis includes correlating the at least two mutations. In some embodiments, the analysis includes classifying data based on a regression. Some embodiments include calculating a quantitative burden based on the mutation burden. Some embodiments of the methods described herein include analyzing a plurality of mutations using skin patch collection methodology for analysis to obtain mutation burden data; algorithmically analyzing the mutation burden data by statistically analyzing the mutation location and frequency; and calculating a quantitative burden based on the analyzed mutations. In some embodiments, the mutation burden data is from mutations as described herein. Some embodiments include comparing the subject's quantitative burden to a quantitative burden range obtained from a population. Some embodiments include outputting the quantitative burden (for example, to a report, health database, healthcare practitioner, or subject). Some embodiments include recommending a skin treatment for the subject (e.g., in the report or health database, or to the healthcare practitioner or patient).

Provided herein are methods of assessing and monitoring mutation burden in a subject. Some embodiments of the methods described herein include producing a quantitative burden for a patient based on one or more mutations in genetic information. In some embodiments, determining a quantitative burden comprises determining a probability that a subject may develop a skin disease based on the one or more mutations. In some instances, a quantitative burden for a patient is in the form of a report.

In some embodiments, producing a quantitative burden comprises applying a mathematical algorithm to the mutation burden In some embodiments, the production of the quantitative burden is performed by a processor and cannot practically be performed in a human mind. For example, in some embodiments, some calculations performed by the algorithm may not be practically performed by the human mind. In some embodiments, the methods described herein provide a significant advantage in computer processing, assessment of disease risk, and patient treatment, over conventional methods. For example, the methods and systems provided herein may provide benefits in patient monitoring over conventional methods of patient monitoring, or aid in speeding up computer processing.

In some embodiments, the quantitative burden incorporates mutation location or frequency in a mutation burden. In some embodiments, the mutation burden is compared to a reference or control mutation burden measurement. In some embodiments, the mutation burden is compared to a reference mutation burden measurement. In some embodiments, the mutation burden is compared to a control mutation burden measurement. In some embodiments, the mutation burden is compared to multiple reference or control mutation burden measurements. In some embodiments, the mutation burden measurement is entered into a model, such as a regression model, relating the to an amount of disease risk. In some embodiments, the mutation burden is entered into multiple models. The reference or control mutation burden measurements can include ranges of values. In some embodiments, the reference or control mutation burden measurement is from a control patient with a known amount of environmental factor exposure. In some embodiments, the quantitative burden is relative to a control quantitative burden, or relative to a baseline (e.g. pre-exposure) quantitative burden. In some instances, a control quantitative burden is generated from a population average.

Disclosed herein, in some embodiments, are methods of producing a quantitative burden. In some embodiments, the method comprises measuring a mutation burden in a skin sample obtained from a subject. Some embodiments include generating a quantitative burden for the subject. Some embodiments include comparing the mutation burden to a model. In some embodiments, the model is derived from mutation burden in skin samples from a cohort of subjects. In some embodiments, the model is derived from amounts environmental factor exposure in the cohort of subjects. In some embodiments, the model is derived from mutation burden in skin samples from a cohort of subjects, and is derived from amounts environmental factor exposure in the cohort of subjects. Some embodiments include generating a quantitative burden for the subject by comparing the mutation burden to a model derived from a mutation burden in skin samples from a cohort of subjects, and derived from amounts of environmental factor exposure in the cohort of subjects. In some embodiments, the model comprises a random forest model. In some embodiments, comprises a boosting model. In some embodiments, the model comprises a lasso model. In some embodiments, the model comprises a logistic model. In some embodiments, the model comprises a random forest model, a boosting model, a lasso model, and/or a logistic model. In some embodiments, the model is derived using regression. In some embodiments, the model is derived using random forest classification. In some embodiments, the model is derived using logistic regression. In some embodiments, the model is derived using quantile classification. In some embodiments, the model is derived using ordinary least squares regression. In some embodiments, the model is derived using classification and regression trees.

In some embodiments, a multivariate analysis is performed to reduce a number of possible variables. In some embodiments, the analysis weighs multiple variables (which may be single target genes or interactions of target genes) based on a p-value or area under the curve (AUC) value of each individual factor. In some embodiments, the analysis puts the variables together to calculate an overall AUC value. As the overall AUC values may change with the number of variables used for the calculation, in some embodiments this produces one or more AUC curves. The one or more AUC curves may be visualized graphically (e.g. with the AUC value on y-axis, and the number of variables on x-axis). In some embodiments, a gene table ranks the importance of each variable from top to bottom (e.g. 1 to 16). Various models may be used for calculation of the overall AUC values with the number of variables. In some embodiments, 1-4 models used (random forest (if), boosting, lasso, logistic). In, for example, 4 models were used, and so 4 AUC curves may be shown in the AUC figures, and 4 columns of variables in some mutation tables. In some embodiments, AUC values on the y-axis include accumulative AUC values, with increased number of variables shown on the x-axis. In some embodiments, a higher AUC may mean a better test (given a better separation of 2 groups examined, e.g., high mutation burden vs. low mutation burden). In some embodiments, the best (or the highest) AUC is picked from the AUC curves (e.g. from AUC curves shown on an AUC figure) (regardless the models), and a number of variables (one-axis) is identified that gives this best AUC. In some embodiments, mutations from the variables will make up a mutation panel for a mutation burden (e.g. a method incorporating mutations). In some embodiments, an overall AUC is calculated, individual mutations are included.

Relationships between the mutation burden and the disease risk may be derived by any of a number of statistical processes or statistical analysis techniques. In some embodiments, logistic regression is used to derive one or more equations of the mathematical algorithm. In some embodiments, linear regression is used to derive one or more equations of the algorithm. In some embodiments, ordinary least squares regression or unconditional logistic regression is used to derive one or more equations of the algorithm. Some embodiments include a computer system that performs a method described herein, or steps of a method described herein. Some embodiments include a computer-readable medium with instructions for performing all or some of the various steps of the methods and systems provided herein. In some embodiments, the logistic regression comprises backward elimination. In some embodiments, the logistic regression comprises Akike information criterion.

Some embodiments include developing or training a model. In some embodiments, the model is an algorithm such as an algorithm for calculating a quantitative burden. In some embodiments, the model is developed by testing candidate mutations in a mutation burden. In some embodiments, the model is developed by testing candidate mutations from skin samples known to have higher risk of disease (e.g., cancer). In some embodiments, the model is developed by testing mutations from skin samples known to have a specific amount of environmental factor exposure. In some embodiments, an analytical method validation (AMV) is performed on a target gene panel. In some embodiments, multiple logistic regression is used to predict disease risk as a function of skin mutation burden. Some embodiments include logarithmic transformation and/or combined through backward elimination with Akaike information criterion (AIC). In some embodiments, a quantitative burden model is obtained by transforming a logistic function in terms of probability to have disease risk. Some embodiments include transforming a logistic function of each mutation to a probability such as a probability of having risk of a disease. Some embodiments include combining one or two logistic functions or models to product the probability. Some embodiments include generating a quantitative burden based on an input of probabilities generated for each mutation analyzed.

In some embodiments, continuous variables are reported as medians with interquartile ranges (IQR), and compared between groups using the Mann-Whitney test. In some embodiments, categorical variables are reported as numbers (n) and percentages (%), and compared between groups using a Fisher's exact test. In some embodiments, a Delong method is used to compute a 95% confidence interval (CI) of AUROC, and/or to compare AUROCs of different target genes on paired samples. In some embodiments, exact binomial confidence limits are used for the 95% CIs of sensitivity and specificity. In some embodiments, the 95% CIs of PLR and NLR are computed. In some embodiments, a pairwise Wilcoxon rank sum test is used for comparing effect size of different variables. In some embodiments, a p value (e.g. one-sided or two-sided) of 0.05 or lower is considered as significant.

In some embodiments, applying the mathematical algorithm to the mutation burden comprises using one, two, three, or more models relating the position, type, or occurrence of the at least one mutation to a quantitative burden. In some embodiments, results are generated from more than one model. In some embodiments, the results comprise a probability such as a probability of a patient developing a disease. In some embodiments, the results generated from each of the more than one model are averaged. In some embodiments, producing an exposure score for the patient comprises using one, two, three, or more models relating mutation burden to a known amount disease risk. In some embodiments, the mathematical algorithm comprises a model relating mutation burden to a known amount of environmental factor exposure or disease risk. In some embodiments, the mathematical algorithm comprises two or more models relating the mutation burden to a known amount of environmental factor exposure. In some embodiments, one or more of the models are derived by using classification and regression trees, and/or one or more of the models are derived by using ordinary least squares regression to model diagnostic specificity. In some embodiments, one or more of the models are derived by using random forest learning classification, and/or one or more of the models are derived by using quantile classification. In some embodiments, one or more of the models are derived by using logistic regression to model diagnostic sensitivity, and/or one or more of the models are derived by using logistic regression to model diagnostic specificity. In some embodiments, the use of two or more models provides an unexpected benefit of increasing sensitivity in relating the quantitative burden to the known amount of environmental factor exposure. In some embodiments, the use of two or more models provides an unexpected benefit of increasing specificity in relating the mutation burden to the known amount of environmental factor exposure.

In some embodiments, the statistical analyses includes a quantile measurement of one or more target genes. Quantiles can be a set of “cut points” that divide a sample of data into groups containing (as far as possible) equal numbers of observations. For example, quartiles can be values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The algorithm can also include the use of percentile ranges of mutation frequencies (e.g., tertiles, quartile, quintiles, etc.), or their cumulative indices (e.g., quartile sums of mutation frequency to obtain quartile sum scores (QSS), etc.) as variables in the statistical analyses (just as with continuous variables).

In some embodiments, the statistical analyses include one or more learning statistical classifier systems. As used herein, the term “learning statistical classifier system” includes a machine learning algorithmic technique capable of adapting to complex data sets (e.g., panel of target genes of interest) and making decisions based upon such data sets. In some embodiments, a single learning statistical classifier system such as a decision/classification tree (e.g., random forest (RF) or classification and regression tree (C&RT)) is used. In some embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as RF, C&RT, boosted trees, etc.), Probably Approximately Correct (PAC) learning, connectionist learning (e.g., neural networks (NN), artificial neural networks (ANN), neuro fuzzy networks (NFN), network structures, the Cox Proportional-Hazards Model (CPHM), perceptrons such as multi-layer perceptrons, multi-layer feed-forward networks, applications of neural networks, Bayesian learning in belief networks, etc., reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees.

Classification and regression trees represent a computer intensive alternative to fitting classical regression models and are typically used to determine the best possible model for a categorical or continuous response of interest based upon one or more predictors. In some embodiments, the statistical methods or models are trained or tested using a cohort of samples (e.g., skin samples) from healthy individuals with and without environmental factor exposure.

In certain aspects, one or more equations of the mathematical algorithm are derived to model diagnostic sensitivity, e.g., the proportion of actual positives that are correctly identified as such. For example, one or more equations can be trained using the data to predict a disease risk with the measured mutation burden. In certain aspects, one or more equations of the mathematical algorithm are derived to model diagnostic specificity, e.g., the proportion of actual negatives that are correctly identified as such. For example, one or more equations can be trained using the data to predict disease risk with the measured mutation burden. In some embodiments, the mathematical algorithm includes two or more equations, one or more of which are derived to model diagnostic sensitivity, and one or more of which are derived to model diagnostic specificity. In certain aspects, the mathematical algorithm applies one or more diagnostic sensitivity equations prior to applying one or more diagnostic specificity equations in a sequence to generate a quantitative burden. In certain aspects, the mathematical algorithm applies one or more diagnostic specificity equations prior to applying one or more diagnostic sensitivity equations in a sequence to generate a quantitative burden. In some embodiments, the algorithm is trained based on skin samples known to have been exposed to environmental factors and known mutation burdens.

Some embodiments of the methods and systems described herein include generating a probability of the patient developing a disease by applying a model to at least one mutation. In some embodiments, the probability is 0%, 1%, 5%, 10%, 15%, 20%, 25%, 30%, 35%, 40%, 45%, 50%, 55%, 60%, 65%, 70%, 75%, 80%, 85%, 90%, 95%, 99%, or 100%. In some embodiments, the probability is 0-10%. In some embodiments, the probability is 10-20%. In some embodiments, the probability is 20-30%. In some embodiments, the probability is 30-40%. In some embodiments, the probability is 40-50%. In some embodiments, the probability is 50-60%. In some embodiments, the probability is 60-70%. In some embodiments, the probability is 70-80%. In some embodiments, the probability is 80-90%. In some embodiments, the probability is 90-100%. Some embodiments include generating a probability for mutation. In some embodiments, each mutation is multiplied by a separate factor. In some embodiments, the probability for each mutation is multiplied by a separate factor. Some embodiments, include generating a probability based on multiple mutations.

In some embodiments, at least one mutation is weighted (e.g., based on type of mutation, location of mutation, or frequency of mutation). In some embodiments, the weight of the mutation is compared to a threshold. In some embodiments, the weight of a mutation is assigned by a computer algorithm. In some embodiments, the weight of a mutation affects how much a particular mutation contributes to calculating a quantitative burden. In some embodiments, the weight of a first mutation is less than the weight of a second mutation. In such cases, the first mutation can be less informative of the quantitative burden than the second mutation. In some embodiments, the weight of a first mutation is greater than the weight of a second mutation level. In such cases, the first mutation can be more informative of disease risk or the quantitative burden than the second mutation. In some embodiments, each mutation is given a separate weight in the mathematical algorithm. For example, one mutation may have a greater impact on the quantitative burden than another mutation.

In some embodiments, the weight is 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 50, or 100, in relation to another of the mutations. In some embodiments, the weight is 0.01-0.1 in relation to another of the mutations. In some embodiments, the weight is 0.1-0.5 in relation to another of the mutations. In some embodiments, the weight is 0.5-1 in relation to another of the mutations. In some embodiments, the weight is 1-1.5 in relation to another of the mutations. In some embodiments, the weight is 1.5-2 in relation to another of the mutations. In some embodiments, the weight is 2-10 in relation to another of the mutations. In some embodiments, the weight is 10-100 in relation to another of the mutations. In some embodiments, the mutations is weighted such that it contributes 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 50, or 100% of the quantitative burden.

Some embodiments of the methods and systems described herein include based on the weight for the probability generated from each mutation, generating an overall probability of the subject's disease risk, or an amount of mutation burden. In some embodiments, the overall probability is 0%, 1%, 5%, 10%, 15%, 20%, 25%, 30%, 35%, 40%, 45%, 50%, 55%, 60%, 65%, 70%, 75%, 80%, 85%, 90%, 95%, 99%, or 100%. In some embodiments, the overall probability is 0-10%. In some embodiments, the overall probability is 10-20%. In some embodiments, the overall probability is 20-30%. In some embodiments, the overall probability is 30-40%. In some embodiments, the overall probability is 40-50%. In some embodiments, the overall probability is 50-60%. In some embodiments, the overall probability is 60-70%. In some embodiments, the overall probability is 70-80%. In some embodiments, the overall probability is 80-90%. In some embodiments, the overall probability is 90-100%.

Some embodiments include the use of an intermediate value for the mutation burden. In some embodiments, the algorithm converts a mutation frequency into an intermediate value for that mutation. In some embodiments, the algorithm converts the level of multiple mutations, or all of the mutations, into intermediate values. In some embodiments, the algorithm converts the mutation burden into a single intermediate value. In some embodiments, the intermediate values are converted by the algorithm into the quantitative burden. In some embodiments, the use of an intermediate value improves the speed of producing the quantitative burden from the mutation burden, thereby increasing the processing speed of a computer or device implementing the mathematical algorithm. In some embodiments, the use of an intermediate value improves a computer technology or other device.

In some embodiments, a mutation burden that is less than a reference or control mutation burden is indicative of disease risk. In some embodiments, a mutation burden that is greater than a reference or control mutation burden is indicative of disease risk. In some embodiments, a mutation burden that is less than a reference or control mutation burden is indicative of a lack of disease risk. In some embodiments, a mutation burden that is greater than a reference or control mutation burden is indicative of a lack of disease risk. In some embodiments, a mutation burden that is less than a reference or control mutation burden is indicative of an amount of disease risk. In some embodiments, a mutation burden that is greater than a reference or control mutation burden is indicative of an amount of disease risk.

In some embodiments, a computer or processor applies a mathematical algorithm to the measured mutation burden. In some embodiments, the quantitative burden is produced by or using a computer or processor. In some embodiments, the computer or processor receives the mutation burden data. In some embodiments, a user enters the mutation burden data, for example into a graphical user interface. In some embodiments, the computer or processor implements the mathematical algorithm to generate the quantitative burden. In some embodiments, the computer or processor performs or is used to perform one, more, or all steps of the method. In some embodiments, the computer or processor displays the quantitative burden. In some embodiments, the computer or processor transmits the quantitative burden, for example over a network to another computer or processor. Some embodiments include receiving the quantitative burden.

Some embodiments of the methods described herein include obtaining or generating a quantitative burden for a subject. Some embodiments include comparing the quantitative burden for the subject to a reference quantitative burden (such as a quantitative burden obtained from a population, or multiple populations). The reference quantitative burden may include a value or a value range for subjects with exposure to environmental factors. The reference quantitative burden may include values or a value range for subjects with various amounts of environmental factor exposure (e.g. quantile amounts of UV exposure or other mutations, and quantitative burden ranges delineating each quantile). The reference quantitative burden may include values or a value range for subjects without environmental factor exposure. Some embodiments include determining an amount of deviation of the quantitative burden for the subject compared to a quantitative burden from a population or a corresponding range. For example, some embodiments include determining a percent of deviation of the quantitative burden for the subject compared to a quantitative burden obtained from a population. In some embodiments, the quantitative burden obtained from a population range thereof includes an average quantitative burden, or a quantile quantitative burden such as a quartile or quintile quantitative burden. Some embodiments include indicating a degree of disease risk for the subject based on the quantitative burden for the subject. Such indications may come in the form of a recommendation, a determination, or a communication about the determination or recommendation. In some instances, a population has an age range of 10-100, 10-75, 10-50, 15-25, 25-35, 30-50, 20-70, 40-75, 50-100, 40-60, or 40-100 years.

In some embodiments, the quantitative burden is informative of disease risk. In some embodiments, the quantitative burden is informative of skin cancer risk. In some embodiments, the quantitative burden is informative of UV skin exposure. In some embodiments, the quantitative burden is informative of an amount of UV skin exposure.

Some embodiments relate to a method comprising one or more of the following steps: Step 1) analyze a plurality of mutations from skin samples collected using skin patch methodology to obtain mutation burden data; Step 2) algorithmically analyze mutation burden data collected in Step 1 using the method in Steps 2A and 2B; Step 2A) statistically analyze a plurality of collected mutation burden data (e.g. from mutations provided herein); Step 2C) combine the mutations and mutation frequency by classification or regression algorithms to calculate a quantitative burden; Step 4) (optional) compare patient quantitative burden to a quantitative burden range obtained from a population; Step 5) output the quantitative burden (e.g., to a report, to a database such as a health database, or to a patient; Step 6) (optional) recommend a treatment; and Step 7) (optional) treat the patient. The plurality of mutations in some instances include one or more mutations as described herein. The plurality of mutations in some instances include one or more mutations as described in Tables 1-5.

Mutations in samples may be processed or analyzed in parallel using high-throughput multiplex methods described herein to quantify a mutation burden (e.g., mass-array, hybridization array, specific probe hybridization, whole genome sequencing, or other method). In some embodiments, methods described herein comprise genotyping. The nucleic acids analyzed from the sample in some instances represent the entire genome or a sub-population thereof (e.g., genomic regions, genes, introns, exons, promoters, intergenic regions). In some instances, these nucleic acids are analyzed from one or more panels which target mutations or groups of mutations. In some instances, methods describe herein comprise detecting one or more mutations in these nucleic acids. In some instances, 25-50,000, 50-50,000, 100-100,000, 25-10,000, 25-5,000 or 300-700 mutations are analyzed. In some instances, at least 300, 400, 500, 750, 1000, 2000, 5000, 10,000, or more than 10,000 mutations are analyzed. In some instances, two or more mutations are used to generate a pattern representative of the quantitative burden. In some examples, a subset of genomic regions will be sequenced to perform a panel analysis of mutations in the subset of genomic regions (or of the whole genome) to output a set of mutations for the sample. For instance, a variety of mutational panels could be utilized, for instance the MSK-IMPACT panel. Accordingly, the result of this process in some instances is an output of a set of mutations based on the subset of sequenced genomic regions or the whole genome. In some instances, the sequence data is transmitted over a network to be stored in a database by a server or further processed on local memory. In some examples, the server may then perform further processing on the sequence data or sequence data files. Further analysis of sequencing data is in some instances used to generate a quantitative burden.

Next, the system may process the set of somatic mutations to output a sample mutation spectrum. The mutational spectrum in some instances is a vector, table, list or other compilation of the number of mutation types. In some instances the vector contains the counts of the 96 mutation types concept from Alexandrov, et al. Nature, 2013, pp 415-421. These 96 mutation types include (1) 5′ flanking base (A, C, G, T); (2) the 6 substitution classes (C>A, C>G, C>T, T>A, T>C, T>G) and (3) 3′ flanking base (A, C, G, T). Taken together these lead to the 96 mutation types classification (4×6×4=96). In some embodiments, other mutational signatures are be developed over different types of mutations such as genomic rearrangements.

After determining the mutational spectrum of the sample, it may be compared to predetermined clusters of mutational spectrums. The predetermined clusters of mutational spectrums in some instances are derived by determining mutational spectrums from the whole genome of various samples, and clustering the samples using, e.g., hierarchical clustering, based on the fractional occurrence of each mutation in a sample. In other examples, the predetermined clusters are determined from samples that have less than the whole genome sequenced (e.g. a subset of genomic regions as described above) and using different clustering methods including k-means clustering, silhouette width, expectation maximization, or other clustering method.

The sample mutational spectrum may be compared to the predetermined clusters using a variety of methods. In some instances, the method comprises a likelihood similarity measure. In some instances other methods are utilized including a likelihood calculated with different probability distributions rather than a binomial distribution (e.g. negative binomial), cosine similarity, or Euclidean distance. Then a matching cluster(s) in some instances is identified. Sequencing data in some instances is down-sampled to the regions covered by targeted genomic regions to simulate panel data. In some instances, the simulation determines a threshold that defines a sufficiently large matching score that yields few samples that are falsely matched.

In other examples, additional matching scores such as cosine similarity are calculated to a signature in the catalog and the magnitude of a signature is calculated with linear decomposition (NNLS) to find magnitude of several signatures simultaneously. In some instances, these methods are effective when the number of mutations is large, but they can improve the robustness of the method when used in combination with matching to a cluster. A multivariate machine learning (ML) model in some instances is trained that combines several features including the matching score to clusters and predicts a final quantitative burden. Simulations in some instances are used in the training.

In other examples, training is done using panel data or simulated panels from other sources rather than WGS (whole-genome sequencing) data, if the status of the signature is known by other identifiers rather than the analysis of WGS data. In some embodiments, the trained ML method is used to predict a final quantitative burden that indicates presence of a specific signature for which the training has been done.

For instance, a trained gradient boosting machine(s) is in some instances utilized to combine the above features or different combinations of the above features to output a final quantitative burden. Some or all measures, including likelihood measures, are in some instances calculated in simulations mentioned above, and are optionally combined to output a final quantitative burden using machine learning methods. For instance, a gradient boosting machine is trained using simulated spectrums and samples from the publicly available whole genome sequenced data, or other data source comprising mutations. In other examples, other types of machine learning algorithms such as random forest, naiive Bayesian, elastic net, support vector machines, lasso, and/or generalized linear regression are utilized to analyze the features.

In some examples, the features that are combined into a single score include: (1) cosine similarity; (2) likelihood similarity measures for signature positive and signature negative clusters; (3) signature exposure calculated with NNLS; (4) likelihood of a given NNLS decomposition compared to other possible decompositions; and (5) total number of mutations.

In some embodiments, these features are combined with a gradient boosting classifier to apply the appropriate weighting to the features. In some examples, certain subsets of the features are more important than other features or subsets of features. Panel-based data the likelihood similarity measures in some instances is the most important or the only features utilized. For WGS data, the linear decomposition features in some instances are the most important but linear decomposition features in some instances are not accurate for panel data (with much smaller numbers of mutations).

The quantitative burden may be utilized to determine whether a patient is likely at risk for certain defects or maladies associated with particular signatures (e.g., cancer). Accordingly, different score thresholds are in some instances set based on the confidence required or desired based on the anticipated action (e.g. treatment). For instance, if a drug with low side impacts is available, the threshold in some instances is set lower and the drug administered as a prophylactic. In some instance, more aggressive treatments are utilized if there is a higher confidence based on the resulting quantitative burden. Having a higher confidence in some instances is more optimal in order to observe a better response to treatment in the selected cohort because of the higher specificity.

### Non-Invasive Sampling

In some embodiments, the adhesive patch from the sample collection kit described herein comprises a first collection area comprising an adhesive matrix and a second area extending from the periphery of the first collection area. The adhesive matrix is located on a skin facing surface of the first collection area. The second area functions as a tab, suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use.

In some embodiments, the first collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent. In some embodiments, the adhesive matrix is configured to adhere cells from the stratum corneum of a skin sample.

The matrix material is sufficiently sticky to adhere to a skin sample. The matrix material is not so sticky that is causes scarring or bleeding or is difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant.

In some embodiments, the adhesive patch comprises a flexible material, enabling the patch to conform to the shape of the skin surface upon application. In some instances, at least the first collection area is flexible. In some instances, the tab is plastic. In an illustrative example, the adhesive patch does not contain latex, silicone, or both. In some embodiments, the adhesive patch is made of a transparent material, so that the skin sampling area of the subject is visible after application of the adhesive patch to the skin surface. The transparency ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical.

In further embodiments, the adhesive patch of this invention is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector.

In some instances, nucleic acids are stable on adhesive patch or patches when stored for a period of time or at a particular temperature. In some instances, the period of time is at least or about 1 day, 2 days, 3 days, 4 days, 5 days, 6 days, 7 days, 2 weeks, 3 weeks, 4 weeks, or more than 4 weeks. In some instances, the period of time is about 7 days. In some instances, the period of time is about 10 days. In some instances, the temperature is at least or about −80° C., −70° C., −60° C., −50° C., −40° C., −20° C., −10° C., −4° C., 0° C., 5° C., 15° C., 18° C., 20° C., 25° C., 30° C., 35° C., 40° C., 45° C., 50° C., or more than 50° C. The nucleic acids on the adhesive patch or patches, in some embodiments, are stored for any period of time described herein and any particular temperature described herein. For example, the nucleic acids on the adhesive patch or patches are stored for at least or about 7 days at about 25° C., 7 days at about 30° C., 7 days at about 40° C., 7 days at about 50° C., 7 days at about 60° C., or 7 days at about 70° C. In some instances, the nucleic acids on the adhesive patch or patches are stored for at least or about 10 days at about −80° C.

The peelable release sheet, in certain embodiments, is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the peelable release sheet is configured to hold about 12 adhesive patches. In some instances, the peelable release sheet is configured to hold about 11 adhesive patches. In some instances, the peelable release sheet is configured to hold about 10 adhesive patches. In some instances, the peelable release sheet is configured to hold about 9 adhesive patches. In some instances, the peelable release sheet is configured to hold about 8 adhesive patches. In some instances, the peelable release sheet is configured to hold about 7 adhesive patches. In some instances, the peelable release sheet is configured to hold about 6 adhesive patches. In some instances, the peelable release sheet is configured to hold about 5 adhesive patches. In some instances, the peelable release sheet is configured to hold about 4 adhesive patches. In some instances, the peelable release sheet is configured to hold about 3 adhesive patches. In some instances, the peelable release sheet is configured to hold about 2 adhesive patches. In some instances, the peelable release sheet is configured to hold about 1 adhesive patch.

Provided herein, in certain embodiments, are methods and compositions for obtaining a sample using an adhesive patch, wherein the adhesive patch is applied to the skin and removed from the skin. After removing the used adhesive patch from the skin surface, the patch stripping method, in some instances, further comprise storing the used patch on a placement area sheet, where the patch remains until the skin sample is isolated or otherwise utilized. In some instances, the used patch is configured to be stored on the placement area sheet for at least 1 week at temperatures between −80° C. and 30° C. In some embodiments, the used patch is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some instances, the placement area sheet comprises a removable liner, provided that prior to storing the used patch on the placement area sheet, the removable liner is removed. In some instances, the placement area sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the placement area sheet is configured to hold about 12 adhesive patches. In some instances, the placement area sheet is configured to hold about 11 adhesive patches. In some instances, the placement area sheet is configured to hold about 10 adhesive patches. In some instances, the placement area sheet is configured to hold about 9 adhesive patches. In some instances, the placement area sheet is configured to hold about 8 adhesive patches. In some instances, the placement area sheet is configured to hold about 7 adhesive patches. In some instances, the placement area sheet is configured to hold about 6 adhesive patches. In some instances, the placement area sheet is configured to hold about 5 adhesive patches. In some instances, the placement area sheet is configured to hold about 4 adhesive patches. In some instances, the placement area sheet is configured to hold about 3 adhesive patches. In some instances, the placement area sheet is configured to hold about 2 adhesive patches. In some instances, the placement area sheet is configured to hold about 1 adhesive patch.

The used patch, in some instances, is stored so that the matrix containing, skin facing surface of the used patch is in contact with the placement area sheet. In some instances, the placement area sheet is a panel of the tri-fold skin sample collector. In some instances, the tri-fold skin sample collector further comprises a panel. In some instances, the tri-fold skin sample collector further comprises a clear panel. In some instances, the tri-fold skin sample collector is labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive patches stored on a peelable release panel. In some instances, the tri-fold skin sample collector further comprises a placement area panel with a removable liner. In some instances, the patch stripping method involves removing an adhesive patch from the tri-fold skin sample collector peelable release panel, applying the adhesive patch to a skin sample, removing the used adhesive patch containing a skin sample and placing the used patch on the placement area sheet. In some instances, the placement area panel is a single placement area panel sheet. In some instances, the identity of the skin sample collected is indexed to the tri-fold skin sample collector or placement area panel sheet by using a barcode or printing patient information on the collector or panel sheet. In some instances, the indexed tri-fold skin sample collector or placement sheet is sent to a diagnostic lab for processing. In some instances, the used patch is configured to be stored on the placement panel for at least 1 week at temperatures between −80° C. and 25° C. In some embodiments, the used patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments, the indexed tri-fold skin sample collector or placement sheet is sent to a diagnostic lab using UPS or FedEx.

In an exemplary embodiment, the patch stripping method further comprises preparing the skin sample prior to application of the adhesive patch. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The patch stripping method, devices, and systems described herein are useful for the collection of a skin sample from a skin lesion. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion. The skin sample may be from a skin lesion or a non-lesional skin area.

In some embodiments, the tape stripping includes collection of a sample from a collection site. The collection site may include any skin site on a subject. Examples of skin sites include a head, facial, neck, shoulder, back, arm, hand, chest, stomach, pelvis, leg, or foot. The collection site may include a facial site. The facial site may include a lip, chin, forehead, nose, cheek, or temple site. The forehead site may include a center forehead, right forehead, left forehead, top forehead, or bottom forehead site. The cheek site may include a right or left cheek. The temple site may include a right or left temple. In some embodiments, a method includes collecting a skin sample from one or more of these areas. Some embodiments include receiving or using a skin sample previously collected from one or more of these sites. In some embodiments, a method may include obtaining or using data from skin samples collected from any of these or other skin areas.

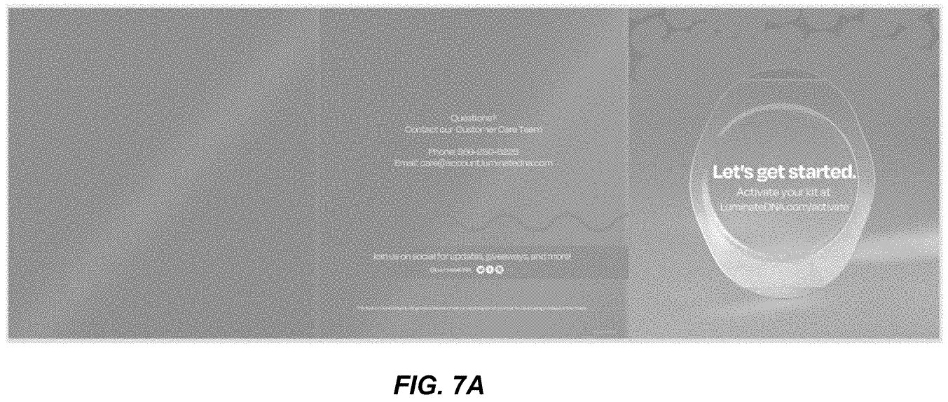

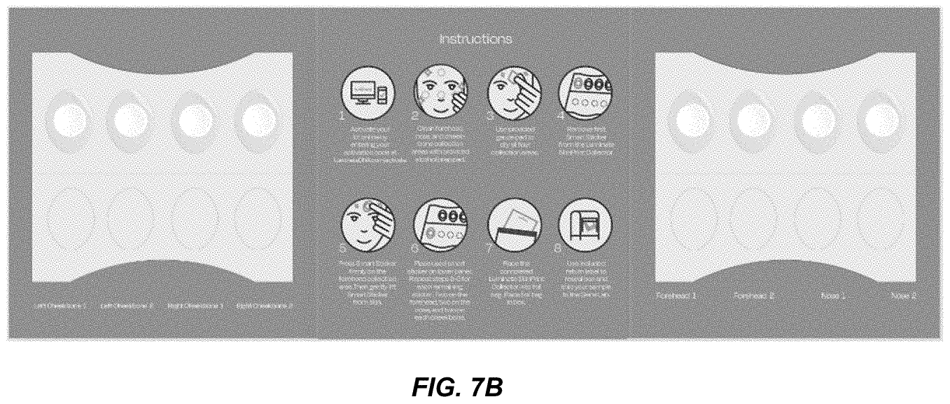

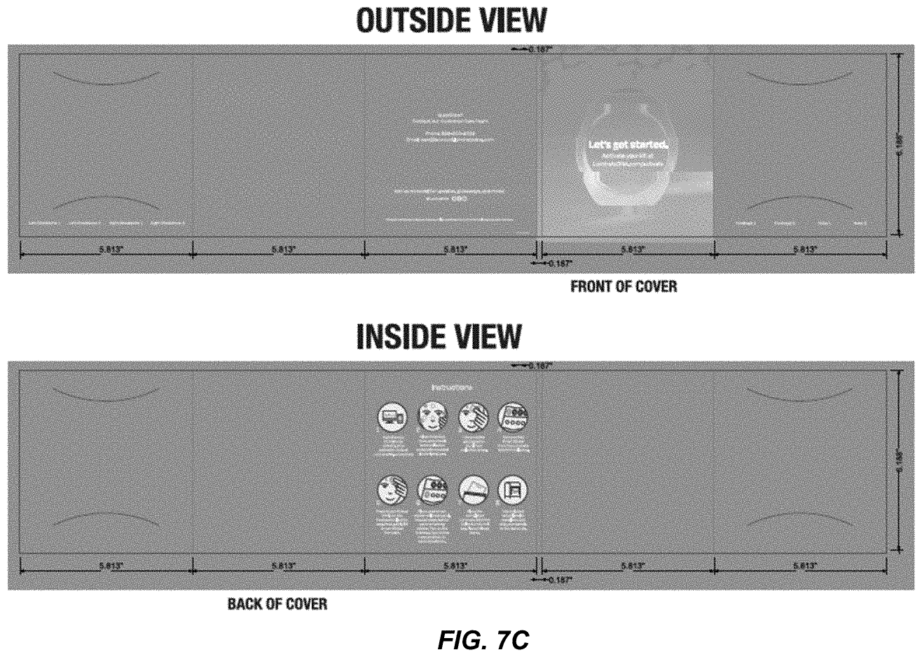

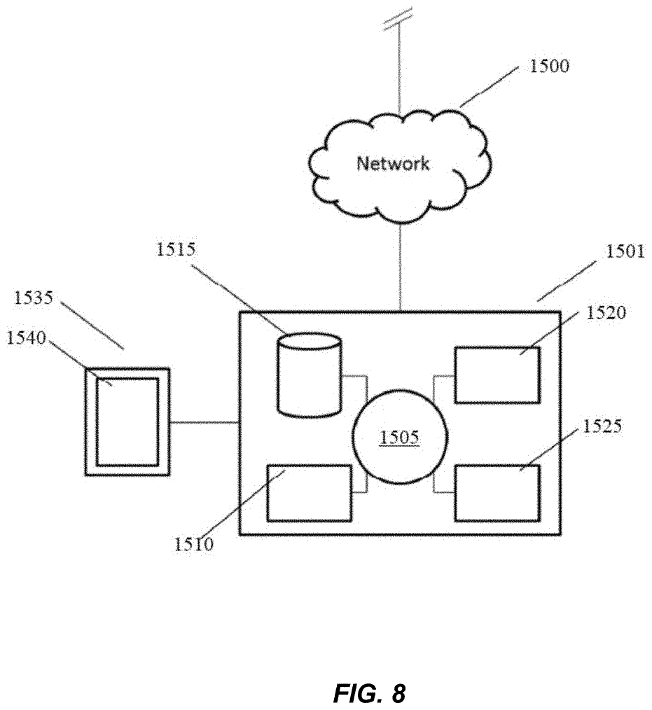

The adhesive skin sample collection kit, in some embodiments, comprises at least one adhesive patch, a sample collector, and an instruction for use sheet. In an exemplary embodiment, the sample collector is a tri-fold skin sample collector comprising a peelable release panel comprising at least one adhesive patch, a placement area panel comprising a removable liner, and a clear panel. The tri-fold skin sample collector, in some instances, further comprises a barcode and/or an area for transcribing patient information. In some instances, the adhesive skin sample collection kit is configured to include a plurality of adhesive patches, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provide the kit operator all of the necessary information for carrying out the patch stripping method. The instructions for use sheet preferably include diagrams to illustrate the patch stripping method. A placement area panel or adhesive patch may appear as included in FIG. 7B or FIG. 7C.

In some instances, the adhesive skin sample collection kit provides all the necessary components for performing the patch stripping method. In some embodiments, the adhesive skin sample collection kit includes a lab requisition form for providing patient information. In some instances, the kit further comprises accessory components. Accessory components include, but are not limited to, a marker, a resealable plastic bag, gloves and a cleansing reagent. The cleansing reagent includes, but is not limited to, an antiseptic such as isopropyl alcohol. In some instances, the components of the skin sample collection kit are provided in a cardboard box.

In some embodiments, the kit includes a skin collection device. In some embodiments, the skin collection device includes a non-invasive skin collection device. In some embodiments, the skin collection device includes an adhesive patch as described herein. In some embodiments, the skin collection device includes a brush. In some embodiments, the skin collection device includes a swab. In some embodiments, the skin collection device includes a probe. In some embodiments, the skin collection device includes a medical applicator. In some embodiments, the skin collection device includes a scraper. In some embodiments, the skin collection device includes an invasive skin collection device such as a needle or scalpel. In some embodiments, the skin collection device includes a needle. In some embodiments, the skin collection device includes a microneedle. In some embodiments, the skin collection device includes a hook.

Disclosed herein, in some embodiments, are kits for collecting cellular or genetic material, or for quantifying mutation burden in a skin sample. In some embodiments, the kit includes an adhesive patch. In some embodiments, the adhesive patch comprises an adhesive matrix configured to adhere skin sample cells from the stratum corneum of a subject. Some embodiments include a nucleic acid isolation reagent. Some embodiments include a plurality of probes that recognize at least one mutation. Disclosed herein, in some embodiments, are kits for determining a mutation burden in a skin sample, comprising: an adhesive patch comprising an adhesive matrix configured to adhere skin sample cells; a nucleic acid isolation reagent; and at least one probe that recognize at least one mutation used to quantify the mutation burden. Disclosed herein, in some embodiments, are kits for determining a mutation burden in a skin sample, comprising: an adhesive patch comprising an adhesive matrix configured to adhere skin sample cells; a sample collector, and instructions for collecting the sample and storing in the collector.

In some embodiments, a kit may include an aspect shown in any of FIG. 7A-7C. For example, the kit may include packaging or instructions as shown, or may consist of the aspects shown in any of the figures. Any aspect of the kit may be used in a method described herein. A kit may use the dimensions or orientation in FIG. 7C.

In some embodiments, a method described herein uses any aspect in any of FIG. 7A-7C. For example, a method may include any of the following: activating a kit using an activation code; cleaning of a skin collection site, for example, using an alcohol cleaning pad; drying the skin collection site, for example, using a gauze strip; removing a skin collection device such as a smart sticker comprising an adhesive patch with an adhesive matrix for collecting a skin sample; pressing the skin collection device against the skin collection site to adhere skin cells (e.g. cells of the stratum corneum) to an adhesive matrix of the skin collection device; adhering skin cells to multiple skin collection devices, perhaps from various skin sites, placement of the skin collection device(s) onto a placement area panel; placement of the placement area panel into a container such as a bag or box; or shipment of the skin sample, e.g. adhered to skin collection device(s) and placed on a placement area panel, to a diagnostic facility. In some instances, the kit comprises instructions for contacting the kit manufacturer, such as by email, phone, fax, or website.

In some embodiments, a skin assessment or skin sample collection kit is sent (e.g. mailed or delivered) to a subject. The kit may be delivered upon being ordered requested by the subject. The order may be made by mail or electronically. In some embodiments, the subject has a subscription, and receives the kit periodically (e.g. every 21-28 days, or every 1, 2, 3, 4, 5, or 6 months). In some instances, a system or method described herein comprises subscribing to a monitoring service; receiving a kit; returning a kit comprising a sample; and receiving a skin mutation burden assessment. Prescription of a monitoring system in some instances is based on a patient's skin risk. In some instances, patients at higher risk for developing a serious skin condition are prescribed a monitoring system. In some instances, monitoring is prescribed to evaluate the result of an ongoing treatment, or monitor a patient after treatment (e.g., for relapse).

The kit may be delivered to a subject based on an assessment or determination that the subject is at risk of skin mutations. For example, the subject may be exposed to environmental factors, chemicals, air pollutants, water contamination, radiation, sun damage, UV light a carcinogen, radioactivity, or X-rays. In some embodiments, the subject has a high-risk job where exposure to any such factor is greater than normal.

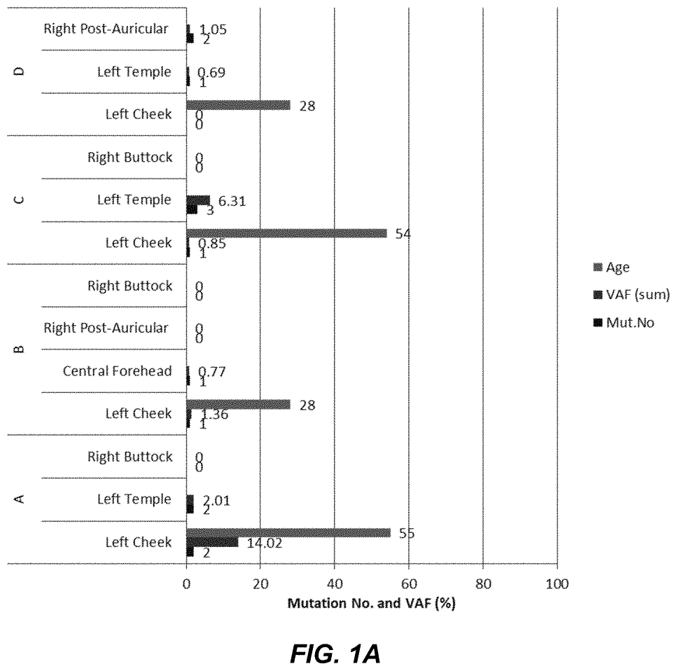

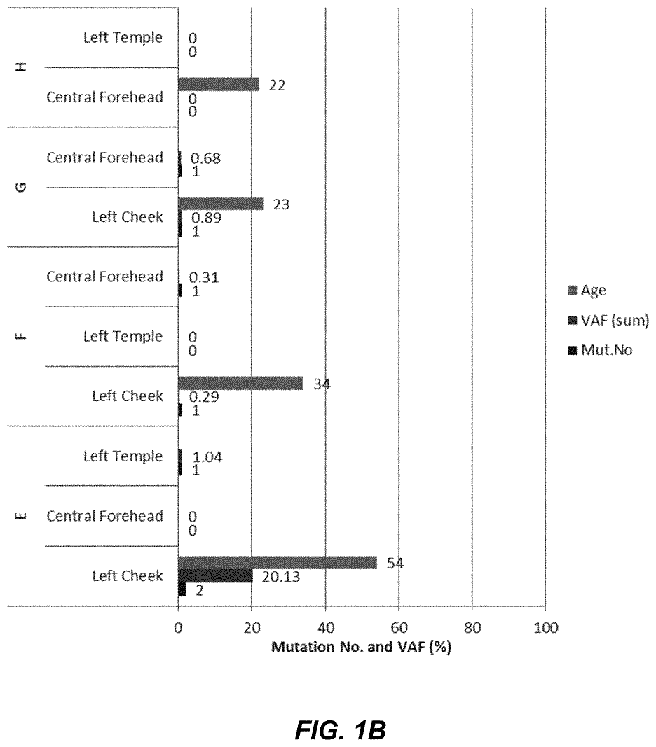

In some embodiments, the kit is labeled for where the skin sample comes from on the subject (e.g., high UV exposure areas versus low UV exposure areas; or specific sampling locations such as the head (e.g., bald or balding), temple, forehead, cheek, ear, or nose). In some embodiments, the adhesive patch is at least 1 cm², at least 2 cm², at least 3 cm², or at least 4 cm², based on the skin sampling location. Patches may be configured for any size or shape. In some instances, patches are configured to adhere to specific areas of the body (e.g., face, head, or other area). In some instances, patches are configured as a single sheet covering the entire face. In some instances, multiple patches are configured to sample skin from the face. In some instances, patches are used as disclosed in FIGS. 11-13 of US 2016/0279401; or FIGS. 1-4 of US 20030167556, incorporated by reference in their entirety.

In some embodiments, a skin collection device such as an adhesive patch comprises a shape. The skin collection device may include 1 shape, or may include multiple shapes. A kit may include skin collection devices having separate shapes, for example 1, 2, 3, 4, 5, 6, 7, 8, 9, 10, or more different shaped collection devices. Examples of shapes include circles, ovals, squares, and the like. A shape may be straight. A shape may be generally composed of straight line segments. For example, the shape may include an angle (e.g. acute angle, obtuse angle, or right angle), balbis, concave polygon, constructible polygon, convex polygon, cyclic polygon, equiangular polygon, equilateral polygon, penrose tile, polyform, regular polygon, simple polygon, or tangential polygon. The shape may include a polygon with a specific number of sides, such as a triangle—3 sides, acute triangle, equilateral triangle, heptagonal triangle, isosceles triangle, golden triangle, obtuse triangle, rational triangle, right triangle, 30-60-90 triangle, isosceles right triangle, kepler triangle, scalene triangle, quadrilateral—4 sides, cyclic quadrilateral, kite, parallelogram, rhombus (equilateral parallelogram), lozenge, rhomboid, rectangle, square (regular quadrilateral), tangential quadrilateral, trapezoid, isosceles trapezoid, pentagon—5 sides, hexagon—6 sides, lemoine hexagon, heptagon—7 sides, octagon—8 sides, nonagon—9 sides, decagon—10 sides, hendecagon—11 sides, dodecagon—12 sides, tridecagon—13 sides, tetradecagon—14 sides, pentadecagon—15 sides, hexadecagon—16 sides, heptadecagon—17 sides, octadecagon—18 sides, enneadecagon—19 sides, icosagon—20 sides, star polygon—there are multiple types of stars, pentagram—star polygon with 5 sides, hexagram—star polygon with 6 sides, star of David, heptagram—star polygon with 7 sides, octagram—star polygon with 8 sides, star of Lakshmi, enneagram—star polygon with 9 sides, decagram—star polygon with 10 sides, hendecagram—star polygon with 11 sides, dodecagram—star polygon with 12 sides, or apeirogon—generalized polygon with countably infinite set of sides. The shape may be curved. The shape may be composed of circular arcs. For example, the shape may include an annulus, arbelos, circle, archimedes' twin circles, bankoff circle, circular triangle, reuleaux triangle, circumcircle, disc, incircle and excircles of a triangle, nine-point circle, circular sector, circular segment, crescent, lens, vesica piscis (fish bladder), lune, quatrefoil, reuleaux polygon, reuleaux triangle, salinon, semicircle, tomahawk, trefoil, triquetra, or heart shape. In some embodiments, the shape may not be composed of circular arcs. For example, the shape may include an Archimedean spiral, astroid, cardioid, deltoid, ellipse, heartagon, lemniscate, oval, cartesian oval, cassini oval, oval of booth, ovoid—similar to an oval, superellipse, taijitu, tomoe, or magatama shape.

The shape may be based on a skin collection area. For example, the skin collection device may include a single large patch, include face mask, be shaped for a forehead (e.g., be kidney shaped), be shaped to go under eyes (e.g. crescent), be shaped to cover at least part of a nose, be shaped to cover at least part of a right cheek, be shaped to cover at least part of a left cheek, may be postauricular, may be shaped to cover at least part of a right or left hand, or may be shaped to cover at least part of a right or left foot.

The shape may include a diameter. The shape may include multiple diameters. The diameter may include a maximal diameter. The diameter may include a minimal diameter. The diameter may include a length. Examples of diameter lengths include about 0.25 cm, about 0.5 cm, about 0.75 cm, about 1 cm, about 1.25 cm, about 1.5 cm, about 1.75 cm, about 2 cm, about 2.25 cm, about 2.5 cm, about 2.75 cm, about 3 cm, about 3.25 cm, about 3.5 cm, about 3.75 cm, about 4 cm, about 4.25 cm, about 4.5 cm, about 4.75 cm, about 5 cm, about 5.25 cm, about 5.5 cm, about 5.75 cm, about 6 cm, about 6.25 cm, about 6.5 cm, about 6.75 cm, about 7 cm, about 7.25 cm, about 7.5 cm, about 7.75 cm, about 8 cm, about 8.25 cm, about 8.5 cm, about 8.75 cm, about 9 cm, about 9.25 cm, about 9.5 cm, about 9.75 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, or about 30 cm. The diameter length may include a range defined by any two of the aforementioned diameter lengths. The diameter length may be at least 0.25 cm, at least 0.5 cm, at least 0.75 cm, at least 1 cm, at least 1.25 cm, at least 1.5 cm, at least 1.75 cm, at least 2 cm, at least 2.25 cm, at least 2.5 cm, at least 2.75 cm, at least 3 cm, at least 3.25 cm, at least 3.5 cm, at least 3.75 cm, at least 4 cm, at least 4.25 cm, at least 4.5 cm, at least 4.75 cm, at least 5 cm, at least 5.25 cm, at least 5.5 cm, at least 5.75 cm, at least 6 cm, at least 6.25 cm, at least 6.5 cm, at least 6.75 cm, at least 7 cm, at least 7.25 cm, at least 7.5 cm, at least 7.75 cm, at least 8 cm, at least 8.25 cm, at least 8.5 cm, at least 8.75 cm, at least 9 cm, at least 9.25 cm, at least 9.5 cm, at least 9.75 cm, at least 10 cm, at least 11 cm, at least 12 cm, at least 13 cm, at least 14 cm, at least 15 cm, at least 16 cm, at least 17 cm, at least 18 cm, at least 19 cm, at least 20 cm, at least 21 cm, at least 22 cm, at least 23 cm, at least 24 cm, at least 25 cm, at least 26 cm, at least 27 cm, at least 28 cm, at least 29 cm, or at least 30 cm. In some embodiments, the diameter length is less than 0.25 cm, less than 0.5 cm, less than 0.75 cm, less than 1 cm, less than 1.25 cm, less than 1.5 cm, less than 1.75 cm, less than 2 cm, less than 2.25 cm, less than 2.5 cm, less than 2.75 cm, less than 3 cm, less than 3.25 cm, less than 3.5 cm, less than 3.75 cm, less than 4 cm, less than 4.25 cm, less than 4.5 cm, less than 4.75 cm, less than 5 cm, less than 5.25 cm, less than 5.5 cm, less than 5.75 cm, less than 6 cm, less than 6.25 cm, less than 6.5 cm, less than 6.75 cm, less than 7 cm, less than 7.25 cm, less than 7.5 cm, less than 7.75 cm, less than 8 cm, less than 8.25 cm, less than 8.5 cm, less than 8.75 cm, less than 9 cm, less than 9.25 cm, less than 9.5 cm, less than 9.75 cm, less than 10 cm, less than 11 cm, less than 12 cm, less than 13 cm, less than 14 cm, less than 15 cm, less than 16 cm, less than 17 cm, less than 18 cm, less than 19 cm, less than 20 cm, less than 21 cm, less than 22 cm, less than 23 cm, less than 24 cm, less than 25 cm, less than 26 cm, less than 27 cm, less than 28 cm, less than 29 cm, or less than 30 cm.

The shape may include a perimeter. The perimeter may include a circumference. The perimeter may include a length. Examples of perimeter lengths include about 0.25 cm, about 0.5 cm, about 0.75 cm, about 1 cm, about 1.25 cm, about 1.5 cm, about 1.75 cm, about 2 cm, about 2.25 cm, about 2.5 cm, about 2.75 cm, about 3 cm, about 3.25 cm, about 3.5 cm, about 3.75 cm, about 4 cm, about 4.25 cm, about 4.5 cm, about 4.75 cm, about 5 cm, about 5.25 cm, about 5.5 cm, about 5.75 cm, about 6 cm, about 6.25 cm, about 6.5 cm, about 6.75 cm, about 7 cm, about 7.25 cm, about 7.5 cm, about 7.75 cm, about 8 cm, about 8.25 cm, about 8.5 cm, about 8.75 cm, about 9 cm, about 9.25 cm, about 9.5 cm, about 9.75 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 35 cm, about 40 cm, about 45 cm, about 50 cm, about 60 cm, about 70 cm, about 80 cm, about 90 cm, or about 100 cm. The perimeter length may include a range defined by any two of the aforementioned perimeter lengths. The perimeter length may be at least 0.25 cm, at least 0.5 cm, at least 0.75 cm, at least 1 cm, at least 1.25 cm, at least 1.5 cm, at least 1.75 cm, at least 2 cm, at least 2.25 cm, at least 2.5 cm, at least 2.75 cm, at least 3 cm, at least 3.25 cm, at least 3.5 cm, at least 3.75 cm, at least 4 cm, at least 4.25 cm, at least 4.5 cm, at least 4.75 cm, at least 5 cm, at least 5.25 cm, at least 5.5 cm, at least 5.75 cm, at least 6 cm, at least 6.25 cm, at least 6.5 cm, at least 6.75 cm, at least 7 cm, at least 7.25 cm, at least 7.5 cm, at least 7.75 cm, at least 8 cm, at least 8.25 cm, at least 8.5 cm, at least 8.75 cm, at least 9 cm, at least 9.25 cm, at least 9.5 cm, at least 9.75 cm, at least 10 cm, at least 11 cm, at least 12 cm, at least 13 cm, at least 14 cm, at least 15 cm, at least 16 cm, at least 17 cm, at least 18 cm, at least 19 cm, at least 20 cm, at least 21 cm, at least 22 cm, at least 23 cm, at least 24 cm, at least 25 cm, at least 26 cm, at least 27 cm, at least 28 cm, at least 29 cm, at least 30 cm, at least 35 cm, at least 40 cm, at least 45 cm, at least 50 cm, at least 60 cm, at least 70 cm, at least 80 cm, at least 90 cm, or at least 100 cm. In some embodiments, the perimeter length is less than 0.25 cm, less than 0.5 cm, less than 0.75 cm, less than 1 cm, less than 1.25 cm, less than 1.5 cm, less than 1.75 cm, less than 2 cm, less than 2.25 cm, less than 2.5 cm, less than 2.75 cm, less than 3 cm, less than 3.25 cm, less than 3.5 cm, less than 3.75 cm, less than 4 cm, less than 4.25 cm, less than 4.5 cm, less than 4.75 cm, less than 5 cm, less than 5.25 cm, less than 5.5 cm, less than 5.75 cm, less than 6 cm, less than 6.25 cm, less than 6.5 cm, less than 6.75 cm, less than 7 cm, less than 7.25 cm, less than 7.5 cm, less than 7.75 cm, less than 8 cm, less than 8.25 cm, less than 8.5 cm, less than 8.75 cm, less than 9 cm, less than 9.25 cm, less than 9.5 cm, less than 9.75 cm, less than 10 cm, less than 11 cm, less than 12 cm, less than 13 cm, less than 14 cm, less than 15 cm, less than 16 cm, less than 17 cm, less than 18 cm, less than 19 cm, less than 20 cm, less than 21 cm, less than 22 cm, less than 23 cm, less than 24 cm, less than 25 cm, less than 26 cm, less than 27 cm, less than 28 cm, less than 29 cm, less than 30 cm, less than 35 cm, less than 40 cm, less than 45 cm, less than 50 cm, less than 60 cm, less than 70 cm, less than 80 cm, less than 90 cm, or less than 100 cm.

The shape may include an area. Examples of areas include about 0.25 cm², about 0.5 cm², about 0.75 cm², about 1 cm², about 1.25 cm², about 1.5 cm², about 1.75 cm², about 2 cm², about 2.25 cm², about 2.5 cm², about 2.75 cm², about 3 cm², about 3.25 cm², about 3.5 cm², about 3.75 cm², about 4 cm², about 4.25 cm², about 4.5 cm², about 4.75 cm², about 5 cm², about 5.25 cm², about 5.5 cm², about 5.75 cm², about 6 cm², about 6.25 cm², about 6.5 cm², about 6.75 cm², about 7 cm², about 7.25 cm², about 7.5 cm², about 7.75 cm², about 8 cm², about 8.25 cm², about 8.5 cm², about 8.75 cm², about 9 cm², about 9.25 cm², about 9.5 cm², about 9.75 cm², about 10 cm², about 11 cm², about 12 cm², about 13 cm², about 14 cm², about 15 cm², about 16 cm², about 17 cm², about 18 cm², about 19 cm², about 20 cm², about 21 cm², about 22 cm², about 23 cm², about 24 cm², about 25 cm², about 26 cm², about 27 cm², about 28 cm², about 29 cm², about 30 cm², about 35 cm², about 40 cm², about 45 cm², about 50 cm², about 60 cm², about 70 cm², about 80 cm², about 90 cm², about 100 cm², about 110 cm², about 120 cm², about 130 cm², about 140 cm², about 150 cm², about 160 cm², about 170 cm², about 180 cm², about 190 cm², or about 200 cm². The areas may include a range defined by any two of the aforementioned areas. The areas may be at least 0.25 cm², at least 0.5 cm², at least 0.75 cm², at least 1 cm², at least 1.25 cm², at least 1.5 cm², at least 1.75 cm², at least 2 cm², at least 2.25 cm², at least 2.5 cm², at least 2.75 cm², at least 3 cm², at least 3.25 cm², at least 3.5 cm², at least 3.75 cm², at least 4 cm², at least 4.25 cm², at least 4.5 cm², at least 4.75 cm², at least 5 cm², at least 5.25 cm², at least 5.5 cm², at least 5.75 cm², at least 6 cm², at least 6.25 cm², at least 6.5 cm², at least 6.75 cm², at least 7 cm², at least 7.25 cm², at least 7.5 cm², at least 7.75 cm², at least 8 cm², at least 8.25 cm², at least 8.5 cm², at least 8.75 cm², at least 9 cm², at least 9.25 cm², at least 9.5 cm², at least 9.75 cm², at least 10 cm², at least 11 cm², at least 12 cm², at least 13 cm², at least 14 cm², at least 15 cm², at least 16 cm², at least 17 cm², at least 18 cm², at least 19 cm², at least 20 cm², at least 21 cm², at least 22 cm², at least 23 cm², at least 24 cm², at least 25 cm², at least 26 cm², at least 27 cm², at least 28 cm², at least 29 cm², at least 30 cm², at least 35 cm², at least 40 cm², at least 45 cm², at least 50 cm², at least 60 cm², at least 70 cm², at least 80 cm², at least 90 cm², at least 100 cm², at least 110 cm², at least 120 cm², at least 130 cm², at least 140 cm², at least 150 cm², at least 160 cm², at least 170 cm², at least 180 cm², at least 190 cm², or at least 200 cm². In some embodiments, the areas is less than 0.25 cm², less than 0.5 cm², less than 0.75 cm², less than 1 cm², less than 1.25 cm², less than 1.5 cm², less than 1.75 cm², less than 2 cm², less than 2.25 cm², less than 2.5 cm², less than 2.75 cm², less than 3 cm², less than 3.25 cm², less than 3.5 cm², less than 3.75 cm², less than 4 cm², less than 4.25 cm², less than 4.5 cm², less than 4.75 cm², less than 5 cm², less than 5.25 cm², less than 5.5 cm², less than 5.75 cm², less than 6 cm², less than 6.25 cm², less than 6.5 cm², less than 6.75 cm², less than 7 cm², less than 7.25 cm², less than 7.5 cm², less than 7.75 cm², less than 8 cm², less than 8.25 cm², less than 8.5 cm², less than 8.75 cm², less than 9 cm², less than 9.25 cm², less than 9.5 cm², less than 9.75 cm², less than 10 cm², less than 11 cm², less than 12 cm², less than 13 cm², less than 14 cm², less than 15 cm², less than 16 cm², less than 17 cm², less than 18 cm², less than 19 cm², less than 20 cm², less than 21 cm², less than 22 cm², less than 23 cm², less than 24 cm², less than 25 cm², less than 26 cm², less than 27 cm², less than 28 cm², less than 29 cm², less than 30 cm², less than 35 cm², less than 40 cm², less than 45 cm², less than 50 cm², less than 60 cm², less than 70 cm², less than 80 cm², less than 90 cm², less than 100 cm², less than 110 cm², less than 120 cm², less than 130 cm², less than 140 cm², less than 150 cm², less than 160 cm², less than 170 cm², less than 180 cm², less than 190 cm², or less than 200 cm².

Biological samples (e.g., skin samples) for analysis may be obtained using non-invasive techniques or minimally invasive techniques. In some instances, a minimally-invasive technique comprises the use of microneedles. In some embodiments, a sample such as a skin sample is collected using one or more microneedles. In some instances, a plurality of microneedles are used to obtain a sample. In some instance, microneedles are polymeric. In some instance, microneedles are coated with a substance (e.g., enzymes, chemical, or other substance) capable of disrupting an extracellular matrix. In some instances, microneedles such as those described in U.S. Pat. No. 10,995,366, incorporated by reference in its entirety, are used to obtain a skin sample. Microneedles in some instances pierce a subject's skin to obtain samples of skin cells, blood, or both. In some instances, microneedles are coated with probes that bind to one or more nucleic acid targets described herein.

Examples of subjects include but are not limited to vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female. In some embodiments, the subject has skin previously exposed to UV light.

### Cellular Material and Sample Process

Provided herein are methods of non-invasive sampling. Such non-invasive methods in some instances provide advantages over traditional biopsy methods, including but not limited to self-application by a patient/subject, increased signal to noise ratio of sample exposed to the skin surface (leading to higher sensitivity and/or specificity), lack of temporary or permanent scarring at the analysis site, lower change of infection, or other advantage.

A skin sample may be obtained from a subject using a collection device (such as an adhesive patch). In some embodiments of the methods described herein, a skin sample is obtained from the subject by applying an adhesive patch to a skin region of the subject. In some embodiments, the skin sample is obtained using an adhesive patch. In some embodiments, the adhesive patch comprises tape. In some embodiments, the skin sample is not obtained with an adhesive patch. In some instances, the skin sample is obtained using a brush. In some instances, the skin sample is obtained using a swab, for example a cotton swab. In some cases, the skin sample is obtained using a probe. In some cases, the skin sample is obtained using a hook. In some instances, the skin sample is obtained using a medical applicator. In some instances, the skin sample is obtained by scraping a skin surface of the subject. In some cases, the skin sample is obtained through excision. In some instances, the skin sample is biopsied. In some embodiments, the skin sample is a biopsy. In some instances, the skin sample is obtained using one or more needles. For example, the needles may be microneedles. In some instances, the biopsy is a needle biopsy, or a microneedle biopsy. In some instances, the skin sample is obtained invasively. In some instances, the skin sample is obtained non-invasively. A skin sample in some instances is obtained iteratively from the same skin area of a subject. In some instances, multiple samples are obtained from a single skin area and pooled prior to analysis.

The methods provided herein may generate samples from various layers of skin. While not wishing to be bound by theory, sampling at the surface of the skin provides results differentiated from that of deeper (invasive, e.g., biopsy) sampling for skin cancer and other disease derived from external/environmental factor interactions (e.g., UV). For example, the quantity of sun exposed cells and number of mutations in some instances results in higher sensitivity or specificity in measuring mutation burden.

In some instances, methods generate samples from the top or superficial layers of skin, which have been exposed to higher levels of one or more environmental factors. In some embodiments, the skin sample comprises cells of the stratum corneum. In some embodiments, the skin sample consists of cells of the stratum corneum. In some instances, non-invasive sampling described herein does not fully disrupt the epidermal:dermal junction. Without being bound by theory, non-invasive sampling described herein does not trigger significant wound healing which normally results from significant damage to the epithelial barrier. In some embodiments, the skin sample comprises at least 80%, 90%, 95%, 97%, 98%, 99%, 99.5%, or at least 99.9% of cells derived from the basal keratinocyte layer. In some embodiments, the skin sample comprises less than 10%, 5%, 3%, 2%, 1%, 0.1%, 0.05%, or less than 0.01% cells derived from the basal keratinocyte layer. In some embodiments, the skin sample does not include the basal layer of the skin. In some embodiments, the skin sample comprises or consists of a skin depth of 10 μm, 50 μm, 100 μm, 150 μm, 200 μm, 250 μm, 300 μm, 350 μm, 400 μm, 450 μm, 500 μm, or a range of skin depths defined by any two of the aforementioned skin depths. In some embodiments, the skin sample comprises or consists of a skin depth of about 10 μm, 50 μm, 100 μm, 150 μm, 200 μm, 250 μm, 300 μm, 350 μm, 400 μm, 450 μm, or about 500 μm. In some embodiments, the skin sample comprises or consists of a skin depth of 50-100 μm. In some embodiments, the skin sample comprises or consists of a skin depth of 100-200 μm. In some embodiments, the skin sample comprises or consists of a skin depth of 200-300 μm. In some embodiments, the skin sample comprises or consists of a skin depth of 300-400 μm. In some embodiments, the skin sample comprises or consists of a skin depth of 400-500 μm.

Non-invasive sampling methods described herein may comprise obtaining multiple skin samples from the same area of skin on an individual using multiple collection devices (e.g., tapes). In some instances, each sample obtained from the same area or substantially the same area results in progressively deeper layers of skin cells. In some instances, multiple samples are pooled prior to analysis. The skin sample may be from one collection device or from multiple collection devices. For example, one collection device may be used to obtain an amount of cellular material described, or the skin samples from multiple collection devices may be used to obtain a given amount of cellular material. For example, skin samples from 2 or more adhesive patches may be pooled to obtain an amount of genetic cellular material sufficient for a method described herein. In some instances, skin samples from at least 2, 3, 4, 5, 6, 8, 10, 12, 16, or more adhesive patches are pooled to obtain an amount of genetic cellular material sufficient for a method described herein. In some instances, skin samples from at least 2-16, 2-12, 2-10, 2-8, 2-6, 2-4, 4-16, 4-12, 4-8, 6-16, or 8-20 adhesive patches are pooled to obtain an amount of genetic cellular material sufficient for a method described herein.

The skin sample may be defined by thickness, or how deep into the skin cells are obtained. In some embodiments, the skin sample is no more than 10 μm thick. In some embodiments, the skin sample is no more than 50 μm thick. In some embodiments, the skin sample is no more than 100 μm thick. In some embodiments, the skin sample is no more than 150 μm thick. In some embodiments, the skin sample is no more than 200 μm thick. In some embodiments, the skin sample is no more than 250 μm thick. In some embodiments, the skin sample is no more than 300 μm thick. In some embodiments, the skin sample is no more than 350 μm thick. In some embodiments, the skin sample is no more than 400 μm thick. In some embodiments, the skin sample is no more than 450 μm thick. In some embodiments, the skin sample is no more than 500 μm thick.

In some embodiments, the skin sample is at least 10 μm thick. In some embodiments, the skin sample is at least 50 μm thick. In some embodiments, the skin sample is at least 100 μm thick. In some embodiments, the skin sample is at least 150 μm thick. In some embodiments, the skin sample is at least 200 μm thick. In some embodiments, the skin sample is at least 250 μm thick. In some embodiments, the skin sample is at least 300 μm thick. In some embodiments, the skin sample is at least 350 μm thick. In some embodiments, the skin sample is at least 400 μm thick. In some embodiments, the skin sample is at least 450 μm thick. In some embodiments, the skin sample is at least 500 μm thick.

In some embodiments, the adhesive patch removes a skin sample from the subject at a depth no greater than 10 μm. In some embodiments, the adhesive patch removes a skin sample from the subject at a depth no greater than 50 μm. In some embodiments, the adhesive patch removes a skin sample from the subject at a depth no greater than 100 μm. In some embodiments, the adhesive patch removes a skin sample from the subject at a depth no greater than 150 μm. In some embodiments, the adhesive patch removes a skin sample from the subject at a depth no greater than 200 μm. In some embodiments, the adhesive patch removes a skin sample from the subject at a depth no greater than 250 μm. In some embodiments, the adhesive patch removes a skin sample from the subject at a depth no greater than 300 μm. In some embodiments, the adhesive patch removes a skin sample from the subject at a depth no greater than 350 μm. In some embodiments, the adhesive patch removes a skin sample from the subject at a depth no greater than 400 μm. In some embodiments, the adhesive patch removes a skin sample from the subject at a depth no greater than 450 μm. In some embodiments, the adhesive patch removes a skin sample from the subject at a depth no greater than 500 μm.

In some embodiments, the adhesive patch removes 1, 2, 3, 4, or 5 layers of stratum corneum from a skin surface of the subject. In some embodiments, the adhesive patch removes a range of layers of stratum corneum from a skin surface of the subject, for example a range defined by any two of the following integers: 1, 2, 3, 4, or 5. In some embodiments, the adhesive patch removes 1-5 layers of stratum corneum from a skin surface of the subject. In some embodiments, the adhesive patch removes 2-3 layers of stratum corneum from a skin surface of the subject. In some embodiments, the adhesive patch removes 2-4 layers of stratum corneum from a skin surface of the subject. In some embodiments, the adhesive patch removes no more than the basal layer of a skin surface from the subject.

Some embodiments include collecting cells from the stratum corneum of a subject, for instance, by using an adhesive tape with an adhesive matrix to adhere the cells from the stratum corneum to the adhesive matrix. In some embodiments, the cells from the stratum corneum comprise T cells or components of T cells. In some embodiments, the cells from the stratum corneum comprise keratinocytes. In some instances, the stratum corneum comprises keratinocytes, melanocytes, fibroblasts, antigen presenting cells (Langerhans cells, dendritic cells), or inflammatory cells (T cells, B cells, eosinophils, basophils). In some embodiments, the skin sample does not comprise melanocytes. In some embodiments, a skin sample is obtained by applying a plurality of adhesive patches to a skin region of a subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a skin lesion.

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. In some cases, the skin sample adhered to the adhesive matrix comprises or consists of cells from the stratum corneum of a subject. For example, the effective or sufficient amount of a skin sample is an amount that removably adheres to a material, such as the matrix or adhesive patch. The adhered skin sample, in certain embodiments, comprises cellular material including nucleic acids. In some instances, the nucleic acid is RNA or DNA. In some instances, the nucleic acid is RNA (e.g. mRNA). An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a genomic analysis. Sufficient amounts of RNA includes, but not limited to, picogram, nanogram, and microgram quantities. In some embodiments, the RNA includes mRNA. In some embodiments, the RNA includes microRNAs. In some embodiments, the RNA includes mRNA and microRNAs.

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. For example, the effective or sufficient amount of a skin sample is an amount that removably adheres to a material, such as the matrix or adhesive patch. The adhered skin sample, in certain embodiments, comprises cellular material including nucleic acids. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a genomic analysis. Sufficient amounts of RNA includes, but not limited to, picogram, nanogram, and microgram quantities.

In some instances, the nucleic acid is a RNA molecule or a fragmented RNA molecule (RNA fragments). In some instances, the RNA is a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free circulating RNA.

In some instances, the nucleic acid is DNA. DNA includes, but not limited to, genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free circulating DNA.

Non-invasive sampling described herein may obtain amounts of nucleic acids. Such nucleic acids in some instances are obtained from obtaining skin using a single collection device. In some instances, nucleic acids are obtained from samples pooled from multiple collection devices. In some instances, nucleic acids are obtained from samples from a single collection device applied to the skin multiple times (1, 2, 3, or 4 times). In additional embodiments, the adhered skin sample comprises cellular material including nucleic acids such as RNA or DNA, in an amount that is at least about 1 picogram. Cellular material in some instances is obtained from skin using a single collection device. In some instances, cellular material is obtained from samples pooled from multiple collection devices. In some instances, cellular material is obtained from samples from a single collection device applied to the skin multiple times (1, 2, 3, or 4 times). In some instances, an amount of cellular material described herein refers to the amount of material pooled from multiple collection devices (e.g., 1-6 devices). In some embodiments, the amount of cellular material is no more than about 1 nanogram. In further or additional embodiments, the amount of cellular material is no more than about 1 microgram. In still further or additional embodiments, the amount of cellular material is no more than about 1 milligram. In still further or additional embodiments, the amount of cellular material is no more than about 1 gram.

A total amount of cellular material may be obtained from a kit (e.g., a kit comprising multiple collection devices each applied to skin). In some instances, cellular material collected in a kit is less than 20 milligrams, less than 10 milligrams, less than 5 milligrams, less than 2 milligrams, less than 1 milligram, less than 500 micrograms, less than 200 micrograms, or less than 100 micrograms. In some instances, the collection device in a kit comprises an adhesive patch. In some instances, each adhesive patch comprises 1 picogram to 2000 micrograms, 1 picogram to 1000 micrograms, 1 picogram to 500 micrograms, 1 picogram to 100 micrograms, or 1 picogram to 10 micrograms per patch of cellular material.

In further or additional embodiments, the amount of cellular material is from about 1 picogram to about 1 gram. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms. In further or additional embodiments, the cellular material comprises an amount that is from about 5 microgram to about 1 gram, from about 1 picograms to about 500 micrograms, from about 1 picograms to about 250 micrograms, from about 1 picograms to about 1 microgram, from about 1 nanogram to about 750 nanograms, or from about 1 nanogram to about 500 nanograms.

In further or additional embodiments, the amount of cellular material is from about 1 picogram to about 1 microgram. In further or additional embodiments, the amount of cellular material, including nucleic acids such as RNA or DNA, comprises an amount that is from about 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms. In some cases, about 3 ng of genomic DNA is sufficient to provide robust variant detection via a detection platform such as mass spectrometry (e.g. MassARRAY) or next generation sequencing (e.g. NextSeq 2000). Some embodiments include at least about 3 ng of a cellular material such as DNA or RNA. In some cases, at least 1 ng of cellular material such as DNA or RNA is sufficient.

In further or additional embodiments, the amount of cellular material is from about 1 picogram to about 1 milligram. In further or additional embodiments, the amount of cellular material, including nucleic acids such as RNA or DNA, comprises an amount that is from about 50 milligrams to about 500 micrograms, from about 100 milligrams about 450 micrograms, from about 100 milligrams about 350 micrograms, from about 100 milligrams about 300 micrograms, from about 120 milligrams about 250 micrograms, from about 150 milligrams about 200 micrograms, from about 5 milligrams to about 500 milligrams, or from about 5 milligrams to about 100 milligrams, or from about 20 milligrams to about 150 milligrams, or from about 1 milligrams to about 20 milligrams, or from about 1 milligram to about 50 milligrams, or from about 1 milligram to about 100 milligrams.

In further or additional embodiments, the amount of cellular material, including nucleic acids such as RNA or DNA, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram.

In further or additional embodiments, the amount of cellular material, including nucleic acids such as RNA or DNA, is less than about 1 gram, is less than about 500 milligrams, is less than about 490 milligrams, is less than about 480 milligrams, is less than about 470 milligrams, is less than about 460 milligrams, is less than about 450 milligrams, is less than about 440 milligrams, is less than about 430 milligrams, is less than about 420 milligrams, is less than about 410 milligrams, is less than about 400 milligrams, is less than about 390 milligrams, is less than about 380 milligrams, is less than about 370 milligrams, is less than about 360 milligrams, is less than about 350 milligrams, is less than about 340 milligrams, is less than about 330 milligrams, is less than about 320 milligrams, is less than about 310 milligrams, is less than about 300 milligrams, is less than about 290 milligrams, is less than about 280 milligrams, is less than about 270 milligrams, is less than about 260 milligrams, is less than about 250 milligrams, is less than about 240 milligrams, is less than about 230 milligrams, is less than about 220 milligrams, is less than about 210 milligrams, is less than about 200 milligrams, is less than about 190 milligrams, is less than about 180 milligrams, is less than about 170 milligrams, is less than about 160 milligrams, is less than about 150 milligrams, is less than about 140 milligrams, is less than about 130 milligrams, is less than about 120 milligrams, is less than about 110 milligrams, is less than about 100 milligrams, is less than about 90 milligrams, is less than about 80 milligrams, is less than about 70 milligrams, is less than about 60 milligrams, is less than about 50 milligrams, is less than about 20 milligrams, is less than about 10 milligrams, or is less than about 5 milligrams.

In some instances, the layers of skin include epidermis, dermis, or hypodermis. The outer layer of epidermis is the stratum corneum layer, followed by stratum lucidum, stratum granulosum, stratum spinosum, and stratum basale. In some instances, the skin sample is obtained from the epidermis layer. In some cases, the skin sample is obtained from the stratum corneum layer. In some instances, the skin sample is obtained from the dermis. In some cases, the skin sample is obtained from the stratum germinativum layer. In some cases, the skin sample is obtained from no deeper than the stratum germinativum layer.

In some instances, cells from the stratum corneum layer are obtained, which comprises keratinocytes. In some instances, cells from the stratum corneum layer comprise T cells or components of T cells. In some cases, melanocytes are not obtained from the skin sample.

The sample may comprise skin cells from a superficial depth of skin using the non-invasive sampling techniques described herein. In some instances, the sample comprises skin cells from about the superficial about 0.01, 0.02, 0.05, 0.08, 0.1, 0.2, 0.3, 0.4 mm of skin. In some instances, the sample comprises skin cells from no more than the superficial about 0.01, 0.02, 0.05, 0.08, 0.1, 0.2, 0.3, 0.4 mm of skin. In some instances, the sample comprises skin cells from at least the superficial about 0.01, 0.02, 0.05, 0.08, 0.1, 0.2, 0.3, or at least 0.4 mm of skin. In some instances, the sample comprises skin cells from the superficial about 0.01-0.1, 0.01-0.2, 0.02-0.1, 0.02-0.2 0.04-0.0.08, 0.02-0.08, 0.01-0.08, 0.05-0.2, or 0.05-0.1 mm of skin. In some instances, the sample comprises skin cells from about the superficial about 0.01, 0.02, 0.05, 0.08, 0.1, 0.2, 0.3, or about 0.4 μm of skin. In some instances, the sample comprises skin cells from no more than the superficial about 0.01, 0.02, 0.05, 0.08, 0.1, 0.2, 0.3, or no more than 0.4 μm of skin. In some instances, the sample comprises skin cells from at least the superficial about 0.01, 0.02, 0.05, 0.08, 0.1, 0.2, 0.3, 0.4 μm of skin. In some instances, the sample comprises skin cells from the superficial about 0.01-0.1, 0.01-0.2, 0.02-0.1, 0.02-0.2 0.04-0.0.08, 0.02-0.08, 0.01-0.08, 0.05-0.2, or 0.05-0.1 μm of skin.

The sample may comprise skin cells a number of skin cell layers, for example the superficial cell layers. In some instances, the sample comprises skin cells from 1-5, 1-10, 1-20, 1-25, 1-50, 1-75, or 1-100 cell layers. In some instances, the sample comprises skin cells from about 1, 2, 3, 4, 5, 8, 10, 12, 15, 20, 22, 25, 30, 35, or about 50 cell layers. In some instances, the sample comprises skin cells from no more than 1, 2, 3, 4, 5, 8, 10, 12, 15, 20, 22, 25, 30, 35, or no more than 50 cell layers.

The sample may comprise skin cells collected from a defined skin area of the subject having a surface area. In some instances the sample comprises skin cells obtained from a skin surface area of 10-300 mm², 10-500 mm², 5-500 mm², 1-300 mm², 5-100 mm², 5-200 mm², or 10- 100 mm². In some instances the sample comprises skin cells obtained from a skin surface area of at least 5, 10, 20, 25, 30, 50, 75, 90, 100, 125, 150, 175, 200, 225, 250, 275, 300, or at least 350 mm². In some instances the sample comprises skin cells obtained from a skin surface area of no more than 5, 10, 20, 25, 30, 50, 75, 90, 100, 125, 150, 175, 200, 225, 250, 275, 300, or no more than 350 mm².

Following extraction of nucleic acids from a biological sample, the nucleic acids, in some instances, are further purified. In some instances, the nucleic acids are RNA. In some instances, the nucleic acids are DNA. In some instances, the RNA is human RNA. In some instances, the DNA is human DNA. In some instances, the RNA is microbial RNA. In some instances, the DNA is microbial DNA. In some instances, cDNA is generated by reverse transcription of RNA. In some instances, human nucleic acids and microbial nucleic acids are purified from the same biological sample. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods for isolating nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 um, 1 um, 1.5 um, 2 um, 2.5 um, 3 um, 3.5 um, 4 um, 4.5 um, 5 um, 5.5 um, 6 um, 6.5 um, 7 um, 7.5 um, 8 um, 8.5 um, 9 um, 9.5 um, 10 um, or more than 10 um.

In some cases, a yield of the nucleic acids products obtained using methods described herein is about 500 picograms or higher, about 600 picograms or higher, about 1000 picograms or higher, about 2000 picograms or higher, about 3000 picograms or higher, about 4000 picograms or higher, about 5000 picograms or higher, about 6000 picograms or higher, about 7000 picograms or higher, about 8000 picograms or higher, about 9000 picograms or higher, about 10000 picograms or higher, about 20000 picograms or higher, about 30000 picograms or higher, about 40000 picograms or higher, about 50000 picograms or higher, about 60000 picograms or higher, about 70000 picograms or higher, about 80000 picograms or higher, about 90000 picograms or higher, or about 100000 picograms or higher.

In some cases, a yield of the nucleic acids products obtained using methods described herein is about 100 picograms, 500 picograms, 600 picograms, 700 picograms, 800 picograms, 900 picograms, 1 nanogram, 5 nanograms, 10 nanograms, 15 nanograms, 20 nanograms, 21 nanograms, 22 nanograms, 23 nanograms, 24 nanograms, 25 nanograms, 26 nanograms, 27 nanograms, 28 nanograms, 29 nanograms, 30 nanograms, 35 nanograms, 40 nanograms, 50 nanograms, 60 nanograms, 70 nanograms, 80 nanograms, 90 nanograms, 100 nanograms, 150 nanograms, 200 nanograms, 250 nanograms, 300 nanograms, 400 nanograms, 500 nanograms, or higher.

In some cases, methods described herein provide less than less than 10%, less than 8%, less than 5%, less than 2%, less than 1%, or less than 0.5% product yield variations between samples.

In some embodiments, a number of cells is obtained for use in a method described herein. Some embodiments include use of an adhesive patch comprising an adhesive comprising a tackiness that is based on the number of cells to be obtained. Some embodiments include use of a number of adhesive patches based on the number of cells to be obtained. Some embodiments include use of an adhesive patch sized based on the number of cells to be obtained. The size and/or tackiness may be based on the type of skin to be obtained. For example, normal looking skin generally provides less cells and RNA yield than flaky skin. In some embodiments, a skin sample is used comprising skin from a subject's temple, forehead, cheek, or nose. In some embodiments, only one patch is used. In some embodiments, only one patch is used per skin area (e.g. skin area on a subject's temple, forehead, cheek, or nose).

In some cases, methods described herein provide a substantially homogenous population of a nucleic acid product. In some cases, methods described herein provide less than 30%, less than 25%, less than 20%, less than 15%, less than 10%, less than 8%, less than 5%, less than 2%, less than 1%, or less than 0.5% contaminants.

In some instances, following extraction, nucleic acids are stored. In some instances, the nucleic acids are stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein are subjected to an amplification reaction following isolation and purification. In some instances, the nucleic acids to be amplified are RNA including, but not limited to, human RNA and human microbial RNA. In some instances, the nucleic acids to be amplified are DNA including, but not limited to, human DNA and human microbial DNA. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR.

### Methods of Treatment

Disclosed herein, in some embodiments, are methods of treating a subject having a specific mutation burden or epigenetic profile (one or more epigenetic markers). In some embodiments, treatments are recommended based on categorization of the subject's mutation burden into one or more bins, classes, categories, qualitative actionable output, numeric actionable output, pathology score, or success rate output. In some embodiments, a mutation burden is correlated with a particular treatment which results in lowering the risk of cancer in an individual. In some instances, a bin is quantitative. In some instances, a bin is qualitative. In some instances, a bin In some instances, the categories comprise high, medium, and low. In some embodiments, the treatment comprises providing a cosmetic regimen. In some embodiments, the treatment comprises providing topical or oral supplements. In some embodiments, the treatment comprises a skin peel (light, moderate, or deep). Some embodiments include monitoring treatment efficacy. In some embodiments, the treatment comprises continuing to periodically monitor the patient using the mutation burden analysis methods described herein.

In some embodiments the treatment is chosen based in part on an aspect of the subject's skin. Some such aspects may include wrinkles, dryness, scaliness, flakiness, redness, or soreness. The treatment may be chosen based on an aspect of the subject's skin tone. In some embodiments, the treatment is chosen primarily based on the subject's mutation burden, such as a mutation burden determined with a kit or a method disclosed herein.

A mutation burden may be used to calculate a quantifiable burden. In some instances, a quantifiable burden is defined categorically as low, medium, or high. In some instances, a subject having a quantifiable burden of low is treated with sun protection sunscreens, supplements, or photolyase treatment. In some instances, a subject having a quantifiable burden of medium is treated with retinoids, light peel, or photodynamic therapy (PDT). In some instances, a subject having a quantifiable burden of high is treated with a moderate or deep peel. Any number of groupings or categories are consistent with the present disclosure.

Some embodiments of the methods described herein comprise a quantifiable burden which indicates an actionable output. In some embodiments, the actionable output determines if a lesion sampled non-invasively should be further analyzed by a medical practitioner such as dermatologist. In some embodiments, the actionable output determines if a lesion sampled non-invasively should be excised. In some embodiments, the actionable output determines if a lesion sampled non-invasively should monitored for changes.

In some instances, a quantifiable burden is defined by an optimal treatment outcome given the signature of a mutation burden. In some instances, a subject having a quantifiable burden of category 1 (or any other class, bin, or grouping) is treated with a sun protection sunscreen. In some instances, a subject having a quantifiable burden of class 2 (or any other category, bin, or grouping) is treated with photolyase treatment. In some instance, a category is associated with optimum treatment using any of the methods described herein. In some instances, 1, 2, 3, 4, 5, 10, 20, 50, or more than 50 categories are assigned based on quantifiable burden.

Some embodiments of the methods described herein comprise making a recommendation or treating a patient in response to the results of a method described herein such as quantifying a mutation burden. For example, some embodiments include providing or recommending a skin treatment. Some embodiments include not providing or not recommending the skin treatment. In some embodiments, the recommendation or treatment relates to a specific sunscreen or moisturizer for prevention of further damage to, for example, topical agents, chemical peels, lasers, over-the-counter products, or prescription products, for specific treatment depending on the level of damage. In some embodiments, the skin treatment is provided or recommended based on the mutation burden established from mutations in one or more target genes.

Described herein, in some embodiments, are methods of treatment that include administering a skin treatment to a subject. In some embodiments, the skin treatment comprises or consists of a skin damage prevention treatment. In some embodiments, the treatment comprises a pharmaceutical composition. In some embodiments, the treatment comprises a steroid treatment. In some embodiments, the treatment comprises a surgery. In some embodiments, the treatment comprises a transplant. In some embodiments, the treatment comprises vitamin A. In some embodiments, the treatment comprises a chemical peel. In some embodiments, the treatment comprises a laser treatment. In some embodiments, the treatment comprises a topical agent. In some embodiments, the treatment comprises an over-the-counter product. In some embodiments, the treatment comprises a prescription, or comprises a prescription product. In some embodiments, the treatment comprises a cosmetic. In some embodiments, the treatment comprises administration of a retinoid. In some embodiments the treatment comprises administration of a sunscreen. In some embodiments the treatment comprises administration of a supplement. In some embodiments the supplement comprises nicotinamide. In some embodiments, the treatment comprises administration of an mTOR inhibitor. In some embodiments, the mTOR inhibitor includes but is not limited to sirolimus, everolimus, zotarolimus, deforolimus, biolimus, or temsirolimus.

Some embodiments include administration of a sunscreen. The sunscreen may comprise a sun protection factor (SPF), such as SPF 8, SPF 10, SPF 15, SPF 20, SPF 30, SPF 40, SPF 50, SPF 60, SPF 70, SPF 80, or SPF 90, or a range of SPFs such as a range defined by any two of the aforementioned SPFs. The SPF may be chosen based on a measurement such as a mutation burden measurement. The SPF may be chosen based on a subject's skin tone.

In some embodiments, the treatment comprises a cosmetic formulation. Some embodiments include providing a cosmetic formulation containing agents for reducing mutation burden described herein. In some embodiments, the cosmetic formulation comprises an emulsion, a cream, a lotion, a solution, an anhydrous base, a paste, a powder, a gel, or an ointment. The emulsion may be an oil-in-water emulsion or a water-in-oil emulsion. Alternatively, the formulation may be a solution, such as an aqueous solution or a hydro-alcoholic solution. In another embodiment, the cosmetic formulation is an anhydrous base, such as a lipstick or a powder. In yet another embodiment, the formulation is comprised within an anti-aging product or a moisturizing product. The cosmetic formulation may further contain one or more of estradiol; progesterone; pregnanalone; coenzyme Q10; methylsolanomethane (MSM); copper peptide (copper extract); plankton extract (phytosome); glycolic acid; kojic acid; ascorbyl palmitate; all trans retinol; azaleic acid; salicylic acid; broparoestrol; estrone; adrostenedione; androstanediols; or sunblocks. In some embodiments, the skin damage treatment comprises a lotion. In some embodiments, the treatment comprises a sunscreen. In some embodiments, the treatment comprises a hydrogel. In some embodiments, the cosmetic formulation is administered topically.

Some embodiments include 1, 2, 3, 4, 5, 6, 7, 8, 9, 10, 11, 12, 13, 14, or 15, or more administrations of the treatment. Some embodiments include a range defined by any two of 1, 2, 3, 4, 5, 6, 7, 8, 9, 10, 11, 12, 13, 14, or 15, administrations of the treatment. Some embodiments include administration daily, weekly, biweekly, or monthly.

In some embodiments, the treatment includes a pharmaceutical composition. In some embodiments, the pharmaceutical composition is sterile. In some embodiments, the pharmaceutical composition includes a pharmaceutically acceptable carrier. In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

Some embodiments include administering a skin treatment. In some embodiments, administering comprises giving, applying or bringing the skin damage treatment into contact with the subject. In some embodiments, administration is accomplished by any of a number of routes. In some embodiments, administration is accomplished by a topical, oral, subcutaneous, intramuscular, intraperitoneal, intravenous, intrathecal or intradermal route.

In some embodiments, the skin treatment comprises a DNA repair enzyme. The methods and devices provided herein, in certain embodiments, involve administering a DNA repair enzyme to a subject in need thereof, such as a subject exposed to an environmental factor described herein. Some embodiments relate to a method of modulating gene or protein expression in the subject. In some embodiments, the DNA repair enzyme is a T4N5 endonuclease. In some embodiments, the DNA repair enzyme is a photolyase.

The treatment may include topical administration. The treatment may include a topical medication. Some examples of topical treatments include antibacterials, anthralin, antifungal agents, benzoyl peroxide, coal tar, corticosteroids, non-steroidal ointments, retinoids, or salicylic acid. The treatment may include antibacterial administration. Antibacterials may include mupirocin or clindamycin. Anthralin may help reduce inflammation or treat psoriasis. Antifungal agents may include Clotrimazole (Lotrimin), ketoconazole (Nizoral), or terbinafine (Lamisil AT). Benzoyl peroxide may be formulated in a cream, gel, wash, or foam. Coal tar may be provided at a strength ranging from 0.5% to 5%. Coal tar or another topical treatment may be administered in a shampoo. Corticosteroids may come in many different forms including foams, lotions, ointments, or creams. Non-steroidal ointment: The ointments crisaborole (Eucrisa) and tacrolimus (Protopic) and the cream pimecrolimus (Elidel) also are prescribed for eczema, including atopic dermatitis. Retinoids may include medications (such as Differin, Retin-A, or Tazorac) formulated as gels, foams, lotions, or creams derived from vitamin A. Salicylic acid may be provided in lotions, gels, soaps, shampoos, washes, or patches.

In some embodiments, the treatment includes an oral or injection treatment. Some such treatments include antibiotics, antifungal agents, antiviral agents, corticosteroids, immunosuppressants, biologics, enzyme inhibitors, or retinoids. Some antibiotics include dicloxacillin, erythromycin, or tetracycline. Oral antifungal drugs may include fluconazole, itraconazole, or terbinafine. Antiviral agents may include acyclovir (Zovirax), famciclovir (Famvir), or valacyclovir (Valtrex). Corticosteroids may include prednisone. Immunosuppressants may include azathioprine (Imuran) or methotrexate (Trexall). Biologics may include adalimumab (Humira), adalimumab-atto (Amjevita), etanercept (Enbrel), etanercept-szzs (Erelzi), infliximab (Remicade), ixekizumab (Taltz), secukinumab (Cosentyx), brodalumab (Siliq), ustekinumab (Stelara), guselkumab (Tremfya), risankizumab (Skyrizi), or tildrakizumab (Ilumya). Enzyme inhibitors may include apremilast (Otezla) or eucrisa (e.g. provided in an ointment). Retinoids may include acitretin (Soriatane).

In some embodiments, the treatment includes administration of a nutraceutical. The nutraceutical may include a bioactive peptide, oligosaccharide, plant polyphenol, carotenoid, vitamin, or polyunsaturated fatty acid. Examples of nutraceuticals include melatonin, lysine, dehydroepiandrosterone, chondroitin, glucosamine, s-adenosylmethionine, omega-3 polyunsaturated fatty acids, alpha-lipoic acid systemic, ubiquinone systemic, tryptophan, lecithin, chondroitin, glucosamine, methylsulfonylmethane, methylsulfonylmethane, red yeast rice systemic, glucosamine systemic, creatine systemic, glutamine systemic, levocarnitine systemic, methionine, lutein, inositol, chondroitin, or betaine.

The treatment may include a sunburn treatment. Some sunburn treatments may include administration of an aloe, acetaminophen, ibuprofen, vinegar, baking soda, cornstarch, oatmeal, coconut oil, tea, witch hazel, ice, cool water, anti-pain medication, anti-itch medication, a corticosteroid cream, a moisturizer, or an essential oil such as lavender or helichrysum.

The treatment may include a cosmeceutical. Cosmeceuticals may include sunscreens which affect photo-aging, antioxidants, hydroxy acids, retinoids (vitamin A), skin lightening agents, botanicals, peptides, proteins, or growth factors. Examples of antioxidants may include alpha-lipoic acid, vitamin C (L-ascorbic acid), nicotinamide (vitamin B3), vitamin E (alpha tocopherol), N-acetyl-glucosamine (NAG), or ubiquinone (CoQ10). Hydroxy acids may include alpha hydroxy acids (AHAs), poly hydroxy acids (PHAs), or beta hydroxy acids (BHAs). AHAs may include glycolic acid, lactic acid, citric acid, mandelic acid, malic acid, tartaric acid, or lactobionic acid. PHAs may include gluconolactone or lactobionic acid. BHA may include salicylic. Skin lightening agents may include hydroquinone, ascorbic acid (vitamin C), kojic acid, azelaic acid, or licorice extract (e.g. glabridin). Botanicals may include plant extracts from leaves, roots, fruits, berries, stems, bark or flowers. Botanicals may include antioxidant, anti-inflammatory and/or skin soothing properties. Examples of botanicals may include soy, curcumin, silymarin, pycnogenol, Ginkgo biloba, green tea extract, grape seed extract, aloe vera, witch hazel, allantoin or ferulic acid. Peptides or protein treatments may include the pentapeptide Pal-KTTKS.

In some embodiments, the treatment includes a topical targeted therapy. For example, the treatment may include administration of a small-molecule kinase inhibitors such as dasatinib or BEZ-235. In some embodiments, the treatment includes administration of 5-fluorouracil.

In some embodiments, the treatment includes one or more vitamins such as B vitamins. Examples may include thiamin (vitamin B1), riboflavin (vitamin B2), niacin (vitamin B3), pantothenic acid, vitamin B6, biotin (vitamin B7), folate, or vitamin B12.

In some embodiments, the treatment improves the subject's skin. For example, the treatment may reduce wrinkliness, dryness, scaliness, flakiness, redness, or soreness. The treatment may reduce a mutation burden in the subject. The improvement or reduction may be in relation to a baseline measurement.

Some embodiments of the methods described herein include obtaining the measurement from a subject. For example, the measurement may be obtained from the subject after treating the subject. In some embodiments, the measurement is obtained in a second sample (such as a skin) obtained from the subject after the treatment is administered to the subject. In some embodiments, the measurement indicates that the mutation burden or an epigenetic profile has been improved.

In some embodiments, the measurement is obtained directly from the subject. In some embodiments, the measurement is obtained in a second sample from the subject. In some embodiments, the measurement is obtained by performing an assay on the second sample obtained from the subject. In some embodiments, the measurement is obtained by an assay, such as an immunoassay, a colorimetric assay, a fluorescence assay, a chromatography (e.g. HPLC) assay, a PCR assay. The measurement may include DNA sequencing such as next generation sequencing.

In some embodiments, the measurement is obtained within 1 hour, within 2 hours, within 3 hours, within 4 hours, within 5 hours, within 6 hours, within 12 hours, within 18 hours, or within 24 hours after the administration of the treatment. In some embodiments, the measurement is obtained within 1 day, within 2 days, within 3 days, within 4 days, within 5 days, within 6 days, or within 7 days after the administration of the treatment. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the treatment. In some embodiments, the measurement is obtained after 1 hour, after 2 hours, after 3 hours, after 4 hours, after 5 hours, after 6 hours, after 12 hours, after 18 hours, or after 24 hours after the administration of the treatment. In some embodiments, the measurement is obtained after 1 day, after 2 days, after 3 days, after 4 days, after 5 days, after 6 days, or after 7 days after the administration of the treatment. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the treatment.

In some embodiments, the treatment reduces a gene burden measurement relative to a baseline gene burden measurement. In some embodiments, the gene burden measurement is decreased by about 2.5% or more, about 5% or more, or about 7.5% or more, relative to the baseline measurement. In some embodiments, the measurement is decreased by about 10% or more, relative to the baseline measurement. In some embodiments, the gene burden measurement is decreased by about 20% or more, about 30% or more, about 40% or more, about 50% or more, about 60% or more, about 70% or more, about 80% or more, about 90% or more, relative to the baseline measurement. In some embodiments, the gene burden measurement is decreased by no more than about 2.5%, no more than about 5%, or no more than about 7.5%, relative to the baseline measurement. In some embodiments, the gene burden measurement is decreased by no more than about 10%, relative to the baseline measurement. In some embodiments, the gene burden measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the gene burden measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the subject is monitored. For example, the subject may be assessed (e.g. for mutation burden in one or more skin areas) periodically. The monitoring may take place every week, 2 weeks, 3 weeks, 1 month, 2 months, 3 months, 6 months, 1 year, 2 years, 3 years, 4 years, or 5 years. In some cases, the subject is monitored every 21-28 days. A usefulness of monitoring every 21-28 days is that new skin cells may be present at that time because skin cells may turn over every 21-28 days. Therefore, a mutation burden may be changed within that time. The monitoring may be based on which treatment is provided to the subject.

Some subjects may be high-risk, such as subjects exposed to a higher amount of mutagens (e.g. UV light, carcinogens, or radioactivity) than an average or typical subject, or immunocompromised subjects. In some embodiments, a high-risk subject is monitored continuously or more often than an average or typical subject. For example, a high-risk subject may be monitored every day, 2 days, 3 days, 4 days, 5 days, 6 days, week, or 2 weeks,

### Subjects

Some aspects relate to a subject. For example, some aspects include quantifying a mutation burden in a subject. Examples of subjects include vertebrates, animals, mammals, dogs, cats, cattle, rodents, mice, rats, primates, monkeys, or humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human. The subject may be male or female.

In some embodiments, the subject is an adult (e.g. at least 18 years old). In some embodiments, the subject is ≥90 years of age. In some embodiments, the subject is ≥85 years of age. In some embodiments, the subject is ≥80 years of age. In some embodiments, the subject is >70 years of age. In some embodiments, the subject is ≥60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is ≥0 years of age.

In some embodiments, the subject is ≤100 years of age. In some embodiments, the subject is ≤90 years of age. In some embodiments, the subject is ≤85 years of age. In some embodiments, the subject is ≤80 years of age. In some embodiments, the subject is ≤70 years of age. In some embodiments, the subject is ≤60 years of age. In some embodiments, the subject is ≤50 years of age. In some embodiments, the subject is ≤40 years of age. In some embodiments, the subject is ≤30 years of age. In some embodiments, the subject is ≤20 years of age. In some embodiments, the subject is ≤10 years of age. In some embodiments, the subject is ≤1 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

In some embodiments, the subject may be immunocompromised. In some embodiments, the subject is a transplant patient. In some embodiments, the subject has an immune system disorder. For example, a transplant patient may be more susceptible to mutations than a non-transplant patient. The subject may be immunocompromised. The subject may suffer from a skin condition such as psoriasis, dermatitis, actinic keratosis. The skin condition may include a skin cancer. The skin cancer may include melanoma, basal cell carcinoma (BCC), or squamous cell carcinoma (SCC).

### Computer Systems

The present disclosure provides computer systems for implementing methods and devices of the present disclosure. FIG. 8 shows a computer system 1501 that is programmed or otherwise configured to operate any method or system described herein (such as any method of cutting a sample collector described herein). The computer system 1501 can regulate various aspects of the present disclosure. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine-readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, enact any of the methods for imparting color to a wearable ocular device as described herein.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL” Generally, the term “about” includes an amount that would be expected to be within experimental error. In some instances, “about” defines a range (inclusive) around the value of +/−10%.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “mutation” refers to a substitution, deletion, insertion, or relative to a reference sequence. In some instances, a mutation occurs in a nucleic acid or a peptide. In some instances, the reference sequence is a control sequence which has been exposed to minimal or no environmental factors which care capable of inducing mutations. In some instances, a reference sequence is obtained from an age-adjusted population of subjects.

### Numbered Embodiments

Provided herein are numbered embodiments 1-99. 1. A method for quantifying a mutation burden in a subject, comprising: a) obtaining a sample from the subject by non-invasive sampling, wherein the sample comprises a one or more of skin cells; b) detecting at least one nucleic acid mutation in the sample; and c) quantifying the mutation burden based on presence, quantity, or absence of the at least one nucleic acid mutation. 2. The method of claim 1, wherein the non-invasive sampling comprises use of an adhesive tape. 3. The method of claim 1 or 2, wherein the sample comprises fewer than 1 gram of cellular material collected. 4. The method of claim 1 or 2, wherein the sample comprises 1 picogram-1 gram of cellular material collected. 5. The method of any one of claims 1-4, wherein the sample comprises no more than 20 milligrams of cellular material collected. 6. The method of any one of claim 1 claims 4, wherein the sample comprises 1 picogram to 20 milligrams of cellular material collected. 7. The method of claim 1 any one of claims 1-4, wherein the sample comprises 1 picogram-500 micrograms of cellular material collected. 8. The method of claim 1 any one of claims 1-4, wherein the sample comprises skin cells from no more than the superficial about 0.1 mm of skin. 9. The method of claim 1 any one of claims 1-4, wherein the sample comprises skin cells from the superficial 10-20 μm of skin. 10. The method of claim 1 any one of claims 1-4, wherein the sample comprises skin cells from fewer than about 100 cell layers. 11. The method of claim 1 any one of claims 1-4, wherein the sample comprises skin cells from 1 to 50 cell layers. 12. The method of claim 1 any one of claims 1-4, wherein the sample comprises cellular material collected using one or more adhesive tapes. 13. The method of claim 1 any one of claims 1-12, wherein the sample comprises skin cells from 1 to 5 cell layers. 14. The method of claim 1 any one of claims 1-7, wherein the sample comprises skin cells obtained no deeper than the stratum germinativum. 15. The method of claim 1 any one of claims 1-14, wherein the sample comprises skin cells obtained from a skin surface area of 10-300 mm2. 16. The method of claim 1 any one of claims 1-15, wherein the sample comprises a majority of skin sampled from a layer of skin exposed to an environmental factor. 17. The method of claim 16, wherein the environmental factor is ultraviolet (UV) light. 18. The method of claim 16, wherein the environmental factor is a chemical mutagen. 19. The method of claim 1 any one of claims 1-18, wherein the method further comprises detecting colonization of the one or more skin cells. 20. The method of claim 1 any one of claims 1-19, wherein the mutation burden comprises a ratio of the skin cells comprising the at least one nucleic acid mutation compared to a total number of cells in the sample. 21. The method of claim 1 any one of claims 1-19, wherein quantifying the mutation burden comprises detecting a copy number of at least 2 for the at least one nucleic acid mutation. 22. The method of any one of claims 16-21, wherein the sample obtained by the non-invasive sampling comprises an increased percentage of cells contacted with the environmental factor compared to a percentage of cells contacted with the environmental factor in a sample obtained by standard biopsy. 23. The method of any one of claims 16-21, wherein the method detects the at least one nucleic acid mutation in the sample obtained by the non-invasive sampling at an increased sensitivity compared to a sensitivity of detecting the at least one nucleic acid mutation in a sample obtained by standard biopsy. 24. The method of claim 22 or 23, wherein the number of nucleic acid mutations per mm2 of skin collected comprises at least 25 mutations. 25. The method of claim 22, wherein the method detects the at least one nucleic acid mutation in the sample obtained by the non-invasive sampling with a sensitivity of at least 3.0%. 26. The method of claim 22, wherein the method detects the at least one nucleic acid mutation in the sample obtained by the non-invasive sampling with a sensitivity of at least 1.0%. 27. The method of claim 1 any one of claims 1-26, wherein the quantifying the mutation burden comprises detecting a variant allele frequency comprising the at least one nucleic acid mutation. 28. The method of claim 1 any one of claims 1-27, wherein the method comprises detecting 5-5,000 nucleic acid mutations in the sample. 29. The method of claim 1 any one of claims 1-27, wherein the method comprises detecting 2-25 nucleic acid mutations in the sample. 30. The method of claim 1 any one of claims 1-27, wherein the method comprises detecting at least 5 nucleic acid mutations in the sample. 31. The method of claim 1 any one of claims 1-27, wherein the method comprises detecting at least 10 nucleic acid mutations in the sample. 32. The method of claim 1 any one of claims 1-27, wherein the at least one mutation is present in at least 1% of the cells in the sample. 33. The method of claim 1 any one of claims 1-27, wherein the at least one mutation is present in at least 5% of the cells in the sample. 34. The method of claim 1 any one of claims 1-27, wherein the at least one mutation is present in at least 10% of the cells in the sample. 35. The method of claim 1 any one of claims 1-31, wherein the at least one nucleic acid mutation is present in TP53, NOTCH1, NOTCH2, NOTCH3, RBM10, PPP2R1A, GNAS, CTNNB1, PIK3CA, PPP6C, HRAS, KRAS, MTOR, SMAD3, LMNA, FGFR3, ZNF750, EPAS1, RPL22, ALDH2, CBFA2T3, CCND1, FAT1, FH, KLF4, CIC, RAC1, PTCH1, or TPM4. 36. The method of claim 35, wherein the at least one nucleic acid mutation is present in TP53. 37. The method of claim 1 any one of claims 1-36, wherein the at least one nucleic acid mutation is a mutation induced by UV light. 38. The method of claim 37, wherein the mutation induced by UV light is a C>T mutation. 39. The method of claim 37, wherein the mutation induced by UV light is a G>A mutation. 40. The method of claim 1 any one of claims 1-39, wherein the sample comprises cells of p53 immunopositive patches (PIPs). 41. The method of claim 40, wherein the method comprises detecting the at least one nucleic acid mutation in the cells of PIPs. 42. The method of claim 1 any one of claims 1-31, wherein the at least one nucleic acid mutation is present in at least one nucleic acid mutation in a MAPK pathway gene. 43. The method of claim 42, wherein the gene of MAPK pathway comprises BRAF, CBL, MAP2K1, NF1, or RAS. 44. The method of claim 1 any one of claims 1-31, wherein quantifying the mutation burden comprises detecting the at least one nucleic acid mutation in a cell cycle regulator. 45. The method of claim 44, wherein the cell cycle regulator is CDKN2A. 46. The method of claim 44, wherein the cell cycle regulator is PPP6C. 47. The method of claim 1 any one of claims 1-31, wherein the at least one nucleic acid mutation is present in an RNA processing gene. 48. The method of claim 47, wherein the RNA processing gene is DDX3X. 49. The method of claim 1 any one of claims 1-31, wherein the at least one nucleic acid mutation in present in a PI3K pathway gene. 50. The method of any one of claims 49, wherein the PI3K pathway gene comprises XIAP, AKT1, TWIST1, BAD, CDKN1A, ABL1, CDH1, TP53, CASP3, PAK1, GAPDH, PIK3CA, FAS, AKT2, FRAP1, FOXO1A, PTK2, CASP9, PTEN, CCND1, NFKB1, GSK3B, MDM2, or CDKN1B. 51. The method of claim 1 any one of claims 1-31, wherein the at least one nucleic acid mutation is present in a chromatin remodeling gene. 52. The method of claim 51, wherein the chromatin remodeling gene is ARID2. 53. The method of claim 1 any one of claims 1-52, wherein the at least one nucleic acid mutation is a driver mutation. 54. The method of claim 1 any one of claims 1-52, wherein the at least one nucleic acid mutation is a passenger mutation. 55. The method of claim 1 any one of claims 1-52, wherein the at least one nucleic acid mutation is present in a transcription regulation region of a gene. 56. The method of claim 55, wherein the transcription regulation region of the gene comprises an enhancer, a silencer, an insulator, an operator, aa promoter, a 5′ untranslated region (5′ UTR), or a 3′ untranslated region (3′UTR). 57. The method of claim 55, wherein the transcription regulation region comprises the promoter. 58. The method of claim 1 any one of claims 1-94, wherein the non-invasive sampling is performed on skin from the subject's head. 59. The method of claim 58, wherein the non-invasive sampling is performed on skin from the subject's face. 60. The method of claim 1 any one of claims 1-59, wherein the one or more skin cells comprises melanocytes. 61. The method of claim 1 any one of claims 1-60, wherein the one or more skin cells comprise keratinocytes. 62. The method of claim 1 any one of claims 1-61, wherein the subject does not exhibit symptoms of cancer. 63. The method of claim 62, wherein the cancer is skin cancer. 64. The method of claim 1 any one claims 1-63, wherein the method further comprises comparing the mutation burden with a reference comprising nucleic acid sequence data obtained from a non-cancerous skin sample. 65. The method of claim 1 any one claims 1-63, wherein the method further comprises comparing the mutation burden with a reference comprising nucleic acid sequence data obtained from a skin sample not exposed to UV light. 66. The method of claim 1 any one of claims 1-65, wherein the method further comprises calculating a quantitative burden based on the mutation burden. 67. The method of claim 66, wherein the method further comprises providing to the subject a report or a recommendation based on the quantitative burden of the subject. 68. A method of reducing skin cancer risk comprising: a) calculating a quantitative burden based on the mutation burden of claim 1 any one of claims 1-67; and b) providing a treatment recommendation based on the quantitative burden. 69. The method of claim 68, wherein the quantitative burden is categorized as low, medium, or high. 70. The method of claim 68 or 69, wherein calculating the quantitative burden comprises use of machine learning. 71. The method of claim 68 any one of claims 68-70, wherein calculating the quantitative burden comprises weighting each mutation of the mutation burden. 72. The method of claim 68 any one of claims 68-71, wherein calculating the quantitative burden comprises correlating each mutation of the mutation burden with skin cancer risk. 73. The method of claim 68 any one of claim 68 or 72, wherein the treatment recommendation comprises use of sun protection sunscreens, supplements, or photolyase treatment. 74. The method of claim 68 any one of claim 68 or 72, wherein the treatment recommendation comprises use retinoids, light peel, or photodynamic therapy (PDT). 75. The method of claim 68 any one of claim 68 or 72, wherein the treatment recommendation comprises moderate or deep peel. 76. A system configured to perform the method of any one of claims 1-67, said system comprising: a) an apparatus for performing non-invasive skin sample collection; b) a nucleic acid sequencing platform; and c) an assay for detecting the at least one nucleic acid mutation. 77. The system of claim 76, wherein the system detects 5-25 nucleic acid mutations. 78. The system of claim 76 or 77, wherein the system detects the at least one nucleic acid mutation with a sensitivity of at least 5%. 79. The system of claim 76 or 77, wherein the system detects the at least one nucleic acid mutation with a sensitivity of at least 1.0%. 80. The system of claim 76 any one of claims 76-79, wherein the system is configured to detect the a least one nucleic acid mutation by qPCR. 81. The system of claim 76 any one of claims 76-79, wherein the system is configured to detect the a least one nucleic acid mutation by allele-specific qPCR. 82. The system of claim 81, wherein the allele-specific qPCR comprises amplification of an allele comprising the at least one nucleic acid mutation. 83. The system of claim 76 any one of claims 76-79, wherein the system is configured to detect the at least one nucleic acid mutation by MALDI-TOF mass spectrometry, sequencing by synthesis, nanopore sequencing, ddPCR, sanger sequencing, or real-time PCR. 84. The system of claim 83, wherein the system is configured to detect the at least one nucleic acid mutation by MALDI-TOF mass spectrometry. 85. The system of claim 76 any one of claims 76-84, wherein the system is configured to detect two or more nucleic acid mutations. 86. The system of claim 85, wherein the system is configured to detect at least 5 nucleic acid mutations. 87. The system of claim 85, wherein the system is configured to detect at least 10 nucleic acid mutations. 88. The system of claim 85, wherein the system is configured to detect at least 40 nucleic acid mutations. 89. The system of claim 85, wherein the system is configured to detect 5-5000 nucleic acid mutations. 90. The system of claim 76 any one of claims 76-89, wherein the system is configured to detect nucleic acid mutations in at least one of TP53, NOTCH1, NOTCH2, CDKN2A, HRAS, or MTOR. 91. A method for quantifying a epigenetic burden in a subject, comprising: a) obtaining a sample from the subject by non-invasive sampling, wherein the sample comprises a one or more skin cells; b) detecting at least epigenetic modification in the sample; and c) quantifying the epigenetic burden based on presence, quantity, or absence of the at least one epigenetic modification. 92. The method of claim 91, wherein the at least one epigenetic modification comprises methylation in a CpG island of a gene or a transcription regulation region of the gene. 93. The method of claim 91 or 92, wherein the at least one epigenetic modification comprises 5-methylcytosine. 94. The method of claim 92, wherein the gene is KRT1, KRT5, KRT6, KRT14, KRT15, KRT16, KRT17, or KRT80. 95. The method of claim 91 any one of claims 91-93, wherein the at least one epigenetic modification comprises N6-methyladenine. 96. A method for quantifying a mutation burden in a subject, comprising: quantifying the mutation burden based on the presence, quantity, or absence of at least one nucleic acid mutation in a sample, wherein the sample comprises one or more of skin cells obtained from the subject by non-invasive sampling. 97. The method of claim 96, further comprising treating the subject. 98. The method of claim 97, wherein treating the subject comprises application or recommendation of sun protection sunscreens, supplements, retinoids, photolyase treatment, photodynamic therapy (PDT), or a skin peal. 99. The method of claim 97, wherein treating the subject comprises generation of report.

These examples are provided for illustrative purposes only and not to limit the scope of the claims provided herein.

### Example 1

Skin samples (N=36) were obtained by non-invasive technique from areas of the face and body (low UV exposure negative control). A mutation panel comprising markers from Table 3 were selected for analysis of mutation burden in the skin samples.

TABLE 3

| No. | Gene | Amino Acid Mutation | CDS_change |
|---|---|---|---|
| 1 | TP53 | G245S | c.733G>A |
| 2 | TP53 | R280K | c.839G>A |
| 3 | TP53 | R248L | c.743G>T |
| 4 | TP53 | G266R | c.796G>A |
| 5 | TP53 | P250L | c.749C>T |
| 6 | TP53 | C238F | c.713G>T |
| 7 | NOTCH1 | E455K | c.1363G>A |
| 8 | NOTCH1 | P391S | c/1171C>T |
| 9 | NOTCH1 | C467F | c.1400G>T |
| 10 | NOTCH1 | P460S | c.1378C>T |
| 11 | NOTCH1 | C467Y | c.1400G>T |
| 12 | NOTCH1 | G427D | c.1280G>A |
| 13 | NOTCH1 | D352N | c.1054G>A |
| 14 | NOTCH1 | S137L | c.410C>T |
| 15 | NOTCH1 | P391L | c.1172C>T |
| 16 | NOTCH1 | S385 | c.1154C>T |
| 17 | NOTCH1 | P460L | c.1379C>T |
| 18 | NOTCH1 | E1453* | c.4357G>T |
| 19 | TP53 | R248Q | c.743G>A |
| 20 | TP53 | R248W | c.742C>T |
| 21 | TP53 | R282W | c.844C>T |
| 22 | TP53 | R196* | c.586G>A |
| 23 | TP53 | R286K | c.856C>T |
| 24 | TP53 | P278S | c.832C>T |
| 25 | NOTCH1 | R365C | c.1093C>T |
| 26 | NOTCH1 | E450K | c.1348G>A |
| 27 | NOTCH1 | E424K | c.1270G>A |
| 28 | NOTCH1 | R353C | c.1057C>T |
| 29 | MTOR | S2215F | c.6644C>T |
| 30 | TP53 | P278L | c.833C>T |
| 31 | TP53 | R248W | c.741_742de1insTT |
| 32 | CDKN2A | R58* | c.172C>T |
| 33 | CDKN2A | P144L | c.341C>T |
| 34 | CDKN2A | R80* | c.283C>T |
| 35 | CDKN2A | W110* | c.330G>A |
| 36 | CDKN2A | P81L | c.242C>T |
| 37 | CDKN2A | Q50* | c.148C>T |
| 38 | CDKN2A | R58* | c.171_172delinsTT |
| 39 | HRAS | G12D | c.35G>A |
| 40 | HRAS | Q61L | c.182A>T |
| 41 | HRAS | G13D | c.38G>A |
| 42 | NOTCH1 | A465T | c.1393G>A |
| 43 | NOTCH1 | n/a | c.4015-1G>A |
| 44 | NOTCH2 | R113* | c.337C>T |

*indicates a change to a non-sense mutation.

Samples were obtained by applying an adhesive-coated path to a subjects skin to obtain skin skills. Each sample was processed by genomic DNA isolation, amplification of marker regions, removal of phosphorylated nucleotides, primer extension, and analysis on a MassARRAY instrument (Agena Biosicence) to identify and quantify mutations.

Variant allele frequencies (VAF) were calculated to quantify the mutation burden, as shown for select mutations and samples in Table 4

TABLE 4

| Sample | MutName | VAF | Cl | Zscore | No. Mut | sum VAF | VAF*N |
|---|---|---|---|---|---|---|---|
| 1 | CDKN2A: pP81L | 11.13 | 0 | 87.01 | 2 | 14.02 | 28.04 |
| 1 | CDKN2A: pQ50Stop | 2.89 | 0 | 9.64 | | | |
| 2 | NOTCH1: pR353C | 1.23 | 0 | 11.42 | 2 | 2.01 | 4.02 |
| 2 | TP53: pR196Stop | 0.78 | 0 | 3 | | | |
| 6 | CDKN2A: pW110Stop | 1.36 | 0 | 4.01 | 1 | 1.36 | 1.36 |
| 7 | NOTCH1: pG427D | 0.77 | 0 | 5.76 | 1 | 0.77 | 0.77 |
| 8 | CDKN2A: pR80Stop | 3.78 | 0 | 7.42 | 7 | 21.48 | 150.36 |
| 8 | NOTCH1: pE1453Stop | 0.2 | 1.87 | 3.06 | | | |
| 8 | NOTCH1: pE455K | 5.94 | 0 | 22.24 | | | |
| 8 | NOTCH1: pP460L | 2.54 | 0 | 21.65 | | | |
| 8 | TP53: pE286K | 7.31 | 2.38 | 75.29 | | | |
| 8 | TP53: pR196Stop | 1.44 | 0 | 5.48 | | | |
| 8 | TP53: pR282W | 0.27 | 1.61 | 9.27 | | | |
| 11 | CDKN2A: pW110Stop | 17.76 | 0 | 47.99 | 2 | 20.13 | 40.26 |
| 11 | TP53: pR248W_2 | 3.37 | 1.71 | 6.3 | | | |
| 13 | CDKN2A: pW110Stop | 1.04 | 0 | 3.12 | 1 | 1.04 | 1.04 |
| 15 | CDKN2A: pR58Stop_2 | 1.78 | 0 | 13.67 | 11 | 35.99 | 395.89 |
| 15 | CDKN2A: pR80Stop | 5.89 | 0 | 11.34 | | | |
| 15 | CDKN2A: pW110Stop | 1.58 | 0 | 4.66 | | | |
| 15 | NOTCH1: pR365C | 0.65 | 0 | 4.54 | | | |
| 15 | NOTCH1: pR385F | 4.86 | 3.19 | 177.99 | | | |
| 15 | TP53: pE286K | 7.59 | 2.44 | 78.29 | | | |
| 15 | TP53: pP250L | 3.63 | 0 | 36.75 | | | |
| 15 | TP53: pP278S | 2.59 | 2.44 | 15.94 | | | |
| 15 | TP53: pR248Q | 2.55 | 1.78 | 36.52 | | | |
| 15 | TP53: pR248W_1 | 3.46 | 0 | 58.14 | | | |
| 15 | TP53: pR282W | 1.41 | 1.86 | 21.96 | | | |
| 16 | NOTCH1: pS137L | 0.69 | 0 | 8.97 | 1 | 0.69 | 0.69 |
| 17 | TP53: pP250L | 0.79 | 0 | 7.96 | 2 | 1.05 | 2.1 |
| 17 | TP53: pP278S | 0.26 | 1.87 | 4.45 | | | |
| 18 | CDKN2A: pP81L | 0.53 | 0 | 4.15 | 8 | 9.65 | 77.2 |
| 18 | CDKN2A: pR80Stop | 2.45 | 0 | 4.94 | | | |
| 18 | NOTCH1: pE1453Stop | 0.28 | 1.89 | 3.47 | | | |
| 18 | TP53: pG266R | 0.98 | 0 | 12.03 | | | |
| 18 | TP53: pP278L | 1.82 | 0 | 22.22 | | | |
| 18 | TP53: pP278S | 0.28 | 1.88 | 4.54 | | | |
| 18 | TP53: pR196Stop | 1.46 | 0 | 5.53 | | | |
| 18 | TP53: pR248W_1 | 1.85 | 0 | 31.19 | | | |

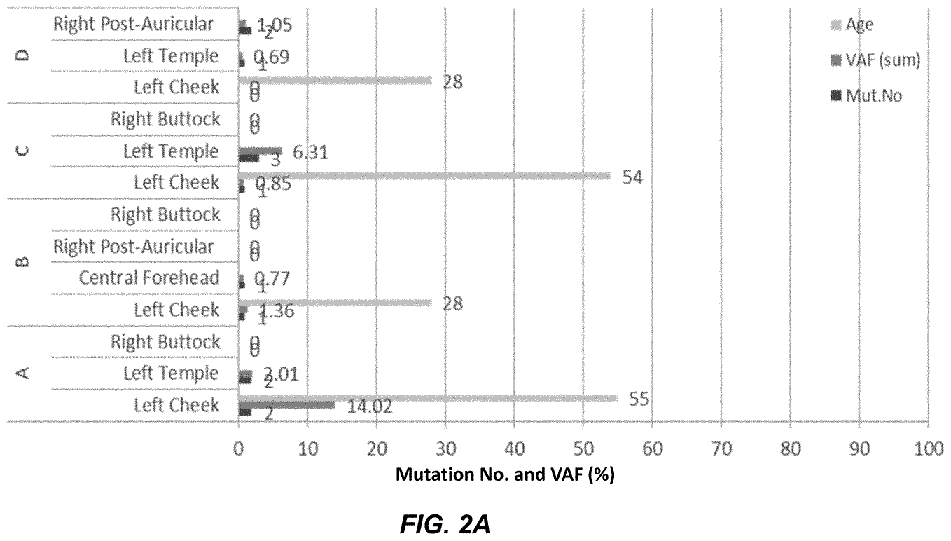

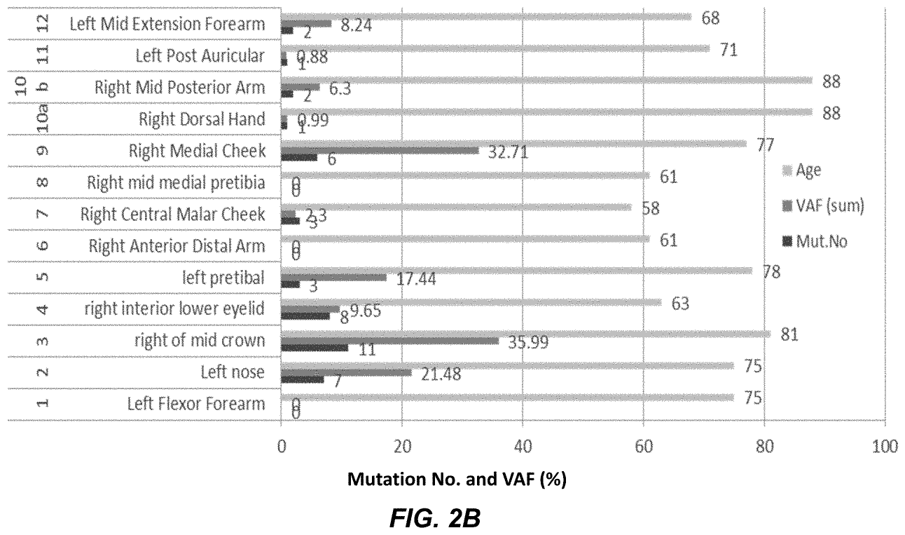

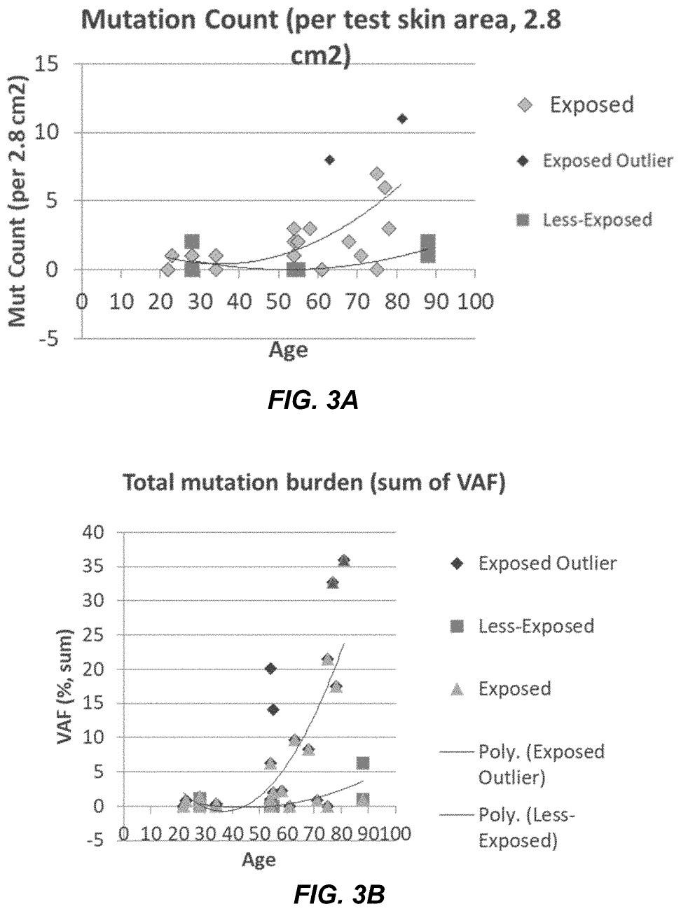

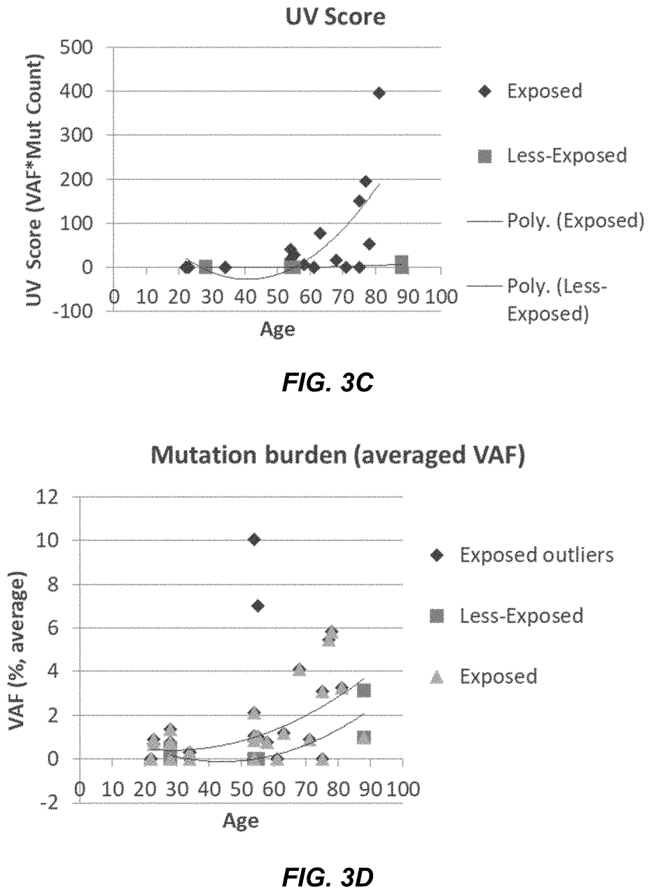

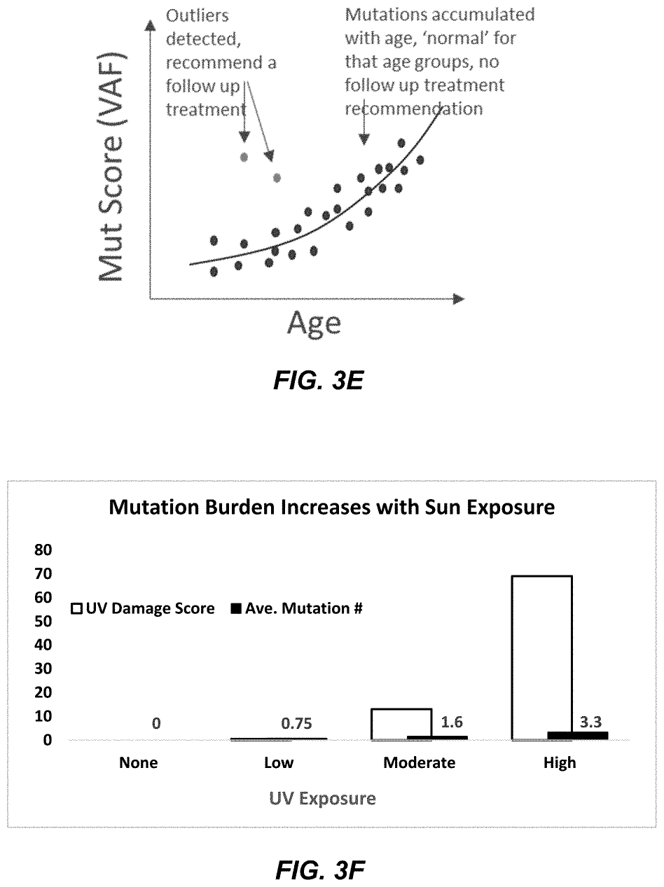

Mutations in sun exposed skins showing the subjects age, variant allele frequency, and mutation number are shown in FIGS. 1A-2B. The mutation count as function of skin test area and total mutation burden are shown in FIG. 3A-3B. A standard curve was generated to differentiate between samples having common mutations accumulated for a certain age and samples having excess mutations (FIG. 3A). Such samples may indicate a patient is at higher risk for future development of skin cancer, and treatment or intervention is required. Samples obtained from a subject's buttocks were used as a non or low-UV exposed control sample. In general, mutation burden increased with sun exposure (FIG. 3F).

### Example 2

Following the general procedures of Example 1, a 16 marker panel was used to quantify mutation burden (Table 5).

TABLE 5

| | | Amino Acid | | Positive |
|---|---|---|---|---|
| No. | Gene | Mutation | CDS_change | Count |
| 37 | CDKN2A | Q50* | c.148C>T | 7 |
| 36 | CDKN2A | P81L | c.242C>T | 3 |
| 35 | CDKN2A | W110* | c.330G>A | 10 |
| 16 | NOTCH1 | S385 | c.1154C>T | 3 |
| 8 | NOTCH1 | P391S | c/1171C>T | 4 |
| 15 | NOTCH1 | P391L | c.1172C>T | 4 |
| 26 | NOTCH1 | E450K | c.1348G>A | 1 |
| 7 | NOTCH1 | E455K | c.1363G>A | 1 |
| 44 | NOTCH2 | R113* | c.337C>T | 1 |
| 1 | TP53 | G245S | c.733G>A | 13 |
| 31 | TP53 | R248W | c.741_742delinsTT | 9 |
| 20 | TP53 | R248W | c.742C>T | 2 |
| 19 | TP53 | R248Q | c.743G>A | 2 |
| 5 | TP53 | P250L | c.749C>T | 1 |
| 4 | TP53 | G266R | c.796G>A | 2 |
| 24 | TP53 | P278S | c.832C>T | 3 |

*indicates a non-sense mutation.

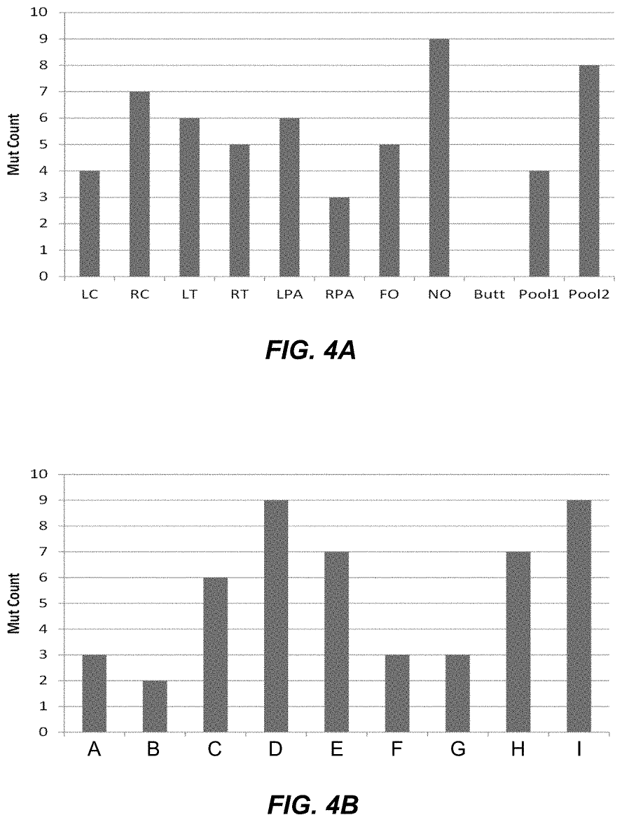

Mutation numbers for specific sample areas using a 16 target panel are shown in FIG. 4A-4B. Analysis of these mutations allowed stratification of sun-exposed skins with various levels of mutation burden.

### Example 3

One or more skin samples are obtained from a subject and the mutation burden of the skin samples is quantified using the general methods of Example 1 or 2. The mutation burden is then categorized as low, medium or high. If any of the samples comprise a higher mutation burden than predicted based on the subject's age, one or more intervention therapies is prescribed to the patient. For example, a patient categorized with low risk mutation burden is prescribed sun protective sunscreens, supplements such as nicotinamide, and/or photolyase. A patient categorized with medium risk mutation burden is treated with retinoids, light peel, and/or PDT. A patient categorized with high risk mutation is treated with medium or deep peel. Additionally, patients may be referred to a clinician based on the mutation burden for additional testing.

### Example 4

One or more skin samples are obtained from a subject and the mutation burden of the skin samples is quantified using the general methods of Example 1-3, with modification. Epigenetic methylation patterns are also quantified for one or more keratin-family genes, such as KRT5, KRT14, KRT15, and/or KRT80.

### Example 5

A non-invasive study was performed. Eighty-four human subjects were sampled for a study. Two stickers per site per subject were collected for total of eight facial sites per subject. The sites investigated were as follows—CF: Centre Forehead; RF: Right Forehead; LF: Left forehead; NO: Nose; RC: Right Cheek; LC: Left Cheek, RT: Right Temple, LT: Left Temple. Non-invasive skin samples were collected from all enrolled subjects using the DermTech adhesive skin collection kit (DermTech, Inc.; La Jolla, Calif.). The study was reviewed and approved by Aspire IRB (Santee, Calif.). All subjects provided written consent prior to enrollment.

Skin samples used in the study were obtained using the non-invasive adhesive skin collection kit (DermTech, Inc.; La Jolla, Calif.) as per the package Insert instructions. The genomic DNA extraction was performed using KingFisher Duo (ThermoFisher; Carlsbad, Calif.) with a bead-based extraction protocol. Post extraction, the genomic DNA was quantified using quantitative real-time polymerase chain reaction (qRT-PCR). The extracted genomic DNA was further processed for variant detection using an ultrasensitive and multiplexed MALDI-TOF mass spectrometry platform, MassARRAY™ (Agena Bioscience; San Diego, Calif.) and/or NextSeq 2000 following the user instructions.

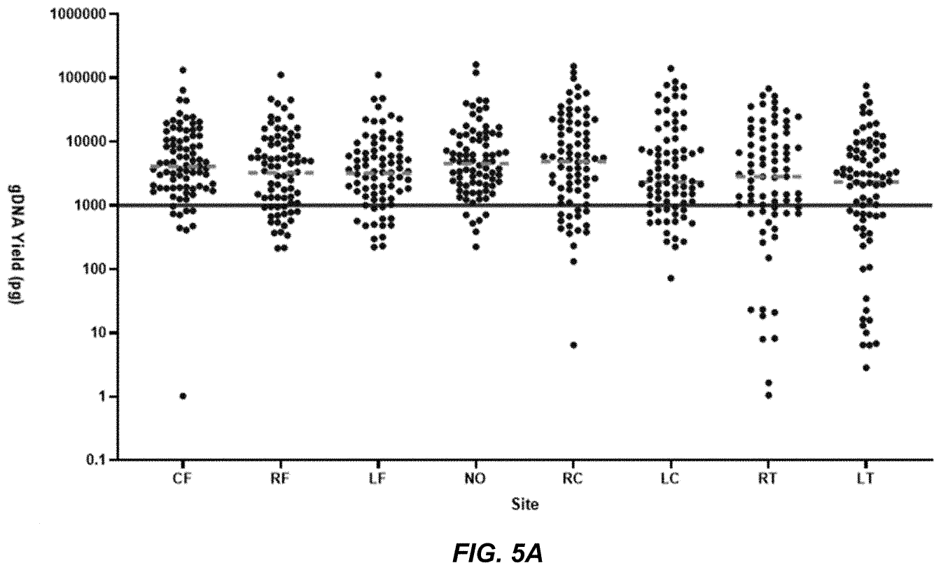

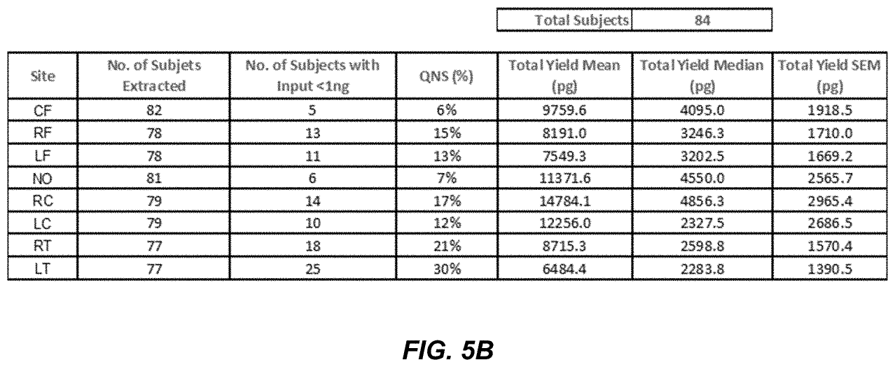

FIG. 5A shows a total genomic DNA (gDNA) comparison across all of the facial sites tested from the cohort of eighty-four subjects. Each dot represents a subject, the horizontal dotted line for each facial site represents median yield, and the solid horizontal line across the data set represents the minimum threshold of 1 ng gDNA that was considered sufficient for the test. The scale for the y-axis is log 10. Sample collection was done using two smart stickers per site per subject. The smart stickers included an adhesive patch with an adhesive matrix on one side of the patch. It may be assumed that 1 of said smart stickers may provide about half as much DNA as the amounts provided for two smart stickers. Quantification of the extracted gDNA was done by quantitative PCR (q-PCR).

FIG. 5B includes a comparison of total genomic DNA yield from each site tested with the percentage QNS (Quantity Not Sufficient). Sample collection was done using two smart stickers per site per subject. Quantification of the extracted gDNA was done by quantitative PCR (q-PCR). QNS % was calculated based on the number of subjects with less than 1 ng of genomic DNA Less than 1 ng of genomic DNA was considered insufficient minimum input in this study.

This study shows that sufficient genomic DNA was extracted from a variety of sample sites and subjects to perform the methods described herein that may include non-invasive sampling.

### Example 6

Table 5 provides counts of specific mutations detected in samples non-invasively collected from various human skin collection sites by tape stripping. Samples were non-invasively collected from eight facial sites from 45 subjects to assess the mutational burden from a panel of 25 UV damage and cancer related mutations. The sites investigated were—CF: Centre Forehead; RF: Right Forehead; LF: Left forehead; NO: Nose; RC: Right Cheek; LC: Left Cheek, RT: Right Temple, LT: Left Temple. The following values indicate the number of samples that had sufficient genomic yield for sequencing from each facial site: CF=42, LC=36, LF=39, LT=40, NO=41, RC=42, RF=42 and RT=42.

TABLE 5
Frequency of mutations at different facial sites

| Assay | RC | LC | RF | LF | RT | LT | CF | NO |
|---|---|---|---|---|---|---|---|---|
| CDKN2A_c.148C>T | 0 | 1 | 0 | 0 | 0 | 0 | 0 | 0 |
| CDKN2A_c.242C>T | 0 | 0 | 0 | 1 | 1 | 0 | 0 | 1 |
| NOTCH1_c.1057C>T | 7 | 9 | 11 | 3 | 9 | 13 | 13 | 13 |
| NOTCH1_c.1093C>T | 4 | 4 | 4 | 2 | 5 | 9 | 2 | 8 |
| NOTCH1_c.1154C>T | 25 | 18 | 16 | 21 | 14 | 18 | 14 | 19 |
| NOTCH1_c.1171C>T_ASO | 5 | 1 | 1 | 1 | 1 | 2 | 2 | 1 |
| NOTCH1_c.1172C>T | 2 | 1 | 0 | 1 | 3 | 3 | 1 | 2 |
| NOTCH1_c.1348G>A | 4 | 8 | 6 | 9 | 9 | 17 | 14 | 3 |
| NOTCH1_c.1363G>A | 4 | 3 | 2 | 6 | 3 | 6 | 4 | 4 |
| NOTCH1_c.1393G>A | 9 | 6 | 0 | 10 | 3 | 5 | 6 | 4 |
| NOTCH1_c.1400G>A | 4 | 5 | 6 | 6 | 2 | 4 | 4 | 4 |
| NOTCH1_c.4357G>T | 0 | 0 | 0 | 0 | 0 | 0 | 0 | 0 |
| NOTCH2_c.337C>T | 5 | 6 | 5 | 7 | 4 | 6 | 8 | 6 |
| TP53_c.586C>T | 4 | 1 | 2 | 1 | 2 | 2 | 6 | 6 |
| TP53_c.733G>A | 0 | 1 | 0 | 0 | 0 | 0 | 0 | 0 |
| TP53_c.741_742DELINSTT_ASO | 4 | 3 | 6 | 6 | 5 | 7 | 7 | 9 |
| TP53_c.742C>T_ASO | 37 | 29 | 31 | 35 | 38 | 40 | 41 | 34 |
| TP53_c.743G>A | 0 | 0 | 0 | 0 | 0 | 0 | 0 | 0 |
| TP53_c.749C>T | 8 | 9 | 1 | 8 | 8 | 10 | 20 | 11 |
| TP53_c.796G>A | 6 | 2 | 6 | 3 | 3 | 6 | 7 | 8 |
| TP53_c.832C>T | 1 | 1 | 1 | 6 | 1 | 3 | 3 | 3 |
| TP53_c.833C>T | 1 | 1 | 0 | 1 | 0 | 1 | 1 | 5 |
| TP53_c.839G>A | 0 | 0 | 0 | 0 | 0 | 2 | 0 | 0 |
| TP53_c.844C>T | 0 | 0 | 1 | 1 | 0 | 1 | 2 | 1 |
| TP53_c.856G>A | 1 | 0 | 2 | 0 | 2 | 1 | 2 | 0 |

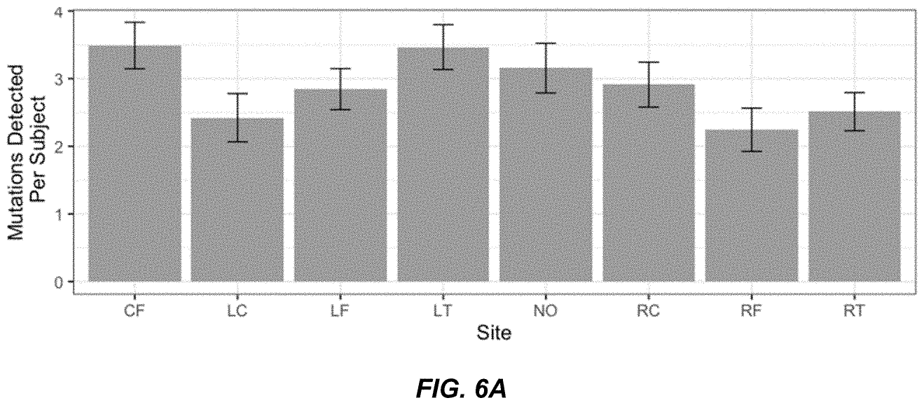

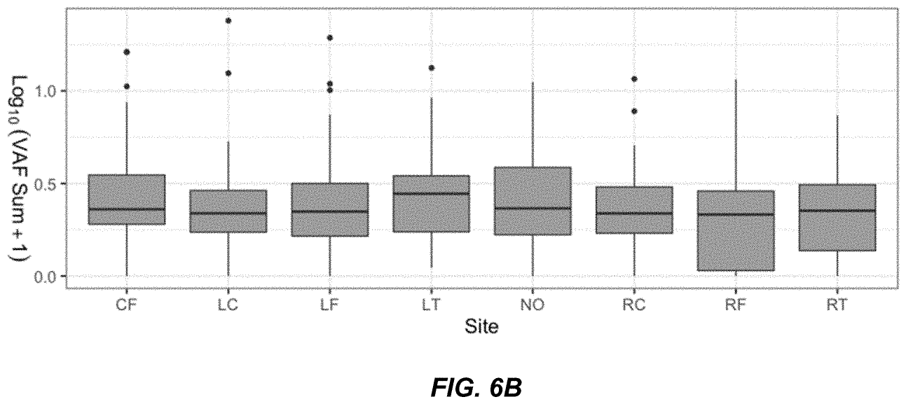

FIGS. 6A and 6B show the distribution and frequency of the mutations as detected on the human face, and includes data from the same samples and mutations as in Table 5.

Based on the data in this example, mutations may be detected at various skin sample collection sites. As is evident by the data, sufficient cellular genetic material may be obtained by non-invasive skin sampling to provide these data. The data also indicate that a mutation burden may be assessed, and that numbers of mutations may be assessed in samples collected from non-invasive skin sampling at various sites.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for quantifying a mutation burden in a subject, comprising:
a) obtaining a sample from the subject by non-invasive sampling, wherein the sample comprises a one or more of skin cells;
b) detecting at least one nucleic acid mutation in the sample; and
c) quantifying the mutation burden based on presence, quantity, or absence of the at least one nucleic acid mutation.

2-7. (canceled)

8. The method of claim 1, wherein the sample comprises skin cells from no more than the superficial about 0.1 mm of skin.

9-11. (canceled)

12. The method of claim 1, wherein the sample comprises cellular material collected using one or more adhesive tapes.

13-15. (canceled)

16. The method of claim 1, wherein the sample comprises a majority of skin sampled from a layer of skin exposed to an environmental factor.

17. The method of claim 16, wherein the environmental factor is ultraviolet (UV) light or chemical mutagen.

18-31. (canceled)

32. The method of claim 1, wherein the at least one mutation is present in at least 1% of the cells in the sample.

33-34. (canceled)

35. The method of claim 1, wherein the at least one nucleic acid mutation is present in TP53, NOTCH1, NOTCH2, NOTCH3, RBM10, PPP2R1A, GNAS, CTNNB1, PIK3CA, PPP6C, HRAS, KRAS, MTOR, SMAD3, LMNA, FGFR3, ZNF750, EPAS1, RPL22, ALDH2, CBFA2T3, CCND1, FAT1, FH, KLF4, CIC, RAC1, PTCH1, or TPM4.

36-67. (canceled)

68. The method of claim 1 for use in reducing skin cancer risk further comprising:
a) calculating a quantitative burden based on the mutation burden of claim 1; and
b) providing a treatment recommendation based on the quantitative burden.

69-72. (canceled)

73. The method of claim 68, wherein the treatment recommendation comprises use of sun protection sunscreens, supplements, or photolyase treatment.

74-75. (canceled)

76. A system configured to perform the method of claim 1, said system comprising:
a) an apparatus for performing non-invasive skin sample collection;
b) a nucleic acid sequencing platform; and
c) an assay for detecting the at least one nucleic acid mutation.

77. The system of claim 76, wherein the system detects 5-25 nucleic acid mutations.

78-82. (canceled)

83. The system of claim 76, wherein the system is configured to detect the at least one nucleic acid mutation by MALDI-TOF mass spectrometry, sequencing by synthesis, nanopore sequencing, ddPCR, sanger sequencing, or real-time PCR.

84. (canceled)

85. The system of claim 76, wherein the system is configured to detect two or more nucleic acid mutations.

86-89. (canceled)

90. The system of claim 76, wherein the system is configured to detect nucleic acid mutations in at least one of TP53, NOTCH1, NOTCH2, CDKN2A, HRAS, or MTOR.

91. A method for quantifying a epigenetic burden in a subject, comprising:
a) obtaining a sample from the subject by non-invasive sampling, wherein the sample comprises a one or more skin cells;
b) detecting at least epigenetic modification in the sample; and
c) quantifying the epigenetic burden based on presence, quantity, or absence of the at least one epigenetic modification.

92. The method of claim 91, wherein the at least one epigenetic modification comprises methylation in a CpG island of a gene or a transcription regulation region of the gene.

93. (canceled)

94. The method of claim 92, wherein the gene is KRT1, KRT5, KRT6, KRT14, KRT15, KRT16, KRT17, or KRT80.

95. (canceled)

96. A method for quantifying a mutation burden in a subject, comprising:
quantifying the mutation burden based on the presence, quantity, or absence of at least one nucleic acid mutation in a sample, wherein the sample comprises one or more of skin cells obtained from the subject by non-invasive sampling.

97. The method of claim 96, further comprising treating the subject.

98. The method of claim 97, wherein treating the subject comprises application or recommendation of sun protection sunscreens, supplements, retinoids, photolyase treatment, photodynamic therapy (PDT), or a skin peal.

99. (canceled)
